(19) United States
(12) Patent Application Publication
(10) Pub. No.: US 2008/0139558 A1
(43) Pub. Date: Jun. 12, 2008

(54) QUINOLONES USEFUL AS INDUCIBLE NITRIC OXIDE SYNTHASE INHIBITORS

(75) Inventors: Nicholas D. Smith, San Diego, CA (US); Jeffrey R. Roppe, Temecula, CA (US); Celine Bonnefous, San Diego, CA (US); Joseph E. Payne, Oceanside, CA (US); Hui Zhuang, San Diego, CA (US); Xiaohong Chen, San Diego, CA (US); Andrew K. Lindstrom, Encinitas, CA (US); Sergio G. Duron, San Diego, CA (US); Christian A. Hassig, Mira Mesa, CA (US); Stewart A. Noble, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignee: KALYPSYS, INC., San Diego, CA (US)

(21) Appl. No.: 11/678,572

(22) Filed: Feb. 23, 2007

Related U.S. Application Data
(60) Provisional application No. 60/776,561, filed on Feb. 24, 2006.
(60) Provisional application No. 60/848,696, filed on Oct. 2, 2006.

(51) Int. Cl. A61K 31/47; A61K 31/5377; A61P 11/06; A61P 17/04; A61P 17/06; A61P 19/02; A61P 19/10; A61P 25/14; A61P 29/02; A61P 3/10; A61P 31/00; A61P 31/12; A61P 31/22; A61P 37/00; A61P 37/08; A61P 9/10; C07D 215/227; C07D 295/00; C07D 471/04

(52) U.S. Cl. 514/235.8; 514/303; 514/312; 544/122; 546/118; 546/156

(57) ABSTRACT

The present invention relates to novel quinolones of Formula I that inhibit inducible NOS synthase together with methods of synthesizing and using the compounds including methods for inhibiting or modulating nitric oxide synthesis and/or lowering nitric oxide levels in a patient by administering the compounds for the treatment of disease.

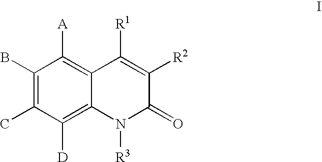

This application claims the benefit of priority of U.S. provisional application No. 60/776,561, filed Feb. 24, 2006 and U.S. provisional application No. 60/848,696, filed Oct. 2, 2006, the disclosures of which are hereby incorporated by reference as if written herein in their entireties.

### FIELD OF THE INVENTION

The present invention is directed to new quinolone compounds and compositions and their application as pharmaceuticals for the treatment of disease. Methods of inhibition of nitric oxide synthase activity in a human or animal subject are also provided for the treatment disease.

### BACKGROUND OF THE INVENTION

Nitric oxide (NO) is involved in the regulation of many physiological processes as well as the pathophysiology of a number of diseases. It is synthesized enzymatically from L-arginine in numerous tissues and cell types by three distinct isoforms of the enzyme NO synthase (NOS). Two of these isoforms, endothelial NOS (eNOS) and neuronal NOS (nNOS) are expressed in a constitutive manner and are calcium/calmodulin dependent. Endothelial NOS is expressed by endothelium and other cell types and is involved in cardiovascular homeostasis. Neuronal NOS is constitutively present in both the central and peripheral nervous system where NO acts a neurotransmitter. Under normal physiological conditions, these constitutive forms of NOS generate low, transient levels of NO in response to increases in intracellular calcium concentrations. These low levels of NO act to regulate blood pressure, platelet adhesion, gastrointestinal motility, bronchomotor tone and neurotransmission. In contrast, the third isoform of NOS, inducible NOS (iNOS), a virtually calcium independent enzyme, is absent in resting cells, but is rapidly expressed in virtually all nucleated mammalian cells in response to stimuli such as endotoxins and/or cytokines. The inducible isoform is neither stimulated by calcium nor blocked by calmodulin antagonists. It contains several tightly bound co-factors, including FMN, FAD and tetrahydrobiopterin. The inducible isoform of nitric oxide synthase (NOS₂ or iNOS) is expressed in virtually all nucleated mammalian cells following exposure to inflammatory cytokines or lipopolysaccharide.

The enzyme iNOS synthase is a homodimer composed of 130 kDa subunits. Each subunit comprises an oxygenase domain and a reductase domain. Importantly, dimerization of the iNOS synthase is required for enzyme activity. If the dimerization mechanism is disrupted, the production of nitric oxide via inducible NOS enzyme is inhibited.

The presence of iNOS in macrophages and lung epithelial cells is significant. Once present, iNOS synthesizes 100-1000 times more NO than the constitutive enzymes synthesize and does so for prolonged periods. This excessive production of NO and resulting NO-derived metabolites (e.g., peroxynitrite) elicit cellular toxicity and tissue damage which contribute to the pathophysiology of a number of diseases, disorders and conditions.

Nitric oxide generated by the inducible form of NOS has also been implicated in the pathogenesis of inflammatory diseases. In experimental animals, hypotension induced by lipopolysaccharide or tumor necrosis factor alpha can be reversed by NOS inhibitors. Conditions which lead to cytokine-induced hypotension include septic shock, hemodialysis and interleukin therapy in cancer patients. An iNOS inhibitor has been shown to be effective in treating cytokine-induced hypotension, inflammatory bowel disease, cerebral ischemia, osteoarthritis, asthma and neuropathies such as diabetic neuropathy and post-herpetic neuralgia.

In addition, nitric oxide localized in high amounts in inflamed tissues has been shown to induce pain locally and to enhance central as well as peripheral stimuli. Because nitric oxide produced by an inflammatory response is thought to be synthesized by iNOS, the inhibition of iNOS dimerization produces both prophylactic and remedial analgesia in patients.

Hence, in situations where the overproduction of nitric oxide is deleterious, it would be advantageous to find a specific inhibitor of iNOS to reduce the production of NO. However, given the important physiological roles played by the constitutive NOS isoforms, it is essential that the inhibition of iNOS has the least possible effect on the activity of eNOS and nNOS.

### SUMMARY OF THE INVENTION

Novel compounds and pharmaceutical compositions that inhibit inducible NOS synthase monomer have been found together with methods of synthesizing and using the compounds including methods for the treatment of iNOS-mediated diseases in a patient by administering the compounds.

The present invention discloses a class of compounds, useful in treating iNOS-mediated disorders and conditions, defined by structural Formula I:

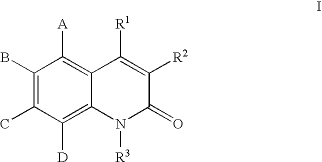

wherein:

R¹ is selected from the group consisting of acyl, alkyl, alkylene, aminoalkyl, amidoalkyl, alkynyl, amido, amino, aminoalkyl, aryl, arylalkyl, arylalkoxy, arylamino, arylaminoalkyl, arylthio, carboxy, cycloalkyl, ester, ether, halo, haloalkoxy, haloalkyl, heteroaryl, heteroarylalkyl, heteroarylamino, heteroarylaminoalkyl, heterocycloalkyl, heterocycloalkylalkyl, hydrazinyl, hydrogen, imino, thio, sulfonate, sulfonylamino and sulfonylaminoalkyl, any of which may be optionally substituted;

R² is selected from the group consisting of acyl, alkoxy, alkoxyalkyl, alkyl, alkylene, alkylamino, alkynyl, alkylimino, amido, amino, aryl, carboxy, cyano, cycloalkyl, ester, halo, haloalkyl, heteroaryl, heterocycloalkyl and hydrogen, any of which may be optionally substituted; or, alternatively, R² may combine with R¹ to form heterocycloalkyl, which may be optionally substituted;

R³ is selected from the group consisting of alkyl, amino, arylalkyl, aryl, cycloalkyl, haloalkyl, heteroarylalkyl, heterocycloalkyl and hydrogen, any of which may be optionally substituted; and

A, B, C and D are each independently selected from the group consisting of acyl, alkoxy, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted; or, alternatively, any two or more A, B, C and D may combine to form aryl, cycloalkyl, heteroaryl or heterocycloalkyl, any of which may be optionally substituted.

Compounds according to the present invention possess useful iNOS inhibiting activity, and may be used in the treatment or prophylaxis of a disease or condition in which iNOS plays an active role. Thus, in broad aspect, the present invention also provides pharmaceutical compositions comprising one or more compounds of the present invention together with a pharmaceutically acceptable carrier, as well as methods of making and using the compounds and compositions. In certain embodiments, the present invention provides methods for inhibiting iNOS. In other embodiments, the present invention provides methods for treating an iNOS-mediated disorder in a patient in need of such treatment comprising administering to said patient a therapeutically effective amount of a compound or composition according to the present invention. The present invention also contemplates the use of compounds disclosed herein for use in the manufacture of a medicament for the treatment of a disease or condition ameliorated by the inhibition of iNOS.

### DETAILED DESCRIPTION OF THE INVENTION

In certain embodiments, the compounds of the present invention have structural Formula II:

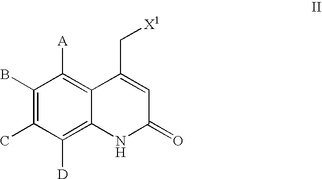

wherein:

X¹ is selected from the group consisting of CR⁴R⁵, N(R⁶)(R⁷), S(O)R⁸, S(O)₂R⁹ or OR¹⁰;

R⁴ and R⁵ are each independently selected from the group consisting of alkyl, amino, arylalkyl, aryl, cycloalkyl, haloalkyl, heteroarylalkyl, heterocycloalkyl and hydrogen, any of which may be optionally substituted;

R⁶ and R⁷ are each independently selected from the group consisting of acyl, alkyl, amino, aryl, cycloalkyl, haloalkyl, heteroaryl, heterocycloalkyl, hydrogen and sulfonyl, any of which may be optionally substituted; or, alternatively, R³ and R⁴ may combine to form heterocycloalkyl or heteroaryl, which may be optionally substituted;

R⁸ and R⁹ are each independently selected from the group consisting of alkyl, amino, arylalkyl, aryl, cycloalkyl, haloalkyl, heteroarylalkyl, heterocycloalkyl and hydrogen, any of which may be optionally substituted;

R¹⁰ is selected from the group consisting of alkyl, amino, arylalkyl, aryl, cycloalkyl, haloalkyl, heteroarylalkyl, heterocycloalkyl and hydrogen, any of which may be optionally substituted; and

A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

The invention further provides for compounds of Formula III:

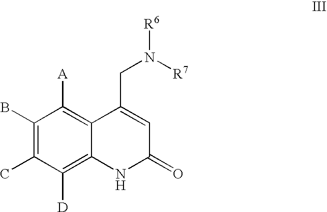

wherein:

R⁶ and R⁷ are each independently selected from the group consisting of acyl, alkyl alkylene, aminoalkyl, alkynyl, amido, amino, aryl, arylthio, carboxy, cycloalkyl, ester, ether, halo, haloalkoxy, haloalkyl, heteroaryl, heterocycloalkyl, hydrogen, thio and sulfonyl, any of which may be optionally substituted; or, alternatively, R¹ and R² may combine to form heterocycloalkyl or heteroaryl, which may be optionally substituted; and

A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

The invention further provides for compounds of Formula IV:

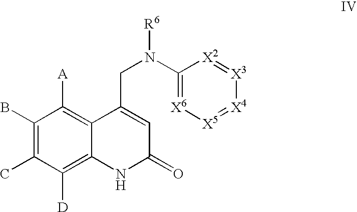

wherein:

X² is selected from the group consisting of CR¹² and N;

X³ is selected from the group consisting of CR¹³ and N;

X⁴ is selected from the group consisting of CR¹⁴ and N;

X⁵ is selected from the group consisting of CR¹⁵ and N;

X⁶ is selected from the group consisting of CR¹⁶ and N;

R¹² and R¹⁶ are each independently selected from the group consisting of alkoxy, acyl, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;

R¹³ and R¹⁵ are each independently selected from the group consisting of acyl, C²⁻⁶ alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;

R¹⁴ is selected from the group selected from the group consisting of C³⁻⁶ alkoxy, acyl, C²⁻⁶ alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;

R⁶ is selected from the group consisting of acyl, alkyl, alkylene, alkynyl, aminosulfonyl, arylthio, benzyl, carboxy, cycloalkyl, ester, ether, furanalkyl, furancarbonyl, haloalkyl, heteroaryl, heteroarylalkyl, aminoheteroaryl, heterocycloalkyl, imidazolecarbonyl, isoxazolecarbonyl, oxazolecarbonyl, pyrazinecarbonyl, thiophenecarbonyl, thiazolecarbonyl, thio and sulfonate, any of which may be optionally substituted; and

A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

The invention further provides for compounds of Formula V:

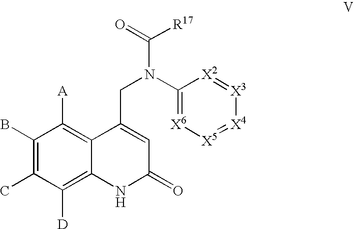

wherein:

X² is selected from the group consisting of CR¹² and N;

X³ is selected from the group consisting of CR¹³ and N;

X⁴ is selected from the group consisting of CR¹⁴ and N;

X⁵ is selected from the group consisting of CR¹⁵ and N;

X⁶ is selected from the group consisting of CR¹⁶ and N;

R¹² and R¹⁶ are each independently selected from the group consisting of alkoxy, acyl, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;

R¹³ and R¹⁵ are each independently selected from the group consisting of acyl, C²⁻⁶ alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;

R¹⁴ is selected from the group consisting of C³⁻⁶ alkoxy, acyl, C²⁻⁶ alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;

R¹⁷ is selected from the group consisting of alkyl, aryl, arylthio, cycloalkyl, heterocycloalkyl, benzimidazole, benzthiazole, benzofuran, benzothiophene, benzo[d][1,3]dioxole, 1H-benzo[d][1,2,3]triazole, 2,3-dihydrobenzofuran, 1,4-dioxane, 1,3-dioxalane, 3,4-dihydro-2H-benzo[b][1,4]dioxepine, 2,2-difluorobenzo[d][1,3]dioxole, isoxazole, isothiazole, indolizine, indole, isoindole, 3H-indoline, indoline, 1H-indazole, isoquinoline, imidazole, 2-imidazoline, imidazolidine, isothiazole, naphthalene, oxazole, 1,2,3-oxadiazole, morpholine, 2H-pyran, 4H-pyran, piperidine, pyridazine, pyrazine, piperazine, phenyl, pyridine, pyrimidine, thiophene, pyrrole, 2H-pyrrole, 2-pyrroline, 3-pyrroline, pyrrolidine, purine, thiazole, pyrazole, 2-pyrazoline, pyrazolidine, quinoline, quinazoline, quinaxaline, 1,2,3-triazole, 1,3,4-thiadiazole and 1,3,5-triazine, any of which may be optionally substituted; and

A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

In certain embodiments, compounds have Formula V wherein:

X² is CR¹²; X³ is CR¹³; X⁴ is CR¹⁴; X⁵ is CR¹⁵; X⁶ is CR¹⁶;

R¹²-R¹⁶ are each independently selected from the group consisting of halo, haloalkoxy, haloalkyl and hydrogen, any of which may be optionally substituted;

R¹⁷ is selected from the group consisting of cycloalkyl, heterocycloalkyl, isothiazole, imidazole, phenyl, pyridine, pyrazole and thiazole, which may be optionally substituted; and

A, B, C and D are each independently selected from the group consisting of halo and hydrogen.

The invention further provides for compounds of Formula VI:

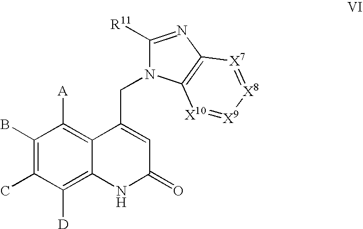

wherein:

X⁷ is selected from the group consisting of CR¹⁷ and N;

X⁸ is selected from the group consisting of CR¹⁸ and N;

X⁹ is selected from the group consisting of CR¹⁹ and N;

X¹⁰ is selected from the group consisting of CR²⁰ and N;

R¹¹ is selected from the group consisting of C₂-C₆ alkyl, aryl, arylthio, arylamino, cycloalkyl, heteroarylamino, heteroarylthio, heterocycloalkyl, benzimidazole, benzthiazole, benzofuran, benzothiophene, benzo[d][1,3]dioxole, 1H-benzo[d][1,2,3]triazole, 2,3-dihydrobenzofuran, 1,4-dioxane, 1,3-dioxalane, 3,4-dihydro-2H-benzo[b][1,4]dioxepine, 2,2-difluorobenzo[d][1,3]dioxole, furan, isoxazole, isothiazole, indolizine, indole, isoindole, 3H-indoline, indoline, 1H-indazole, isoquinoline, imidazole, 2-imidazoline, imidazolidine, isothiazole, naphthalene, oxazole, 1,2,3-oxadiazole, morpholine, 2H-pyran, 4H-pyran, piperidine, pyridazine, pyrazine, piperazine, phenyl, pyridine, pyrimidine, thiophene, pyrrole, 2H-pyrrole, 2-pyrroline, 3-pyrroline, pyrrolidine, purine, thiazole, pyrazole, 2-pyrazoline, pyrazolidine, quinoline, quinazoline, quinaxaline, 1,2,3-triazole, 1,3,4-thiadiazole and 1,3,5-triazine, any of which may be optionally substituted;

R¹⁷-R²⁰ are each independently selected from the group consisting of alkoxy, acyl, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted; and

A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

In certain embodiments, compounds have Formula VI wherein:

X⁷ is CR¹⁷; X⁸ is CR¹⁸; X⁹ is CR¹⁹; X¹⁰ is CR²⁰;

R¹¹ is selected from the group consisting of alkyl, cycloalkyl, heterocycloalkyl, isothiazole, imidazole, phenyl, pyridine, pyrazole and thiazole, which may be optionally substituted;

R¹⁷-R²⁰ are each independently selected from the group consisting of halo, haloalkoxy, haloalkyl and hydrogen, any of which may be optionally substituted; and

A, B, C and D are each independently selected from the group consisting of halo and hydrogen.

The invention further provides for compounds of Formula VII:

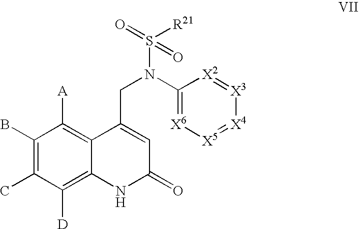

wherein:

X² is selected from the group consisting of CR¹² and N;

X³ is selected from the group consisting of CR¹³ and N;

X⁴ is selected from the group consisting of CR¹⁴ and N;

X⁵ is selected from the group consisting of CR¹⁵ and N;

X⁶ is selected from the group consisting of CR¹⁶ and N;

R¹²-R¹⁶ are each independently selected from the group consisting of alkoxy, acyl, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;

R²¹ is selected from the group consisting of alkyl, aryl, arylthio, arylamino, cycloalkyl, heteroarylamino, heteroarylthio, heterocycloalkyl, benzimidazole, benzthiazole, benzofuran, benzothiophene, benzo[d][1,3]dioxole, 1H-benzo[d][1,2,3]triazole, 2,3-dihydrobenzofuran, 1,4-dioxane, 1,3-dioxalane, 3,4-dihydro-2H-benzo[b][1,4]dioxepine, 2,2-difluorobenzo[d][1,3]dioxole, furan, isoxazole, isothiazole, indolizine, indole, isoindole, 3H-indoline, indoline, 1H-indazole, isoquinoline, imidazole, 2-imidazoline, imidazolidine, isothiazole, naphthalene, oxazole, 1,2,3-oxadiazole, morpholine, 2H-pyran, 4H-pyran, piperidine, pyridazine, pyrazine, piperazine, phenyl, pyridine, pyrimidine, thiophene, pyrrole, 2H-pyrrole, 2-pyrroline, 3-pyrroline, pyrrolidine, purine, thiazole, pyrazole, 2-pyrazoline, pyrazolidine, quinoline, quinazoline, quinaxaline, 1,2,3-triazole, 1,3,4-thiadiazole and 1,3,5-triazine, any of which may be optionally substituted; and

A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

In certain embodiments, compounds have Formula VII wherein:

X² is CR¹²; X³ is CR¹³; X⁴ is CR¹⁴; X⁵ is CR¹⁵; X⁶ is CR¹⁶;

R¹²-R¹⁶ are each independently selected from the group consisting of halo, haloalkoxy, haloalkyl and hydrogen, any of which may be optionally substituted;

R²¹ is selected from the group consisting of alkyl, cycloalkyl, heterocycloalkyl, isothiazole, imidazole, phenyl, pyridine, pyrazole and thiazole, which may be optionally substituted; and

A, B, C and D are each independently selected from the group consisting of halo and hydrogen.

The invention further provides for compounds of Formula VIII:

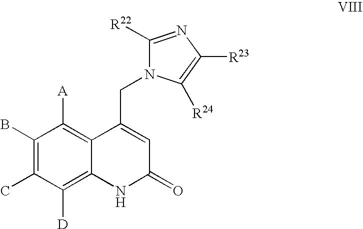

wherein:

R²²-R²⁴ are each independently selected from the group consisting of alkyl, aryl, arylthio, arylamino, cycloalkyl, heteroarylamino, heteroarylthio, heterocycloalkyl, benzimidazole, benzthiazole, benzofuran, benzothiophene, benzo[d][1,3]dioxole, 1H-benzo[d][1,2,3]triazole, 2,3-dihydrobenzofuran, 1,4-dioxane, 1,3-dioxalane, 3,4-dihydro-2H-benzo[b][1,4]dioxepine, 2,2-difluorobenzo[d][1,3]dioxole, furan, isoxazole, isothiazole, indolizine, indole, isoindole, 3H-indoline, indoline, 1H-indazole, isoquinoline, imidazole, 2-imidazoline, imidazolidine, isothiazole, naphthalene, oxazole, 1,2,3-oxadiazole, morpholine, 2H-pyran, 4H-pyran, piperidine, pyridazine, pyrazine, piperazine, phenyl, pyridine, pyrimidine, thiophene, pyrrole, 2H-pyrrole, 2-pyrroline, 3-pyrroline, pyrrolidine, purine, thiazole, pyrazole, 2-pyrazoline, pyrazolidine, quinoline, quinazoline, quinaxaline, 1,2,3-triazole, 1,3,4-thiadiazole and 1,3,5-triazine, any of which may be optionally substituted; and

A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

In certain embodiments, compounds have Formula VIII wherein:

R²²-R²⁴ are each independently selected from the group consisting of alkyl, cycloalkyl, heterocycloalkyl, isothiazole, imidazole, phenyl, pyridine, pyrazole and thiazole, which may be optionally substituted; and

A, B, C and D are each independently selected from the group consisting of halo and hydrogen.

The invention further provides for compounds of any one of Formulas IX or X:

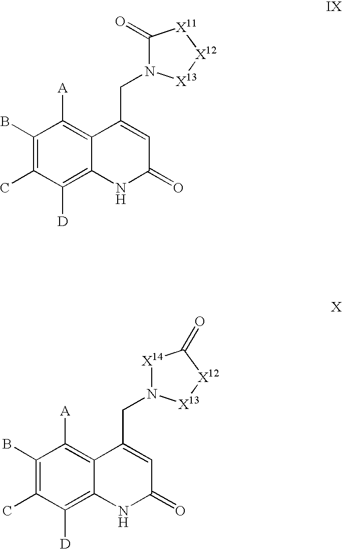

wherein:

X¹¹ is selected from the group consisting of CR²⁵ and N;

X¹² is selected from the group consisting of CR²⁶ and N;

X¹³ is selected from the group consisting of CR²⁷ and N;

X¹⁴ is selected from the group consisting of CR²⁸ and N;

R²⁵-R²⁸ are each independently selected from the group consisting of alkyl, aryl, arylthio, arylamino, cycloalkyl, heteroarylamino, heteroarylthio, heterocycloalkyl, benzimidazole, benzthiazole, benzofuran, benzothiophene, benzo[d][1,3]dioxole, 1H-benzo[d][1,2,3]triazole, 2,3-dihydrobenzofuran, 1,4-dioxane, 1,3-dioxalane, 3,4-dihydro-2H-benzo[b][1,4]dioxepine, 2,2-difluorobenzo[d][1,3]dioxole, furan, isoxazole, isothiazole, indolizine, indole, isoindole, 3H-indoline, indoline, 1H-indazole, isoquinoline, imidazole, 2-imidazoline, imidazolidine, isothiazole, naphthalene, oxazole, 1,2,3-oxadiazole, morpholine, 2H-pyran, 4H-pyran, piperidine, pyridazine, pyrazine, piperazine, phenyl, pyridine, pyrimidine, thiophene, pyrrole, 2H-pyrrole, 2-pyrroline, 3-pyrroline, pyrrolidine, purine, thiazole, pyrazole, 2-pyrazoline, pyrazolidine, quinoline, quinazoline, quinaxaline, 1,2,3-triazole, 1,3,4-thiadiazole and 1,3,5-triazine, any of which may be optionally substituted; and

A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

The invention further provides for compounds of any one of Formulas IX or X wherein:

R²⁵-R²⁸ are each independently selected from the group consisting of alkyl cycloalkyl, heterocycloalkyl, isothiazole, imidazole, phenyl, pyridine, pyrazole and thiazole, which may be optionally substituted; and

A, B, C and D are each independently selected from the group consisting of halo and hydrogen.

The invention further provides that compounds of Formula I may exist as tautomeric isomers including Formula XI.

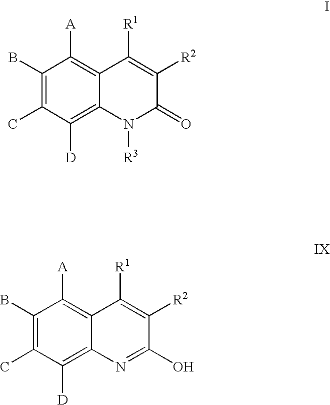

The invention provides for compounds of Formulas I-XI for use in the inhibition of iNOS for the treatment of disease.

The invention provides for compounds of Formulas I-XI administered in combination with another therapeutic agent.

The invention provides for compounds of Formulas I-XI for use as a medicament.

The invention provides for compounds of Formulas I-XI for use in the manufacture of a medicament for the prevention or treatment of a disease or condition ameliorated by the inhibition of iNOS.

The invention provides for a pharmaceutical composition comprising a compound of any of Formulas I-XI together with a pharmaceutically acceptable carrier, useful for the treatment or prevention of a iNOS-mediated disease.

The invention provides for a method of inhibition of iNOS comprising contacting iNOS with a compound of any of Formulas I-XI.

The invention provides for a method of treatment of a iNOS-mediated disease comprising the administration of a therapeutically effective amount of a compound of any of Formulas I-XI to a patient in need thereof, wherein said disease is selected from the group consisting of pruritis, psoriasis, uveitis, type 1 diabetes, diabetic nephropathy, septic shock, inflammatory pain, neuropathic pain, herpes zoster, postherpetic neuralgia, diabetic neuropathy, chronic low back pain, complex regional pain syndrome, fibromyalgia, migraine, rheumatoid arthritis, osteoarthritis, gouty arthritis, inflammatory bowel disease, asthma, COPD, allergic rhinitis, diabetic retinopathy, immune complex diseases, multiple sclerosis, alzheimer's disease, parkinson's disease, ischemic brain edema, toxic shock syndrome, heart failure, ulcerative colitis, atherosclerosis, glomerulonephritis, Paget's disease, osteoporosis, inflammatory sequelae of viral infections, retinitis, oxidant induced lung injury, restless leg syndrome, eczema, periodontal disease, gingivitis, acute allograft rejection and infection caused by invasive microorganisms which produce NO.

As used herein, the terms below have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH —,(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—)

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl(hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [C(O)H] and in combination is a C(O) group.

The term “carboxy,” as used herein, refers to C(O)OH or the corresponding “carboxylate” anion, such as is in a carboxylic acid salt. An “O-carboxy” group refers to a RC(O)O group, where R is as defined herein. A “C-carboxy” group refers to a —C(O)OR groups where R is as defined herein.

The term “cyano,” as used herein, alone or in combination, refers to CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃. The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groupsinclude carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N —O—.

The phrase “in the main chain” refers to the longest contiguous or adjacent chain of carbon atoms starting at the point of attachment of a group to the compounds of this invention.

The term “isocyanato” refers to a NCO group.

The term “isothiocyanato” refers to a NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—. The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an SH group.

The term “thiocarbonyl,” as used herein, when alone includes thioformyl C(S)H and in combination is a C(S) group.

The term “N-thiocarbamyl” refers to an ROC(S)NR′ group, with R and R′ as defined herein.

The term “O-thiocarbamyl” refers to a OC(S)NRR′, group with R and R′ as defined herein.

The term “thiocyanato” refers to a CNS group.

The term “trihalomethanesulfonamido” refers to a X₃CS(O)₂NR group with X is a halogen and R as defined herein.

The term “trihalomethanesulfonyl” refers to a X₃CS(O)₂ group where X is a halogen.

The term “trihalomethoxy” refers to a X₃CO group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers.

The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “inducible nitric oxide synthase inhibitor” or “iNOS inhibitor” is used herein to refer to a compound that exhibits an IC₅₀ with respect to iNOS of no more than about 100 μM and more typically not more than about 50 μM, as measured in the biological activity assay described generally hereinbelow. “IC₅₀” is that concentration of inhibitor which reduces the activity of an enzyme (e.g., iNOS) to half-maximal level. Representative compounds of the present invention have been discovered to exhibit inhibitory activity against iNOS. Compounds of the present invention preferably exhibit an IC₅₀ with respect to iNOS of no more than about 10 μM, more preferably, no more than about 5 μM, even more preferably not more than about 1 μM, and most preferably, not more than about 200 nM, as measured in the assays described herein. The phrase “therapeutically effective” is intended to qualify the amount of active ingredients used in the treatment of a disease or disorder. This amount will achieve the goal of reducing or eliminating the said disease or disorder.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefitrisk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, palmate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Gels for topical or transdermal administration of compounds of the subject invention may comprise, generally, a mixture of volatile solvents, nonvolatile solvents, and water. The volatile solvent component of the buffered solvent system may preferably include lower (C1-C6) alkyl alcohols, lower alkyl glycols and lower glycol polymers. More preferably, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. Preferably, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess will result in a lack of bioavailability due to poor release of drug from solvent mixture. The buffer component of the buffered solvent system may be selected from any buffer commonly used in the art; preferably, water is used. The preferred ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastimes comprising the active ingredient in a basis such as gelatin and glycerin or sucrose and acacia.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention with: a) corticosteroids including betamethasone dipropionate (augmented and nonaugemented), betamethasone valerate, clobetasol propionate, diflorasone diacetate, halobetasol propionate, amcinonide, dexosimethasone, fluocinolone acetononide, fluocinonide, halocinonide, clocortalone pivalate, dexosimetasone, and flurandrenalide; b) non-steroidal anti-inflammatory drugs including diclofenac, ketoprofen, and piroxicam; c) muscle relaxants and combinations thereof with other agents, including cyclobenzaprine, baclofen, cyclobenzaprine/lidocaine, baclofen/cyclobenzaprine, and cyclobenzaprine/lidocaine/ketoprofen; d) anaesthetics and combinations thereof with other agents, including lidocaine, lidocaine/deoxy-D-glucose (an antiviral), prilocalne, and EMLA Cream [Eutectic Mixture of Local Anesthetics (lidocaine 2.5% and prilocalne 2.5%; an emulsion in which the oil phase is a eutectic mixture of lidocaine and prilocalne in a ratio of 1:1 by weight. This eutectic mixture has a melting point below room temperature and therefore both local anesthetics exist as a liquid oil rather then as crystals)]; e) expectorants and combinations thereof with other agents, including guaifenesin and guaifenesin/ketoprofen/cyclobenzaprine; f) antidepressants including tricyclic antidepressants (e.g., amitryptiline, doxepin, desipramine, imipramine, amoxapine, clomipramine, nortriptyline, and protriptyline), selective serotonin/norepinephrine reuptake inhibitors including (e.g., duloxetine and mirtazepine), and selective norepinephrine reuptake inhibitors (e.g., nisoxetine, maprotiline, and reboxetine), selective serotonin reuptake inhibitors (e.g., fluoxetine and fluvoxamine); g) anticonvulsants and combinations thereof, including gabapentin, carbamazepine, felbamate, lamotrigine, topiramate, tiagabine, oxcarbazepine, carbamezipine, zonisamide, mexiletine, gabapentin/clonidine, gabapentin/carbamazepine, and carbamazepine/cyclobenzaprine; h) antihypertensives including clonidine; i) opioids including loperamide, tramadol, morphine, fentanyl, oxycodone, levorphanol, and butorphanol; j) topical counter-irritants including menthol, oil of wintergreen, camphor, eucalyptus oil and turpentine oil; k) topical cannabinoids including selective and non-selective CB1/CB2 ligands; and other agents, such as capsaicin.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating iNOS-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of iNOS-mediated disorders.

Compounds of the subject invention are useful in treating nitric oxide synthase-mediated disease, disorders and conditions, and are particularly suitable as inhibitors of nitric oxide synthase. The compounds of the present invention are useful to treat patients with neuropathy or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The compounds are also useful in the treatment of pain associated with acute herpes zoster (shingles), postherpetic neuralgia (PHN), and associated pain syndromes such as ocular pain. The compounds are further useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like. The compounds are also useful for the treatment of pain-related disorders such as tactile allodynia and hyperalgesia. The pain may be somatogenic (either nociceptive or neuropathic), acute and/or chronic. The nitric oxide inhibitors of the subject invention are also useful in conditions where NSAIDs, morphine or fentanyl opiates and/or other opioid analgesics would traditionally be administered.

Furthermore, the compounds of the subject invention can be used in the treatment or prevention of opiate tolerance in patients needing protracted opiate analgesics, and benzodiazepine tolerance in patients taking benzodiazepines, and other addictive behavior, for example, nicotine addiction, alcoholism, and eating disorders. Moreover, the compounds and methods of the present invention are useful in the treatment or prevention of drug withdrawal symptoms, for example treatment or prevention of symptoms of withdrawal from opiate, alcohol, or tobacco addiction.

In addition, the compounds of the subject invention can be used to treat insulin resistance and other metabolic disorders such as atherosclerosis that are typically associated with an exaggerated inflammatory signaling.

The present invention encompasses therapeutic methods using novel selective iNOS inhibitors to treat or prevent respiratory disease or conditions, including therapeutic methods of use in medicine for preventing and treating a respiratory disease or condition including: asthmatic conditions including allergen-induced asthma, exercise-induced asthma, pollution-induced asthma, cold-induced asthma, and viral-induced-asthma; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchioectasis cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus and hypoxia.

Other disorders or conditions which can be advantageously treated by the compounds of the present invention include inflammation. The compounds of the present invention are useful as anti-inflammatory agents with the additional benefit of having significantly less harmful side effects. The compounds are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compounds are also useful in treating osteoporosis and other related bone disorders. These compounds can also be used to treat gastrointestinal conditions such as reflux esophagitis, diarrhea, inflammatory bowel disease, Crohn's disease, gastritis, irritable bowel syndrome and ulcerative colitis.

The compounds may also be used in the treatment of pulmonary inflammation, such as that associated with viral infections and cystic fibrosis. In addition, compounds of invention are also useful in organ transplant patients either alone or in combination with conventional immunomodulators. Yet further, the compounds of the invention are useful in the treatment of pruritis and vitaligo.

The compounds of the present invention are also useful in treating tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontis, hypersensitivity, swelling occurring after injury, ischemias including myocardial ischemia, cardiovascular ischemia, and ischemia secondary to cardiac arrest, and the like.

The compounds of the subject invention are also be useful for the treatment of certain diseases and disorders of the nervous system. Central nervous system disorders in which nitric oxide inhibition is useful include cortical dementias including Alzheimer's disease, central nervous system damage resulting from stroke, ischemias including cerebral ischemia (both focal ischemia, thrombotic stroke and global ischemia (for example, secondary to cardiac arrest), and trauma. Neurodegenerative disorders in which nitric oxide inhibition is useful include nerve degeneration or nerve necrosis in disorders such as hypoxia, hypoglycemia, epilepsy, and in cases of central nervous system (CNS) trauma (such as spinal cord and head injury), hyperbaric oxygen convulsions and toxicity, dementia e.g. pre-senile dementia, and AIDS-related dementia, cachexia, Sydenham's chorea, Huntington's disease, Parkinson's Disease, amyotrophic lateral sclerosis (ALS), Korsakoffs disease, imbecility relating to a cerebral vessel disorder, sleeping disorders, schizophrenia, depression, depression or other symptoms associated with Premenstrual Syndrome (PMS), and anxiety.

Furthermore, the compounds of the present invention are also useful in inhibiting NO production from L-arginine including systemic hypotension associated with septic and/or toxic hemorrhagic shock induced by a wide variety of agents; therapy with cytokines such as TNF, IL-1 and IL-2; and as an adjuvant to short term immunosuppression in transplant therapy. These compounds can also be used to treat allergic rhinitis, respiratory distress syndrome, endotoxic shock syndrome, and atherosclerosis.

Still other disorders or conditions advantageously treated by the compounds of the subject invention include the prevention or treatment of hypreproliferative diseases, especially cancers. Hematological and non-hematological malignancies which may be treated or prevented include but are not limited to multiple myeloma, acute and chronic leukemias including Acute Lymphocytic Leukemia (ALL), Chronic Lymphocytic Leukemia (CLL), and Chronic Myelogenous Leukemia (CLL), lymphomas, including Hodgkin's lymphoma and non-Hodgkin's lymphoma (low, intermediate, and high grade), as well as solid tumors and malignancies of the brain, head and neck, breast, lung, reproductive tract, upper digestive tract, pancreas, liver, renal, bladder, prostate and colorectal. The present compounds and methods can also be used to treat the fibrosis, such as that which occurs with radiation therapy. The present compounds and methods can be used to treat subjects having adenomatous polyps, including those with familial adenomatous polyposis (FAP). Additionally, the present compounds and methods can be used to prevent polyps from forming in patients at risk of FAP.

The compounds of the subject invention can be used in the treatment of ophthalmic diseases, such as glaucoma, retinal ganglion degeneration, ocular ischemia, retinitis, retinopathies, uveitis, ocular photophobia, and of inflammation and pain associated with acute injury to the eye tissue. Specifically, the compounds can be used to treat glaucomatous retinopathy and/or diabetic retinopathy. The compounds can also be used to treat post-operative inflammation or pain as from ophthalmic surgery such as cataract surgery and refractive surgery.

Moreover, compounds of the subject invention may be used in the treatment of menstrual cramps, dysmenorrhea, premature labor, tendonitis, bursitis, skin-related conditions such as psoriasis, eczema, burns, sunburn, dermatitis, pancreatitis, hepatitis, and the like. Other conditions in which the compounds of the subject invention provide an advantage in inhibiting nitric oxide inhibition include diabetes (type I or type II), congestive heart failure, myocarditis, atherosclerosis, and aortic aneurysm.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds of the subject invention may also be used to prevent tissue damage when therapeutically combined with antibacterial or antiviral agents.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein.

### General Synthetic Methods for Preparing Compounds

The following schemes can be used to practice the present invention.

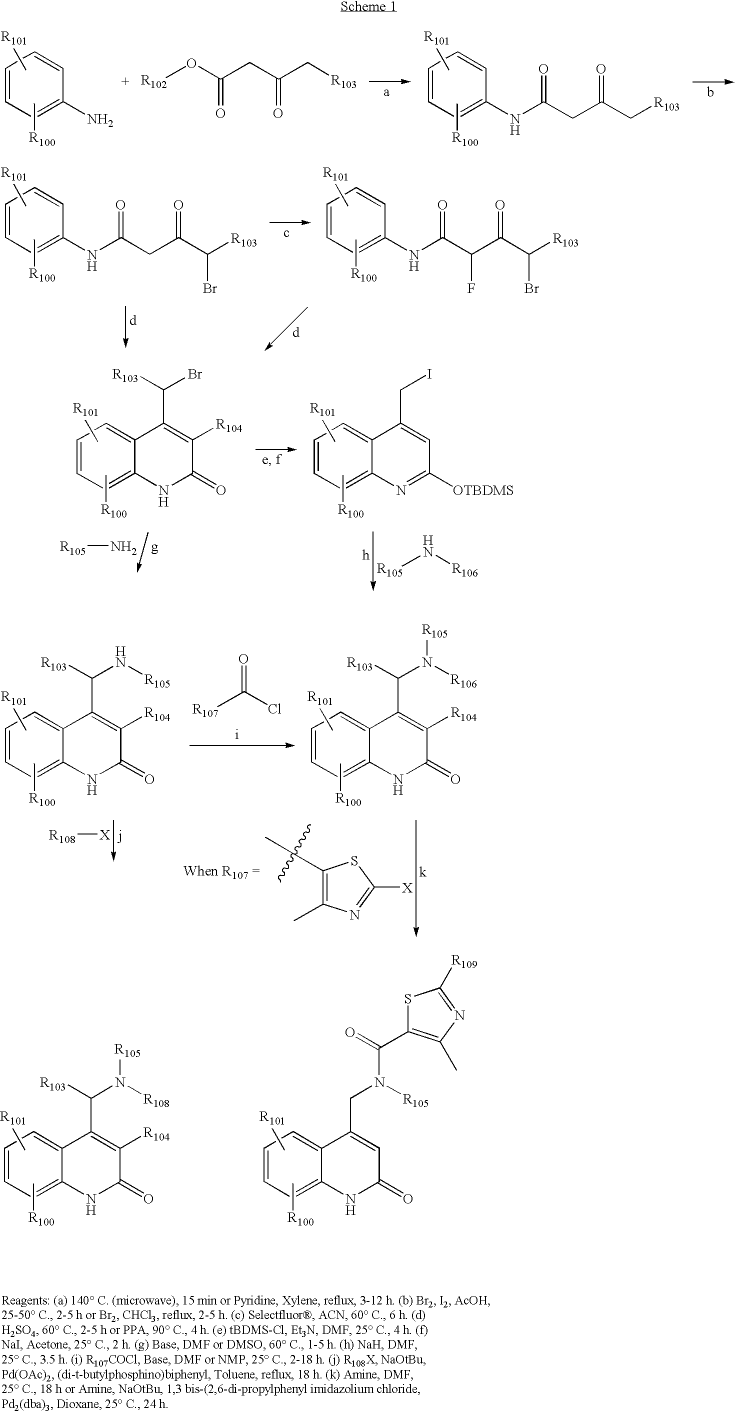

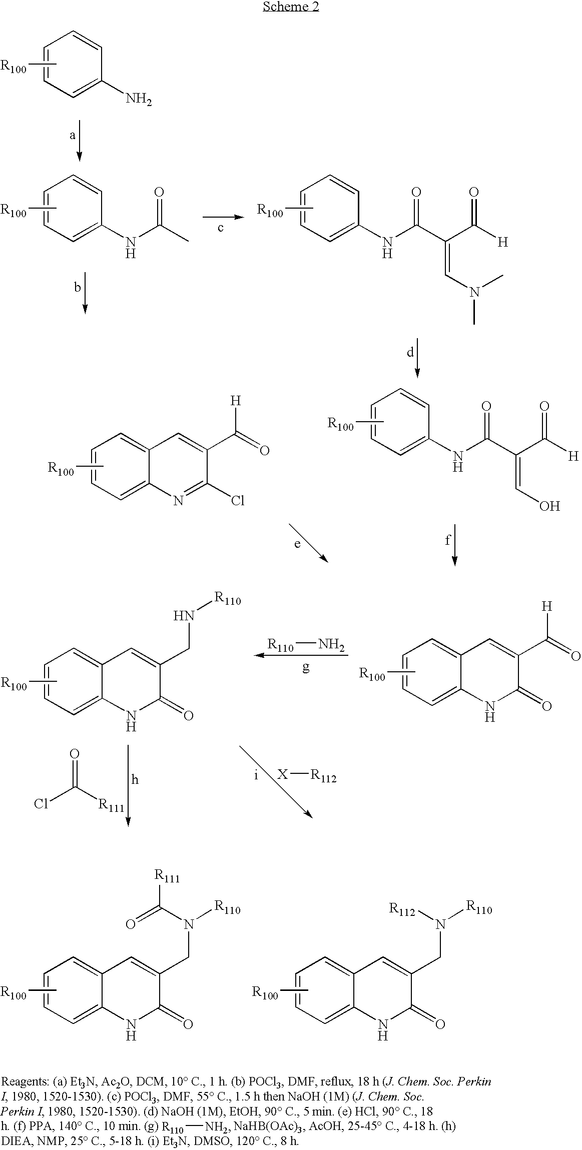

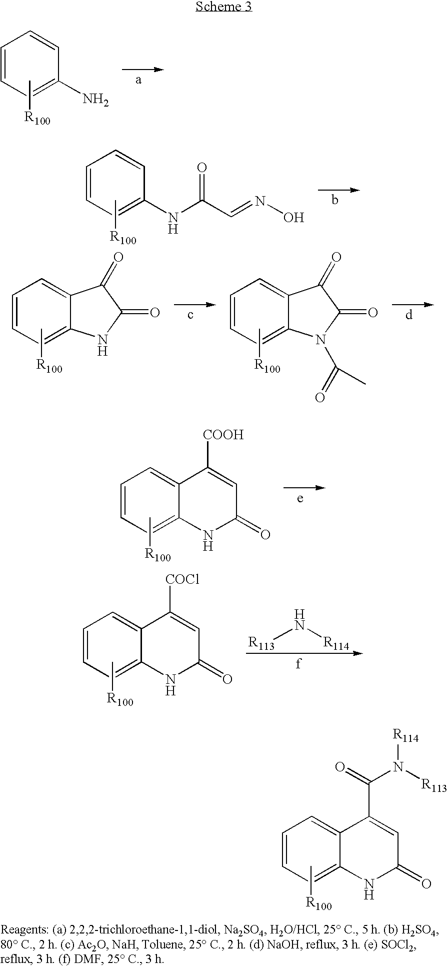

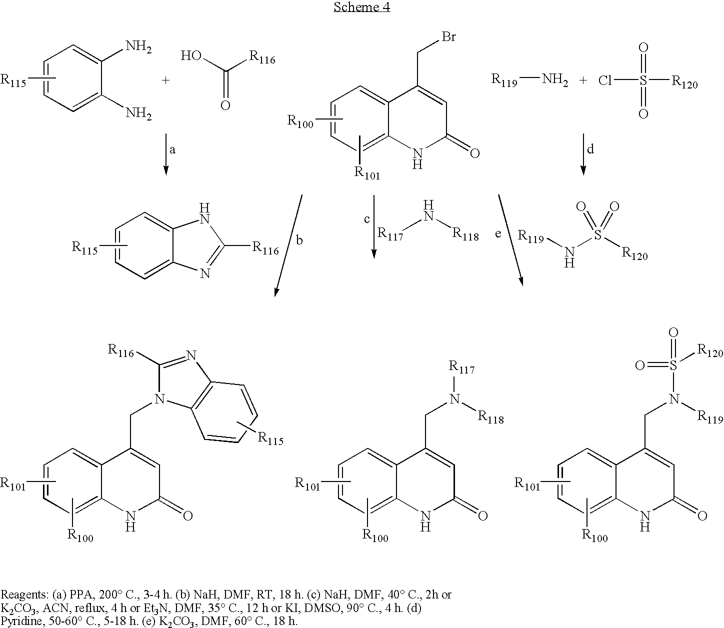

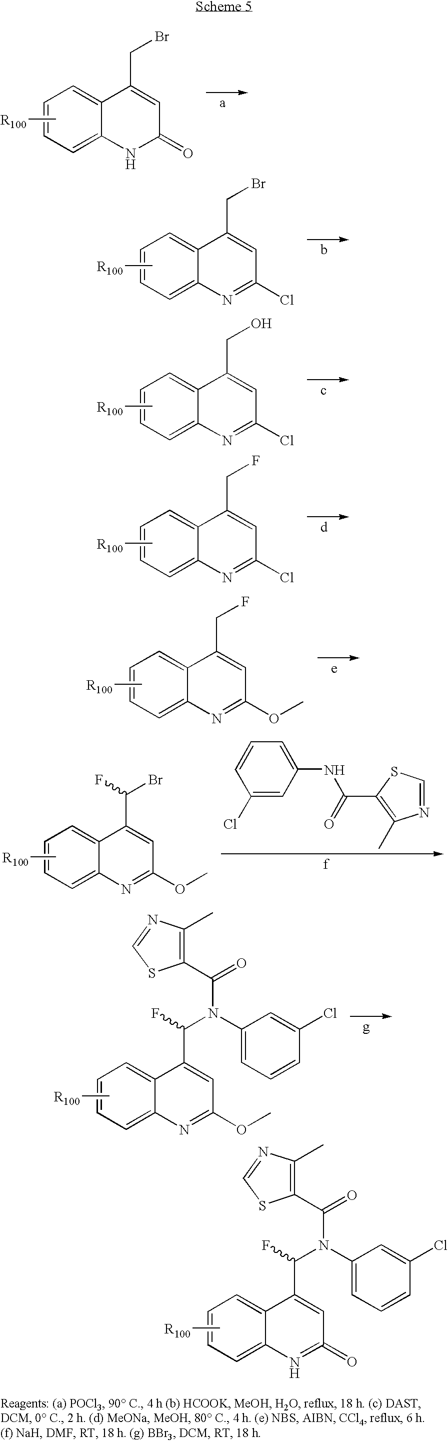

The invention is further illustrated by the following examples.

### EXAMPLE 1

### N-((2-Oxo-1,2-dihydroquinolin-4-yl)methyl)-N-phenylfuran-2-carboxamide

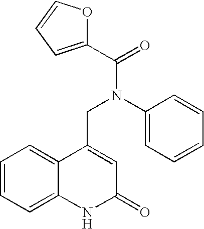

### Step 1: 3-Oxo-N-phenylbutanamide

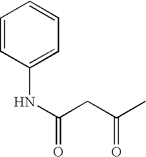

Aniline (18.4 g, 197.85 mmol) in xylene (40 mL) was added to a stirring solution of pyridine (0.05 mL). The resulting solution was allowed to react, with stirring, for 0.5 h while the temperature was maintained at reflux under a nitrogen atmosphere. A solution of ethyl 3-oxobutanoate (30 g, 230.77 mmol) and a drop of pyridine in xylene (20 mL) were added dropwise while stirring over 4 min. The reaction mixture was stirred for an additional 3 h while the temperature was maintained at reflux. The mixture was concentrated by evaporation under vacuum and the residue was cooled in a H₂O/ice bath. The solid was filtered and washed with xylene (1×20 mL) to afford 8.2 g (23%) of 3-oxo-N-phenylbutanamide as a white solid.

### Step 2: 4-Bromo-3-oxo-N-phenylbutanamide

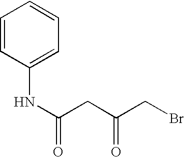

A solution of 3-oxo-N-phenylbutanamide (5.4 g, 30.51 mmol) was dissolved in CHCl₃ (15 mL). The resulting reaction mixture was refluxed and a solution of Br₂ (1.6 mL) in CHCl₃ (15 mL) was then added dropwise over 1.5 h. The reaction mixture was stirred for an additional 30 min at reflux. The reaction mixture was then cooled in an ice/salt bath followed by filtration to afford 1.2 g (15%) of 4-bromo-3-oxo-N-phenylbutanamide as a white solid.

### Step 3: 4-(Bromomethyl)-quinolin-2(1H)-one

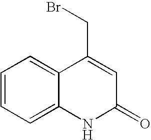

4-bromo-3-oxo-N-phenylbutanamide (1.2 g, 4.69 mmol) was added dropwise to H₂SO₄ (18 mL) over 0.5 h. The reaction mixture was stirred for an additional 1 h at 40° C. The reaction mixture was then poured into 30 mL of H₂O/ice followed by filtration to afford 0.7 g (64%) of 4-(bromomethyl)quinolin-2(1H)-one as a white solid.

### Step 4: 4-((Phenylamino)methyl)quinolin-2(1H)-one

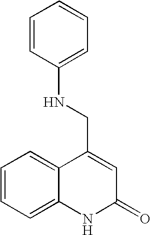

K₂CO₃ (230 mg, 1.67 mmol) and aniline (90 mg, 0.97 mmol) were added to a solution of 4-(bromomethyl)quinolin-2(1H)-one (200 mg, 0.84 mmol) in DMF (15 mL) and the resulting mixture was stirred at 60° C. for 1 h. The reaction mixture was then poured into 100 ml of EtOAC and was washed with of brine (3×50 mL). The solvent was removed and the residue was purified by silica gel flash column chromatography (50% ethyl acetate in petroleum ether) to afford 0.1 g (47.6%) of 4-((phenylamino)methyl)quinolin-2(1H)-one as a yellow solid.

### Step 5: N-((2-Oxo-1,2-dihydroquinolin-4-yl)methyl)-N-phenylfuran-2-carboxamide

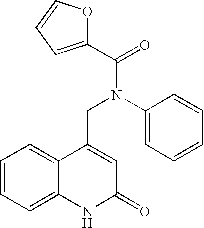

Furan-2-carbonyl chloride (460 mg, 3.51 mmol) was added dropwise to a cooled (0° C.) solution of 4-((phenylamino)methyl)quinolin-2(1H)-one (800 mg, 3.20 mmol) in DMF (100 mL). Et₃N (650 mg, 6.44 mmol) was then added dropwise and the resulting solution was allowed to react, with stirring, for 3 h while at RT. The reaction mixture was then poured into 400 mL of EtOAc and was washed with a saturated NaHCO₃ solution (2×300 mL) and with (2×300 mL) of brine. The mixture was dried over MgSO₄ and concentrated by evaporation under vacuum. The residue was purified by chromatography on silica gel (eluting with a 1:1.5 PE:EtOAc) to give 320 mg (29.1%) of N-((2-oxo-1,2-dihydroquinolin-4-yl)methyl)-N-phenylfuran-2-carboxamide as a white solid. ¹H NMR (400 MHz, DMSO-d₆) δ 11.24 (s, 1H), 7.91 (d, 1H), 7.37 (d, 1H), 7.34 (m, 3H), 7.33 (m, 1H), 7.27 (m, 1H), 7.11 (m, 2H), 7.00 (t, 1H), 6.57 (s, 1H), 6.22 (m, 1H), 5.79 (d, 1H), 5.34 (s, 2H).

### EXAMPLE 2

### N-(4-Chlorophenyl)-N-((2-oxo-1,2-dihydroquinolin-4-yl)methyl)furan-2-carboxamide

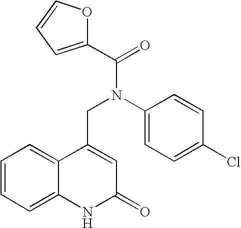

N-(4-Chlorophenyl)-N-((2-oxo-1,2-dihydroquinolin-4-yl)methyl)furan-2-carboxamide was synthesized as described in EXAMPLE 1, Step 4-5 using 4-(bromomethyl)quinolin-2(1H)-one, 4-chloroaniline, and furan-2-carbonyl chloride as starting materials. ¹H NMR (400 MHz, DMSO-d₆) δ 11.7 (s, 1H), 7.80 (d, 1H), 7.69 (d, 1H), 7.51 (m, 1H), 7.41 (d, 2H), 7.31 (d, 1H), 7.23 (d, 1H), 7.22 (d, 2H), 6.45 (m, 1H), 6.30 (s, 1H), 6.17 (m, 1H), 5.26 (s, 2H)

### EXAMPLE 3

### N-[(2-Oxo-1,2-dihydroquinolin-4-yl)methyl]-N-phenylacetamide

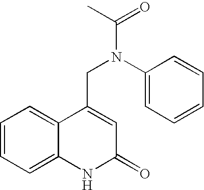

N-[(2-Oxo-1,2-dihydroquinolin-4-yl)methyl]-N-phenylacetamide was synthesized as described in EXAMPLE 1, Step 5 using 4-((phenylamino)methyl)quinolin-2(1H)-one and acetyl chloride as starting materials. ¹H NMR (400 MHz, DMSO-d₆) δ 11.7 (s, 1H), 7.75 (d, 1H), 7.49 (d, 1H), 7.38 (m, 1H), 7.31 (m, 5H), 7.17 (m, 1H), 6.27 (s, 1H), 5.05 (s, 2H), 1.88 (s, 3H). LCMS: 293.0 (M+H)⁺.

### EXAMPLE 4

### N-((2-Oxo-1,2-dihydroquinolin-4-yl)methyl)-N-phenylpropionamide

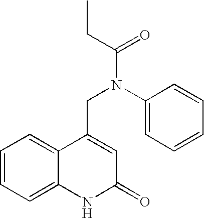

N-((2-Oxo-1,2-dihydroquinolin-4-yl)methyl)-N-phenylpropionamide was synthesized as described in EXAMPLE 1, Step 5 using 4-(anilinomethyl)-quinolin-2(1H)-one and propionyl chloride as starting materials. ¹HNMR (400 MHz, DMSO-d₆) δ 11.7 (s, 1H), 7.74 (d, 1H), 7.31 (m, 2H), 7.28 (d, 1H), 7.24 (d, 1H), 7.12 (m, 1H), 7.10 (d, 2H), 6.95 (d, 1H), 6.25 (s, 1H), 5.09 (s, 2H), 2.10 (m, 2H), 0.94 (m, 3H). LCMS: 306 (M)⁺.

### EXAMPLE 5

### N-((2-Oxo-1,2-dihydroquinolin-4-yl)methyl)-N-phenylisobutyramide

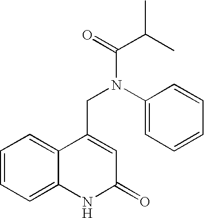

N-((2-Oxo-1,2-dihydroquinolin-4-yl)methyl)-N-phenylisobutyramide was synthesized as described in EXAMPLE 1, Step 5 using 4-(anilinomethyl)-quinolin-2(1H)-one and isobutyryl chloride as starting materials. LCMS: 321 (M+H)⁺.

### EXAMPLE 6

### N-[(2-Oxo-1,2-dihydroquinolin-4-yl)methyl]-N-phenylbenzamide

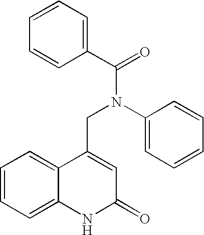

N-[(2-Oxo-1,2-dihydroquinolin-4-yl)methyl]-N-phenylbenzamide was synthesized as described in EXAMPLE 1, Step 5 using 4-((phenylamino)methyl)quinolin-2(1H)-one and benzoyl chloride as starting materials. ¹H NMR (400 MHz, DMSO-d₆) δ 11.7 (s, 1H), 7.95 (d, 2H), 7.64 (d, 2H), 7.59 (d, 1H), 7.51 (s, 1H), 7.44 (m, 2H), 7.28 (d, 1H), 7.24 (m, 2H), 7.12 (m, 1H), 7.00 (m, 1H), 6.95 (m, 1H), 6.40 (s, 1H), 5.35 (s, 2H). LCMS: 355.0 (M+H)⁺.

### EXAMPLE 7

### N-[(2-Oxo-1,2-dihydroquinolin-4-yl)methyl]-N-phenylthiophene-2-carboxamide

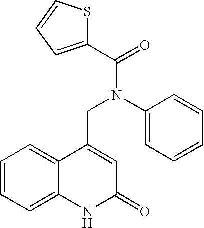

N-[(2-oxo-1,2-dihydroquinolin-4-yl)methyl]-N-phenylthiophene-2-carboxamide was synthesized as described in EXAMPLE 1, Step 5 using 4-((phenylamino)methyl)quinolin-2(1H)-one and thiophene-2-carbonyl chloride as starting materials. ¹H NMR (400 MHz, DMSO-d₆) δ 11.7 (s, 1H), 7.82 (d, 1H), 7.67 (d, 1H), 7.49 (d, 1H), 7.35 (m, 4H), 7.23 (m, 2H), 6.88 (m, 1H), 6.64 (m, 1H), 6.32 (m, 2H), 5.27 (s, 2H). LCMS: 361.0 (M+H)⁺.

### EXAMPLE 8

### N-Methyl-N-[(2-oxo-1,2-dihydroquinolin-4-yl)methyl]-2-furamide

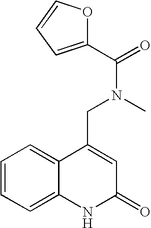

### Step 1: N-Methyl-2-furamide

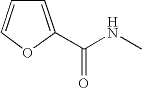

Furan-2-carbonyl chloride (1.93 g, 14.79 mmol) was added dropwise over 0.5 h to a solution of methanamine hydrochloride (1 g, 14.81 mmol) in DCM (50 mL) at 0° C. Et₃N (3 g) was then added over 10 min with stirring and the reaction mixture was stirred at RT for 12 h. The reaction mixture was then concentrated and dried to afford 1 g (54%) of N-methyl-2-furamide as a yellow oil.

### Step 2: N-Methyl-N-[(2-oxo-1,2-dihydroquinolin-4-yl)methyl]-2-furamide

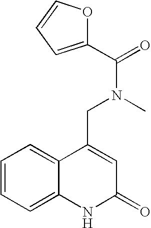

Sodium hydride (180 mg, 4.50 mmol) and 4-(bromomethyl)quinolin-2(1H)-one (500 mg, 2.10 mmol) were added to a solution of N-methylfuran-2-carboxamide (260 mg, 2.08 mmol) in DMF (20 mL). The reaction mixture was then stirred for 2 h at RT before being concentrated and the residue purified by silica gel flash column chromatography (5% MeOH in DCM) to afford 170 mg (29%) of N-methyl-N-((2-oxo-1,2-dihydroquinolin-4-yl)methyl)-2-furaxamide as a white solid. ¹H NMR (400 MHz, DMSO-d₆) δ 11.74 (s, 1H), 7.75 (m, 3H), 7.53 (m, 1H), 7.34 (m, 1H), 7.23 (m, 1H), 7.20 (m, 1H), 6.20 (s, 1H), 3.24 (s, 2H), 2.47 (s, 3H). LCMS: 283.0 (M+H)⁺.

### EXAMPLE 9

### N-Isopropyl-N-[(2-oxo-1,2-dihydroquinolin-4-yl)methyl]-2-furamide

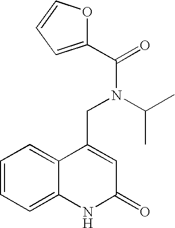

### Step 1: 4-[(Isopropylamino)methyl]quinolin-2(1H)-one

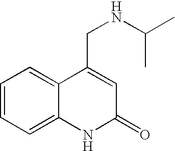

Propan-2-amine (7.47 g, 126.61 mmol) was added to a solution of 4-(bromomethyl)quinolin-2(1H)-one (1 g, 4.22 mmol) in DMF (100 mL), followed by K₂CO₃ (590 mg, 4.28 mmol). The reaction mixture was stirred overnight at 0° C. in a H₂O/ice bath then at RT. The reaction mixture was concentrated and washed with acetone (3×150 mL) to afford 1.1 g (crude) of 4-((isopropylamino)methyl) quinolin-2(1H)-one as a white solid.

### Step 2: N-Isopropyl-N-[(2-oxo-1,2-dihydroquinolin-4-yl)methyl]-2-furamide

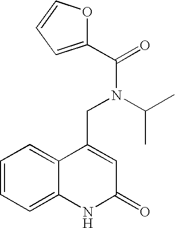

N-isopropyl-N-[(2-oxo-1,2-dihydroquinolin-4-yl)methyl]-2-furamide was synthesized as described in EXAMPLE 1, Step 5 using 4-[(isopropylamino)methyl]quinolin-2(1H)-one and furan-2-carbonyl chloride as starting materials. ¹H NMR (300 MHz, DMSO-d₆) δ 11.69 (s, 1H), 7.89 (m, 2H), 7.54 (dd, 1H), 7.36 (dd, 1H), 7.22 (dd, 1H), 7.00 (s, 1H), 6.60 (s, 1H), 6.17 (s, 1H), 4.67 (m, 1H), 3.31 (s, 2H), 1.23 (d, 6H). LCMS: 311.1 (M+H)⁺.

### EXAMPLE 10

### N-(4-Methoxyphenyl)-N-[(2-oxo-1,2-dihydroquinolin-4-yl)methyl]-2-furamide

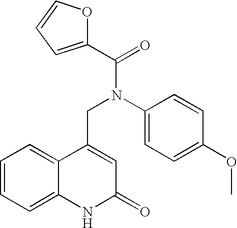

N-(4-Methoxyphenyl)-N-[(2-oxo-1,2-dihydroquinolin-4-yl)methyl]-2-furamide was synthesized as described in EXAMPLE 1, Step 4-5 using 4-(bromomethyl)quinolin-2(1H)-one, 4-methoxyaniline, and furan-2-carbonyl chloride as starting materials. ¹H NMR (400 MHz, DMSO-d₆) δ 11.74 (s, 1H), 7.93 (d, 1H), 7.80 (d, 1H), 7.49 (m, 1H), 7.31 (d, 1H), 7.23 (d, 1H), 7.17 (m, 3H), 7.09 (d, 2H), 6.91 (m, 1H), 6.28 (s, 1H), 5.72 (s, 2H), 3.71 (s, 3H). LCMS: 375.1 (M+H)⁺.

### EXAMPLE 11

### N-(4-Methylphenyl)-N-[(2-oxo-1,2-dihydroquinolin-4-yl)methyl]-2-furamide

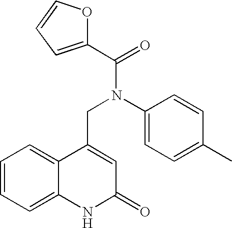

N-(4-Methylphenyl)-N-[(2-oxo-1,2-dihydroquinolin-4-yl)methyl]-2-furamide was synthesized as described in EXAMPLE 1, Step 4-5 using 4-(bromomethyl)quinolin-2(1H)-one, 4-methylaniline, and furan-2-carbonyl chloride as starting materials. ¹H NMR (300 MHz, DMSO-d₆) δ 11.7 (s, 1H), 7.81 (d, 1H), 7.69 (d, 1H), 7.49 (m, 1H), 7.32 (d, 1H), 7.29 (m, 1H), 7.18 (d, 2H), 7.04 (d, 2H), 6.39 (d, 1H), 6.27 (m, 1H), 5.79 (s, 1H), 5.21 (s, 2H), 2.26 (s, 3H). LCMS: 359.0 (M+H)⁺.

### EXAMPLE 12

### N-Benzyl-N-[(2-oxo-1,2-dihydroquinolin-4-yl)methyl]-2-furamide

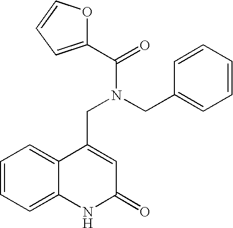

N-Benzyl-N-[(2-oxo-1,2-dihydroquinolin-4-yl)methyl]-2-furamide was synthesized as described in EXAMPLE 1, Step 4-5 using 4-(bromomethyl)quinolin-2(1H)-one, phenylmethanamine, and furan-2-carbonyl chloride as starting materials. ¹H NMR (300 MHz, DMSO-d₆) δ 11.7 (s, 1H), 8.85 (d, 1H), 7.97 (d, 1H), 7.69 (d, 1H), 7.52 (m, 2H), 7.23 (d, 1H), 7.12 (m, 1H), 7.06 (m, 3H), 6.95 (m, 1H), 6.61 (m, 1H), 6.47 (s, 1H), 4.86 (s, 2H), 4.0 (s, 2H). LCMS: 359.0 (M+H)⁺.

### EXAMPLE 13

### N-[(2-Oxo-1,2-dihydroquinolin-4-yl)methyl]-N-pyridin-4-yl-2-furamide

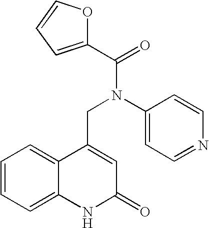

### Step 1: N-(Pyridin-4-yl)-2-furamide

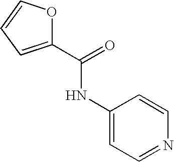

A solution of furan-2-carbonyl chloride (2.77 g, 21.22 mmol) in DMF (20 ml) was added dropwise over 0.5 h to a solution of pyridin-4-amine (2 g, 21.25 mmol) in DMF (30 ml) at 0° C. The reaction mixture was then stirred at RT for 2 h. The solvent was removed and the residue was purified by silica gel flash column chromatography (5% MeOH in dichloromethane) to afford 3 g (75%) of N-(pyridin-4-yl)-2-furamide as a white solid.

### Step 2: N-[(2-Oxo-1,2-dihydroquinolin-4-yl)methyl]-N-(pyridin-4-yl)-2-furamide

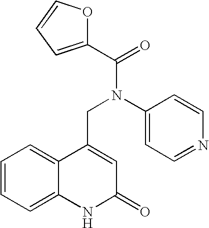

Sodium hydride (40 mg, 1 mmol) and 4-(bromomethyl)quinolin-2(1H)-one (700 mg, 2.94 mmol) were added to a solution of N-(pyridin-4-yl)furan-2-carboxamide (200 mg, 1.06 mmol) in DMF (25 ml). The reaction mixture was stirred for 2 h at 40° C. The solvent was removed and the residue was purified by silica gel flash column chromatography (10% MeOH in dichloromethane) to afford 0.35 g (95%) of N-((2-oxo-1,2-dihydroquinolin-4-yl)methyl)-N-(pyridin-4-yl)-2-furamide as a white solid. LCMS: 346.0 (M+H)⁺.

### EXAMPLE 14

### N-(3-Chlorophenyl)-N-[(2-oxo-1,2-dihydroquinolin-4-yl)methyl]-2-furamide

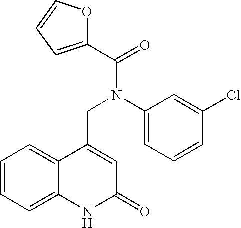

N-(3-Chlorophenyl)-N-[(2-oxo-1,2-dihydroquinolin-4-yl)methyl]-2-furamide was synthesized as described in EXAMPLE 1, Step 4-5 using 4-(bromomethyl)quinolin-2(1H)-one, 3-chloroaniline, and furan-2-carbonyl chloride as starting materials. ¹H NMR (300 MHz, CDCl₃) δ 11.7 (s, 1H), 7.80 (d, 1H), 7.69 (d, 1H), 7.51 (m, 1H), 7.41 (m, 1H), 7.32 (s, 1H), 7.31 (d, 1H), 7.23 (d, 1H), 7.22 (d, 1H), 7.02 (d, 1H), 6.45 (m, 1H), 6.30 (s, 1H), 6.17 (m, 1H), 5.26 (s, 2H).

### EXAMPLE 15

### 4-[(Methyl-phenyl-amino)-methyl]-1H-quinolin-2-one

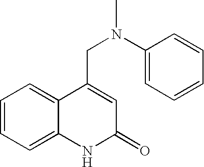

N-Methylaniline (120 μL, 1.1 mmol) was added to a stirred mixture of 4-(bromomethyl)quinolin-2(1H)-one (238 mg, 1.0 mmol) and DIEA (700 μL, 4.0 mmol) in DMF (10 mL) at RT. The resulting mixture was warmed to 50° C. and stirred for 3 hours, then cooled to RT and poured in to ice H₂O (100 mL). The resulting precipitate was filtered and washed with an additional 20 mL ice H₂O. The residue was then dissolved in DCM, dried (MgSO₄), filtered, and concentrated to afford 4-[(methyl-phenyl-amino)-methyl]-1H-quinolin-2-one (189 mg) as awhite solid. ¹HNMR (400 MHz, DMSO-d₆) δ 11.66 (s, 1H), 7.76 (d, 1H), 7.52 (dd, 1H), 7.34 (d, 1H), 7.15 (m, 3H), 6.64 (m, 3H), 6.00 (s, 1H), 4.81 (s, 2H), 3.06 (s, 3H). LCMS: 265.4 (M+H)⁺.

### EXAMPLE 16

### N-((8-Methyl-2-oxo-1,2-dihydroquinolin-4-yl)methyl)-N-phenylfuran-2-carboxamide

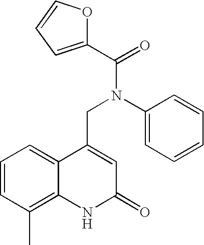

N-((8-Methyl-2-oxo-1,2-dihydroquinolin-4-yl)methyl)-N-phenylfuran-2-carboxamide was synthesized as described in EXAMPLE 1 using o-toluidine as a starting material. ¹H NMR (300 MHz, DMSO-d₆) δ 10.85 (s, 1H), 7.72 (d, 1H), 7.64 (d, 2H), 7.24 (m, 2H), 7.23 (d, 1H), 7.09 (s, 1H), 7.00 (m, 1H), 6.92 (d, 1H), 6.83 (m, 1H), 6.61 (m, 2H), 5.88 (s, 1H), 5.26 (s, 2H), 2.39 (s, 3H). LCMS: 359.0 (M+H)⁺.

### EXAMPLE 17

### N-((8-Fluoro-2-oxo-1,2-dihydroquinolin-4-yl)methyl)-N-phenylfuran-2-carboxamide

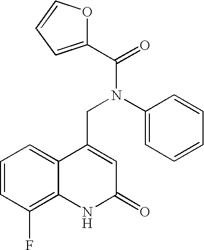

N-((8-Fluoro-2-oxo-1,2-dihydroquinolin-4-yl)methyl)-N-phenylfuran-2-carboxamide was synthesized as described in EXAMPLE 1 using 2-fluoroaniline as a starting material. ¹H NMR (400 MHz, DMSO-d₆) δ 11.75 (s, 1H), 7.72 (d, 1H), 7.64 (d, 2H), 7.24 (m, 3H), 7.05 (d, 1H), 7.00 (m, 1H), 6.93 (m, 1H), 6.83 (d, 1H), 6.61 (m, 1H), 5.89 (s, 1H), 5.28 (s, 2H). LCMS: 363.0 (M+H)⁺.

### EXAMPLE 18

### N-((6-Fluoro-2-oxo-1,2-dihydroquinolin-4-yl)methyl)-N-phenylfuran-2-carboxamide

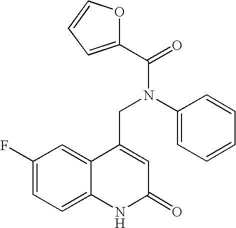

N-((6-Fluoro-2-oxo-1,2-dihydroquinolin-4-yl)methyl)-N-phenylfuran-2-carboxamide was synthesized as described in EXAMPLE 1 using 4-fluoroaniline as a starting material. ¹H NMR (400 MHz, DMSO-d₆) δ 11.86 (s, 1H), 7.72 (d, 1H), 7.64 (d, 2H), 7.57 (d, 1H), 7.24 (m, 3H), 7.00 (m, 1H), 6.99 (s, 1H), 6.83 (d, 1H), 6.61 (m, 1H), 5.89 (s, 1H), 5.25 (s, 2H). LCMS: 363.0 (M+H)⁺.

### EXAMPLE 19

### N-((6-Methoxy-2-oxo-1,2-dihydroquinolin-4-yl)methyl)-N-phenylfuran-2-carboxamide

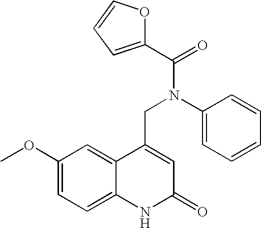

N-((6-Methoxy-2-oxo-1,2-dihydroquinolin-4-yl)methyl)-N-phenylfuran-2-carboxamide was synthesized as described in EXAMPLE 1 using 4-methoxyaniline as a starting material. ¹H NMR (400 MHz, DMSO-d₆) δ 11.67 (s, 1H), 7.68 (d, 1H), 7.48 (d, 1H), 7.34 (d, 2H), 7.23 (d, 1H), 7.17 (m, 2H), 7.00 (m, 1H), 6.79 (s, 1H), 6.63 (d, 1H), 6.61 (m, 1H), 5.85 (s, 1H), 5.26 (s, 2H), 3.79 (s, 3H). LCMS: 375.0 (M+H)⁺.

### EXAMPLE 20

### N-((7-Methyl-2-oxo-1,2-dihydroquinolin-4-yl)methyl)-N-phenylfuran-2-carboxamide

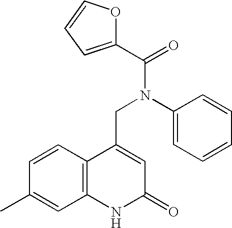

N-((7-Methyl-2-oxo-1,2-dihydroquinolin-4-yl)methyl)-N-phenylfuran-2-carboxamide was synthesized as described in EXAMPLE 1 using 3-methylaniline as a starting material. ¹H NMR (400 MHz, DMSO-d₆) δ 11.75 (s, 1H), 7.72 (d, 1H), 7.64 (d, 2H), 7.39 (s, 1H), 7.24 (d, 2H), 7.23 (d, 1H), 7.16 (d, 1H), 7.00 (m, 1H), 6.75 (d, 1H), 6.61 (m, 1H), 5.86 (s, 1H), 4.96 (s, 2H), 2.35 (s, 3H). LCMS: 359.0 (M+H)⁺.

### EXAMPLE 21

### N-((6-Methyl-2-oxo-1,2-dihydroquinolin-4-yl)methyl)-N-phenylfuran-2-carboxamide

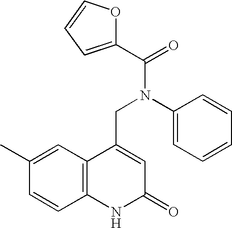

N-((6-Methyl-2-oxo-1,2-dihydroquinolin-4-yl)methyl)-N-phenylfuran-2-carboxamide was synthesized as described in EXAMPLE 1 using 4-methylaniline as a starting material. ¹H NMR (400 MHz, DMSO-d₆) δ 11.75 (s, 1H), 7.92 (d, 2H), 7.72 (d, 1H), 7.47 (d, 1H), 7.23 (d, 1H), 7.08 (s, 1H), 7.00 (m, 1H), 6.92 (d, 1H), 6.61 (m, 1H), 6.39 (m, 2H), 5.90 (s, 1H), 5.24 (s, 2H), 2.34 (s, 3H). LCMS 359.0 (M+H)⁺.

### EXAMPLE 22

### 4-(((Furan-2-ylmethyl)(phenyl)amino)methyl)quinolin-2(1H)-one

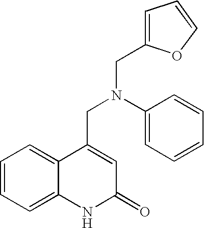

### Step 1: 2-(Chloromethyl)furan

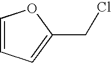

A solution of SOCl₂(13.1 g, 110.08 mmol) in CHCl₃ (50 mL) was added dropwise to a solution of furan-2-ylmethanol (9.8 g, 100 mmol) and triethylamine (20.2 g, 200 mmol) in CHCl₃ (100 mL) at 15-20° C. The resulting reaction mixture was stirred at RT for 1 h before being washed with H₂O (3×200 mL). The organic layer was dried over Na₂SO₄, filtered, and evaporated to dryness. The final product was purified by distillation under reduced pressure (20 mm Hg) and the fraction was collected at 40-50° C. This resulted in 1.5 g (13%) of 2-(chloromethyl)furan as a yellow liquid.

### Step 2: N-(Furan-2-ylmethyl)aniline

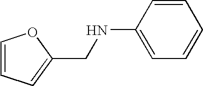

A mixture of aniline (2 g, 21.48 mmol), 2-(chloromethyl)furan (5 g, 42.90 mmol), and K₂CO₃ (5 g, 36.18 mmol) in CH₃CN (50 mL) was refluxed for 4 h. The mixture was concentrated by evaporation under vacuum using a rotary evaporator to afford a residue that was partitioned between H₂O (50 mL) and EtOAc (50 mL). The aqueous layer was extracted with EtOAc (3×50 mL). The organics were combined, dried over Na₂SO₄, filtered, and evaporated to dryness. The residue was purified by column chromatography on silica gel eluting 1:100 EtOAc/Hexanes to afford 0.4 g (10%) of N-(furan-2-ylmethyl)benzenamine as a yellow oil. LCMS 174 (M+H)⁺.

### Step 3: 4-(((Furan-2-ylmethyl)(phenyl)amino)methyl)quinolin-2(1H)-one

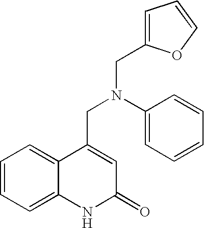

A mixture of 4-(bromomethyl)quinolin-2(1H)-one (470 mg, 1.96 mmol), N-(furan-2-ylmethyl)benzenamine (690 mg, 3.95 mmol), and K₂CO₃ (550 mg, 3.95 mmol) in DMF (30 mL) was stirred at 80° C. for 8 h. Solids residue were filtered and the filtrate was concentrated by evaporation under vacuum using a rotary evaporator to afford 0.2 g (28%) of 4-(((furan-2-ylmethyl)(phenyl)amino)methyl)quinolin-2(1H)-one as a yellow solid. LCMS: 331 (M+H)⁺.

### EXAMPLE 23

### 4-(((2-(Furan-2-yl)ethyl)(phenyl)amino) methyl)quinolin-2(1H)-one

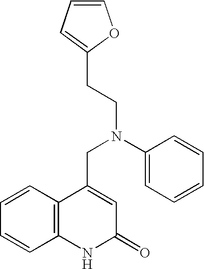

### Step 1: 2-(Furan-2-yl)ethanol

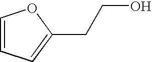

A solution of n-BuLi (42 mL, 3.65 M) in THF (147 mL) was cooled to 25° C.; and was added to furan (10 g, 147 mmol). The solution was stirred at −15° C. for 4 h, before ethylene oxide (10 mL) was added. Stirring was continued for another 1 h at −15° C. and then 12 h at RT. The solution was poured into ice/H₂O and extracted with ether (2×200 mL). The ether layers were washed with water followed by brine and dried over Na₂SO₄. The mixture was concentrated by evaporation under vacuum to afford 10 g (55%) of 2-(furan-2-yl)ethanol as a red oil.

### Step 2: 2-(Furan-2-yl)ethyl 4-methylbenzenesulfonate

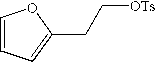

4-Methylbenzene-1-sulfonyl chloride (16.9 g, 88.95 mmol) was added to a solution of 2-(furan-2-yl)ethanol (10 g, 89.29 mmol) in CHCl₃ (30 mL). Pyridine (20 mL) was the added and the reaction mixture was allowed to react, with stirring, for 2 h while the temperature was maintained at −40° C. in a ice/salt bath. Adjustment of the pH to 7 was accomplished by the addition of HCl (4M). The resulting solution was extracted with DCM (2×200 mL), dried over Na₂SO₄, and concentrated by evaporation under vacuum. This resulted in 7 g (27%) of 2-(furan-2-yl)-ethyl 4-methylbenzenesulfonate as a red oil. ¹H NMR (400 MHz, CDCl₃) δ 7.73 (d, 2H), 7.34 (d, 2H), 7.25 (d, 1H), 6.30 (m, 1H), 6.10 (d, 1H), 3.86 (d, 2H), 2.89 (d, 2H), 2.44 (s, 3H).

### Step 3: 2-(2-Iodoethyl)furan

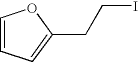

Sodium iodide (7.9 g, 52.14 mmol) was added to a solution of 2-(furan-2-yl)ethyl 4-methylbenzenesulfonate (7 g, 26.05 mmol) in acetone (50 mL) The resulting solution was allowed to react, with stirring, for 2 h while the temperature was maintained at 35° C. A filtration was performed and the filtrate was concentrated by evaporation under vacuum to give 5 g (78%) of 2-(2-iodoethyl)furan as a red oil.

### Step 4: N-(2-(Furan-2-yl)ethyl)benzenamine

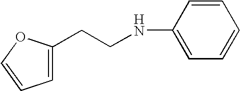

Aniline (2.1 g, 22.35 mmol), K₂CO₃ (6.2 g, 44.48 mmol) were added to a solution of 2-(2-iodoethyl)furan (5 g, 22.30 mmol) in acetone (50 mL). The resulting solution was allowed to react, with stirring, for 2 h at 45° C. A filtration was performed and the filtrate was concentrated by evaporation under vacuum to give 3 g (65%) of N-(2-(furan-2-yl)ethyl)benzenamine as a red oil. LCMS: 188 (M+H)⁺.

### Step 5: 4-(((2-(Furan-2-yl)ethyl)(phenyl)amino) methyl)quinolin-2(1H)-one

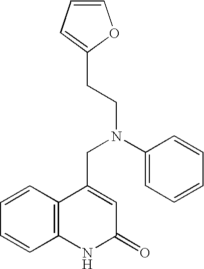

N-(2-(Furan-2-yl)ethyl)benzenamine (450 mg, 2.41 mmol) and Et₃N (400 mg, 3.96 mmol) was added to a solution of 4-(bromomethyl)quinolin-2(1H)-one (470 mg, 1.98 mmol) in DMF (30 mL). The resulting solution was allowed to react, with stirring, for 12 h at 35° C. The mixture was concentrated by evaporation followed by adjustment of the pH to 4 (accomplished by the addition of 1N HCl). The resulting solution was extracted with DCM (3×50 mL), dried over Na₂SO₄, and concentrated to afford 60 mg (8%) of 4-(((2-(furan-2-yl)ethyl)(phenyl)amino) methyl)quinolin2(1H)-one as a brown solid. ¹H NMR (400 MHz, CDCl₃) δ 12.2 (s, 1H), 8.20 (d, 1H), 8.14 (d, 1H), 7.99 (m, 1H), 7.73 (m, 1H), 7.26 (d, 1H), 7.08 (m, 2H), 6.64 (s, 1H), 6.59 (d, 2H), 6.18 (m, 1H), 5.88 (d, 1H), 4.65 (s, 2H), 3.69 (d, 2H), 2.87 (d, 2H). LCMS: 345.0 (M+H)⁺.

### EXAMPLE 24

### 4-(((3-(Furan-2-yl)propyl)(phenyl)amino)rmethyl)quinolin-2(1H)-one

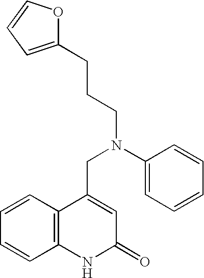

### Step 1: (E)-3-(Furan-2-yl)acrylic acid

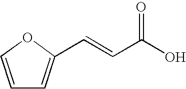

Acetic anhydride (21.25 g, 208.33 mmol) and K₂CO₃ (57.46 g, 419.42 mmol) were added to furan-2-carbaldehyde (20 g, 208.33 mmol). The resulting solution was allowed to react, with stirring, for 3 h at 90° C. Adjustment of the pH to 3 was accomplished by the addition of HCl (10%). The resulting solution was extracted with EtOAc (3×300 mL). The organics were combined and washed with water (3×100 mL), dried over Na₂SO₄, and concentrated by evaporation under vacuum to give 18 g (50%) of (E)-3-(furan-2-yl)acrylic acid as a light yellow solid.

### Step 2: 3-(Furan-2-yl)propanoic acid

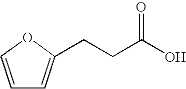

Pd/C (2 g, 0.96 mmol) was added to a solution of (E)-3-(furan-2-yl)acrylic acid (17 g, 110.87 mmol) in HOAc (200 mL) under nitrogen. Then nitrogen protection was removed and hydrogen atmosphere was introduced into the reaction mixture. The resulting solution was allowed to react, with stirring, overnight while the temperature was maintained at 20° C. A filtration was performed and the filtrate concentrated by evaporation. The residue was dissolved in 300 mL of EtOAC, washed with water (2×50 mL), dried over Na₂SO₄, and concentrated by evaporation to afford 8 g (41%) of 3-(furan-2-yl)propanoic acid as a white solid. ¹H NMR (400 MHz, CDCl₃) δ 7.31 (d, 1H), 6.33 (d, 1H), 6.04 (d, 1H), 2.98 (d, 2H), 2.74 (m, 2H). LCMS: 139.0 (M+H)⁺.

### Step 3: 3-(Furan-2-yl)-N-phenylpropanamide

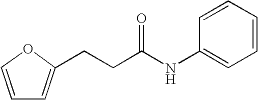

A solution of 3-(furan-2-yl)propanoic acid (1.5 g, 9.64 mmol) in DCM (15 mL) was added dropwise to a cooled solution (0° C.) of N-((cyclohexylimino)methylene) cyclohexanamine (4.42 g, 21.42 mmol) in DCM (15 mL). Aniline (1.29 g, 13.85 mmol) was then added to this mixture and the resulting solution was allowed to react overnight at RT. A filtration was performed and the filtrate was concentrated to afford 1.4 g (54%) of 3-(furan-2-yl)-N-phenylpropanamide as a light yellow oil.

### Step 4: N-(3-(Furan-2-yl)propyl)benzenamine

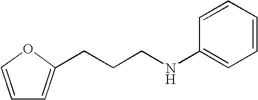

LiAlH₄ (100 mg, 2.63 mmol) was added in several batches to a cooled (0° C.) solution of 3-(furan-2-yl)-N-phenylpropanamide (200 mg, 0.84 mmol) in THF (10 mL). The resulting solution was allowed to react, with stirring, for 3 h. The reaction mixture was then quenched by water (10 mL). The resulting solution was extracted with EtOAc (1×20 ml), dried over Na₂SO₄ and concentrated. The residue obtained was purified by chromatography on silica gel (1:40 EtOAc/PE) to afford 0.1 g (53%) of N-(3-(furan-2-yl)propyl)benzenamine as a light yellow liquid. ¹H NMR (400 MHz, CDCl₃) δ 7.52 (d, 1H), 7.06 (m, 1H), 6.55 (m, 1H), 6.32 (m, 1H), 6.12 (m, 1H), 6.10 (m, 1H), 3.00 (m, 2H), 2.7 (m, 2H), 1.85 (m, 2H). LCMS: 202.0 (M+H)⁺.

### Step 5: 4-(((3-(Furan-2-yl)propyl)(phenyl)amino)methyl)quinolin-2(1H)-one

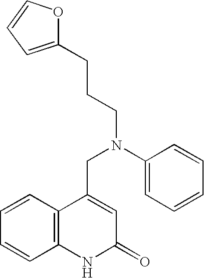

4-(((3-(Furan-2-yl)propyl)(phenyl)amino)methyl)quinolin-2(1H)-one was synthesized as described in EXAMPLE 22, Step 3 using 4-(bromomethyl)quinolin-2(1H)-one and N-(3-(furan-2-yl)propyl)benzenamine as starting materials. ¹H NMR (400 MHz, DMSO-d₆) δ 11.00 (s, 1H), 7.59 (d, 1H), 7.28 (d, 1H), 7.21 (m, 1H), 7.12 (d, 1H), 7.08 (m, 2H), 6.95 (m, 1H), 6.60 (m, 3H), 6.47 (s, 1H), 6.18 (m, 1H), 5.88 (m, 1H), 4.02 (s, 2H), 3.35 (m, 2H), 2.40 (m, 2H), 1.85 (m, 2H). LCMS: 359.0 (M+H)⁺.

### EXAMPLE 25

### N-((2-Oxo-1,2-dihydroquinolin-4-yl)methyl)-N,2-diphenylacetamide

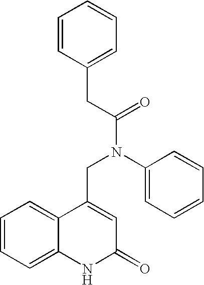

N-[(2-Oxo-1,2-dihydroquinolin-4-yl)methyl]-N-2-diphenylbenzamide was synthesized as described in EXAMPLE 1, Step 5 using 4-(anilinomethyl)-quinolin-2(1H)-one and 2-phenylacetyl chloride as starting materials. LCMS: 368 (M)⁺.

### EXAMPLE 26

### N-((2-Oxo-1,2-dihydroquinolin-4-yl)methyl)-N-phenylfuran-3-carboxamide

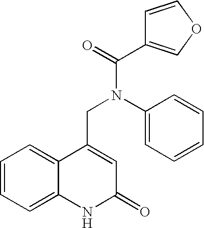

Oxalyl chloride (86 μL, 1,0 mmol) was added to a solution of furan-3-carboxylic acid (94 mg, 0.84 mmol) and DMF (7 μL, 0.084 mmol) in dry DCM (10 mL) at RT. The resulting mixture was left to stir for 2 h, after which time it was concentrated to dryness under reduced pressure, and redissolved in NMP (2 mL). The resulting solution was added at RT to a separate mixture of 4-((phenylamino)methyl)quinolin-2(1H)-one (100 mg, 0.4 mmol) and DIEA (350 μL, 2.0 mmol) in NMP (3 mL). After 14 h, the reaction mixture was treated with neat propylamine (100 μL, 1.2 mmol), stirred for an additional 30 min at RT, then purified directly via reversed-phase semi-preparative HPLC to afford 40 mg (30%) of N-((2-oxo-1,2-dihydroquinolin-4-yl)methyl)-N-phenylfuran-3-carboxamide as a white solid. ¹H NMR (400 MHz, DMSO-d₆) δ 11.70 (s, 1H), 7.81 (d, 1H), 7.51 (m, 2H), 7.29-7.38 (m, 4H), 7.22-7.19 (m, 4H), 6.29 (s, 1H), 5.99 (s, 1H), 5.25 (s, 2H). LCMS: 345.5 (M+H)⁺.

### EXAMPLE 27

N-((2-Oxo-1,2-dihydroquinolin-4-yl)methyl)-N-phenylthiazole-5-carboxamide

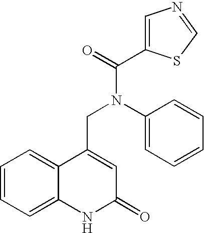

N-((2-Oxo-1,2-dihydroquinolin-4-yl)methyl)-N-phenylthiazole-5-carboxamide was synthesized as described in EXAMPLE 26 using 4-((phenylamino)methyl)quinolin-2(1H)-one and thiazole-5-carboxylic acid as starting materials. ¹H NMR (400 MHz, DMSO-d₆) δ 11.72 (s, 1H), 9.07 (s, 1H), 7.81 (d, 1H), 7.50 (m, 2H), 7.41-7.37 (m, 3H), 7.33-7.26 (m, 3H), 7.19 (dd, 1H), 6.34 (s, 1H), 5.29 (s, 2H). LCMS: 362.2 (M+H)⁺.

### EXAMPLE 28

### N-((2-Oxo-1,2-dihydroquinolin-4-yl)methyl)-N-phenylthiazole-4-carboxamide

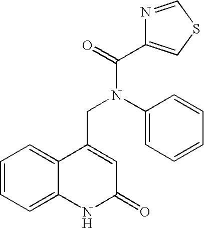

N-((2-Oxo-1,2-dihydroquinolin-4-yl)methyl)-N-phenylthiazole-4-carboxamide was synthesized as described in EXAMPLE 26 using 4-((phenylamino)methyl)quinolin-2(1H)-one and thiazole-4-carboxylic acid as starting materials. ¹H NMR (400 MHz, DMSO-d₆) δ 11.72 (s, 1H), 8.87 (s, 1H), 8.02 (s, 1H), 7.85 (d, 1H) 7.50 (dd, 1H), 7.31 (d, 1H), 7.21-7.07 (m, 6H), 6.43 (s, 1H), 5.37 (s, 2H). LCMS: 362.2 (M+H)⁺.

### EXAMPLE 29

### 4-Methyl-N-((2-oxo-1,2-dihydroquinolin-4-yl)methyl)-N-phenylthiazole-5-carboxamide

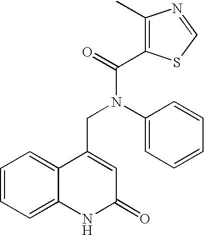

4-Methyl-N-((2-oxo-1,2-dihydroquinolin-4-yl)methyl)-N-phenylthiazole-5-carboxamide was synthesized as described in EXAMPLE 26 using 4-((phenylamino)methyl)quinolin-2(1H)-one and 4-methyl-5-thiazole carboxylic acid as starting materials. ¹H NMR (400 MHz, DMSO-d₆) δ 11.73 (s, 1H), 8.89 (s, 1H), 7.86 (d, 1H), 7.51 (dd, 1H), 7.32-7.20 (m, 5H), 7.16-7.12 (m, 2H), 6.32 (s, 1H), 5.33 (s, 2H), 2.42 (s, 3H). LCMS: 376.1 (M+H)⁺.

### EXAMPLE 30

### 3,5-Dimethyl-N-((2-oxo-1,2-dihydroquinolin-4-yl)methyl)-N-phenylisoxazole-4-carboxamide

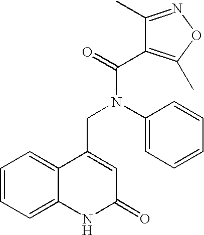

3,5-Dimethyl-N-((2-oxo-1,2-dihydroquinolin-4-yl)methyl)-N-phenylisoxazole-4-carboxamide was synthesized as described in EXAMPLE 26 using 4-((phenylamino)methyl)quinolin-2(1H)-one and 3,5-dimethylisoxazole-4-carboxylic acid as starting materials. ¹H NMR (400 MHz, DMSO-d₆) δ 11.71 (s, 1H), 7.85 (d, 1H), 7.51 (d, 1H), 7.32-7.12 (m, 7H), 6.29 (s, 1H), 5.37 (s, 2H), 2.08 (s, 3H), 2.06 (s, 3H). LCMS: 374.4 (M+H)⁺.

### EXAMPLE 31

### 5-Methyl-N-((2-oxo-1,2-dihydroquinolin-4-yl)methyl)-N-phenylisoxazole-4-carboxamide

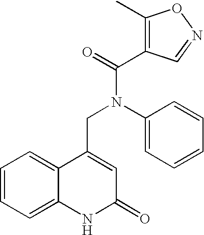

5-Methyl-N-((2-oxo-1,2-dihydroquinolin-4-yl)methyl)-N-phenylisoxazole-4-carboxamide was synthesized as described in EXAMPLE 26 using 4-((phenylamino)methyl)quinolin-2(1H)-one and 5-methylisoxazole-4-carboxylic acid as starting materials. ¹H NMR (400 MHz, DMSO-d₆) δ 11.69 (s, 1H), 7.82 (d, 1H), 7.49 (dd, 1H), 7.38-7.16 (m, 8H), 6.36 (s, 1H), 5.29 (s, 2H), 2.52 (s, 3H). LCMS: 360.6 (M+H)⁺.

### EXAMPLE 32

### N-((2-Oxo-1,2-dihydroquinolin-4-yl)methyl)-N-phenylpicolinamide

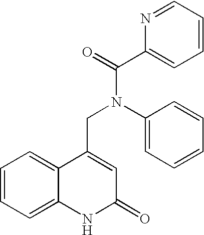

N-((2-Oxo-1,2-dihydroquinolin-4-yl)methyl)-N-phenylpicolinamide was synthesized as described in EXAMPLE 26 using 4-((phenylamino)methyl)quinolin-2(1H)-one and picolinic acid as starting materials. ¹H NMR (400 MHz, DMSO-d₆) δ 11.73 (s, 1H), 8.31 (d, 1H), 7.86 (d, 1H), 7.78 (dd, 1H), 7.57 (d, 1H), 7.52 (dd, 1H), 7.34-7.04 (m, 8H), 6.52 (s, 1H), 5.36 (s, 2H). LCMS: 356.1 (M+H)⁺.

### EXAMPLE 33

### N-((2-Oxo-1,2-dihydroquinolin-4-yl)methyl)-N-phenylnicotinamide

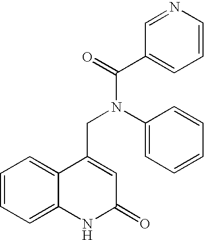

N-((2-Oxo-1,2-dihydroquinolin-4-yl)methyl)-N-phenylnicotinamide was synthesized as described in EXAMPLE 26 using 4-((phenylamino)methyl)quinolin-2(1H)-one and nicotinic acid as starting materials. ¹H NMR (400 MHz, DMSO-d₆) δ 11.73 (s, 1H), 8.56 (s, 1H), 8.52 (d, 1H), 7.84 (m, 2H), 7.51 (dd, 1H), 7.41 (m, 1H), 7.31 (d, 1H), 7.24-7.14 (m, 6H), 6.46 (s, 1H), 5.38 (s, 2H). LCMS: 356.2 (M+H)⁻.

### EXAMPLE 34

### N-((2-Oxo-1,2-dihydroquinolin-4-yl)methyl)-N-phenylisonicotinamide

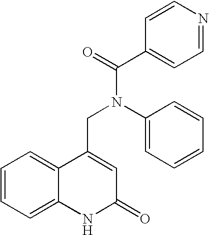

N-((2-Oxo-1,2-dihydroquinolin-4-yl)methyl)-N-phenylisonicotinamide was synthesized as described in EXAMPLE 26 using 4-((phenylamino)methyl)quinolin-2(1H)-one and isonicotinic acid as starting materials. ¹H NMR (400 MHz, DMSO-d₆) δ 11.71 (s, 1H), 8.45 (d, 2H), 7.84 (d, 1H), 7.51 (dd, 1H), 7.31 (d, 1H), 7.26-7.11 (m, 8H), 6.40 (s, 1H), 5.35 (s, 2H). LCMS: 356.1 (M+H)⁺.

### EXAMPLE 35

### 5-Methyl-N-((2-oxo-1,2-dihydroquinolin-4-yl)methyl)-N-phenylpyrazine-2-carboxamide

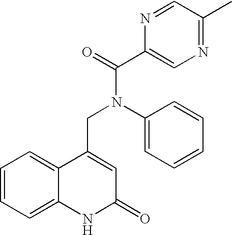

5-Methyl-N-((2-oxo-1,2-dihydroquinolin-4-yl)methyl)-N-phenylpyrazine-2-carboxamide was synthesized as described in EXAMPLE 26 using 4-((phenylamino)methyl)quinolin-2(1H)-one and 5-methyl-2-pyrazinecarboxylic acid as starting materials. ¹H NMR (400 MHz, DMSO-d₆) δ 11.73 (s, 1H), 8.70 (s, 1H), 8.28 (s, 1H), 7.85 (d, 1H), 7.51 (dd, 1H), 7.31 (d, 1H), 7.21-7.07 (m, 6H), 6.50 (s, 1H), 5.38 (s, 2H), 2.40 (s, 3H). LCMS: 371.5 (M+H)⁺.

### EXAMPLE 36

### N-((2-Oxo-1,2-dihydroquinolin-4-yl)methyl)-N-phenylpyrazine-2-carboxamide

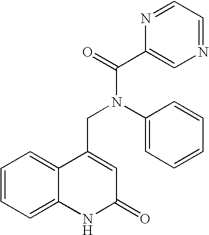

N-((2-Oxo-1,2-dihydroquinolin-4-yl)methyl)-N-phenylpyrazine-2-carboxamide was synthesized as described in EXAMPLE 26 using 4-((phenylamino)methyl)quinolin-2(1H)-one and pyrazinecarboxylic acid as starting materials. ¹H NMR (400 MHz, DMSO-d₆) δ 11.73 (s, 1H), 8.84 (s, 1H), 8.54 (s, 1H), 8.41 (s, 1H), 7.86 (d, 1H), 7.50 (dd, 1H), 7.31 (d, 1H), 7.08-7.25 (m, 6H), 6.51 (s, 1H), 5.39 (s, 2H). LCMS: 357.5 (M+H)⁺.

### EXAMPLE 37

### 2-Methyl-N-((2-oxo-1,2-dihydroquinolin-4-yl)methyl)-N-phenylnicotinamide

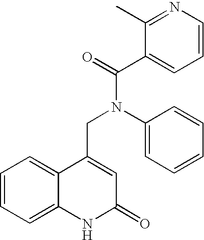

2-Methyl-N-((2-oxo-1,2-dihydroquinolin-4-yl)methyl)-N-phenylnicotinamide was synthesized as described in EXAMPLE 26 using 4-((phenylamino)methyl)quinolin-2(1H)-one and 2-methylnicotinic acid as starting materials. ¹H NMR (400 MHz, DMSO-d₆) δ 11.75 (s, 1H), 8.46 (d, 1H), 7.90 (m, 2H), 7.52 (dd, 1H), 7.32 (m, 2H), 7.24 (dd, 1H), 7.20-7.06 (m, 5H), 6.36 (s, 1H), 5.39 (s, 2H), 2.58 (s, 3H). LCMS: 370.1 (M+H)⁺.

### EXAMPLE 38

### 3-Methyl-N-((2-oxo-1,2-dihydroquinolin-4-yl)methyl)-N-phenylpicolinamide

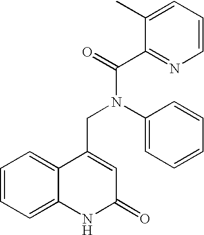

3-Methyl-N-((2-oxo-1,2-dihydroquinolin-4-yl)methyl)-N-phenylpicolinamide was synthesized as described in EXAMPLE 26 using 4-((phenylamino)methyl)quinolin-2(1H)-one and 3-methylpicolinic acid as starting materials. ¹H NMR (400 MHz, DMSO-d₆) δ 11.75 (s, 1H), 8.29 (d, 1H), 7.90 (d, 1H), 7.72 (d, 1H), 7.52 (dd, 1H), 7.36-7.28 (m, 2H), 7.23 (dd, 1H), 7.03-7.16 (m, 5H), 6.51 (s, 1H), 5.40 (s, 2H), 2.30 (s, 3H). LCMS: 370.2 (M+H)⁺.

### EXAMPLE 39

### N-(3-Chloro-4-fluorophenyl)-4-methyl-N-((2-oxo-1,2-dihydroquinolin-4-yl)methyl)thiazole-5-carboxamide

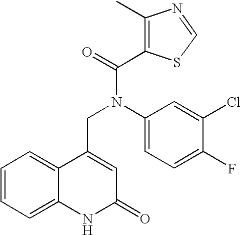

### Step 1: 4-((3-Chloro-4-fluorophenylamino)methyl)quinolin-2(1H)-one

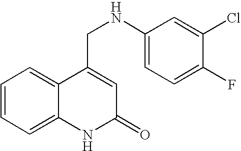

4-((3-chloro-4-fluorophenylamino)methyl)quinolin-2(1H)-one was synthesized as described in EXAMPLE 1, step 4 using 4-(bromomethyl)quinolin-2(1H)-one and 3-chloro-4-fluoroaniline as starting materials. LCMS: 303 (M+H)⁺.

### Step 2: N-(3-Chloro-4-fluorophenyl)-4-methyl-N-((2-oxo-1,2-dihydroquinolin-4-yl)methyl)thiazole-5-carboxamide

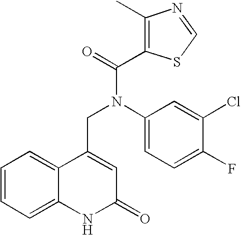

N-(3-Chloro-4-fluorophenyl)-4-methyl-N-((2-oxo-1,2-dihydroquinolin-4-yl)methyl)thiazole-5-carboxamide was synthesized as described in EXAMPLE 26 using 4-((3-chloro-4-fluorophenylamino)methyl)quinolin-2(1H)-one and 4-methylthiazole-5-carboxylic acid as starting materials. LCMS: 428 (M+H)⁺.

### EXAMPLE 40

### 4-Methyl-N-((2-oxo-1,2-dihydroquinolin-4-yl)methyl)-N-(3-(trifluoromethyl)phenyl)thiazole-5-carboxamide

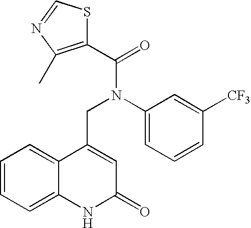

4-Methyl-N-((2-oxo-1,2-dihydroquinolin-4-yl)methyl)-N-(3-(trifluoromethyl)phenyl)thiazole-5-carboxamide was synthesized as described in EXAMPLE 39 using 4-(bromomethyl)quinolin-2(1H)-one, 3-(trifluoromethyl)aniline, and 4-methylthiazole-5-carboxylic acid as starting materials. LCMS: 443 (M)⁺.

### EXAMPLE 41

### 4-Methyl-N-((2-oxo-1,2-dihydroquinolin-4-yl)methyl)-N-(3,3-difluorophenyl)thiazole-5-carboxamide

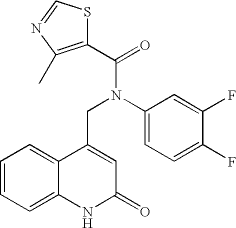

4-Methyl-N-((2-oxo-1,2-dihydroquinolin-4-yl)methyl)-N-(3,3-difluorophenyl)thiazole-5-carboxamide was synthesized as described in EXAMPLE 39 using 4-(bromomethyl)quinolin-2(1H)-one, 3,4-difluoroaniline, and 4-methylthiazole-5-carboxylic acid as starting materials. LCMS: 411 (M)⁺.

### EXAMPLE 42

### N-(3-Chlorophenyl)-N-((8-fluoro-2-oxo-1,2-dihydroquinolin-4-yl)methyl)-4-methylthiazole-5-sulfonamide

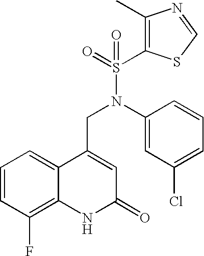

### Step 1: 4-methylthiazole-5-sulfonyl chloride

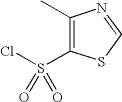

Sulfurochloridic acid (50 mL) was added dropwise to 4-methylthiazole (10 g, 99.85 mmol) with stirring, while maintaining the temperature at RT followed by addition of PCl₅ (10 g, 47.54 mmol). The resulting solution was allowed to react, with stirring, for 4 h while the temperature was maintained at 140° C. (reaction progress monitored by TLC (EtOAc/PE=1:1)). The reaction mixture was then quenched by the adding 500 g of ice/salt. The resulting solution was extracted with EtOAc (3×500 mL), the organics were combined, and dried over Na₂SO₄. The solvent was removed to afford 2.5 g (12%) of 4-methylthiazole-5-sulfonyl chloride as a brown solid.

### Step 2: N-(3-Chlorophenyl)-4-methylthiazole-5-sulfonamide

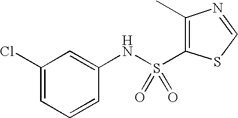

A mixture 4-methylthiazole-5-sulfonyl chloride (500 mg, 2.54 mmol) 3-chloroaniline (320 mg, 2.52 mmol), and pyridine (30 mL) heated to 50° C. for 5 h. The reaction progress was monitored by TLC (EtOAc/PE=1:1). The mixture was concentrated by evaporation and the residue was purified by column chromatography on silica gel eluting with a 1:5 EtOAc/PE solvent system. This resulted in 0.4 g (55%) of N-(3-chlorophenyl)-4-methylthiazole-5-sulfonamide as a white solid.

### Step 3: N-(3-Chlorophenyl)-N-((8-fluoro-2-oxo-1,2-dihydroquinolin-4-yl)methyl)-4-methylthiazole-5-sulfonamide

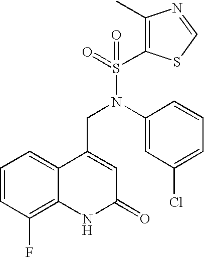

A mixture of N-(3-chlorophenyl)-4-methylthiazole-5-sulfonamide (370 mg, 1.28 mmol), 4-(bromomethyl)-8-fluoroquinolin-2(1H)-one (328 mg, 1.29 mmol), and K₂CO₃ (0.178 g, 1.29 mmol) in DMF (30 mL) was heated to 60° C. until the reaction was done (reaction progress monitored by TLC (EtOAc/PE=1:1). The mixture was concentrated, the residue was dissolved in EtOAc (200 mL), washed with water (2×50 mL), and dried over Na₂SO₄. The crude was purified by column chromatography on silica gel (eluting with 1:1 EtOAc/PE solvent system). This resulted in 100 mg (17%) of N-(3-chlorophenyl)-N-((8-fluoro-2-oxo-1,2-dihydroquinolin-4-yl)methyl)-4-methylthiazole-5-sulfonamide as a white solid. ¹H NMR (300 MHz, DMSO-d₆) δ 11.71 (s, 1H), 9.39 (s, 1H), 7.83 (d, 1H), 7.18-7.48 (m, 6H), 6.52 (s, 1H), 5.15 (s, 2H), 2.28 (s, 3H). LCMS: 464 (M+H)⁺.

### EXAMPLE 43

### N-(3-Chlorophenyl)-N-((8-fluoro-2-oxo-1,2-dihydroquinolin-4-yl)methyl)-4-methylthiazole-5-carboxamide

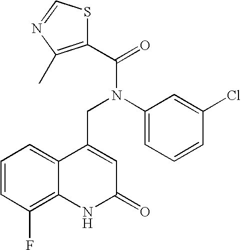

### Step 1: N-(2-Fluorophenyl)-3-oxobutanamide

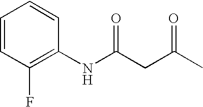

2-Fluoroaniline (40.0 g, 0.36 mol) was combined at room temperature with methyl acetoacetate (54 mL, 0.50 mol) in a 500 mL round bottom flask containing a stir bar. The flask was capped with a reflux condenser and heated with stirring to 140° C. After 18 hrs, the mixture was cooled to room temperature and diluted with 400 mL diethyl ether. The resulting solution was washed with water (100 mL), 1N HCl (50 mL), and brine (50 mL), then dried over MgSO₄, filtered and concentrated under reduced pressure. The resulting crude mixture was then purified by silica gel column chromatography, eluting with 40% EtOAc in hexanes to afford the product N-(2-fluorophenyl)-3-oxobutanamide (27.21 g, 38%) as a white crystalline solid. LCMS: 196.1 (M+H)⁺.

### Step 2: 4-Bromo-N-(2-fluorophenyl)-3-oxobutanamide

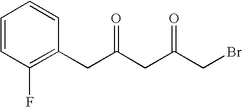

N-(2-Fluorophenyl)-3-oxobutanamide (27.2 g, 139.5 mmol) was stirred at room temperature in glacial AcOH (70 mL). To this was added (dropwise via addition funnel over 20 min) a solution of Br₂ (7-9 mL, 1.1 eq) and 12 (1.4 g, 5.6 mmol) in AcOH (110 mL). After 5 h of monitoring by TLC, the reaction was determined to have stalled with significant product formation. The mixture was then concentrated to ˜20% volume, and worked-up via EtOAc/H₂O extraction. Purification by column chromatography on silica gel column (10% to 20% EtOAc in hexanes) afforded 26 g (68% yield) of 4-bromo-N-(2-fluorophenyl)-3-oxobutanamide.

### Step 3: 4-(Bromomethyl)-8-fluoroquinolin-2(1H)-one

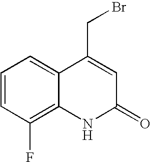

4-(Bromomethyl)-8-fluoroquinolin-2(1H)-one was synthesized as described in EXAMPLE 1, Step 3 using 4-bromo-N-(2-fluorophenyl)-3-oxobutanamide as a starting material.

### Step 4: 4-((3-Chlorophenylamino)methyl)-8-fluoroquinolin-2(1H)-one

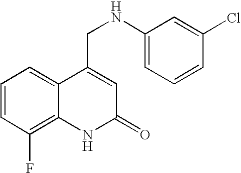

To a stirred solution of 3-chloroaniline (1.68 mL, 16.0 mmol) in DMSO (30 mL) at 50° C. was added solid 4-(bromomethyl)-8-fluoroquinolin-2(1H)-one (1.09 g, 4.0 mmol) in one portion. Solid material dissolved immediately. After 40 min, LCMS and TLC analysis showed full product conversion. The reaction was cooled to RT and poured in to 250 mL of ice/H₂O slurry. The resulting product precipitate was collected via vacuum filtration. The filer cake was washed with 3×10 mL portions of 0.1 N HCl and 1×50 mL portion of H₂O to afford 4-((3-chlorophenylamino)methyl)-8-fluoroquinolin-2(1H)-one.

### Step 5: N-(3-Chlorophenyl)-N-((8-fluoro-2-oxo-1,2-dihydroquinolin-4-yl)methyl)-4-methylthiazole-5-carboxamide

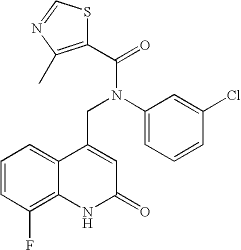

N-(3-Chlorophenyl)-N-((8-fluoro-2-oxo-1,2-dihydroquinolin-4-yl)methyl)-4-methylthiazole-5-carboxamide was synthesized as described in EXAMPLE 26 using 4-((3-chlorophenylamino)methyl)-8-fluoroquinolin-2(1H)-one and 4-methylthiazole-5-carboxylic acid as starting materials. ¹H NMR (400 MHz, DMSO-d₆) δ 11.76 (s, 1H), 8.93 (s, 1H), 7.65 (d, 1H), 7.47-7.41 (m, 2H), 7.30-7.19 (m, 3H), 7.07 (d, 1H), 6.43 (s, 1H), 5.35 (s, 2H), 2.42 (s, 3H). LCMS: 428.1 (M+H)⁺.

### EXAMPLE 44

### N-(3-Chlorophenyl)-N-((8-fluoro-2-oxo-1,2-dihydroquinolin-4-yl)methyl)thiazole-5-carboxamide

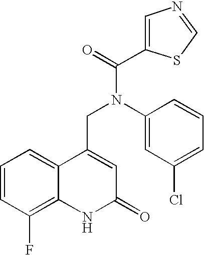

N-(3-Chlorophenyl)-N-((8-fluoro-2-oxo-1,2-dihydroquinolin-4-yl)methyl)thiazole-5-carboxamide was synthesized as described in EXAMPLE 26 using 4-((3-chlorophenylamino)methyl)-8-fluoroquinolin-2(1H)-one and thiazole-5-carboxylic acid as starting materials. ¹H NMR (400 MHz, DMSO-d₆) δ 11.75 (s, 1H), 9.11 (s, 1H), 7.67-7.57 (m, 3H), 7.47-7.37 (m, 3H), 7.25-7.18 (m, 2H), 6.47 (s, 1H), 5.30 (s, 2H). LCMS: 413.8 (M+H)⁺.

### EXAMPLE 45

### 4-(((3-Chlorophenyl)((4-methylthiazol-5-yl)methyl)amino)methyl)-8-fluoroquinolin-2(1H)-one

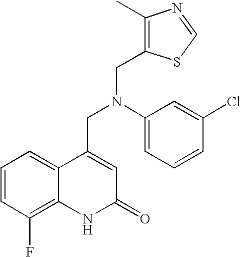

### Step 1: N-(3-Chlorophenyl)-4-methylthiazole-5-carboxamide

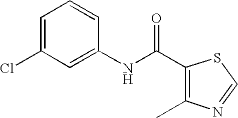

Oxalyl chloride (510 μL, 5.85 mmol) was added to a solution of 4-methylthiazole-5-carboxylic acid (643 mg, 4.5 mmol) and DMF (35 μL, 0.50 mmol) in dry DCM (30 mL) at RT. The resulting mixture was left to stir for 2 h, after which time it was concentrated to dryness under reduced pressure, and redissolved in NMP (2 mL). The resulting solution was added to a separate mixture of 3-chloroaniline (474 μL, 4.5 mmol), triethylamine (1.7 mL, 12.0 mmol), and DMAP (cat., ˜30 mg) in dry DCM (40 mL) at RT. The reaction was monitored and determined to be complete by TLC after 2 h, at which time it was diluted with 1:1 hexanes:EtOAc (400 mL) and poured in to a separatory funnel. The resulting crude mixture was washed with 5% brine (3×50 mL), 1N HCl (50 mL), and H₂O (100 mL), then dried over MgSO₄, filtered, and concentrated to dryness under reduced pressure to afford N-(3-chlorophenyl)-4-methylthiazole-5-carboxamide (1.04 g, 91%) as a tan solid that was used in the next step without further purification.

### Step 2: 3-Chloro-N-((4-methylthiazol-5-yl)methyl)aniline

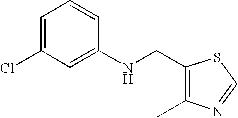

Lithium aluminum hydride (200 mg, 5.26 mmol) was added to a stirred solution of N-(3-chlorophenyl)-4-methylthiazole-5-carboxamide (708 mg, 2.8 mmol) in dry THF at RT. The reaction was warmed to reflux and its progress was monitored by TLC analysis. After 2 h, the mixture was cooled to RT and diluted with DCM (500 mL). To this was added sodium sulfate decahydrate (˜80 g), and the resulting slurry was stirred vigorously for 2 hrs. The resulting liquid was decanted, dried over MgSO₄, filtered, and concentrated to dryness under reduced pressure. The resulting crude residue was filtered through a plug of silica gel, eluting with 70% EtOAc in hexanes to afford 3-chloro-N-((4-methylthiazol-5-yl)methyl)aniline (463 mg, 70%) as a tan solid.

### Step 3: 4-(((3-Chlorophenyl)((4-methylthiazol-5-yl)methyl)amino)methyl)-8-fluoroquinolin-2(1H)-one

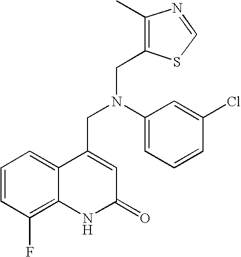

3-Chloro-N-((4-methylthiazol-5-yl)methyl)aniline (45 mg, 0.19 mmol), potassium iodide (16 mg, 0.1 mmol), and 4-(bromomethyl)-8-fluoroquinolin-2(1H)-one (25 mg, 0.1 mmol) were combined in dry DMSO (2 mL) at RT. The mixture was warmed to 90° C. and left to stir for 4 h, after which time the reaction was determined to be complete by TLC analysis. The cooled mixture was poured in to water (50 mL) and partitioned with EtOAc (50 mL). The aqueous layer was washed with EtOAc (3×50 mL each). The combined organic extracts were then dried over MgSO₄, filtered, and concentrated to dryness under reduced pressure. The resulting crude residue was purified by silica gel column chromatography, eluting with 40% acetonitrile in DCM to afford 4-(((3-chlorophenyl)((4-methylthiazol-5-yl)methyl)amino)methyl)-8-fluoroquinolin-2(1H)-one (12 mg, 30%) as a tan solid. ¹H NMR (400 MHz, CDCl₃) δ 10.04 (s, 1H), 8.66 (s, 1H), 7.43-7.11 (m, 4H), 6.79 (d, 1H), 6.69-6.55 (m, 3H), 4.73 (s, 2H), 4.70 (s, 2H), 2.43 (s, 3H). LCMS: 413.9 (M+H)⁺.

### EXAMPLE 46

### N-(3-Chlorophenyl)-N-((8-fluoro-2-oxo-1,2-dihydroquinolin-4-yl)methyl)-1-methyl-1H-imidazole-4-carboxamide

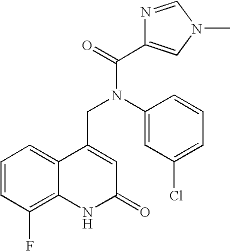

### Intermediate A

### 2-(tert-Butyldimethylsilyloxy)-8-fluoro-4-(iodomethyl)quinoline

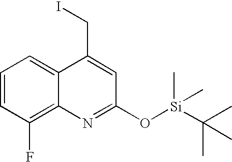

### Step 1: 2-(tert-Butyldimethylsilyloxy)-4-(chloromethyl)-8-fluoroquinoline

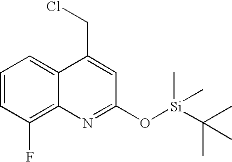

To a stirred solution of 4-(bromomethyl)-8-fluoroquinolin-2(1H)-one (1.28 g, 5.0 mmol) in DMF (50 mL) at room temperature was added tert-butyldimethylsilyl chloride (1.51 g, 10.0 mmol) followed by triethylamine (2.4 mL, 17.5 mmol). After 4 hrs, the reaction mixture was poured in to ice H₂O (500 mL), and the resulting precipitate was collected by vacuum filtration. The filter cake was washed with an additional 100 mL portion of H₂O, then left to dry in a vacuum dessicator for 18 hrs to afford 2-(tert-butyldimethylsilyloxy)-4-(chloromethyl)-8-fluoroquinoline (1.42 g, 88%) as a tan solid.

### Step 2: 2-(tert-Butyldimethylsilyloxy)-8-fluoro-4-(iodomethyl)quinoline

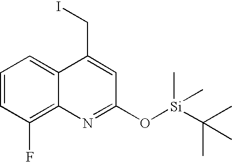

Sodium iodide (157 mg, 1.05 mmol) was added to a stirred solution of 2-(tert-butyldimethylsilyloxy)-4-(chloromethyl)-8-fluoroquinoline (325 mg, 1.0 mmol) in dry acetone at RT. After 2 h, the heterogeneous mixture was diluted with DCM (200 mL), dried over MgSO₄, filtered, and concentrated under reduced pressure to afford 2-(tert-butyldimethylsilyloxy)-8-fluoro-4-(iodomethyl)quinoline (390 mg, 94%) as an orange solid.

### Intermediate B

### N-(3-Chlorophenyl)-1-methyl-1H-imidazole-4-carboxamide

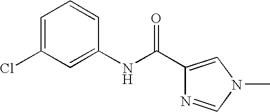

O-(7-Azabenzotriazol-1-yl)-N,N,N′,N′-tetramethyluronium hexafluorophosphate (HATU, 912 mg, 2.4 mmol) was added to a stirred mixture of 1-methyl-1H-imidazole-4-carboxylic acid (252 mg, 2.0 mmol), 3-chloroaniline (273 μL, 2.6 mmol), and triethylamine (1.1 mL, 8.0 mmol) in DMF (10 mL). After 18 h, the reaction mixture was diluted with 1:1 hexanes:EtOAc (200 mL), washed with 5% brine (3×50 mL portions), dried over MgSO₄, filtered and concentrated under reduced pressure. The resulting crude residue was purified by silica gel column chromatography, eluting with 10% acetonitrile in DCM to afford N-(3-Chlorophenyl)-1-methyl-1H-imidazole-4-carboxamide (358 mg, 76%) as a tan solid.

### SYNTHESIS OF EXAMPLE 46

### N-(3-Chlorophenyl)-N-((8-fluoro-2-oxo-1,2-dihydroquinolin-4-yl)methyl)-1-methyl-1H-imidazole-4-carboxamide

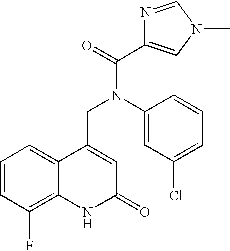

Sodium hydride (19 mg, 0.50 mmol) was added to a stirred solution of Intermediate B (100 mg, 0.42 mmol) in DMF (6 mL) at RT. After 1 h, Intermediate A (208 mg, 0.50 mmol) was added in one portion. The resulting mixture was stirred for 2.5 h, then diluted with 1:1 hexanes:EtOAc (100 mL), washed with water (50 mL), 5% NaHCO₃ (50 mL), and brine, then dried over MgSO₄, filtered and concentrated under reduced pressure. The crude residue was purified by automated reversed-phase semi-preparative HPLC to afford N-(3-chlorophenyl)-N-((8-fluoro-2-oxo-1,2-dihydroquinolin-4-yl)methyl)-1-methyl-1H-imidazole-4-carboxamide (29 mg, 17%) as a white solid. ¹H NMR (400 MHz, DMSO-d₆) δ 11.74 (s, 1H), 8.42 (s, 1H), 7.61 (d, 1H), 7.50-7.33 (m, 4H), 7.21-7.17 (m, 2H), 6.96 (s, 1H), 6.45 (s, 1H), 5.37 (s, 2H), 3.63 (s, 3H). LCMS: 411.1 (M+H)⁺.

### EXAMPLE 47

### N-(3-Chlorophenyl)-N-((8-fluoro-2-oxo-1,2-dihydroquinolin-4-yl)methyl)-1-methyl-1H-imidazole-2-carboxamide

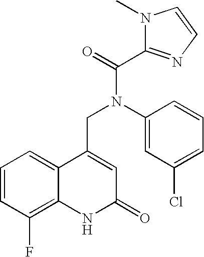

N-(3-Chlorophenyl)-N-((8-fluoro-2-oxo-1,2-dihydroquinolin-4-yl)methyl)-1-methyl-1H-imidazole-2-carboxamide was synthesized as described in EXAMPLE 46 using 1-methyl-1H-imidazole-2-carboxylic acid as starting material in the synthesis of Intermediate B. ¹H NMR (400 MHz, DMSO-d₆) δ 11.75 (s, 1H), 7.65 (d, 1H), 7.43 (dd, 1H), 7.35 (s, 1H), 7.26-7.18 (m, 4H), 7.07 (d, 1H), 6.76 (s, 1H), 6.51 (s, 1H), 5.43 (s, 2H), 3.85 (s, 3H). LCMS: 411.2 (M+H)⁺.

### EXAMPLE 48

### N-(3-Chlorophenyl)-N-((8-fluoro-2-oxo-1,2-dihydroquinolin-4-yl)methyl)-1-methyl-1H-imidazole-5-carboxamide

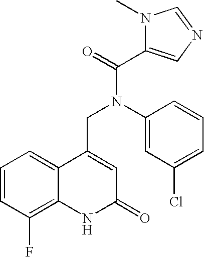

N-(3-Chlorophenyl)-N-((8-fluoro-2-oxo-1,2-dihydroquinolin-4-yl)methyl)-1-methyl-1H-imidazole-5-carboxamide was synthesized as described in EXAMPLE 46 using 1-methyl-1H-imidazole-5-carboxylic acid as starting material in the synthesis of Intermediate B. ¹H NMR (400 MHz, DMSO-d₆) δ 11.74 (s, 1H), 7.70 (s, 1H), 7.63 (d, 1H), 7.52 (s, 1H), 7.44 (dd, 1H), 7.39-7.31 (m, 2H), 7.16-7.27 (m, 2H), 6.47 (s, 1H), 6.17 (s, 1H), 5.28 (s, 2H), 3.83 (s, 3H). LCMS: 411.2 (M+H)⁺.

### EXAMPLE 49

### N-((8-Chloro-2-oxo-1,2-dihydroquinolin-4-yl)methyl)-N-(3-chlorophenyl)-4-methylthiazole-5-carboxamide

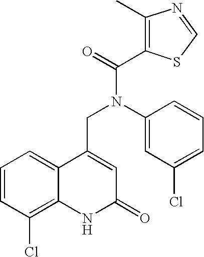

N-((8-Chloro-2-oxo-1,2-dihydroquinolin-4-yl)methyl)-N-(3-chlorophenyl)-4-methylthiazole-5-carboxamide was synthesized as described in EXAMPLE 43 using 2-chloroaniline as starting material. ¹H NMR (400 MHz, DMSO-d₆) δ 10.97 (s, 1H), 8.92 (s, 1H), 7.82 (d, 1H), 7.68 (d, 1H), 7.47 (s, 1H), 7.32-7.23 (m, 3H), 7.08 (d, 1H), 6.45 (s, 1H), 5.36 (s, 2H), 2.42 (s, 3H). LCMS: 444 (M+H)⁺.

### EXAMPLE 50

### N-(3-Chlorophenyl)-N-((8-fluoro-5-methyl-2-oxo-1,2-dihydroquinolin-4-yl)methyl)-4-methylthiazole-5-carboxamide

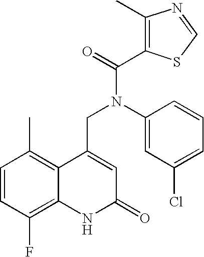

N-(3-Chlorophenyl)-N-((8-fluoro-5-methyl-2-oxo-1,2-dihydroquinolin-4-yl)methyl)-4-methylthiazole-5-carboxamide was synthesized as described in EXAMPLE 43 using 2-fluoro-5-methylaniline as starting material. ¹H NMR (400 MHz, DMSO-d₆) δ 11.59 (s, 1H), 8.98 (s, 1H), 7.54 (s, 1H), 7.35-7.25 (m, 4H), 7.00 (d, 1H), 6.47 (s, 1H), 5.49 (s, 2H), 2.74 (s, 3H), 2.45 (s, 3H). LCMS: 442 (M+H)⁺.

### EXAMPLE 51

### N-(3-Chlorophenyl)-N-((7-fluoro-2-oxo-1,2-dihydroquinolin-4-yl)methyl)-4-methylthiazole-5-carboxamide

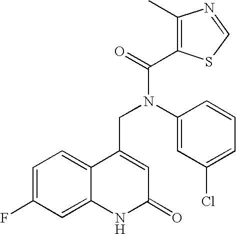

N-(3-Chlorophenyl)-N-((7-fluoro-2-oxo-1,2-dihydroquinolin-4-yl)methyl)-4-methylthiazole-5-carboxamide was synthesized as described in EXAMPLE 43 using 3-fluoroaniline as starting material. ¹H NMR (400 MHz, DMSO-d₆) δ 11.83 (s, 1H), 8.94 (s, 1H), 7.90 (t, 1H), 7.46 (s, 1H), 7.30 (m, 2H), 7.13-7.06 (m, 3H), 6.36 (s, 1H), 5.35 (s, 2H).

### EXAMPLE 52

### N-(3-Chlorophenyl)-N-((5,8-difluoro-2-oxo-1,2-dihydroquinolin-4-yl)methyl)-4-methylthiazole-5-carboxamide

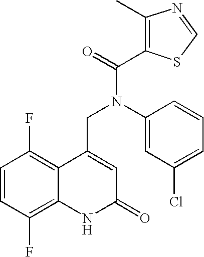

N-(3-Chlorophenyl)-N-((5,8-difluoro-2-oxo-1,2-dihydroquinolin-4-yl)methyl)-4-methylthiazole-5-carboxamide was synthesized as described in EXAMPLE 43 using 2,5-difluoroaniline as starting material. Note: Step 3 was done using PPA instead of H₂SO₄. This resulted in the formation of two cyclized products that were not separated until the last step. The separation was done by preparative HPLC and the desired product was the minor one of the two. ¹H NMR (400 MHz, DMSO-d₆) δ 11.95 (s, 1H), 8.97 (s, 1H), 7.55 (s, 1H), 7.48 (m, 1H), 7.31 (m, 2H), 7.25 (m, 1H), 7.06 (m, 1H), 6.51 (s, 1H), 5.33 (s, 2H), 2.44 (s, 3H). LCMS: 445 (M)⁺.

### EXAMPLE 53

### N-(3-Chlorophenyl)-N-((7,8-difluoro-2-oxo-1,2-dihydroquinolin-4-yl)methyl)-4-methylthiazole-5-carboxamide

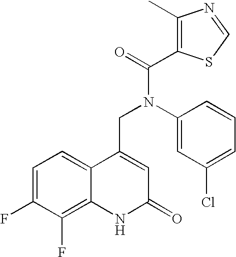

N-(3-Chlorophenyl)-N-((7,8-difluoro-2-oxo-1,2-dihydroquinolin-4-yl)methyl)-4-methylthiazole-5-carboxamide was synthesized as described in EXAMPLE 43 using 2,3-difluoroaniline as starting material. ¹H NMR (400 MHz, DMSO-d₆) δ 12.03 (s, 1H), 8.95 (s, 1H), 7.70 (m, 1H), 7.49 (s, 1H), 7.37-7.28 (m, 3H), 7.09 (d, 1H), 6.39 (s, 1H), 5.35 (s, 2H), 2.43 (s, 3H). LCMS: 446 (M+H)⁺.

### EXAMPLE 54

### N-(3-Chlorophenyl)-2-fluoro-N-((8-fluoro-2-oxo-1,2-dihydroquinolin-4-yl)methyl)benzamide

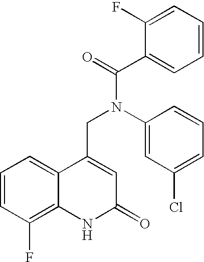

To a stirred solution of 4-((3-chlorophenylamino)methyl)-8-fluoroquinolin-2(1H)-one (0.5 mmol, 150 mg) in N-methyl-2-pyrrolidinone (NMP, 4 mL) at 0° C. was added DIEA (2.5 mmol, 0.4 mL), followed by slow addition of 2-fluorobenzoyl chloride (1.5 mmol, 0.18 mL). The reaction was stirred for 10 min at 0° C., then 14 h at RT. The reaction was again cooled to 0° C., at which time propylamine (1.5 mmol, 0.12 mL) was slowly added via syringe. The cold bath was removed and the reaction mixture was stirred at RT for 1 h. The reaction mixture was poured into EtOAc/Hexane (1:1, 20 mL) and the solution was washed with brine (5×20 mL). The residue obtained after evaporation of the solvent was purified by reverse phase chromatography to afford 66 mg of N-(3-chlorophenyl)-2-fluoro-N-((8-fluoro-2-oxo-1,2-dihydroquinolin-4-yl)methyl)benzamide as a white solid. ¹H NMR (400 MHz, DMSO-d₆) δ 11.75 (s, 1H), 7.68 (d, 1H), 7.50-7.31 (m, 4H), 7.22-7.17 (m, 4H), 7.11 (t, 1H), 7.01 (s, 1H), 6.51 (s, 1H), 5.37 (s, 2H). LCMS: 425 (M+H)⁺.

### EXAMPLE 55

### N-(3-Chlorophenyl)-3-fluoro-N-((8-fluoro-2-oxo-1,2-dihydroquinolin-4-yl)methyl)benzamide

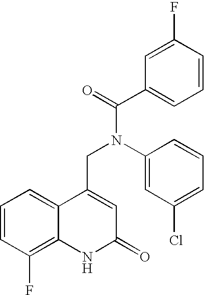

N-(3-Chlorophenyl)-3-fluoro-N-((8-fluoro-2-oxo-1,2-dihydroquinolin-4-yl)methyl)benzamide was synthesized as described in EXAMPLE 54 using 4-((3-chlorophenylamino)methyl)-8-fluoroquinolin-2(1H)-one and 3-fluorobenzoyl chloride as starting materials. ¹H NMR (400 MHz, DMSO-d₆) δ 11.75 (s, 1H), 7.67 (d, 1H), 7.46-7.42 (m, 2H), 7.33-7.14 (m, 7H), 7.08 (s, 1H), 6.51 (s, 1H), 5.37 (s, 2H). LCMS: 425 (M+H)⁺.

### EXAMPLE 56

### N-(3-Chlorophenyl)-4-fluoro-N-((8-fluoro-2-oxo-1,2-dihydroquinolin-4-yl)methyl)benzamide

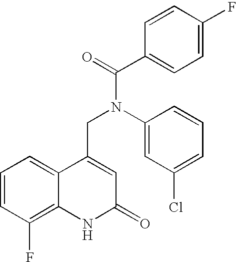

N-(3-Chlorophenyl)-4-fluoro-N-((8-fluoro-2-oxo-1,2-dihydroquinolin-4-yl)methyl)benzamide was synthesized as described in EXAMPLE 54 using 4-((3-chlorophenylamino)methyl)-8-fluoroquinolin-2(1H)-one and 4-fluorobenzoyl chloride as starting materials. ¹H NMR (400 MHz, DMSO-d₆) δ 11.75 (s, 1H), 7.68 (d, 1H), 7.46-7.39 (m, 4H), 7.24-7.21 (m, 4H), 7.13 (t, 1H), 7.04 (s, 1H), 6.50 (s, 1H), 5.37 (s, 2H). LCMS: 425 (M+H)⁺.

### EXAMPLE 57

### N-(3-Chlorophenyl)-N-((8-fluoro-2-oxo-1,2-dihydroquinolin-4-yl)methyl)-2-methylbenzamide

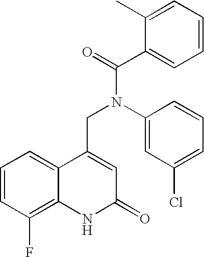

N-(3-Chlorophenyl)-N-((8-fluoro-2-oxo-1,2-dihydroquinolin-4-yl)methyl)-2-methylbenzamide was synthesized as described in EXAMPLE 54 using 4-((3-chlorophenylamino)methyl)-8-fluoroquinolin-2(1H)-one and 2-methylbenzoyl chloride as starting materials. ¹H NMR (400 MHz, DMSO-d₆) δ 11.75 (s, 1H), 7.73 (s, 1H), 7.45 (t, 1H), 7.30-7.26 (m, 2H), 7.14 (m, 5H), 7.02 (m, 1H), 6.92 (s, 1H), 6.44 (s, 1H), 5.41 (s, 2H), 2.30 (s, 3H).

### EXAMPLE 58

### N-(3-Chlorophenyl)-N-((8-fluoro-2-oxo-1,2-dihydroquinolin-4-yl)methyl)-3-methylbenzamide

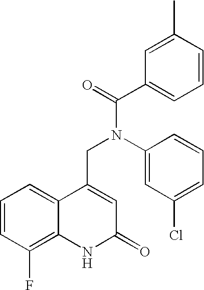

N-(3-Chlorophenyl)-N-((8-fluoro-2-oxo-1,2-dihydroquinolin-4-yl)methyl)-3-methylbenzamide was synthesized as described in EXAMPLE 54 using 4-((3-chlorophenylamino)methyl)-8-fluoroquinolin-2(1H)-one and 3-methylbenzoyl chloride as starting materials. ¹H NMR (400 MHz, DMSO-d₆) δ 11.75 (s, 1H), 7.68 (d, 1H), 7.46 (t, 1H), 7.35 (s, 1H), 7.25-7.02 (m, 8H), 6.45 (s, 1H), 5.37 (s, 2H), 2.22 (s, 3H).

### EXAMPLE 59

### N-(3-Chlorophenyl)-N-((8-fluoro-2-oxo-1,2-dihydroquinolin-4-yl)methyl)-4-methylbenzamide

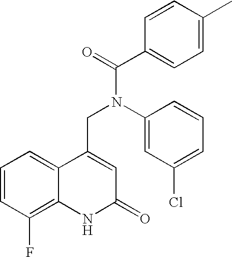

N-(3-Chlorophenyl)-N-((8-fluoro-2-oxo-1,2-dihydroquinolin-4-yl)methyl)-4-methylbenzamide was synthesized as described in EXAMPLE 54 using 4-((3-chlorophenylamino)methyl)-8-fluoroquinolin-2(1H)-one and 4-methylbenzoyl chloride as starting materials. ¹H NMR (400 MHz, DMSO-d₆) δ 11.75 (s, 1H), 7.68 (d, 1H), 7.46 (t, 1H), 7.36 (s, 1H), 7.27-7.20 (m, 5H), 7.10 (d, 2H), 7.01 (s, 1H), 6.47 (s, 1H), 5.36 (s, 2H), 2.25 (s, 3H).

### EXAMPLE 60

### N-(3-Chlorophenyl)-N-((8-fluoro-2-oxo-1,2-dihydroquinolin-4-yl)methyl)-2-methoxybenzamide

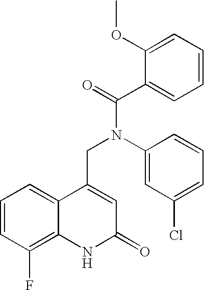

N-(3-Chlorophenyl)-N-((8-fluoro-2-oxo-1,2-dihydroquinolin-4-yl)methyl)-2-methoxybenzamide was synthesized as described in EXAMPLE 54 using 4-((3-chlorophenylamino)methyl)-8-fluoroquinolin-2(1H)-one and 2-methoxybenzoyl chloride as starting materials. ¹H NMR (400 MHz, DMSO-d₆) δ 11.76 (s, 1H), 7.68 (d, 1H), 7.47 (t, 1H), 7.31-7.15 (m, 6H), 6.98 (s, 1H), 6.91-6.84 (m, 2H), 6.69 (s, 1H), 5.31 (s, 2H), 3.64 (s, 3H). LCMS: 437 (M+H)⁺.

### EXAMPLE 61

### N-(3-Chlorophenyl)-N-((8-fluoro-2-oxo-1,2-dihydroquinolin-4-yl)methyl)-3-methoxybenzamide

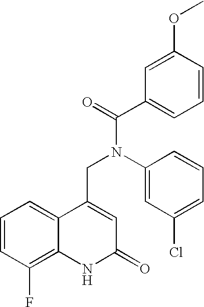

N-(3-Chlorophenyl)-N-((8-fluoro-2-oxo-1,2-dihydroquinolin-4-yl)methyl)-3-methoxybenzamide was synthesized as described in EXAMPLE 54 using 4-((3-chlorophenylamino)methyl)-8-fluoroquinolin-2(1H)-one and 3-methoxybenzoyl chloride as starting materials. ¹H NMR (400 MHz, DMSO-d₆) δ 11.75 (s, 1H), 7.68 (d, 1H), 7.45 (t, 1H), 7.39 (s, 1H), 7.25-7.17 (m, 4H), 7.05 (m, 1H), 6.93-6.88 (m, 3H), 6.49 (s, 1H), 5.36 (s, 2H), 3.66 (s, 3H). LCMS: 437 (M+H)⁺.

### EXAMPLE 62

### N-(3-Chlorophenyl)-N-((8-fluoro-2-oxo-1,2-dihydroquinolin-4-yl)methyl)-4-methoxybenzamide

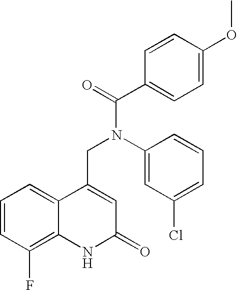

N-(3-Chlorophenyl)-N-((8-fluoro-2-oxo-1,2-dihydroquinolin-4-yl)methyl)-4-methoxybenzamide was synthesized as described in EXAMPLE 54 using 4-((3-chlorophenylamino)methyl)-8-fluoroquinolin-2(1H)-one and 4-methoxybenzoyl chloride as starting materials. ¹H NMR (400 MHz, DMSO-d₆) δ 11.74 (s, 1H), 7.68 (d, 1H), 7.46 (t, 1H), 7.37-7.31 (m, 3H), 7.25-7.20 (m, 3H), 7.02 (m, 1H), 6.83 (m, 2H), 6.47 (s, 1H), 5.36 (s, 2H), 3.73 (s, 3H). LCMS: 437 (M+H)⁺.

### EXAMPLE 63

### Methyl 2-((3-chlorophenyl)((8-fluoro-2-oxo-1,2-dihydroquinolin-4-yl)methyl)amino)-2-oxoacetate

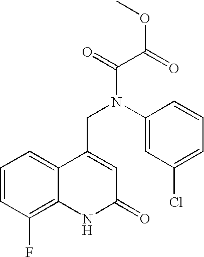

Methyl 2-((3-chlorophenyl)((8-fluoro-2-oxo-1,2-dihydroquinolin-4-yl)methyl)amino)-2-oxoacetate was synthesized as described in EXAMPLE 54 using 4-((3-chlorophenylamino)methyl)-8-fluoroquinolin-2(1H)-one and methyl chlorooxoacetate as starting materials. ¹H NMR (400 MHz, CDCl₃) δ 10.75 (s, 1H), 7.52 (d, 1H), 7.32-7.16 (m, 5H), 7.01 (d, 1H), 6.55 (s, 1H), 5.17 (s, 2H), 3.62 (s, 3H). LCMS: 388.8 (M+H)⁺.

### EXAMPLE 64

### N-(3-Chlorophenyl)-2-cyano-N-((8-fluoro-2-oxo-1,2-dihydroquinolin-4-yl)methyl)benzamide

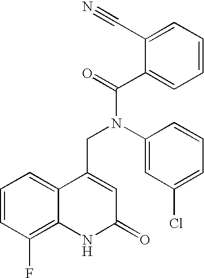

N-(3-Chlorophenyl)-2-cyano-N-((8-fluoro-2-oxo-1,2-dihydroquinolin-4-yl)methyl)benzamide was synthesized as described in EXAMPLE 26 using 4-((3-chlorophenylamino)methyl)-8-fluoroquinolin-2(1H)-one and 2-cyanobenzoic acid as starting materials. ¹H NMR (400 MHz, DMSO-d₆) δ 11.75 (s, 1H), 7.83 (d, 1H), 7.75 (d, 1H), 7.56 (m, 1H), 7.49 (m, 3H), 7.39 (s, 1H), 7.24-7.19 (m, 3H), 6.94 (s, 1H), 5.55 (s, 1H), 5.44 (s, 2H). LCMS: 432 (M+H)⁺.

### EXAMPLE 65

### N-(3-Chlorophenyl)-3-cyano-N-((8-fluoro-2-oxo-1,2-dihydroquinolin-4-yl)methyl)benzamide

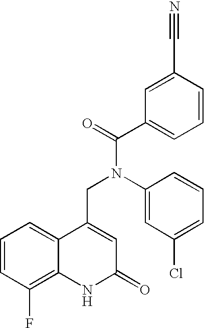

N-(3-Chlorophenyl)-3-cyano-N-((8-fluoro-2-oxo-1,2-dihydroquinolin-4-yl)methyl)benzamide was synthesized as described in EXAMPLE 26 using 4-((3-chlorophenylamino)methyl)-8-fluoroquinolin-2(1H)-one and 3-cyanobenzoic acid as starting materials. ¹H NMR (400 MHz, DMSO-d₆) δ 11.73 (s, 1H), 7.89 (s, 1H), 7.78 (d, 1H), 7.67-7.60 (m, 2H), 7.50-7.40 (m, 3H), 7.15-7.27 (m, 3H), 7.10 (s, 1H), 6.57 (s, 1H), 5.36 (s, 2H). LCMS: 431.9 (M+H)⁺.

### EXAMPLE 66

### N-(3-Chlorophenyl)-4-cyano-N-((8-fluoro-2-oxo-1,2-dihydroquinolin-4-yl)methyl)benzamide

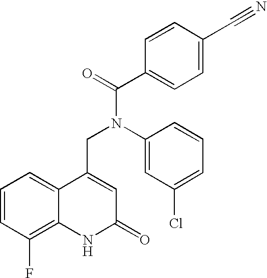

N-(3-Chlorophenyl)-4-cyano-N-((8-fluoro-2-oxo-1,2-dihydroquinolin-4-yl)methyl)benzamide was synthesized as described in EXAMPLE 26 using 4-((3-chlorophenylamino)methyl)-8-fluoroquinolin-2(1H)-one and 4-cyanobenzoic acid as starting materials. ¹H NMR (400 MHz, DMSO) δ 11.74 (s, 1H), 7.76 (d, 2H), 7.65 (d, 1H), 7.54 (d, 2H), 7.44 (m, 2H), 7.24-7.17 (m, 3H), 7.05 (d, 1H), 6.53 (s, 1H), 5.37 (s, 2H). LCMS: 431.8 (M+H)⁺.

### EXAMPLE 67

### N-(3-Chlorophenyl)-N-((8-fluoro-2-oxo-1,2-dihydroquinolin-4-yl)methyl)picolinamide

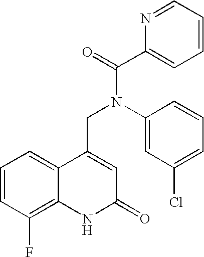

N-(3-Chlorophenyl)-N-((8-fluoro-2-oxo-1,2-dihydroquinolin-4-yl)methyl)picolinamide was synthesized as described in EXAMPLE 26 using 4-((3-chlorophenylamino)methyl)-8-fluoroquinolin-2(1H)-one and picolinic acid as starting materials. ¹H NMR (400 MHz, CDCl₃) δ 8.49 (d, 1H), 7.91 (t, 1H), 7.65 (m, 2H), 7.38 (m, 3H), 7.12 (m, 3H), 6.92 (m, 2H), 5.42 (s, 2H). LCMS: 408.3 (M+H)⁺.

### EXAMPLE 68

### N-(3-Chlorophenyl)-N-((8-fluoro-2-oxo-1,2-dihydroquinolin-4-yl)methyl)nicotinamide

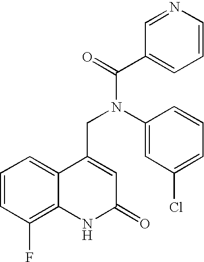

N-(3-Chlorophenyl)-N-((8-fluoro-2-oxo-1,2-dihydroquinolin-4-yl)methyl)nicotinamide was synthesized as described in EXAMPLE 26 using 4-((3-chlorophenylamino)methyl)-8-fluoroquinolin-2(1H)-one and nicotinic acid as starting materials. ¹H NMR (400 MHz, CDCl₃ and CD₃OD) δ 8.49 (m, 1H), 8.44 (d, 1H), 7.67 (m, 1H), 7.53 (m, 1H), 7.27 (t, 1H), 7.15 (m, 5H), 6.82 (d, 1H), 6.58 (s, 1H), 5.30 (s, 2H). LCMS: 408.4 (M+H)⁺.

### EXAMPLE 69

### N-(3-Chlorophenyl)-N-((8-fluoro-2-oxo-1,2-dihydroquinolin-4-yl)methyl)isonicotinamide

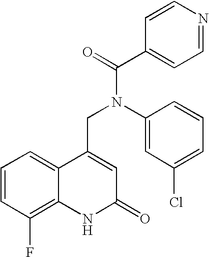

N-(3-Chlorophenyl)-N-((8-fluoro-2-oxo-1,2-dihydroquinolin-4-yl)methyl)isonicotinamide was synthesized as described in EXAMPLE 26 using 4-((3-chlorophenylamino)methyl)-8-fluoroquinolin-2(1H)-one and isonicotinic acid as starting materials. ¹H NMR (400 MHz, CDCl₃ and CD₃OD) δ 8.44 (d, 2H), 7.52 (d, 1H), 7.27 (m, 1H), 7.19 (d, 2H), 7.12 (m, 3H), 6.81 (d, 2H), 6.57 (s, 1H), 5.42 (s, 2H). LCMS: 408.2 (M+H)⁺.

### EXAMPLE 70

### N-(3-Chlorophenyl)-N-((8-fluoro-2-oxo-1,2-dihydroquinolin-4-yl)methyl)pyrazine-2-carboxamide

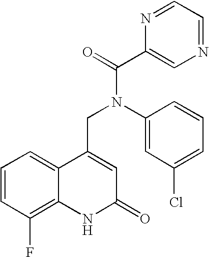

N-(3-Chlorophenyl)-N-((8-fluoro-2-oxo-1,2-dihydroquinolin-4-yl)methyl)pyrazine-2-carboxamide was synthesized as described in EXAMPLE 26 using 4-((3-chlorophenylamino)methyl)-8-fluoroquinolin-2(1H)-one and pyrazinecarboxylic acid as starting materials. ¹H NMR (400 MHz, DMSO-d₆) δ 11.78 (s, 1H), 8.97 (s, 1H), 8.60 (s, 1H), 8.42 (s, 1H), 7.67 (d, 1H), 7.48-7.38 (m, 2H), 7.27-7.12 (m, 3H), 7.07 (s, 1H), 6.60 (s, 1H), 5.42 (s, 2H). LCMS: 408.9 (M+H)⁺.

### EXAMPLE 71

### N-(3-Chlorophenyl)-N-((8-fluoro-2-oxo-1,2-dihydroquinolin-4-yl)methyl)-2-methylnicotinamide

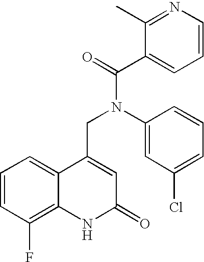

N-(3-Chlorophenyl)-N-((8-fluoro-2-oxo-1,2-dihydroquinolin-4-yl)methyl)-2-methylnicotinamide was synthesized as described in EXAMPLE 26 using 4-((3-chlorophenylamino)methyl)-8-fluoroquinolin-2(1H)-one and 2-methylnicotinic acid as starting materials. ¹H NMR (400 MHz, CDCl₃ and CD₃OD) δ 8.27 (d, 1H), 7.63 (d, 1H), 7.45 (d, 1H), 7.30 (m, 1H), 7.18 (m, 1H), 6.98 (m, 4H), 6.70 (d, 1H), 6.50 (s, 1H), 5.40 (s, 2H), 2.45 (s, 3H); LCMS: 422.2 (M+H)⁺.

### EXAMPLE 72

### N-(3-Chlorophenyl)-N-((8-fluoro-2-oxo-1,2-dihydroquinolin-4-yl)methyl)-4-methylnicotinamide

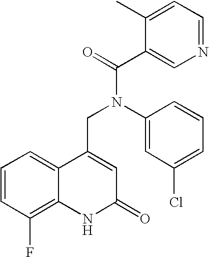

N-(3-Chlorophenyl)-N-((8-fluoro-2-oxo-1,2-dihydroquinolin-4-yl)methyl)-4-methylnicotinamide was synthesized as described in EXAMPLE 26 using 4-((3-chlorophenylamino)methyl)-8-fluoroquinolin-2(1H)-one and 4-methylnicotinic acid as starting materials. ¹H NMR (400 MHz, DMSO-d₆) δ 10.50 (s, 1H), 8.44 (d, 1H), 8.34 (s, 1H), 8.27 (d, 1H), 7.85 (d, 1H), 7.30 (m, 3H), 7.18 (m, 2H), 7.08 (m, 1H), 6.53 (s, 1H), 5.51 (s, 2H), 2.42 (s, 3H). LCMS: 422.2 (M+H)⁺.

### EXAMPLE 73

### N-(3-Chlorophenyl)-N-((8-fluoro-2-oxo-1,2-dihydroquinolin-4-yl)methyl)-3-methylpicolinamide

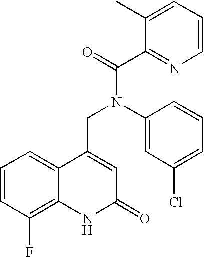

N-(3-Chlorophenyl)-N-((8-fluoro-2-oxo-1,2-dihydroquinolin-4-yl)methyl)-3-methylpicolinamide was synthesized as described in EXAMPLE 26 using 4-((3-chlorophenylamino)methyl)-8-fluoroquinolin-2(1H)-one and 3-methylpicolinic acid as starting materials. ¹H NMR (400 MHz, CDCl₃ and CD₃OD) δ 8.30 (d, 1H), 8.08 (d, 1H), 7.68 (d, 1H), 7.55 (d, 1H), 7.38 (d, 1H), 7.20 (m, 2H), 6.90 (m, 2H), 6.75 (d, 1H), 6.55 (s, 1H), 5.30 (s, 2H), 2.75 (s, 3H). LCMS: 422.2 (M+H)⁺.

### EXAMPLE 74

### N-(3-Chlorophenyl)-2-(dimethylamino)-N-((8-fluoro-2-oxo-1,2-dihydroquinolin-4-yl)methyl)benzamide

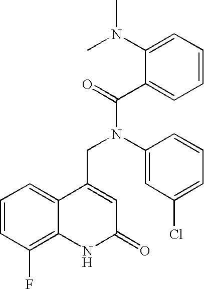

N-(3-Chlorophenyl)-2-(dimethylamino)-N-((8-fluoro-2-oxo-1,2-dihydroquinolin-4-yl)methyl)benzamide was synthesized as described in EXAMPLE 26 using 4-((3-chlorophenylamino)methyl)-8-fluoroquinolin-2(1H)-one and 2-(dimethylamino)benzoic acid as starting materials. ¹H NMR (400 MHz, CDCl₃) δ 7.66 (d, 1H), 7.28 (m, 2H), 7.20 (m, 2H), 7.07 (m, 1H), 7.01 (d, 1H), 6.94 (t, 2H), 6.86 (s, 1H), 6.75 (m, 2H), 5.32 (s, 2H), 2.64 (s, 6H). LCMS: 452.3 (M+H)⁺.

### EXAMPLE 75

### N-(3-Chlorophenyl)-3-(dimethylamino)-N-((8-fluoro-2-oxo-1,2-dihydroquinolin-4-yl)methyl)benzamide

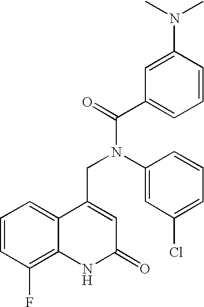

N-(3-Chlorophenyl)-3-(dimethylamino)-N-((8-fluoro-2-oxo-1,2-dihydroquinolin-4-yl)methyl)benzamide was synthesized as described in EXAMPLE 26 using 4-((3-chlorophenylamino)methyl)-8-fluoroquinolin-2(1H)-one and 3-(dimethylamino)benzoic acid as starting materials. ¹H NMR (400 MHz, CDCl₃) δ 7.62 (d, 1H), 7.35 (t, 1H), 7.26 (m, 1H), 7.19 (t, 1H), 7.12 (m, 4H), 7.04 (d, 1H), 6.98 (d, 2H), 6.90 (d, 1H), 6.78 (s, 1H), 5.37 (s, 2H), 2.90 (s, 6H). LCMS: 452.4 (M+H)⁺.

### EXAMPLE 76

### N-(3-Chlorophenyl)-4-(dimethylamino)-N-((8-fluoro-2-oxo-1,2-dihydroquinolin-4-yl)methyl)benzamide

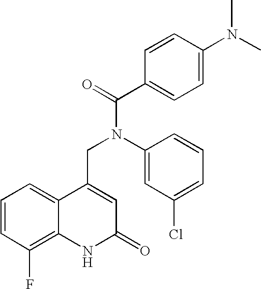

N-(3-Chlorophenyl)-4-(dimethylamino)-N-((8-fluoro-2-oxo-1,2-dihydroquinolin-4-yl)methyl)benzamide was synthesized as described in EXAMPLE 26 using 4-((3-chlorophenylamino)methyl)-8-fluoroquinolin-2(1H)-one and 4-(dimethylamino)benzoic acid as starting materials. ¹H NMR (400 MHz, DMSO-d₆) δ 7.62 (d, 1H), 7.35 (m, 3H), 7.12 (m, 3H), 7.10 (m, 3H), 6.91 (m, 1H), 6.78 (s, 1H), 5.37 (s, 2H), 2.92 (s, 6H). LCMS: 452.2 (M+H)⁺.

### EXAMPLE 77

### 4-(((3-Chlorophenyl)(pyridin-2-yl)amino)methyl)-8-fluoroquinolin-2(1H)-one

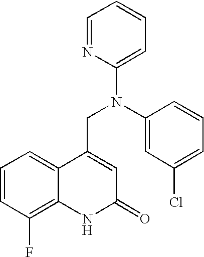

Sodium tert-butoxide (190 mg, 2 mmol) was added to 4-((3-chlorophenylamino)methyl)-8-fluoroquinolin-2(1H)-one, (302 mg, 1 mmol), 2-chloropyridine (100 μL, 1 mmol), Pd(OAc)₂ (20 mg, 0.03 mmol), and 2-(di-t-butylphosphino)biphenyl (18 mg, 0.06 mmol) in toluene (2 mL) under nitrogen. The resulting reaction mixture was stirred at 110° C. for 18 h, whereupon it was cooled to RT. The reaction mixture was poured into EtOAc/H₂O (1:1, 50 mL). The organic layer was separated and the aqueous layer washed with EtOAc (2×); the combined organic layers were dried over Na₂SO₄, filtered and concentrated to a solid. This was purified by column chromatography on silica gel (20% to 80% EtOAc in hexanes) to afford 37 mg (10% yield) of 4-(((3-chlorophenyl)(pyridin-2-yl)amino)methyl)-8-fluoroquinolin-2(1H)-one. ¹H NMR (400 MHz, DMSO-d₆) δ 11.68 (s, 1H), 8.16 (m, 1H), 7.68 (d, 1H), 7.57 (m, 1H), 7.41 (m, 3H), 7.25 (m, 1H), 7.21 (m, 2H), 6.81 (m, 2H), 6.30 (s, 1H), 5.47 (s, 2H). LCMS: 380.0 (M+H)⁺.

### EXAMPLE 78

### 4-(((3-Chlorophenyl)(isoquinolin-1-yl)amino)methyl)-8-fluoroquinolin-2(1H)-one

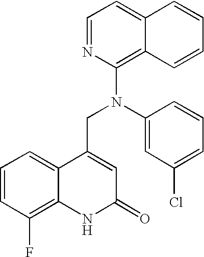

4-(((3-Chlorophenyl)(isoquinolin-1-yl)amino)methyl)-8-fluoroquinolin-2(1H)-one was synthesized as described in EXAMPLE 77 using 4-((3-chlorophenylamino)methyl)-8-fluoroquinolin-2(1H)-one and 1-chloroisoquinoline as starting materials. ¹H NMR (400 MHz, CDCl₃) δ 8.00-6.5 (m, 14H), 4.80 (m, 2H). LCMS: 429.9 (M+H)⁺.

### EXAMPLE 79

### 8-Fluoro-4-((2-(4-methylthiazol-5-yl)-1H-benzo[d]imidazol-1-yl)methyl)quinolin-2(1H)-one

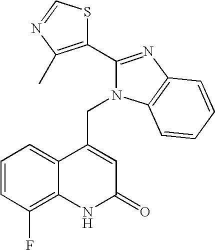

### Step 1: 5-(1H-Benzo[d]imidazol-2-yl)-4-methylthiazole

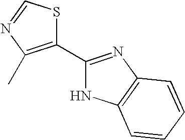

o-Phenylenediamine (0.43 g, 4 mmol) and 4-methylthiazole-5-carboxylic acid (0.57 g, 4 mmol) were suspended in polyphosphoric acid (5 mL) under nitrogen and heated to 125° C. for 48 h, whereupon the reaction mixture was cooled to RT. The reaction mixture was poured carefully into ice/H₂O (100 mL) and extracted with EtOAc (2×100 mL). The pH of the aqueous layer was then taken to 8 using NaOH (10M) and extracted with EtOAc (2×100 mL). The combined organic layers were dried over Na₂SO₄, filtered and concentrated to a solid. This was purified by column chromatography on silica gel column (50% to 100% EtOAc in hexanes) to afford a solid. This was recrystallized from hot EtOAc/hexane (1/1) to give 5-(1H-benzo[d]imidazol-2-yl)-4-methylthiazole as a crystalline solid 100 mg (13% yield). ¹H NMR (400 MHz, DMSO-d₆) δ 12.64 (s, 1H), 9.10 (s, 1H), 7.64 (m, 1H), 7.51 (m, 1H), 7.21 (m, 2H), 2.78 (s, 3H). LCMS: 216.0 (M+H)⁺.

### Step 2: 8-Fluoro-4-((2-(4-methylthiazol-5-yl)-1H-benzo[d]imidazol-1-yl)methyl)quinolin-2(1H)-one

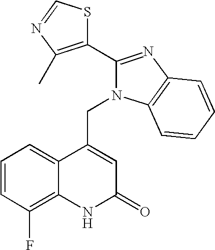

Sodium hydride (60%, 35 mg, 0.88 mmol) was added to a DMF solution of -(1H-benzo[d]imidazol-2-yl)-4-methylthiazole (86 mg, 0.4 mmol) at RT. Gas was evolved. After 15 min, 4-(bromomethyl)-8-fluoroquinolin-2(1H)-one (123 mg, 0.48 mmol) was added as a solid to give a brown solution. After stirring at RT for 18 h, the reaction mixture was poured into EtOAc/brine (1:1, 50 mL). The organic layer was separated and washed with brine (3×); the combined organic layers were dried over Na₂SO₄, filtered and concentrated to a yellow solid. This was purified by reverse phase column chromatography (20% to 100% ACN in H₂O) to afford 25 mg (16% yield) of 8-fluoro-4-((2-(4-methylthiazol-5-yl)-1H-benzo[d]imidazol-1-yl)methyl)quinolin-2(1H)-one. ¹H NMR (400 MHz, DMSO-d₆) δ 11.81 (s, 1H), 9.12 (s, 1H), 7.83 (m, 1H), 7.68 (d, 1H), 7.64 (m, 1H), 7.51 (m, 1H), 7.35 (m, 2H), 7.25 (m, 1H), 5.30 (s, 2H), 2.52 (s, 3H). LCMS: 391.0 (M+H)⁺.

### EXAMPLE 80

### N-(3-Chlorophenyl)-N-((8-fluoro-2-oxo-1,2-dihydroquinolin-4-yl)methyl)isobutyramide

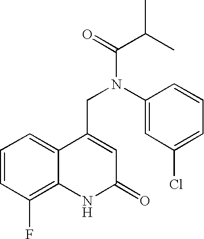

N-(3-Chlorophenyl)-N-((8-fluoro-2-oxo-1,2-dihydroquinolin-4-yl)methyl)isobutyramide was synthesized as described in EXAMPLE 54 using 4-(((3-Chlorophenyl)(isoquinolin-1-yl)amino)methyl)-8-fluoroquinolin-2(1H)-one and isobutyryl chloride as starting materials. ¹H NMR (400 MHz, DMSO-d₆) δ 11.76 (s, 1H), 7.54 (d, 1H), 7.47-7.35 (d, 4H), 7.20-7.10 (m, 2H), 6.28 (s, 1H), 5.08 (s, 2H), 2.48 (m, 1H), 0.96 (d, 6H). LCMS: 372.9 (M+H)⁺.

### EXAMPLE 81

### N-(3-Chlorophenyl)-N-((8-fluoro-2-oxo-1,2-dihydroquinolin-4-yl)methyl)cyclopropanecarboxamide

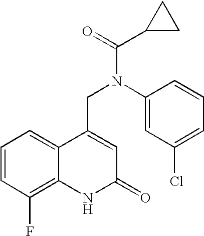

N-(3-Chlorophenyl)-N-((8-fluoro-2-oxo-1,2-dihydroquinolin-4-yl)methyl)cyclopropane carboxamide was synthesized as described in EXAMPLE 54 using 4-(((3-Chlorophenyl) (isoquinolin-1-yl)amino)methyl)-8-fluoroquinolin-2(1H)-one and cyclopropanecarbonyl chloride as starting materials. ¹H NMR (400 MHz, DMSO-d₆) δ 11.77 (s, 1H), 7.55 (d, 1H), 7.45-7.30 (m, 4H), 7.22-7.05 (m, 2H), 6.25 (s, 1H), 5.10 (s, 2H), 1.42 (m, 1H), 0.93-0.82 (m, 4H). LCMS: 370.9 (M+H)⁺.

### EXAMPLE 82

### N-(3-Chlorophenyl)-N-((8-fluoro-2-oxo-1,2-dihydroquinolin-4-yl)methyl)-1-naphthamide

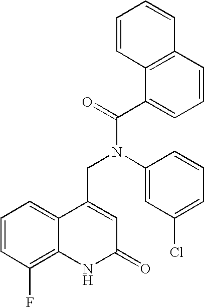

N-(3-Chlorophenyl)-N-((8-fluoro-2-oxo-1,2-dihydroquinolin-4-yl)methyl)-1-naphthamide was synthesized as described in EXAMPLE 54 using 4-((3-chlorophenylamino)methyl)-8-fluoroquinolin-2(1H)-one and 1-naphthoyl chloride as starting materials. ¹H NMR (400 MHz, DMSO-d₆) δ 11.81 (s, 1H), 8.01 (d, 1H), 7.91-7.83 (m, 3H), 7.63-7.34 (m, 7H), 7.03 (m, 2H), 6.84 (s, 1H), 6.52 (s, 1H), 5.51 (s, 2H). LCMS: 457 (M+H)⁺.

### EXAMPLE 83

### N-(3-Chlorophenyl)-N-((8-fluoro-2-oxo-1,2-dihydroquinolin-4-yl)methyl)-2-naphthamide

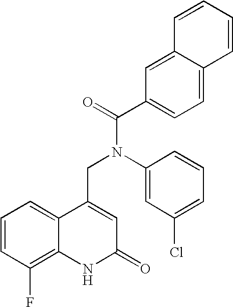

N-(3-Chlorophenyl)-N-((8-fluoro-2-oxo-1,2-dihydroquinolin-4-yl)methyl)-2-naphthamide was synthesized as described in EXAMPLE 54 using 4-((3-chlorophenylamino)methyl)-8-fluoroquinolin-2(1H)-one and 2-naphthoyl chloride as starting materials. ¹H NMR (400 MHz, DMSO-d₆) δ 11.75 (s, 1H), 8.03 (s, 1H), 7.88 (t, 2H), 7.79 (d, 1H), 7.73 (d, 1H), 7.56-7.39 (m, 5H), 7.25 (m, 1H), 7.16 (s, 2H), 7.06 (s, 1H), 6.56 (s, 1H), 5.44 (s, 2H). LCMS: 457 (M+H)⁺.

### EXAMPLE 84

### N-(3-Chlorophenyl)-N-((8-fluoro-2-oxo-1,2-dihydroquinolin-4-yl)methyl)quinoline-6-carboxamide

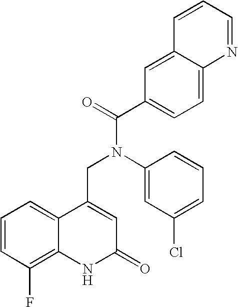

N-(3-Chlorophenyl)-N-((8-fluoro-2-oxo-1,2-dihydroquinolin-4-yl)methyl)quinoline-6-carboxamide was synthesized as described in EXAMPLE 26 using 4-((3-chlorophenylamino)methyl)-8-fluoroquinolin-2(1H)-one and quinoline-6-carboxylic acid as starting materials. ¹H NMR (400 MHz, DMSO-d₆, TFA salt) δ 11.77 (s, 1H), 8.95 (s, 1H), 8.43 (d, 1H), 8.15 (s, 1H), 7.90 (d, 1H), 7.72 (m, 2H), 7.61 (m, 1H), 7.48 (m, 2H), 7.26 (m, 1H), 7.17-7.08 (m, 3H), 6.58 (s, 1H), 5.45 (s, 2H). LCMS: 458 (M+H)⁺.

### EXAMPLE 85

### N-(3-Chloro-4-fluorophenyl)-N-((8-fluoro-2-oxo-1,2-dihydroquinolin-4-yl)methyl)-4-methylthiazole-5-carboxamide

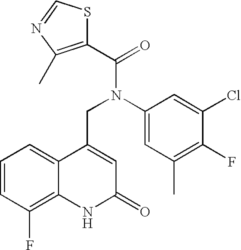

N-(3-Chloro-4-fluorophenyl)-N-((8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-chloro-4-fluoroaniline, and 4-methylthiazole-5-carboxylic acid as starting materials. ¹H NMR (400 MHz, DMSO-d₆) δ 11.72 (s, 1H), 8.93 (s, 1H), 7.78-7.66 (m, 2H), 7.57-7.09 (m, 4H), 6.46 (s, 1H), 5.32 (s, 2H), 2.48 (s, 3H). LCMS: 446 (M+H)⁺.

### EXAMPLE 86

### N-Cyclopropyl-N-((7,8-fluoro-2-oxo-1,2-dihydroquinolin-4-yl)methyl)-4-methylthiazole-5-carboxamide

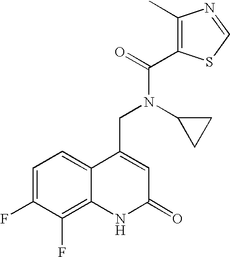

N-Cyclopropyl-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, cyclopropyl amine, and thiazole-5-carboxylic acid as starting materials. ¹H NMR (400 MHz, DMSO-d₆) δ 12.08 (s, 1H), 9.13 (s, 1H), 7.62-7.57 (m, 1H), 7.33-7.31 (m, 1H), 6.36 (s, 1H), 4.87 (s, 2H), 3.33-3.16 (m, 5H), 2.49 (s, 3H). LCMS: 376.1 (M+H)⁺.

### EXAMPLE 87

N-Cyclopropyl-N-((8-fluoro-2-oxo-1,2-dihydroquinolin-4-yl)methyl)-4-methylthiazole-5-carboxamide

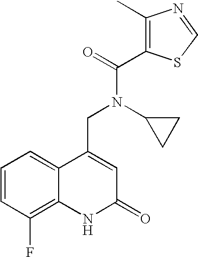

N-Cyclopropyl-N-((8-fluoro-2-oxo-1,2-dihydroquinolin-4-yl)methyl)-4-methylthiazole-5-carboxamide was synthesized as described in EXAMPLE 43 using 4-(bromomethyl)-8-fluoroquinolin-2(1H)-one, cyclopropyl amine, and thiazole-5-carboxylic acid as starting materials. ¹H NMR (400 MHz, DMSO-d₆) δ 11.82 (s, 1H), 9.12 (s, 1H), 7.57-7.42 (m, 2H), 7.45-7.26 (m, 1H), 6.39 (s, 1H), 4.89 (s, 2H), 3.49-3.38 (m, 5H), 2.48 (s, 3H). LCMS: 358.1 (M+H)⁺.

### EXAMPLE 88

### N-(3-Chloro-6-fluorophenyl)-N-((7,8-fluoro-2-oxo-1,2-dihydroquinolin-4-yl)methyl)-4-methylthiazole-5-carboxamide

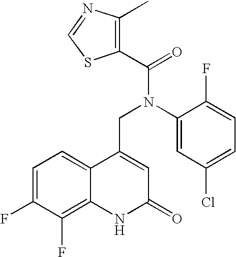

N-(3-Chloro-6-fluorophenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-chloro-6-fluoroaniline, and 4-methylthiazole-5-carboxylic acid as starting materials. ¹H NMR (400 MHz, DMSO-d₆) δ 12.10 (s, 1H), 8.95 (s, 1H), 7.90-7.85 (d, 1H), 7.75-7.65 (m, 1H), 7.55-7.45 (m, 1H), 7.30-7.25 (m, 1H), 7.25-7.20 (m, 1H), 6.45 (s, 1H), 5.18 (s, 2H), 2.48 (s, 3H). LCMS: 464.1 (M+H)⁺.

### EXAMPLE 89

### N-(3-Chloro-4-fluorophenyl)-N-((7,8-fluoro-2-oxo-1,2-dihydroquinolin-4-yl)methyl)-4-methylthiazole-5-carboxamide

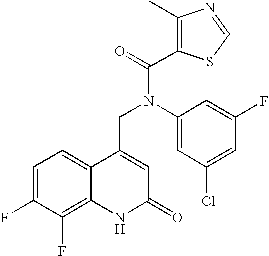

N-(3-Chloro-4-fluorophenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-chloro-5-fluoroaniline, and 4-methylthiazole-5-carboxylic acid as starting materials. ¹H NMR (400 MHz, DMSO-d₆) δ 12.02 (s, 1H), 9.08 (s, 1H), 7.94 (s, 1H), 7.75-7.65 (m, 1H), 7.25-7.15 (m, 2H), 6.35 (s, 1H), 5.18 (s, 2H), 2.48 (s, 3H). LCMS: 464.1 (M+H)⁺.

### EXAMPLE 90

### N-(2-Fluorophenyl)-N-((7,8-fluoro-2-oxo-1,2-dihydroquinolin-4-yl)methyl)-4-methylthiazole-5-carboxamide

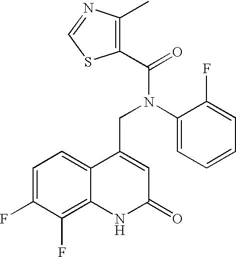

N-(2-Fluorophenyl)-N-((7,8-fluoro-2-oxo-1,2-dihydroquinolin-4-yl)methyl)-4-methylthiazole-5-carboxamidewas synthesized as described in EXAMPLE 43 using 4-(bromomethyl)-7,8-difluoroquinolin-2(1H)-one, 2-fluoroaniline, and 4-methylthiazole-5-carboxylic acid as starting materials. ¹H NMR (400 MHz, DMSO-d₆) δ 12.05 (s, 1H), 8.97 (s, 1H), 7.80-7.55 (m, 1H), 7.21-7.15 (m, 5H), 6.33 (s, 1H), 5.28 (s, 2H), 2.48 (s, 3H). LCMS: 430.1 (M+H)⁺.

### EXAMPLE 91

### N-(3-Methoxylphenyl)-N-((8-fluoro-2-oxo-1,2-dihydroquinolin-4-yl)methyl)-4-methylthiazole-5-carboxamide

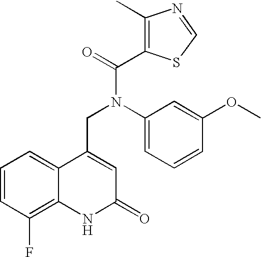

N-(3-Methoxylphenyl)-N-((8-fluoro-2-oxo-1,2-dihydroquinolin-4-yl)methyl)-4-methylthiazole-5-carboxamide was synthesized as described in EXAMPLE 43 using 4-(bromomethyl)-8-fluoroquinolin-2(1H)-one, 3-methoxyaniline, and thiazole-5-carboxylic acid as starting materials. ¹H NMR (400 MHz, DMSO-d₆) δ 11.77 (s, 1H), 8.89 (s, 1H), 7.76 (d, 1H), 7.57-7.42 (m, 1H), 7.39-7.11 (m, 2H), 6.80 (m, 2H), 6.70-6.65 (m, 1H), 6.40 (s, 1H), 5.32 (s, 2H), 3.63 (s, 3H), 2.48 (s, 3H). LCMS: 424.1 (M+H)⁺.

### EXAMPLE 92

### N-(3,4-Difluorophenyl)-N-((8-fluoro-2-oxo-1,2-dihydroquinolin-4-yl)methyl)-4-methylthiazole-5-carboxamide

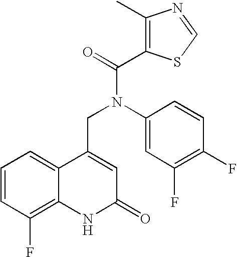

N-(3,4-Difluorophenyl)-N-((8-fluoro-2-oxo-1,2-dihydroquinolin-4-yl)methyl)-4-methylthiazole-5-carboxamide was synthesized as described in EXAMPLE 43 using 4-(bromomethyl)-8-fluoroquinolin-2(1H)-one, 3,4-difluoroaniline, and 4-methylthiazole-5-carboxylic acid as starting materials. LCMS: 430 (M+H)⁺.

### EXAMPLE 93

### N-(3-Methylphenyl)-N-((8-fluoro-2-oxo-1,2-dihydroquinolin-4-yl)methyl)-4-methylthiazole-5-carboxamide

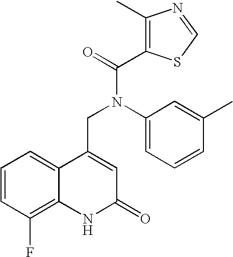

N-(3-Methylphenyl)-N-((8-fluoro-2-oxo-1,2-dihydroquinolin-4-yl)methyl)-4-methylthiazole-5-carboxamide was synthesized as described in EXAMPLE 43 using 4-(bromomethyl)-8-fluoroquinolin-2(1H)-one, 3-methylaniline, and thiazole-5-carboxylic acid as starting materials. ¹H NMR (400 MHz, DMSO-d₆) δ 11.79 (s, 1H), 8.88 (s, 1H), 7.70 (d, 1H), 7.57-7.41 (m, 2H), 7.14-7.07 (m, 3H), 6.91 (m, 1H), 6.38 (s, 1H), 5.30 (s, 2H), 2.49 (s, 3H), 2.20 (s, 3H). LCMS: 408.71 (M+H)⁺.

### EXAMPLE 94

### N-(3-Methylphenyl)-N-((7,8-fluoro-2-oxo-1,2-dihydroquinolin-4-yl)methyl)-4-methylthiazole-5-carboxamide

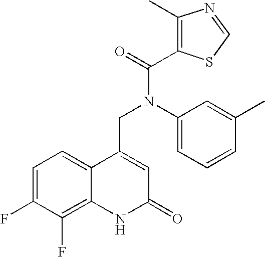

N-(3-Methylphenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-methylaniline, and thiazole-5-carboxylic acid as starting materials. ¹H NMR (400 MHz, DMSO-d₆) δ 12.08 (s, 1H), 8.96 (s, 1H), 7.75-7.72 (m, 1H), 7.46-7.38 (m, 1H), 7.18-7.14 (m, 3H), 6.90-6.88 (m, 1H), 6.42 (s, 1H), 5.34 (s, 2H), 2.48 (s, 3H), 2.19 (s, 3H). LCMS: 426.1 (M+H)⁺.

### EXAMPLE 95

### N-(3-Cyanophenyl)-N-((8-fluoro-2-oxo-1,2-dihydroquinolin-4-yl)methyl)-4-methylthiazole-5-carboxamide

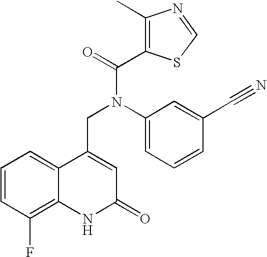

N-(3-Cyanophenyl)-N-((8-fluoro-2-oxo-1,2-dihydroquinolin-4-yl)methyl)-4-methylthiazole-5-carboxamide was synthesized as described in EXAMPLE 43 using 4-(bromomethyl)-8-fluoroquinolin-2(1H)-one, 3-cyanoaniline, and thiazole-5-carboxylic acid as starting materials. ¹H NMR (400 MHz, DMSO-d₆) δ 11.77 (s, 1H), 8.93 (s, 1H), 7.96 (d, 1H), 7.77-7.61 (m, 2H), 7.29-7.21 (m, 3H), 7.39 (d, 1H), 6.44 (s, 1H), 5.37 (s, 2H), 2.48 (s, 3H). LCMS: 419.1 (M+H)⁺.

### EXAMPLE 96

### N-(3-Chloro-2-fluorophenyl)-N-((7,8-fluoro-2-oxo-1,2-dihydroquinolin-4-yl)methyl)-4-methylthiazole-5-carboxamide

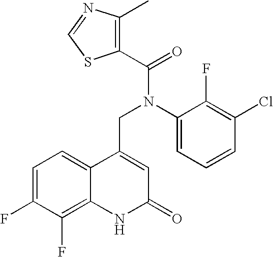

N-(3-Chloro-2-fluorophenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-chloro-2-fluoroaniline, and 4-methylthiazole-5-carboxylic acid as starting materials. LCMS: 464.0 (M+H)⁺.

### EXAMPLE 97

### N-Phenyl-N-((8-fluoro-2-oxo-1,2-dihydroquinolin-4-yl)methyl)-4-methylthiazole-5-carboxamide

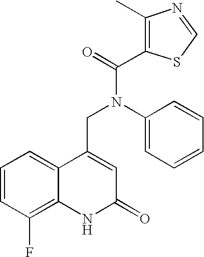

N-Phenyl-N-((8-fluoro-2-oxo-1,2-dihydroquinolin-4-yl)methyl)-4-methylthiazole-5-carboxamide was synthesized as described in EXAMPLE 43 using 4-(bromomethyl)-8-fluoroquinolin-2(1H)-one, aniline, and thiazole-5-carboxylic acid as starting materials. LCMS: 394.1 (M+H)⁺.

### EXAMPLE 98

### N-(3-Chloro-4-fluorophenyl)-2-(dimethylamino)-N-((8-fluoro-2-oxo-1,2-dihydroquinolin-4-yl)methyl)-4-methylthiazole-5-carboxamide

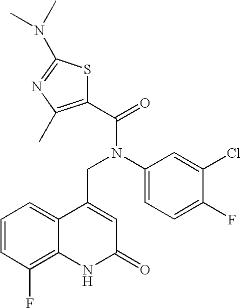

### Step 1: Ethyl 2-chloro-3-oxobutanoate

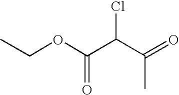

Sulfuryl dichloride (114 g, 843.79 mmol) was added dropwise over 2 h to a cooled (0-5° C.) solution of ethyl 3-oxobutanoate (100 g, 767.63 mmol) in DCM (1000 mL). The resulting solution was allowed to react overnight at RT. The resulting mixture was washed with H₂O (2×1000 ml), dried over Na₂SO₄, and concentrated to afford 110 g (83%) of ethyl 2-chloro-3-oxobutanoate as light yellow oil.

### Step 2: Ethyl 2-amino-5-methylthiazole-4-carboxylate

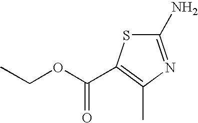

Thiourea (47 g, 616.83 mmol) was added to a solution of ethyl 2-chloro-3-oxobutanoate (100 g, 577.19 mmol) in ethanol (1000 mL). The resulting solution was allowed to react for 2 h at reflux. The reaction mixture was cooled in a water/ice bath. A filtration was performed to afford 105 g (93%) of ethyl 2-amino-5-methylthiazole-4-carboxylate as a light yellow solid.

### Step 3: 2-Amino-4-methylthiazole-5-carboxylic acid

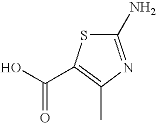

Ethyl 2-amino-4-methylthiazole-5-carboxylate (1 g, 4.83 mmol) was added to a solution of lithium hydroxide (260 mg, 10.75 mmol) in H₂O (50 ml) and was stirred for 5 h at 50° C. The resulting solution was extracted with EtOAc (3×100 mL). The organics were combined, dried over Na₂SO₄, and evaporated to afford 0.4 g (crude) of 2-amino-4-methylthiazole-5-carboxylic acid as a white solid. LCMS: 159 (M+H)⁺.

### Step 4: 2-Bromo-4-methylthiazole-5-carboxylic acid

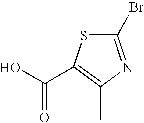

Copper (I) bromide (2.1 g, 14.62 mmol) and t-BuONO (6.5 g, 62.97 mmol) were added to a solution of 2-amino-4-methylthiazole-5-carboxylic acid (2 g, 11.38 mmol) in ACN (60 mL). The resulting solution was stirred for 2 h at reflux. The mixture was poured into water (100 mL) and extracted with EtOAc (3×100 mL). The organics were combined, dried over Na₂SO₄, and evaporated to afford 2 g (63%) of 2-bromo-4-methylthiazole-5-carboxylic acid as a yellow solid.

### Step 5: 2-Chloro-4-methylthiazole-5-carbonyl chloride

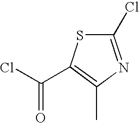

Sulfuryl dichloride (25 mL) was added to 2-bromo-4-methylthiazole-5-carboxylic acid (3 g, 13.51 mmol) and the resulting solution was stirred for 3 h at reflux. The mixture was then concentrated to afford 4 g (crude) of 2-chloro-4-methylthiazole-5-carbonyl chloride as a brown liquid.

### Step 6: 2-Chloro-N-(3-chloro-4-fluorophenyl)-N-((8-fluoro-2-oxo-1,2-dihydroquinolin-4-yl)methyl)-4-methylthiazole-5-carboxamide

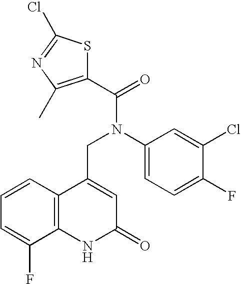

2-Chloro-N-(3-chloro-4-fluorophenyl)-N-((8-fluoro-2-oxo-1,2-dihydroquinolin-4-yl)methyl)-4-methylthiazole-5-carboxamide was synthesized as described in EXAMPLE 26 using 4-((3-chloro-4-fluorophenylamino)methyl)quinolin-2(1H)-one and 2-chloro-4-methylthiazole-5-carbonyl chloride as starting materials. LCMS: 480 (M+H)⁺.

### Step 7: N-(3-Chloro-4-fluorophenyl)-2-(dimethylamino)-N-((8-fluoro-2-oxo-1,2-dihydroquinolin-4-yl)methyl)-4-methylthiazole-5-carboxamide

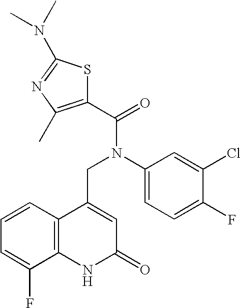

Dimethylamine hydrochloride (51 mg, 0.63 mmol) was added to a solution of 2-chloro-N-(3-chloro-4-fluorophenyl)-N-((8-fluoro-2-oxo-1,2-dihydroquinolin-4-yl)methyl)-4-methylthiazole-5-carboxamide (300 mg, 0.62 mmol) in DMF (30 mL) and the resulting solution was stirred overnight at RT. A filtration was performed, the filtrate was concentrated, and the residue was purified by column chromatography on silica gel (EtOAc:PE=1:5) to afford 200 mg (65%) of N-(3-chloro-4-fluorophenyl)-2-(dimethylamino)-N-((8-fluoro-2-oxo-1,2-dihydroquinolin-4-yl)methyl)-4-methylthiazole-5-carboxamide as a white solid. ¹H NMR (400 MHz, DMSO-d₆) δ 11.71 (s, 1H), 7.61 (t, 2H), 7.36 (m, 2H), 7.18 (m, 2H), 6.36 (s, 1H), 5.23 (s, 2H), 2.90 (s, 6H), 2.31 (s, 3H). LCMS: 489.0 (M+H)⁺.

### EXAMPLE 99

### 2-Bromo-4-methyl-N-((2-oxo-1,2-dihydroquinolin-4-yl)methyl)-N-phenylthiazole-5-carboxamide

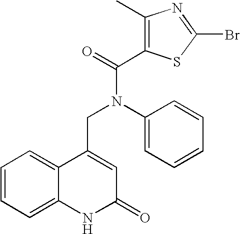

2-Bromo-4-methyl-N-((2-oxo-1,2-dihydroquinolin-4-yl)methyl)-N-phenylthiazole-5-carboxamide was synthesized as described in EXAMPLE 26 using 4-((phenylamino)methyl)quinolin-2(1H)-one and 2-bromo-4-methyl-5-thiazole carboxylic acid as starting materials. ¹H NMR (400 MHz, DMSO-d₆) δ 11.73 (s, 1H), 7.82 (d, 1H), 7.51 (dd, 1H), 7.35-7.29 (m, 4H), 7.19-7.25 (m, 3H), 6.32 (s, 1H), 5.31 (s, 2H), 2.39 (d, 3H). LCMS: 454.1 (M+H)⁺.

### EXAMPLE 100

### 2-(Dimethylamino)-4-methyl-N-((2-oxo-1,2-dihydroquinolin-4-yl)methyl)-N-phenylthiazole-5-carboxamide

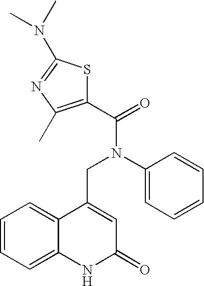

2-Bromo-4-methyl-N-((2-oxo-1,2-dihydroquinolin-4-yl)methyl)-N-phenylthiazole-5-carboxamide (1 eq.) in anhydrous dioxane was sequentially treated with dimethylamine (1.5 eq.), 1,3 bis-(2,6-dipropylphenyl imidazolium chloride (0.4 eq.), Sodium t-butoxide (2 eq.), and tris(dibenzylideneacetone)dipalladium (0.1 eq.). The reaction vessel was purged with nitrogen and evacuated with vacuum several times to render an inert atmosphere. The reaction mixture was stirred at RT for 24 h, after which the mixture was filtered and the residue was purified to afford pale-yellow powder (49%). LCMS: 419 (M+H)⁺.

### EXAMPLE 101

### 4-Methyl-2-morpholino-N-((2-oxo-1,2-dihydroquinolin-4-yl)methyl)-N-phenylthiazole-5-carboxamide

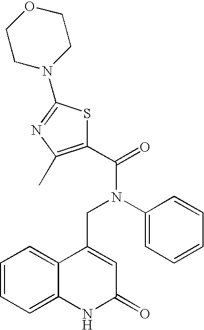

4-Methyl-2-morpholino-N-((2-oxo-1,2-dihydroquinolin-4-yl)methyl)-N-phenylthiazole-5-carboxamide was synthesized as described in EXAMPLE 100 using 2-bromo-4-methyl-N-((2-oxo-1,2-dihydroquinolin-4-yl)methyl)-N-phenylthiazole-5-carboxamide and morpholine as starting materials. LCMS: 461 (M+H)⁺.

### EXAMPLE 102

### 4-Methyl-2-(4-methylpiperazin-1-yl)-N-((2-oxo-1,2-dihydroquinolin-4-yl)methyl)-N-phenylthiazole-5-carboxamide

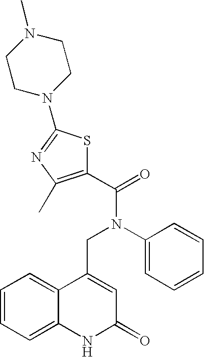

4-Methyl-2-(4-methylpiperazin-1-yl)-N-((2-oxo-1,2-dihydroquinolin-4-yl)methyl)-N-phenylthiazole-5-carboxamide was synthesized as described in EXAMPLE 100 using 2-bromo-4-methyl-N-((2-oxo-1,2-dihydroquinolin-4-yl)methyl)-N-phenylthiazole-5-carboxamide and 1-methylpiperazine as starting materials. LCMS: 474 (M+H)⁺.

### EXAMPLE 103

### 6-(Benzyl((6-methyl-2-oxo-1,2-dihydroquinolin-3-yl)methyl)amino)nicotinonitrile

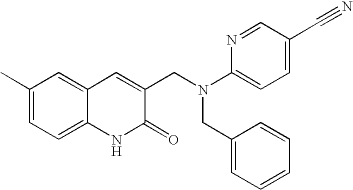

### Step 1: N-p-Tolylacetamide

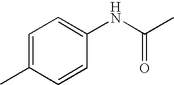

Triethylamine (34 g, 336 mmol) was added to p-toluidine (30 g, 279.98 mmol) in DCM (500 mL). The mixture was cooled to 10° C. and acetyl chloride (26.4 g, 336.31 mmol) was added dropwise while stirring. The reaction mixture was stirred at this temperature for 1 h. It was then washed with 2% HCl (1×500 mL), NaHCO₃ (1×500 mL), and with brine (1×500 mL). The organic layer was then dried over MgSO₄ and concentrated by evaporation under vacuum using a rotary evaporator. This resulted in 33 g (79%) of N-p-tolylacetamide as a yellow solid. LCMS: 148 (M+H)⁺.

### Step 2: 2-Chloro-6-methylquinoline-3-carbaldehyde

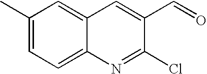

Phosphoryl trichloride (237.2 g, 1.55 mol) was added dropwise with stirring, while cooling to 0° C. to N,N-dimethylformamide (40.4 g, 552.74 mmol). N-p-Tolylacetamide (33 g, 221.19 mmol) was then added and the resulting solution was allowed to react, with stirring, overnight while the temperature was maintained at reflux. The reaction mixture was then quenched by the adding 3000 ml of H₂O/ice. Adjustment of the pH to 9 was accomplished by the addition of Na₂CO₃. The resulting solution was extracted with DCM (3×3000 mL). The organics were combined, dried over MgSO₄, and concentrated by evaporation under vacuum using a rotary evaporator. The residue was purified by column chromatography on silica gel eluting with a 1:10 EtOAc:hexanes to afford 15.3 g (34%) of 2-chloro-6-methylquinoline-3-carbaldehyde as a yellow solid. LCMS: 206 (M+H)⁺.

### Step 3: 6-Methyl-2-oxo-1,2-dihydroquinoline-3-carbaldehyde

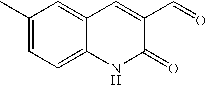

2-Chloro-6-methylquinoline-3-carbaldehyde (15.0 g, 72.94 mmol) and hydrochloric acid (800 ml) were stirred overnight at 90° C. A filtration was performed. This resulted in 13.4 g (98%) of 6-methyl-2-oxo-1,2-dihydroquinoline-3-carbaldehyde as a yellow solid. LCMS: 188 (M+H)⁺.

### Step 4: 3-((Benzylamino)methyl)-6-methylquinolin-2(1H)-one

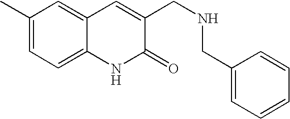

A mixture of 6-methyl-2-oxo-1,2-dihydroquinoline-3-carbaldehyde (1.5 g, 8.01 mmol), phenylmethanamine (1.43 g, 8.00 mmol), acetic acid (1 mL) in THF (50 mL) was stirred at 45° C. for 30 min. NaHB(OCOCH₃)₃ (2.55 g, 12.03 mmol) was then added in several batches. The resulting solution was allowed to react, with stirring, overnight while the temperature was maintained at 45° C. A filtration was performed and the filtrate was concentrated by evaporation under vacuum using a rotary evaporator. The resulting mixture was washed with DCM (1×40 mL) and the resulting solid was filtered and dried to yield 1 g (45%) of 3-((benzylamino)methyl)-6-methylquinolin-2(1H)-one as a light yellow solid. LCMS: 279 (M+H)⁺.

### Step 5: 6-(Benzyl((6-methyl-2-oxo-1,2-dihydroquinolin-3-yl)methyl)amino)nicotinonitrile

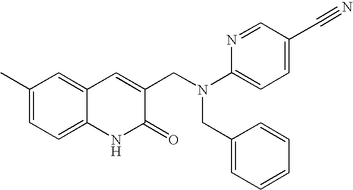

A mixture of 3-((benzylamino)methyl)-6-methylquinolin-2(1H)-one (200 mg, 0.72 mmol), 6-chloronicotinonitrile (120 mg, 0.87 mmol), and triethylamine (220 mg, 2.17 mmol) in DMSO (15 mL) was heated to 120° C. for 8 h. DMSO was removed by distillation and the resulting residue was purified by column chromatography eluting on silica gel (20:1 DCM/EtOAc) to afford 30 mg (11%) of 6-(benzyl((6-methyl-2-oxo-1,2-dihydroquinolin-3-yl)methyl)amino)nicotinonitrile as a white solid. ¹HNMR (300 MHz, DMSO-d₆) δ 11.83 (s, 1H), 8.51 (s, 1H), 7.82 (d, 1H), 7.43 (d, 1H), 7.34 (m, 2H), 7.29 (d, 1H), 7.26 (s, 1H), 7.23 (d, 1H), 7.20 (d, 2H), 6.73 (d, 1H), 4.95 (s, 2H), 4.62 (s, 2H), 2.27 (s, 3H). LCMS: 381 (M+H)⁺.

### EXAMPLE 104

### N-(3-Chloro-4-fluorophenyl)-8-fluoro-N-((4-methylthiazol-5-yl)methyl)-2-oxo-1,2-dihydroquinoline-4-carboxamide

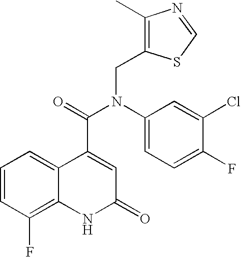

### Intermediate A

### 3-Chloro-4-fluoro-N-((4-methylthiazol-5-yl)methyl)aniline

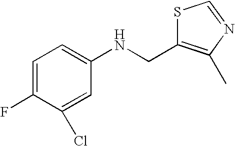

### Step 1: (4-Methylthiazol-5-yl)methanol

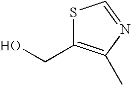

LiAlH₄ (34.21 mmol) was added to a solution of ethyl 4-methylthiazole-5-carboxylate (17.02 mmol) in THF (150 mL) and the resulting solution was allowed to react at RT for 2 h. EtOAc and H₂O were then added and the aqueous layer was extracted with EtOAc (4×100 mL). The organics were combined, dried over Na₂SO₄, and concentrated to afford 1 g of crude (4-methylthiazol-5-yl)methanol as a yellow liquid.

### Step 2: 5-(Bromomethyl)-4-methylthiazole

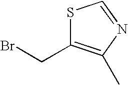

PBr₃ (8.69 mmol) was added to a solution of 4-methylthiazol-5-yl)methanol (8.70 mmol) in DCM (50 mL) and the resulting reaction mixture was stirred at RT for 1 h. It was then poured into ice/H₂O (50 mL) and was extracted with DCM (70×3 mL). The organics were combined, dried over anhydrous Na₂SO₄, and evaporated to afford 0.6 g of crude 5-(bromomethyl)-4-methylthiazole as a yellow liquid.

### Step 3: 3-Chloro-4-fluoro-N-((4-methylthiazol-5-yl)methyl)aniline

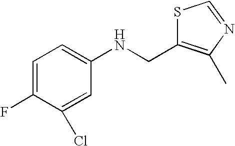

A mixture of 3-chloro-4-fluoroaniline (1.69 mmol), 5-(bromomethyl)-4-methylthiazole (1.69 mmol), and triethylamine (1.68 mmol) in DCM (50 mL) was stirred at RT for 1 h. The reaction mixture was then washed with H₂O, dried over Na₂SO₄, and concentrated to afford 200 mg of crude 3-chloro-4-fluoro-N-((4-methylthiazole-5-yl)methyl)aniline as a light yellow liquid. LCMS: 257 (M+H)⁺.

### Intermediate B

### 8-Fluoro-2-oxo-1,2-dihydroquinoline-4-carbonyl chloride

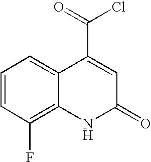

### Step 1: (E)-N-(2-Fluorophenyl)-2-(hydroxyimino)acetamide

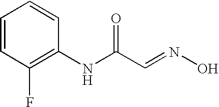

A mixture of 2,2,2-trichloroethane-1,1-diol (41.6 g, 252.12 mmol), 2-fluorobenzenamine (20 g, 180.18 mmol), Na₂SO₄ (143.3 g, 1.01 mol) in water (400 mL)/HCl (30 mL) was stirred at RT for 5 h. NH₂OH.HCl (46 g, 666.67 mmol) was then added and the resulting solution was stirred for 1 h at 60° C. After cooling, the solide was filtered and dried to afford 10 g of crude N-(2-fluorophenyl)-2-(hydroxyimino)acetamide as a brown solid. LCMS: 183 (M+H)⁺.

### Step 2: 7-Fluoroindoline-2,3-dione

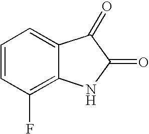

N-(2-Fluorophenyl)-2-(hydroxyimino)acetamide (30 g, 164.84 mmol) in H₂SO₄ (100 mL) was stirred at 80° C. for 2 h. The reaction mixture was poured into ice/water and the red solid was filtered and dried to afford 25 g (90%) of 7-fluoroindoline-2,3-dione as a brick red solid. LCMS: 166 (M+H)⁺.

### Step 3: 8-Fluoro-2-oxo-1,2-dihydroquinoline-4-carboxylic acid

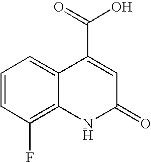

A mixture of 7-fluoroindoline-2,3-dione (5 g, 30.30 mmol), Ac₂O (3.1 g, 30.39 mmol), and sodium hydride (730 mg, 30.42 mmol) in toluene (50 mL) was stirred at RT for 2 h. The mixture was poured into ice/water containing Na₂CO₃. The resulting solution was extracted with EtOAc (3×100 mL). The organics were combined and concentrated by evaporation under vacuum using a rotary evaporator. The crude product was dissolved in NaOH(2N) (100 mL) and refluxed for 3 h. After cooling, the mixture was acidified with diluted HCl. A filtration was performed and this resulted in 2.3 g (37%) of 8-fluoro-2-oxo-1,2-dihydroquinoline-4-carboxylic acid as a brown solid. LCMS: 209 (M+H)⁺.

### Step 4: 8-Fluoro-2-oxo-1,2-dihydroquinoline-4-carbonyl chloride

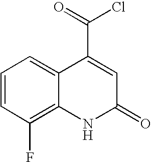

Into a 100 ml round bottom flask, was placed A mixture of 8-fluoro-2-oxo-1,2-dihydroquinoline-4-carboxylic acid (1 g, 4.83 mmol) in sulfuryl dichloride (50 mL) was refluxed for 3 h. The mixture was concentrated by evaporation under vacuum using a rotary evaporator to afford 0.8 g of 8-fluoro-2-oxo-1,2-dihydroquinoline-4-carbonyl chloride as a yellow solid.

### SYNTHESIS OF EXAMPLE 104

### N-(3-Chloro-4-fluorophenyl)-8-fluoro-N-((4-methylthiazol-5-yl)methyl)-2-oxo-1,2-dihydroquinoline-4-carboxamide

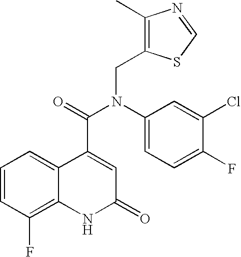

A mixture of Intermediate A (0.89 mmol) and Intermediate B (1.78 mmol) in DMF (30 mL) was stirred at RT for 3 h. The mixture was concentrated to dryness and the residue was purified by column chromatography on silica gel (1:5 EtOAc/hexanes) to afford N-(3-chloro-4-fluorophenyl)-8-fluoro-N-((4-methylthiazol-5-yl)methyl)-2-oxo-1,2-dihydroquinoline-4-carboxamide as an off-white solid. LCMS: 446 (M+H)⁺.

### EXAMPLE 105

### N-(3-Chlorophenyl)-8-fluoro-2-oxo-N-(thiazol-4-ylmethyl)-1,2-dihydroquinoline-4-carboxamide

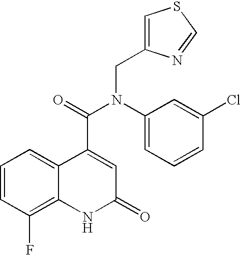

### Step 1: Ethyl 2-aminothiazole-4-carboxylate

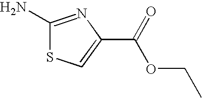

A mixture of thiourea (15.68 g, 206.32 mmol) and ethyl 3-bromo-2-oxopropanoate (40 g, 206.19 mmol) in EtOH (250 mL) was refluxed for 4 h. The reaction mixture was then concentrated by evaporation under vacuum using a rotary evaporator to afford 27 g of crude of ethyl 2-aminothiazole-4-carboxylate as a yellow solid.

### Step 2: Ethyl 2-bromothiazole-4-carboxylate

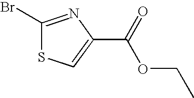

A mixture of ethyl 2-aminothiazole-4-carboxylate (10 g, 58.14 mmol), t-BuONO (30 g, 291.26 mmol), and CuBr (12.5 g, 87.41 mmol) in CH₃CN (150 mL) was refluxed for 2 h. The reaction mixture was then concentrated by evaporation under vacuum using a rotary evaporator followed by addition of water (200 mL). The aqueous layer was extracted with EtOAc (4×150 mL). The organics were combined, dried over Na₂SO₄, and evaporated to give 15 g of crude ethyl 2-bromothiazole-4-carboxylate as a yellow solid.

### Step 3: 3-Chloro-N-(thiazol-4-ylmethyl)aniline

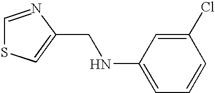

3-Chloro-N-(thiazol-4-ylmethyl)aniline was synthesized as described in EXAMPLE 104, Step 1-3 using ethyl 2-bromothiazole-4-carboxylate and 3-chloroaniline as starting materials. LCMS: 225 (M+H)⁺.

### Step 4: N-(3-Chlorophenyl)-8-fluoro-2-oxo-N-(thiazol-4-ylmethyl)-1,2-dihydroquinoline-4-carboxamide

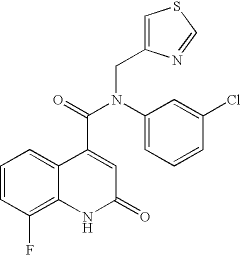

N-(3-Chlorophenyl)-8-fluoro-2-oxo-N-(thiazol-4-ylmethyl)-1,2-dihydroquinoline-4-carboxamide was synthesized as described in EXAMPLE 104, Step 4 using 3-chloro-N-(thiazol-4-ylmethyl)aniline and 8-fluoro-2-oxo-1,2-dihydroquinoline-4-carbonyl chloride as starting materials. LCMS: 414 (M+H)⁺.

### EXAMPLE 106

### 4-((4-(2-Fluorophenyl)piperazin-1-yl)methyl)quinolin-2(1H)-one

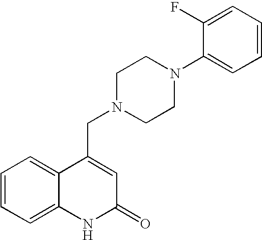

A mixture of 1-(2-fluorophenyl)piperazine (0.25 mmol), -(bromomethyl)quinolin-2(1H)-one (0.25 mmol), and triethylamine (0.25 mmol) in DCM (25 mL) was stirred for 5 h at RT. The mixture was then washed with water, dried over Na₂SO₄, and concentrated to afford 4-((4-(2-fluorophenyl)piperazin-1-yl)methyl)quinolin-2(1H)-one as a pale-yellow dry film. LCMS: 338 (M+H)⁺.

### EXAMPLE 107

### N-((8-Bromo-5-fluoro-2-oxo-1,2-dihydroquinolin-4-yl)methyl)-N-(3-chlorophenyl)-4-methylthiazole-5-carboxamide

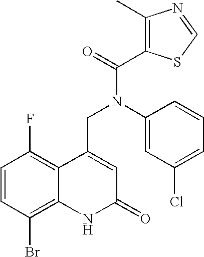

N-((8-Bromo-5-fluoro-2-oxo-1,2-dihydroquinolin-4-yl)methyl)-N-(3-chlorophenyl)-4-methylthiazole-5-carboxamide was synthesized as described in EXAMPLE 43 using 2-bromo-5-fluoroaniline as starting material. ¹H NMR (400 MHz, DMSO-d₆) δ 10.59 (s, 1H), 8.97 (s, 1H), 7.90 (m, 1H), 7.56 (s, 1H), 7.34-7.31 (m, 2H), 7.27 (m, 1H), 7.10 (m, 1H), 6.54 (s, 1H), 5.36 (s, 2H), 2.44 (s, 3H). LCMS: 505 (M)⁺.

### EXAMPLE 108

### N-(3-Chlorophenyl)-N-((3,8-difluoro-2-oxo-1,2-dihydroquinolin-4-yl)methyl)-4-methylthiazole-5-carboxamide

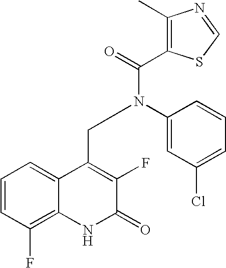

### Step 1: 4-Bromo-2-fluoro-N-(2-fluorophenyl)-3-oxobutanamide

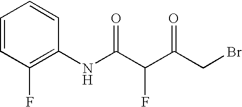

A mixture of 4-bromo-N-(2-fluorophenyl)-3-oxobutanamide (1 g, 3.65 mmol) and Selectfluor® (1.7 g, 4.74 mmol) in ACN (30 mL) was heated to 60° C. for 2 h. The reaction mixture was cooled to room temperature and the solvent was removed. The residue was partitioned between DCM and water. Purification by flash chromatography on silica gel afforded 564 mg (53%) of 4-bromo-2-fluoro-N-(2-fluorophenyl)-3-oxobutanamide as a yellow solid. ¹H NMR (400 MHz, CDCl₃) δ 8.22 (s, 1H), 8.19 (m, 1H), 7.16-7.11 (m, 3H), 5.84-5.71 (d, 1H), 4.43-4.21 (dd, 2H).

### Step 2: 4-(Bromomethyl)-3,8-difluoroquinolin-2(1H)-one

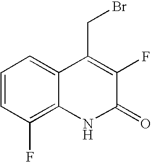

4-(Bromomethyl)-3,8-difluoroquinolin-2(1H)-one was synthesized as described in EXAMPLE 43, Step 3 using 4-bromo-2-fluoro-N-(2-fluorophenyl)-3-oxobutanamide as a starting material. ¹H NMR (400 MHz, DMSO-d₆) δ 12.50 (s, 1H), 7.71 (d, 1H), 7.47 (t, 1H), 7.31 (m, 1H), 4.91 (s, 2H). LCMS: 273 (M+H)⁺.

### Step 3: 4-((3-Chlorophenylamino)methyl)-3,8-difluoroquinolin-2(1H)-one

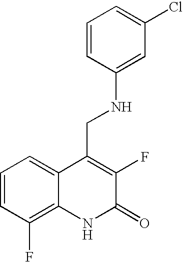

4-((3-Chlorophenylamino)methyl)-3,8-difluoroquinolin-2(1H)-one was synthesized as described in EXAMPLE 43, Step 4 using 4-(bromomethyl)-3,8-difluoroquinolin-2(1H)-one and 3-chloroaniline as starting materials. LCMS: 321 (M+H)⁺.

### Step 4: N-(3-Chlorophenyl)-N-((3,8-difluoro-2-oxo-1,2-dihydroquinolin-4-yl)methyl)-4-methylthiazole-5-carboxamide

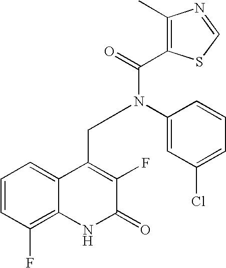

N-(3-Chlorophenyl)-N-((3,8-difluoro-2-oxo-1,2-dihydroquinolin-4-yl)methyl)-4-methylthiazole-5-carboxamide was synthesized as described in EXAMPLE 43, Step 5 using 4-((3-chlorophenylamino)methyl)-3,8-difluoroquinolin-2(1H)-one and 4-methylthiazole-5-carboxylic acid as starting materials. ¹H NMR (400 MHz, DMSO-d₆) δ 12.38 (s, 1H), 8.88 (s, 1H), 7.79 (d, 1H), 7.45 (t, 1H), 7.38-7.30 (m, 3H), 7.19 (t, 1H), 6.77 (d, 1H), 5.44 (s, 2H), 2.42 (s, 3H). LCMS: 446 (M+H)⁺.

### EXAMPLE 109

### N-(3-Chlorophenyl)-N-((6,7-difluoro-2-oxo-1,2-dihydroquinolin-4-yl)methyl)-4-methylthiazole-5-carboxamide

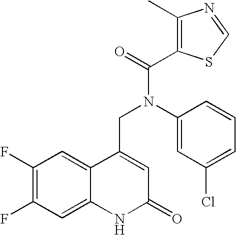

N-(3-Chlorophenyl)-N-((6,7-difluoro-2-oxo-1,2-dihydroquinolin-4-yl)methyl)-4-methylthiazole-5-carboxamide was synthesized as described in EXAMPLE 43 using 3,4-difluoroaniline as starting material ¹H NMR (400 MHz, DMSO-d₆) δ 11.87 (s, 1H), 8.94 (s, 1H), 7.97-7.92 (m, 1H), 7.50 (s, 1H), 7.34-7.23 (m, 3H), 7.08 (d, 1H), 6.39 (s, 1H), 5.33 (s, 2H), 2.44 (s, 3H). LCMS: 446 (M+H)⁺.

### EXAMPLE 110

### N-(3-Chlorophenyl)-N-((7,8-difluoro-2-oxo-1,2-dihydroquinolin-4-yl)methyl)-4-methyl-1,2,3-thiadiazole-5-carboxamide

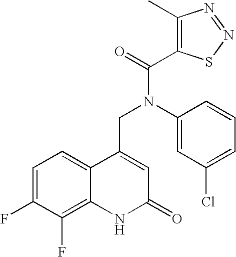

N-(3-Chlorophenyl)-N-((7,8-difluoro-2-oxo-1,2-dihydroquinolin-4-yl)methyl)-4-methyl-1,2,3-thiadiazole-5-carboxamide was synthesized as described in EXAMPLE 43, Step 5 using 4-((3-chlorophenylamino)methyl)-7,8-difluoroquinolin-2(1H)-one and 4-methyl-1,2,3-thiadiazole-5-carboxylic acid as starting materials. ¹H NMR (400 MHz, DMSO-d₆) δ 12.03 (s, 1H), 7.68 (m, 1H), 7.64 (s, 1H), 7.38-7.27 (m, 3H), 7.19 (m, 1H), 6.55 (s, 1H), 5.36 (s, 2H), 2.66 (s, 3H). LCMS: 446 (M)⁺.

### EXAMPLE 111

### N-(3-Chlorophenyl)-N-((5-fluoro-2-oxo-1,2-dihydroquinolin-4-yl)methyl)-4-methylthiazole-5-carboxamide

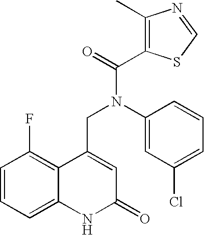

A mixture of N-((8-bromo-5-fluoro-2-oxo-1,2-dihydroquinolin-4-yl)methyl)-N-(3-chlorophenyl)-4-methylthiazole-5-carboxamide (180 mg, 0.36 mmol) and Pd/C in MeOH (5 mL) was hydrogenated overnight (with a balloon of hydrogen). The Pd/C was removed and the solvent was evaporated to dryness. The residue was purified by preparative HPLC (ACN/water) to afford 5.5 mg (3%) of N-(3-chlorophenyl)-N-((5-fluoro-2-oxo-1,2-dihydroquinolin-4-yl)methyl)-4-methylthiazole-5-carboxamide as a white solid. ¹H NMR (400 MHz, DMSO-d₆) δ 11.99 (s, 1H), 8.97 (s, 1H), 7.54-7.51 (m, 2H), 7.33 (m, 2H), 7.25 (m, 1H), 7.16 (d, 1H), 7.04 (d, 1H), 6.44 (s, 1H), 5.35 (s, 2H), 2.44 (s, 3H). LCMS: 427 (M)⁺.

### EXAMPLE 112

### N-(3-Chlorophenyl)-N-((7,8-difluoro-2-oxo-1,2-dihydroquinolin-4-yl)methyl)-1-methyl-1H-pyrazole-5-carboxamide

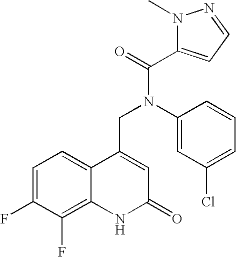

N-(3-Chlorophenyl)-N-((7,8-difluoro-2-oxo-1,2-dihydroquinolin-4-yl)methyl)-1-methyl-1H-pyrazole-5-carboxamide was synthesized as described in EXAMPLE 46 using N-(3-chlorophenyl)-1-methyl-1H-pyrazole-5-carboxamide and 4-(bromomethyl)-7,8-difluoroquinolin-2(1H)-one (for INTERMEDIATE A) as starting materials. ¹H NMR (400 MHz, DMSO-d₆) δ 12.02 (s, 1H), 7.70 (m, 1H), 7.54 (s, 1H), 7.34-7.28 (m, 3H), 7.23 (s, 1H), 7.18 (d, 1H), 6.45 (s, 1H), 5.69 (s, 1H), 5.33 (s, 2H), 2.87 (s, 3H). LCMS: 428 (M)⁺.

### EXAMPLE 113

### N-(3-Chlorophenyl)-N-(1-(8-fluoro-2-oxo-1,2-dihydroquinolin-4-yl)ethyl)-4-methylthiazole-5-carboxamide

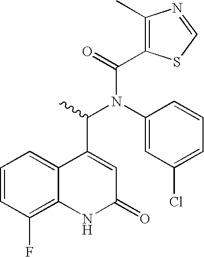

N-(3-Chlorophenyl)-N-1-(8-fluoro-2-oxo-1,2-dihydroquinolin-4-yl)ethyl)-4-methylthiazole-5-carboxamide was synthesized (as a racemic mixture) as described in EXAMPLE 43 using 2-fluoroaniline and ethyl 3-oxopentanoate as starting materials. LCMS: 442 (M+H)⁺.

### EXAMPLE 114

### N-(3-Chlorophenyl)-N-((3,8-difluoro-2-oxo-1,2-dihydroquinolin-4-yl)methyl)-4-methylnicotinamide

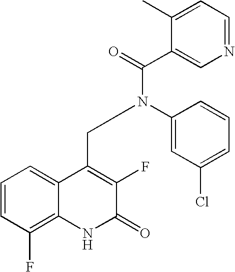

N-(3-Chlorophenyl)-N-((3,8-difluoro-2-oxo-1,2-dihydroquinolin-4-yl)methyl)-4-methylnicotinamide was synthesized as described in EXAMPLE 108 using 4-((3-chlorophenylamino)methyl)-3,8-difluoroquinolin-2(1H)-one and 4-methylnicotinic acid as starting materials. ¹H NMR (400 MHz, DMSO-d₆) δ 12.41 (s, 1H), 8.58 (s, 1H), 8.46 (d, 1H), 7.82 (d, 1H), 7.56 (d, 1H), 7.51-7.47 (m, 2H), 7.35 (m, 1H), 7.22 (d, 1H), 7.11 (t, 1H), 6.79 (d, 1H), 5.48 (s, 2H), 2.41 (s, 3H). LCMS: 440 (M+H)⁺.

### EXAMPLE 115

### N-(3-Chlorophenyl)-4-methyl-N-((3,7,8-trifluoro-2-oxo-1,2-dihydroquinolin-4-yl)methyl)thiazole-5-carboxamide

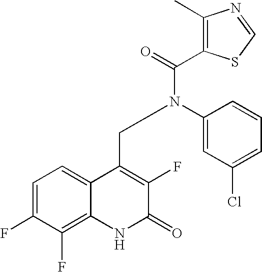

N-(3-Chlorophenyl)-4-methyl-N-((3,7,8-trifluoro-2-oxo-1,2-dihydroquinolin-4-yl)methyl)thiazole-5-carboxamide was synthesized as described in EXAMPLE 108 using 4-bromo-N-(2,3-difluorophenyl)-3-oxobutanamide as a starting material. ¹H NMR (400 MHz, DMSO-d₆) δ 12.63 (s, 1H), 8.88 (s, 1H), 7.81 (m, 1H), 7.48-7.42 (m, 2H), 7.32 (d, 1H), 7.19 (t, 1H), 6.79 (d, 1H), 5.42 (s, 2H), 2.42 (s, 3H). LCMS: 464 (M+H)⁺.

### EXAMPLE 116

### N-(3-Chlorophenyl)-4-methyl-N-((3,7,8-trifluoro-2-oxo-1,2-dihydroquinolin-4-yl)methyl)nicotinamide

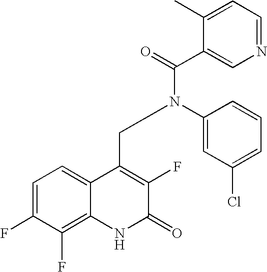

N-(3-Chlorophenyl)-4-methyl-N-((3,7,8-trifluoro-2-oxo-1,2-dihydroquinolin-4-yl)methyl)nicotinamide was synthesized as described in EXAMPLE 108 using 4-bromo-N-(2,3-difluorophenyl)-3-oxobutanamide and 4-methylnicotinic acid as starting materials. ¹H NMR (400 MHz, DMSO-d₆) δ 12.63 (s, 1H), 8.22 (m, 2H), 7.86 (m, 1H), 7.50 (m, 1H), 7.42 (s, 1H), 7.19-7.05 (m, 3H), 6.73 (d, 1H), 5.47 (s, 2H), 2.25 (s, 3H). LCMS: 458 (M+H)⁺.

### EXAMPLE 117

### N-(3-Chlorophenyl)-N-((8-fluoro-1-methyl-2-oxo-1,2-dihydroquinolin-4-yl)methyl)-4-methylthiazole-5-carboxamide

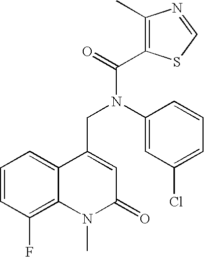

Sodium hydride (60% in mineral oil, 5 mg, 0.12 mmol) was added to a suspension of N-(3-chlorophenyl)-N-((8-fluoro-2-oxo-1,2-dihydroquinolin-4-yl)methyl)-4-methylthiazole-5-carboxamide (25 mg, 0.06 mmol) in DCM (2 mL). Dimethyl sulfate (6.6 μL, 0.07 mmol) was then added and the resulting mixture was stirred at RT for 18 h. The solvent was removed and the residue was purified by preparative HPLC (ACN/water) to afford 12 mg (46%) of N-(3-chlorophenyl)-N-((8-fluoro-1-methyl-2-oxo-1,2-dihydroquinolin-4-yl)methyl)-4-methylthiazole-5-carboxamide. ¹H NMR (400 MHz, DMSO-d₆) δ 8.94 (s, 1H), 7.74 (d, 1H), 7.54 (t, 1H), 7.49 (m, 1H), 7.35-7.26 (m, 3H), 7.10 (d, 1H), 6.55 (s, 1H), 5.36 (s, 2H), 3.73 (d, 3H), 2.43 (s, 3H).

### EXAMPLE 118

### 8-Fluoro-4-((2-(pyridin-3-yl)-1H-benzo[d]imidazol-1-yl)methyl)quinolin-2(1H)-one

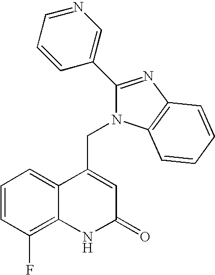

### Step 1: 2-(Pyridin-3-yl)-1H-benzo[d]imidazole

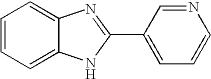

A mixture of benzene-1,2-diamine (2 g, 18.5 mmol) and nicotinic acid (2.5 g, 20.3 mmol) in polyphosphoric acid was heated to 200° C. for 2 h. The hot mixture was carefully poured into a mixture of ice/NaOH (1M) while stirring. The grey solid was filtered and dried for 18 h to yield 3 g (83%) of 2-(pyridin-3-yl)-1H-benzo[d]imidazole as a light grey solid. ¹H NMR (400 MHz, DMSO-d₆) δ 9.35 (s, 1H), 8.69 (d, 1H), 8.52 (d, 1H), 7.67-7.60 (m, 3H), 7.26 (m, 2H). LCMS: 196 (M+H)⁺.

### Step 2: 8-Fluoro-4-((2-(pyridin-3-yl)-1H-benzo[d]imidazol-1-yl)methyl)quinolin-2(1H)-one

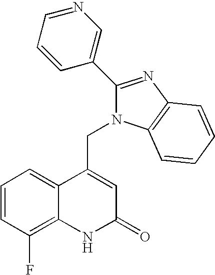

Sodium hydride (60% in mineral oil, 117 mg, 2.9 mmol) was added to 2-(pyridin-3-yl)-1H-benzo[d]imidazole (191 mg, 0.98 mmol) in DMF (5 mL) and stirred at RT for 15 min. 4-(Bromomethyl)-8-fluoroquinolin-2(1H)-one (300 mg, 1.2 mmol) was then added as a solid and the resulting mixture was stirred at RT for 18 h. The crude mixture was purified by preparative HPLC (ACN/water) to yield 60 mg (16%) of 8-fluoro-4-((2-(pyridin-3-yl)-1H-benzo[d]imidazol-1-yl)methyl)quinolin-2(1H)-one as a pale yellow. ¹H NMR (400 MHz, DMSO-d₆, TFA salt) δ 11.86 (s, 1H), 8.93 (s, 1H), 8.72 (d, 1H), 8.13 (d, 1H), 7.86 (d, 1H), 7.69 (d, 1H), 7.64 (d, 1H), 7.59-7.57 (m, 2H), 7.40-7.36 (m, 2H), 7.26 (m, 1H), 5.94 (s, 2H), 5.53 (s, 1H). LCMS: 371 (M+H)⁺.

### EXAMPLE 119

### 7,8-Difluoro-4-((2-(4-methylthiazol-5-yl)-1H-benzo[d]imidazol-1-yl)methyl)quinolin-2(1H)-one

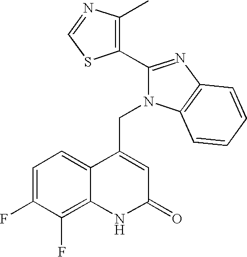

7,8-Difluoro-4-((2-(4-methylthiazol-5-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 5-(1H-benzo[d]imidazol-2-yl)-4-methylthiazole as starting materials. ¹H NMR (400 MHz, DMSO-d₆) δ 12.11 (s, 1H), 9.14 (s, 1H), 7.85 (d, 1H), 7.73 (m, 1H), 7.64 (d, 1H), 7.39-7.35 (m, 3H), 5.82 (s, 2H), 5.30 (s, 1H), 2.52 (s, 3H). LCMS: 408 (M)⁺.

### EXAMPLE 120

### 7,8-Difluoro-4-((2-(pyridin-3-yl)-1H-benzo[d]imidazol-1-yl)methyl)quinolin-2(1H)-one

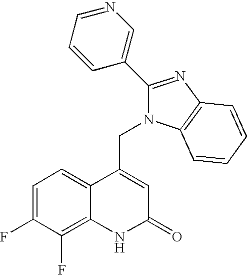

7,8-Difluoro-4-((2-(pyridin-3-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(pyridin-3-yl)-1H-benzo[d]imidazole as starting materials. ¹H NMR (400 MHz, DMSO-d₆, HCl salt) δ 12.10 (s, 1H), 8.99 (s, 1H), 8.81 (d, 1H), 8.26 (d, 1H), 7.91 (d, 1H), 7.72-7.67 (m, 3H), 7.52-7.46 (m, 2H), 7.39-7.32 (m, 1H), 5.98 (s, 2H), 5.71 (s, 1H). LCMS: 388 (M)⁺.

### EXAMPLE 121

### 7,8-Difluoro-4-((2-(4-methylpyridin-3-yl)-1H-benzo[d]imidazol-1-yl)methyl)quinolin-2(1H)-one

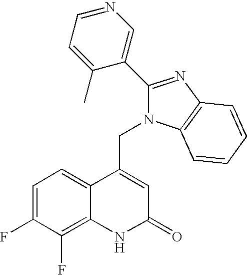

7,8-Difluoro-4-((2-(4-methylpyridin-3-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(4-methylpyridin-3-yl)-1H-benzo[d]imidazole as starting materials. ¹H NMR (400 MHz, DMSO-d₆, HCl salt) δ 12.10 (s, 1H), 8.88 (s, 1H), 8.78 (d, 1H), 7.90 (d, 1H), 7.83 (d, 1H), 7.71 (d, 1H), 7.59-7.55 (m, 1H), 7.51-7.44 (m, 2H), 7.33-7.26 (m, 1H), 5.82 (s, 2H), 5.64 (s, 1H), 2.42 (s, 3H). LCMS: 403 (M+H)⁺.

### EXAMPLE 122

### 7,8-Difluoro-4-((2-(1-methyl-1H-imidazol-5-yl)-1H-benzo[d]imidazol-1-yl)methyl)quinolin-2(1H)-one

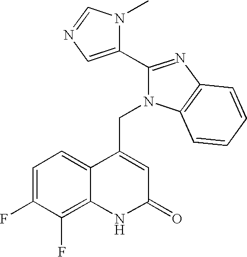

7,8-Difluoro-4-((2-(1-methyl-1H-imidazol-5-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(1-methyl-1H-imidazol-5-yl)-1H-benzo[d]imidazole as starting materials. ¹H NMR (400 MHz, DMSO-d₆, HCl salt) δ 12.09 (s, 1H), 9.27 (s, 1H), 7.90-7.86 (m, 2H), 7.72-7.61 (m, 2H), 7.43-7.36 (m, 3H), 5.94 (s, 2H), 5.22 (s, 1H), 4.01 (s, 3H). LCMS: 392 (M+H)⁺.

### EXAMPLE 123

### 4-((5-Chloro-2-(pyridin-3-yl)-1H-benzo[d]imidazol-1-yl)methyl)-7,8-difluoroquinolin-2(1H)-one and 4-((6-chloro-2-(pyridin-3-yl)-1H-benzo[d]imidazol-1-yl)methyl)-7,8-difluoroquinolin-2(1H)-one

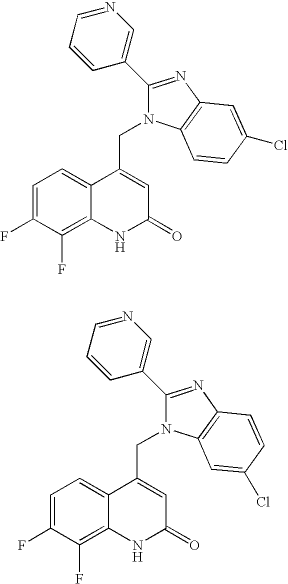

4-((5-Chloro-2-(pyridin-3-yl)-1H-benzo[d]imidazol-1-yl)methyl)-7,8-difluoroquinolin-2(1H)-one and 4-((6-chloro-2-(pyridin-3-yl)-1H-benzo[d]imidazol-1-yl)methyl)-7,8-difluoroquinolin-2(1H)-one were synthesized as a 1:1 mixture as described in EXAMPLE 118 using 4-(bromomethyl)-7,8-difluoroquinolin-2(1H)-one and 5-chloro-2-(pyridin-3-yl)-1H-benzo[d]imidazole as starting materials. LCMS (TFA salt): 423 (M+H)⁺.

### EXAMPLE 124

### 4-((2-(4-Methylthiazol-5-yl)-1H-benzo[d]imidazol-1-yl)methyl)quinolin-2(1H)-one

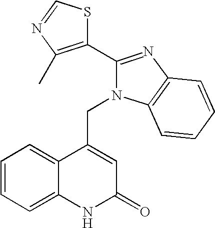

4-((2-(4-Methylthiazol-5-yl)-1H-benzo[d]imidazol-1-yl)methyl)quinolin-2(1H)-one was synthesized as described in EXAMPLE 118 using 4-(bromomethyl)quinolin-2(1H)-one and 5-(1H-benzo[d]imidazol-2-yl)-4-methylthiazole as starting materials. ¹H NMR (400 MHz, DMSO-d₆) δ 11.80 (s, 1H), 9.12 (s, 1H), 7.83 (d, 2H), 7.64 (d, 1H), 7.56 (t, 1H), 7.38-7.34 (m, 3H), 7.24 (t, 1H), 5.83 (s, 2H), 5.30 (s, 1H), 2.50 (s, 3H). LCMS: 372 (M)⁺.

### EXAMPLE 125

### 4-((2-(Pyridin-3-yl)-1H-benzo[d]imidazol-1-yl)methyl)quinolin-2(1H)-one

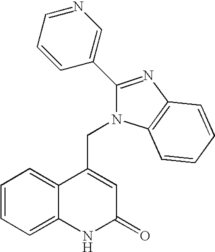

4-((2-(Pyridin-3-yl)-1H-benzo[d]imidazol-1-yl)methyl)quinolin-2(1H)-one was synthesized as described in EXAMPLE 118 using 4-(bromomethyl)quinolin-2(1H)-one and 2-(pyridin-3-yl)-1H-benzo[d]imidazole as starting materials. ¹H NMR (400 MHz, DMSO-d₆, TFA salt) δ 11.83 (s, 1H), 8.94 (s, 1H), 8.73 (d, 1H), 8.15 (d, 1H), 7.87 (d, 2H), 7.65-7.58 (m, 3H), 7.41-7.37 (m, 3H), 7.27 (t, 1H), 5.96 (s, 2H), 5.47 (s, 1H). LCMS: 352 (M)⁺.

### EXAMPLE 126

### 4-((5-Chloro-2-(4-methylthiazol-5-yl)-1H-benzo[d]imidazol-1-yl)methyl)-7,8-difluoroquinolin-2(1H)-one and 4-((6-chloro-2-(4-methylthiazol-5-yl)-1H-benzo[d]imidazol-1-yl)methyl)-7,8-difluoroquinolin-2(1H)-one

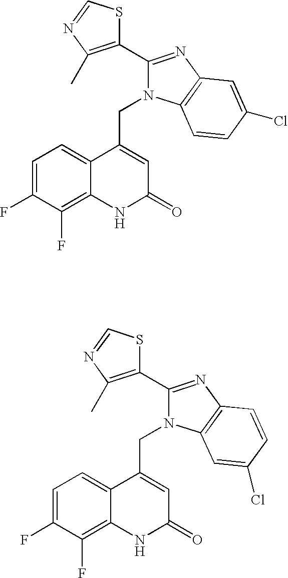

4-((5-Chloro-2-(4-methylthiazol-5-yl)-1H-benzo[d]imidazol-1-yl)methyl)-7,8-difluoroquinolin-2(1H)-one and 4-((6-chloro-2-(4-methylthiazol-5-yl)-1H-benzo[d]imidazol-1-yl)methyl)-7,8-difluoroquinolin-2(1H)-one were synthesized as a 1:1 mixture as described in EXAMPLE 118 using 4-(bromomethyl)-7,8-difluoroquinolin-2(1H)-one and 5-(5-chloro-1H-benzo[d]imidazol-2-yl)-4-methylthiazole as starting materials. LCMS: 443 (M+H)⁺.

### EXAMPLE 127

### 7,8-Difluoro-4-((2-(pyridin-2-yl)-1H-benzo[d]imidazol-1-yl)methyl)quinolin-2(1H)-one

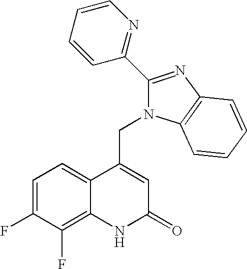

7,8-Difluoro-4-((2-(pyridin-2-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(pyridin-2-yl)-1H-benzo[d]imidazole as starting materials. ¹H NMR (400 MHz, DMSO-d₆, HCl salt) δ 8.48 (d, 1H), 8.41 (d, 1H), 8.01 (t, 1H), 7.86 (d, 2H), 7.67 (d, 1H), 7.48 (t, 1H), 7.43-7.38 (m, 3H), 6.44 (s, 2H), 5.28 (s, 1H). LCMS: 388 (M)⁺.

### EXAMPLE 128

### 7,8-Difluoro-4-((2-(pyridin-4-yl)-1H-benzo[d]imidazol-1-yl)methyl)quinolin-2(1H)-one

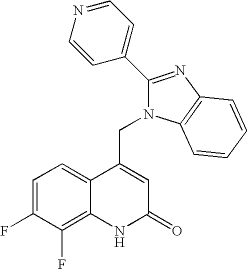

7,8-Difluoro-4-((2-(pyridin-4-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(pyridin-4-yl)-1H-benzo[d]imidazole as starting materials. ¹H NMR (400 MHz, DMSO-d₆, HCl salt) δ 12.06 (s, 1H), 8.67 (d, 2H), 7.81 (d, 1H), 7.69 (m, 3H), 7.54 (d, 1H), 7.34-7.29 (m, 3H), 5.89 (s, 2H), 5.34 (s, 1H). LCMS: 389 (M+H)⁺.

### EXAMPLE 129

### 7,8-Difluoro-4-((2-(pyridin-3-ylmethyl)-1H-benzo[d]imidazol-1-yl)methyl)quinolin-2(1H)-one

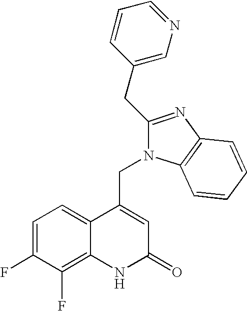

### Step 1: 2-(Pyridin-3-ylmethyl)-1H-benzo[d]imidazole

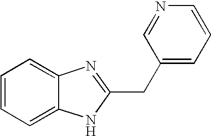

A mixture of 2-(pyridin-3-yl)acetic acid (1.5 g, 8.7 mmol) and benzene-1,2-diamine (312 mg, 2.9 mmol) was heated to 140° C. for 3 h and cooled to RT overnight. The black residue was partitioned between DCM and aqueous saturated sodium bicarbonate and the organic layer was washed with aqueous saturated sodium bicarbonate (2×) and water (2×). The solvent was removed and the residue was purified by silica gel flash column chromatography (Hexanes/EtOAc) to afford 304 mg (50%) of 2-(pyridin-3-ylmethyl)-1H-benzo[d]imidazole as a brown solid. ¹H NMR (400 MHz, DMSO-d₆) δ 8.35 (d, 2H), 7.65-7.54 (m, 3H), 7.29-7.15 (m, 3H), 4.20 (s, 2H).

### Step 2: 7,8-Difluoro-4-((2-(pyridin-3-ylmethyl)-1H-benzo[d]imidazol-1-yl)methyl)quinolin-2(1H)-one

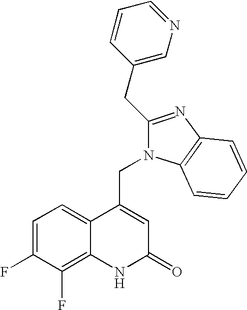

7,8-Difluoro-4-((2-(pyridin-3-ylmethyl)-1H-benzo[d]imidazol-1-yl)methyl)quinolin-2(1H)-one was synthesized as described in EXAMPLE 118, Step 2 using 4-(bromomethyl)-7,8-difluoroquinolin-2(1H)-one and 2-(pyridin-3-ylmethyl)-1H-benzo[d]imidazole as starting materials. ¹H NMR (400 MHz, DMSO-d₆, HCl salt) δ 12.09 (s, 1H), 9.00 (s, 1H), 8.80 (d, 1H), 8.52 (d, 1H), 7.93 (d, 1H), 7.82-7.72 (m, 3H), 7.49-7.43 (m, 3H), 6.16 (s, 2H), 5.35 (s, 1H), 4.82 (s, 2H). LCMS: 402 (M)⁺.

### EXAMPLE 130

### 8-Fluoro-5-methyl-4-((2-(4-methylthiazol-5-yl)-1H-benzo[d]imidazol-1-yl)methyl)quinolin-2(1H)-one

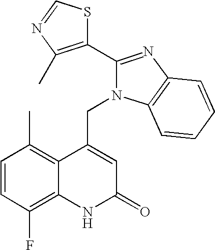

8-Fluoro-5-methyl-4-((2-(4-methylthiazol-5-yl)-1H-benzo[d]imidazol-1-yl)methyl)quinolin-2(1H)-one was synthesized as described in EXAMPLE 118 using 4-(bromomethyl)-8-fluoro-5-methylquinolin-2(1H)-one and 5-(1H-benzo[d]imidazol-2-yl)-4-methylthiazole as starting materials. ¹H NMR (400 MHz, DMSO-d₆) δ 11.64 (s, 1H), 9.10 (s, 1H), 7.85-7.81 (m, 1H), 7.74-7.70 (m, 1H), 7.38-7.33 (m, 3H), 7.04 (m, 1H), 5.97 (s, 2H), 5.16 (s, 1H), 2.76 (s, 3H), 2.55 (s, 3H). LCMS: 404 (M)⁺.

### EXAMPLE 131

### 7-Fluoro-4-((2-(4-methylthiazol-5-yl)-1H-benzo[d]imidazol-1-yl)methyl)quinolin-2(1H)-one

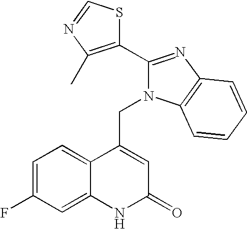

7-Fluoro-4-((2-(4-methylthiazol-5-yl)-1H-benzo[d]imidazol-1-yl)methyl)quinolin-2(1H)-one was synthesized as described in EXAMPLE 118 using 4-(bromomethyl)-7-fluoroquinolin-2(1H)-one and 5-(1H-benzo[d]imidazol-2-yl)-4-methylthiazole as starting materials. ¹H NMR (400 MHz, DMSO-d₆) δ 11.89 (s, 1H), 9.12 (s, 1H), 7.93-7.89 (m, 1H), 7.81 (d, 1H), 7.62 (d, 1H), 7.36-7.33 (m, 2H), 7.16-7.07 (m, 2H), 5.81 (s, 2H), 5.22 (s, 1H), 2.52 (s, 3H). LCMS: 391 (M+H)⁺.

### EXAMPLE 132

### 8-Chloro-4-((2-(4-methylthiazol-5-yl)-1H-benzo[d]imidazol-1-yl)methyl)quinolin-2(1H)-one

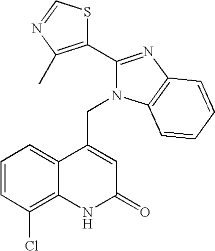

8-Chloro-4-((2-(4-methylthiazol-5-yl)-1H-benzo[d]imidazol-1-yl)methyl)quinolin-2(1H)-one was synthesized as described in EXAMPLE 118 using 4-(bromomethyl)-8-chloroquinolin-2(1H)-one and 5-(1H-benzo[d]imidazol-2-yl)-4-methylthiazole as starting materials. ¹H NMR (400 MHz, DMSO-d₆) δ 11.10 (s, 1H), 9.10 (s, 1H), 7.87-7.81 (m, 2H), 7.74 (d, 1H), 7.63 (d, 1H), 7.38-7.25 (m, 3H), 5.84 (s, 2H), 5.35 (s, 1H), 2.55 (s, 3H). LCMS: 407 (M+H)⁺.

### EXAMPLE 133

### 6,7-Difluoro-4-((2-(4-methylthiazol-5-yl)-1H-benzo[d]imidazol-1-yl)methyl)quinolin-2(1H)-one

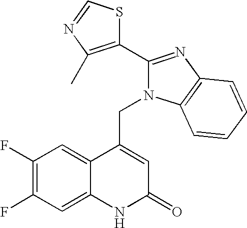

6,7-Difluoro-4-((2-(4-methylthiazol-5-yl)-1H-benzo[d]imidazol-1-yl)methyl)quinolin-2(1H)-one was synthesized as described in EXAMPLE 118 using 4-(bromomethyl)-6,7-difluoroquinolin-2(1H)-one and 5-(1H-benzo[d]imidazol-2-yl)-4-methylthiazole as starting materials. ¹H NMR (400 MHz, DMSO-d₆) δ 11.93 (s, 1H), 9.13 (s, 1H), 8.00 (m, 1H), 7.82 (d, 1H), 7.59 (d, 1H), 7.36-7.26 (m, 3H), 5.78 (s, 2H), 5.26 (s, 1H), 2.55 (s, 3H). LCMS: 409 (M+H)⁺.

### EXAMPLE 134

### 6-Methyl-4-((2-(4-methylthiazol-5-yl)-1H-benzo[d]imidazol-1-yl)methyl)quinolin-2(1H)-one

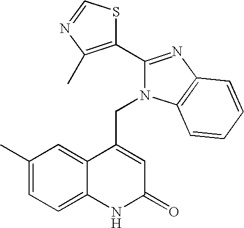

6-Methyl-4-((2-(4-methylthiazol-5-yl)-1H-benzo[d]imidazol-1-yl)methyl)quinolin-2(1H)-one was synthesized as described in EXAMPLE 118 using 4-(bromomethyl)-6-methylquinolin-2(1H)-one and 5-(1H-benzo[d]imidazol-2-yl)-4-methylthiazole as starting materials. ¹H NMR (400 MHz, DMSO-d₆) δ 11.71 (s, 1H), 9.12 (s, 1H), 7.81 (d, 1H), 7.64-7.60 (m, 2H), 7.40-7.26 (m, 3H), 7.23 (d, 1H), 5.80 (s, 2H), 5.24 (s, 1H), 2.55 (s, 3H), 2.35 (s, 3H). LCMS: 387 (M+H)⁺.

### EXAMPLE 135

### 7,8-Difluoro-4-((2-phenyl-1H-benzo[d]imidazol-1-yl)methyl)quinolin-2(1H)-one

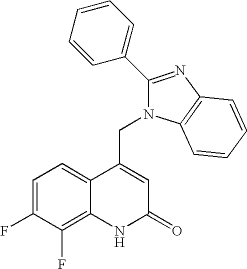

7,8-Difluoro-4-((2-phenyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-phenyl-1H-benzo[d]imidazole as starting materials. ¹H NMR (400 MHz, DMSO-d₆, HCl salt) δ 12.05 (s, 1H), 7.89 (d, 1H), 7.77-7.48 (m, 9H), 7.34 (m, 1H), 5.93 (s, 2H), 5.82 (s, 1H). LCMS: 388 (M+H)⁺.

### EXAMPLE 136

### 7,8-Difluoro-4-((2-isopropyl-1H-benzo[d]imidazol-1-yl)methyl)quinolin-2(1H)-one

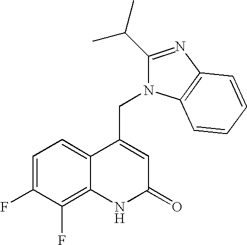

7,8-Difluoro-4-((2-isopropyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-isopropyl-1H-benzo[d]imidazole as starting materials. ¹H NMR (400 MHz, DMSO-d₆, HCl salt) δ 12.12 (s, 1H), 7.87 (d, 1H), 7.79-7.75 (m, 2H), 7.59-7.42 (m, 3H), 6.11 (s, 2H), 5.51 (s, 1H), 3.58 (m, 1H), 1.41 (s, 3H), 1.39 (s, 3H). LCMS: 354 (M+H)⁺.

### EXAMPLE 137

### 7,8-Difluoro-4-((2,5,6-trimethyl-1H-benzo[d]imidazol-1-yl)methyl)quinolin-2(1H)-one

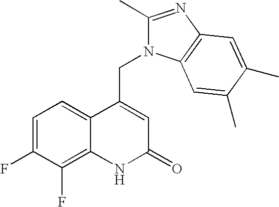

7,8-difluoro-4-((2,5,6-trimethyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2,5,6-trimethyl-1H-benzo[d]imidazole as starting materials. ¹H NMR (400 MHz, DMSO-d₆) δ 12.11 (s, 1H), 7.75-7.70 (m, 1H), 7.62 (s, 1H), 7.57 (s, 1H), 7.44 (q, 1H), 5.99 (s, 2H), 5.45 (s, 1H), 2.74 (s, 3H), 2.37 (s, 3H), 2.30 (s, 3H). LCMS: 353.64 (M+H)⁺.

### EXAMPLE 138

### 8-Fluoro-4-((2-(4-methylthiazol-5-yl)-1H-indol-1-yl)methyl)quinolin-2-ol

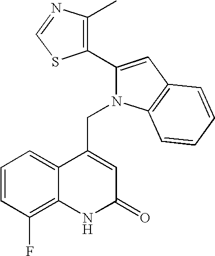

### Step 1: tert-Butyl 1H-indole-1-carboxylate

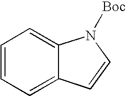

Sodium hydride (7.5 mg, 0.31 mmol) was added to a solution of 1H-indole (40 g, 341.44 mmol) in THF (500 mL) at −10° C. and then the mixture was stirred for 1 h at RT. To the above was added (Boc)₂O (70 g, 320.73 mmol) in several batches with stirring. The resulting solution was stirred at RT for 4 h (reaction progress monitored by TLC (EtOAc/PE=10:1)). The reaction mixture was then quenched by the adding 200 g of H₂O/ice. The resulting solution was extracted with EtOAc (2×400 mL) and the organic layers were combined and dried over Na₂SO₄. This resulted in 60 g (92%) of tert-butyl 1H-indole-1-carboxylate as yellow liquid.

### Step 2: 1-(tert-Butoxycarbonyl)-1H-indol-2-ylboronic acid

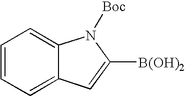

Lithium diisopropylamide (140 mL) was added dropwise to a mixture of tert-butyl 1H-indole-1-carboxylate (26 g, 119.67 mmol) and triisopropyl borate (30 g, 159.57 mmol) in ethoxyethane (500 mL) at −70° C. The resulting solution was stirred at −70° C. (reaction progress monitored by TLC (EtOAc/PE=1:5)). The reaction mixture was then quenched by the adding 200 g of water/ice. Adjustment of the pH to 7 was accomplished by the addition of HCl (10%). The resulting solution was extracted with Ether (1×100 mL) and the organic layers were combined and dried over Na₂SO₄ to afford 13 g (67%) of 1-(tert-butoxycarbonyl)-1H-indol-2-ylboronic acid as a white solid.

### Step 3: tert-Butyl 2-(4-methylthiazol-5-yl)-1H-indole-1-carboxylate

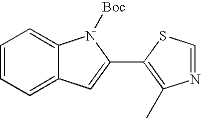

A mixture of 5-bromo-4-methylthiazole (400 mg, 2.26 mmol), tert-butyl 1H-indole-1-carboxylate (2400 mg, 11.06 mmol), Na₂CO₃ (400 mg, 3.77 mmol), and Pd(PPh₃)₄ (50 mg) in DME/water (10:1, 22 mL) was stirred at 60° C. for 18 h. The mixture was concentrated by evaporation under vacuum using a rotary evaporator. The residue was purified by column chromatography on silica gel (eluting with 1:100 EtOAc/PE solvent system) to afford 200 mg (25%) of tert-butyl 2-(4-methylthiazol-5-yl)-1H-indole-1-carboxylate as a yellow solid.

### Step 4: 2-(4-Methylthiazol-5-yl)-1H-indole

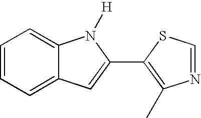

A mixture of tert-butyl 2-(4-methylthiazol-5-yl)-1H-indole-1-carboxylate (300 mg, 0.86 mmol) and TFA (5 mL) in DCM (10 mL) was stirred at RT for 18 h. The mixture was concentrated under vacuum to afford 100 mg (49%) of 2-(4-methylthiazol-5-yl)-1H-indole as a yellow solid.

### Step 5: 8-Fluoro-4-((2-(4-methylthiazol-5-yl)-1H-indol-1-yl)methyl)quinolin-2-ol

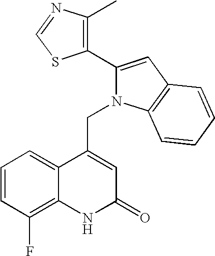

Sodium hydride (500 mg, 20.83 mmol) was added to 2-(4-methylthiazol-5-yl)-1H-indole (100 mg, 0.47 mmol) in DMF (10 mL). To the above was added 4-(bromomethyl)-8-fluoroquinolin-2-ol (400 mg, 1.57 mmol) in several batches, while maintaining the content at RT. The resulting solution was stirred for 4 h at RT. The mixture was concentrated and the residue was purified by column chromatography on silica gel (eluting with 1:100 EtOAc/PE solvent system) to afford 60 mg (26%) of 8-fluoro-4-((2-(4-methylthiazol-5-yl)-1H-indol-1-yl)methyl)quinolin-2-ol as a yellow solid. LCMS: 390 (M+H)⁺.

### EXAMPLE 139

### N-(3-Chlorophenyl)-N-((8-fluoro-2-oxo-1,2-dihydroquinolin-4-yl)methyl)-4-methyl-1,2,3-thiadiazole-5-carboxamide

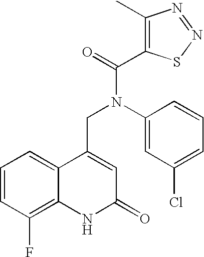

N-(3-Chlorophenyl)-N-((8-fluoro-2-oxo-1,2-dihydroquinolin-4-yl)methyl)-4-methyl-1,2,3-thiadiazole-5-carboxamide was synthesized as described in EXAMPLE 26 using 4-((3-chlorophenylamino)methyl)-8-fluoroquinolin-2(1H)-one and 4-methyl-1,2,3-thiadiazole-5-carboxylic acid as starting materials. ¹H NMR (400 MHz, DMSO-d₆) δ 11.76 (s, 1H), 7.66-7.60 (m, 2H), 7.44 (dd, 1H), 7.33 (d, 1H), 7.29-7.15 (m, 3H), 6.58 (s, 1H), 5.36 (s, 2H), 2.64 (s, 3H). LCMS: 429 (M+H)⁺.

### EXAMPLE 140

### N-(3-Chlorophenyl)-N-((8-fluoro-2-oxo-1,2-dihydroquinolin-4-yl)methyl)-1,2,3-thiadiazole-4-carboxamide

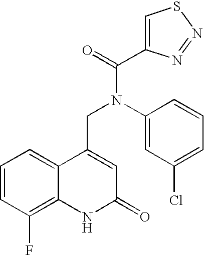

N-(3-Chlorophenyl)-N-((8-fluoro-2-oxo-1,2-dihydroquinolin-4-yl)methyl)-1,2,3-thiadiazole-4-carboxamide was synthesized as described in EXAMPLE 26 using 4-((3-chlorophenylamino) methyl)-8-fluoroquinolin-2(1H)-one and 1,2,3-thiadiazole-5-carboxylic acid as starting materials. ¹H NMR (400 MHz, DMSO-d₆) δ 11.76 (s, 1H), 9.49 (s, 1H), 7.68 (d, 1H), 7.48-7.42 (m, 2H), 7.26-7.09 (m, 4H), 6.59 (s, 1H), 5.45 (s, 2H). LCMS: 415.2 (M+H)⁺.

### EXAMPLE 141

### N-(3-Chlorophenyl)-N-((7,8-difluoro-2-oxo-1,2-dihydroquinolin-4-yl)methyl)-1-methyl-1H-imidazole-5-carboxamide

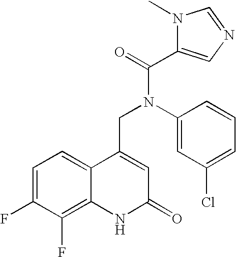

N-(3-Chlorophenyl)-N-((7,8-difluoro-2-oxo-1,2-dihydroquinolin-4-yl)methyl)-1-methyl-1H-imidazole-5-carboxamide was synthesized as described in EXAMPLE 46 using 4-(bromomethyl)-7,8-difluoroquinolin-2(1H)-one as the starting material in the synthesis of the intermediate A described in Step 1 of that sequence. ¹H NMR (400 MHz, DMSO-d₆) δ 12.00 (s, 1H), 7.71-7.64 (m, 2H), 7.53 (s, 1H), 7.36-7.22 (m, 4H), 6.43 (s, 1H), 6.17 (s, 1H), 5.27 (s, 2H), 3.83 (s, 3H). LCMS: 428.9 (M+H)⁺.

### EXAMPLE 142

### N-(3-Chlorophenyl)-N-((8-fluoro-2-oxo-1,2-dihydroquinolin-4-yl)methyl)-4-(trifluoromethyl)thiazole-5-carboxamide

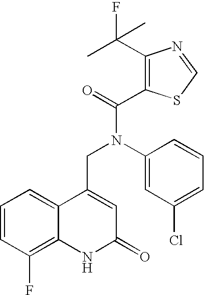

### Step 1: 4-(Trifluoromethyl)thiazole-5-carboxylic acid

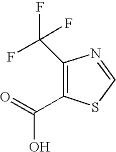

2-Amino-4-(trifluoromethyl)thiazole-5-carboxylic acid (424 mg, 2.0 mmol) was dissolved with warming in 85% phosphoric acid (14 mL). The resulting solution was cooled to −10° C. and a solution of NaNO₂ (828 mg, 12.0 mmol) in water (3 mL) was added slowly (5 min) below the surface of the solution. After 30 min, the resulting foamy orange mixture was transferred to a beaker containing 50% aqueous H₃PO₂ (10 mL). After 2 h, TLC analysis (10% MeOH in EtOAc) revealed the disappearance of starting material and a new more polar spot. The mixture was diluted with water (100 mL), adjusted to pH 5 with 5N NaOH, and extracted with EtOAc (3×100 mL). The combined organic layers were dried over MgSO₄, filtered, and concentrated to dryness under reduced pressure. The product 4-(trifluoromethyl)thiazole-5-carboxylic acid (340 mg, 86%) was determined to be sufficiently pure by ¹H NMR to use in the next step. ¹H NMR (400 MHz, DMSO-d₆) δ 9.32 (s, 1H).

### Step 2: N-(3-Chlorophenyl)-4-(trifluoromethyl)thiazole-5-carboxamide

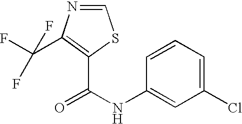

O-(7-Azabenzotriazol-1-yl)-N,N,N,N-tetramethyluronium hexafluorophosphate (HATU, 260 mg, 0.68 mmol) was added to a stirred mixture of 4-(trifluoromethyl)thiazole-5-carboxylic acid (113 mg, 0.57 mmol), 3-chloroaniline (78 μL, 0.74 mmol) and triethylamine (160 μL, 1.14 mmol) in DMF (6 mL). After 4 h, the reaction was determined to be complete by TLC and LCMS analysis. The mixture was worked up by aqueous extraction and purified via chromatography on silica gel, eluting with 50% EtOAc in hexanes to afford N-(3-chlorophenyl)-4-(trifluoromethyl)thiazole-5-carboxamide (100 mg, 57%) as a tan solid. LCMS: 306.7 (M+H)⁺.

### Step 3: N-(3-Chlorophenyl)-N-((8-fluoro-2-oxo-1,2-dihydroquinolin-4-yl)methyl)-4-(trifluoromethyl)thiazole-5-carboxamide

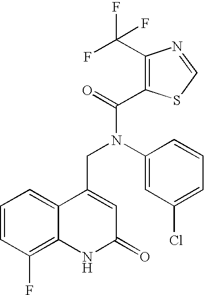

N-(3-Chlorophenyl)-N-((8-fluoro-2-oxo-1,2-dihydroquinolin-4-yl)methyl)-4-(trifluoromethyl)thiazole-5-carboxamide was synthesized as described in EXAMPLE 46 using N-(3-chlorophenyl)-4-(trifluoromethyl)thiazole-5-carboxamide and 4-(bromomethyl)-8-fluoroquinolin-2(1H)-one as starting materials. ¹H NMR (400 MHz, DMSO-d₆) δ 11.76 (s, 1H), 9.14 (s, 1H), 7.63 (d, 1H), 7.48-7.41 (m, 2H), 7.31-7.19 (m, 3H), 6.95 (m, 1H), 6.45 (s, 1H), 5.36 (s, 2H). LCMS: 481.6 (M+H)⁺.

### EXAMPLE 143

### N-(3-Chlorophenyl)-N-((8-fluoro-2-oxo-1,2-dihydroquinolin-4-yl)methyl)cyclopentanecarboxamide

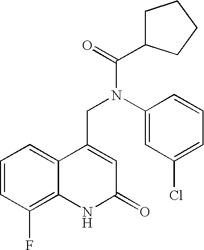

N-(3-Chlorophenyl)-N-((8-fluoro-2-oxo-1,2-dihydroquinolin-4-yl)methyl)cyclopentane carboxamide was synthesized as described in EXAMPLE 43 using 4-((3-chlorophenylamino) methyl)-8-fluoroquinolin-2(1H)-one and cyclopentanecarboxylic acid as starting materials. ¹H NMR (400 MHz, DMSO-d₆) δ 11.72 (s, 1H), 7.55-7.05 (m, 7H), 6.28 (s, 1H), 5.07 (s, 2H), 2.59 (m, 1H), 1.72-1.28 (m, 8H). LCMS: 398.8 (M+H)⁺.

### EXAMPLE 144

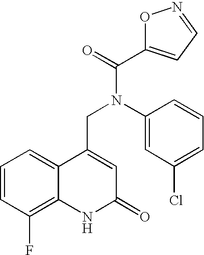

N-(3-Chlorophenyl)-N-((8-fluoro-2-oxo-1,2-dihydroquinolin-4-yl)methyl)isoxazole-5-carboxamide was synthesized as described in EXAMPLE 43 using 4-((3-chlorophenylamino) methyl)-8-fluoroquinolin-2(1H)-one and isoxazole-5-carboxylic acid as starting materials. ¹H NMR (400 MHz, DMSO-d₆) δ 11.78 (s, 1H), 8.56 (s, 1H), 7.62-7.15 (m, 8H), 6.48 (s, 1H), 5.33 (s, 2H). LCMS: 397.8 (M+H)⁺.

### EXAMPLE 145

### N-(3-Chlorophenyl)-N-((8-fluoro-2-oxo-1,2-dihydroquinolin-4-yl)methyl)-3,5-dimethylisoxazole-4-carboxamide

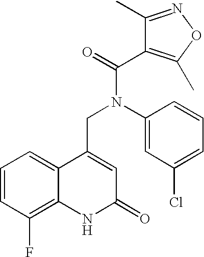

N-(3-Chlorophenyl)-N-((8-fluoro-2-oxo-1,2-dihydroquinolin-4-yl)methyl)-3,5-dimethylisoxazole-4-carboxamide was synthesized as described in EXAMPLE 43 using 4-((3-chlorophenylamino) methyl)-8-fluoroquinolin-2(1H)-one and 3,5-dimethylisoxazole-4-carboxylic acid as starting materials. ¹H NMR (400 MHz, DMSO-d₆) δ 11.77 (s, 1H), 7.65 (d, 1H), 7.50-7.40 (m, 2H), 7.30-7.18 (m, 3H), 7.07-7.02 (m, 1H), 6.37 (s, 1H), 5.37 (s, 2H), 2.10 (d, 6H). LCMS: 425.8 (M+H)⁺.

### EXAMPLE 146

### N-(3-Chlorophenyl)-N-((7,8-difluoro-2-oxo-1,2-dihydroquinolin-4-yl)methyl)-3,5-dimethylisoxazole-4-carboxamide

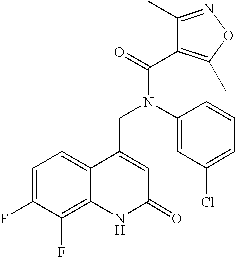

N-(3-Chlorophenyl)-N-((7,8-difluoro-2-oxo-1,2-dihydroquinolin-4-yl)methyl)-3,5-dimethylisoxazole-4-carboxamide was synthesized as described in EXAMPLE 43 using 4-((3-chlorophenylamino)methyl)-7,8-difluoroquinolin-2(1H)-one and 3,5-dimethylisoxazole-4-carboxylic acid as starting materials. ¹H NMR (400 MHz, DMSO-d₆) δ 12.00 (s, 1H), 7.68 (m, 1H), 7.50 (s, 1H), 7.40-7.22 (m, 3H), 7.04 (m, 1H), 6.32 (s, 1H), 5.37 (s, 2H), 2.09 (d, 6H). LCMS: 443.7 (M+H)⁺.

### EXAMPLE 147

### N-(3-Chlorophenyl)-N-((7,8-difluoro-2-oxo-1,2-dihydroquinolin-4-yl)methyl)cyclopropanecarboxamide

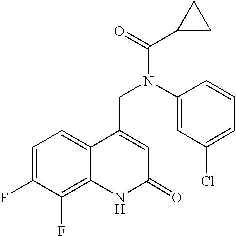

N-(3-Chlorophenyl)-N-((7,8-difluoro-2-oxo-1,2-dihydroquinolin-4-yl)methyl)cyclopropanecarboxamide was synthesized as described in EXAMPLE 43 using 4-((3-chlorophenylamino)methyl)-7,8-difluoroquinolin-2(1H)-one and cyclopropanecarboxylic acid as starting materials. ¹H NMR (400 MHz, DMSO-d₆) δ 11.95 (s, 1H), 7.61-7.38 (m, 6H), 6.23 (s, 1H), 5.12 (s, 2H), 1.42 (m, 1H), 0.93-0.68 (m, 4H). LCMS: 388.9 (M+H)⁺.

### EXAMPLE 148

### N-(3-Chlorophenyl)-N-((7,8-difluoro-2-oxo-1,2-dihydroquinolin-4-yl)methyl)-4-methylnicotinamide

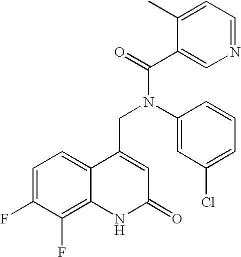

N-(3-Chlorophenyl)-N-((7,8-difluoro-2-oxo-1,2-dihydroquinolin-4-yl)methyl)-4-methylnicotinamide was synthesized as described in EXAMPLE 43 using 4-((3-chlorophenylamino)methyl)-7,8-difluoroquinolin-2(1H)-one and 4-methylnicotinic acid as starting materials. ¹H NMR (400 MHz, DMSO-d₆; TFA salt) δ 12.01 (s, 1H), 8.46 (s, 1H), 8.35 (d, 1H), 7.72 (m, 1H), 7.46 (s, 1H), 7.40-6.90 (m, 5H), 6.42 (s, 1H), 5.35 (s, 2H), 2.32 (s, 3H). LCMS: 440.2 (M+H)⁺.

### EXAMPLE 149

### N-(3-Chlorophenyl)-N-((8-fluoro-2-oxo-1,2-dihydroquinolin-4-yl)methyl)-4-(trifluoromethyl)nicotinamide

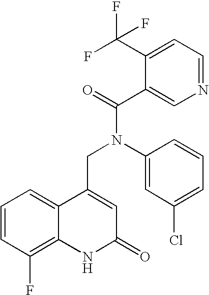

N-(3-Chlorophenyl)-N-((8-fluoro-2-oxo-1,2-dihydroquinolin-4-yl)methyl)-4-(trifluoromethyl)nicotinamide was synthesized as described in EXAMPLE 43 using 4-((3-chlorophenylamino) methyl)-8-fluoroquinolin-2(1H)-one and 4-(trifluoromethyl)nicotinic acid as starting materials. ¹H NMR (400 MHz, DMSO-d₆) δ 12.01 (s, 1H), 8.80-8.60 (m, 2H), 7.80-6.40 (m, 9H), 5.40 (s, 2H). LCMS: 475.7 (M+H)⁺.

### EXAMPLE 150

### N-(3-Chlorophenyl)-N-((8-fluoro-2-oxo-1,2-dihydroquinolin-4-yl)methyl)-4-methylisoxazole-5-carboxamide

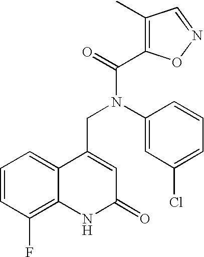

N-(3-Chlorophenyl)-N-((8-fluoro-2-oxo-1,2-dihydroquinolin-4-yl)methyl)-4-methylisoxazole-5-carboxamide was synthesized as described in EXAMPLE 43 using 4-((3-chlorophenylamino) methyl)-8-fluoroquinolin-2(1H)-one and 4-methylisoxazole-5-carboxylic acid as starting materials. LCMS: 411.7 (M+H)⁺.

### EXAMPLE 151

### N-(3-Chloro-4-fluorophenyl)-N-((7,8-difluoro-2-oxo-1,2-dihydroquinolin-4-yl)methyl)-4-methylnicotinamide

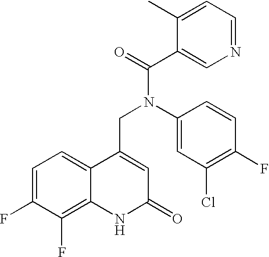

N-(3-Chloro-4-fluorophenyl)-N-((7,8-difluoro-2-oxo-1,2-dihydroquinolin-4-yl)methyl)-4-methylnicotinamide was synthesized as described in EXAMPLE 43 using 4-((3-chloro-4-fluorophenylamino)methyl)-7,8-difluoroquinolin-2(1H)-one and 4-methylnicotinic acid as starting materials. ¹H NMR (400 MHz, DMSO-d₆; HCl salt) δ 8.96 (s, 1H), 8.61 (d, 1H), 7.88-7.68 (m, 3H), 7.40-7.12 (m, 3H), 6.53 (s, 1H), 5.37 (s, 2H), 3.14 (s, 3H). LCMS: 457.8 (M+H)⁺.

### EXAMPLE 152

### N-(3-Chloro-4-fluorophenyl)-N-((7,8-difluoro-2-oxo-1,2-dihydroquinolin-4-yl)methyl)-5-((dimethylamino)methyl)isoxazole-4-carboxamide

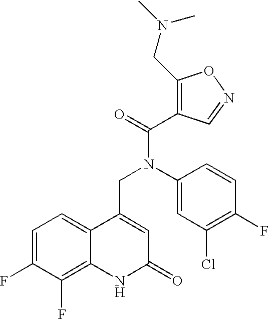

### Step 1: N-(3-Chloro-4-fluorophenyl)-5-(chloromethyl)-N-((7,8-difluoro-2-oxo-1,2-dihydroquinolin-4-yl)methyl)isoxazole-4-carboxamide

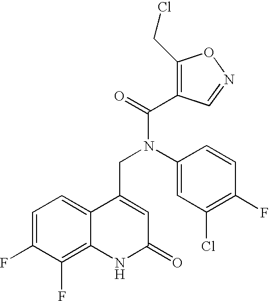

N-(3-Chloro-4-fluorophenyl)-5-(chloromethyl)-N-((7,8-difluoro-2-oxo-1,2-dihydroquinolin-4-yl)methyl)isoxazole-4-carboxamide was synthesized as described in EXAMPLE 43 using 4-((3-chlorophenylamino)methyl)-7,8-difluoroquinolin-2(1H)-one and 5-(chloromethyl)isoxazole-4-carboxylic acid as starting materials. LCMS: 482.2 (M+H)⁺.

### Step 2: N-(3-Chloro-4-fluorophenyl)-N-((7,8-difluoro-2-oxo-1,2-dihydroquinolin-4-yl)methyl)-5-((dimethylamino)methyl)isoxazole-4-carboxamide

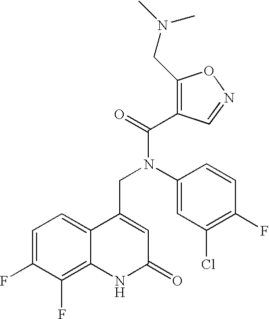

To a solution of N-(3-chloro-4-fluorophenyl)-5-(chloromethyl)-N-((7,8-difluoro-2-oxo-1,2-dihydroquinolin-4-yl)methyl)isoxazole-4-carboxamide (482 mg, 1 mmol) in dimethylacetamide (4 mL), was added dimethylamine (10 mL of 2M in THF, 20 mmol). The reaction mixture was heated at 50° C. for 1 h. The reaction mixture was cooled to RT, then poured into a separatory funnel containing phosphate buffer (25 mL, 1N, pH9) and DCM (50 mL). The organic layer was concentrated. Purification by preparative HPLC (Gradient: 5% to 100% acetonitrile:water, 0.1% TFA) gave N-(3-chloro-4-fluorophenyl)-N-((7,8-difluoro-2-oxo-1,2-dihydroquinolin-4-yl)methyl)-5-((dimethylamino)methyl)isoxazole-4-carboxamide (60 mg) as an off-white solid. ¹H NMR (400 MHz, CDCl₃; TFA salt) δ 7.43-7.36 (m, 2H), 7.20-6.90 (m, 4H), 6.40 (s, 1H), 5.08 (s, 2H), 4.65 (s, 2H), 2.84 (s, 6H). LCMS: 490.9 (M+H)⁺.

### EXAMPLE 153

### N-(3-Chlorophenyl)-N-((7,8-difluoro-2-oxo-1,2-dihydroquinolin-4-yl)methyl)-4-methyloxazole-5-carboxamide

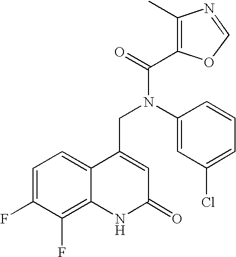

N-(3-Chlorophenyl)-N-((7,8-difluoro-2-oxo-1,2-dihydroquinolin-4-yl)methyl)-4-methyloxazole-5-carboxamide was synthesized as described in EXAMPLE 43 using 4-((3-chlorophenylamino)methyl)-7,8-difluoroquinolin-2(1H)-one and 4-methyloxazole-5-carboxylic acid as starting materials. ¹H NMR (400 MHz, DMSO-d₆) δ 12.00 (s, 1H), 8.14 (s, 1H), 7.70-7.10 (m, 6H), 6.36 (s, 1H), 5.32 (s, 2H), 2.28 (s, 3H). LCMS: 429.7 (M+H)⁺.

### EXAMPLE 154

### 4-Chloro-N-(3-chlorophenyl)-N-((7,8-difluoro-2-oxo-1,2-dihydroquinolin-4-yl)methyl)nicotinamide

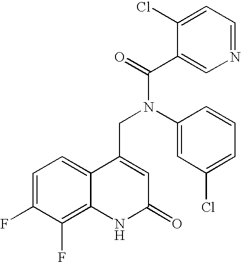

4-Chloro-N-(3-chlorophenyl)-N-((7,8-difluoro-2-oxo-1,2-dihydroquinolin-4-yl)methyl)nicotinamide was synthesized as described in EXAMPLE 43 using 4-((3-chlorophenylamino)methyl)-7,8-difluoroquinolin-2(1H)-one and 4-chloronicotinic acid as starting materials. ¹H NMR (400 MHz, CDCl₃) δ 8.43-8.36 (m, 2H), 7.83-7.75 (m, 1H), 7.35-7.05 (m, 5H), 6.84 (d, 1H), 6.53 (s, 1H), 5.35 (s, 2H). LCMS: 460.7 (M+H)⁺.

### EXAMPLE 155

### N-(3-Chlorophenyl)-N-((7,8-difluoro-2-oxo-1,2-dihydroquinolin-4-yl)methyl)-5-methylnicotinamide

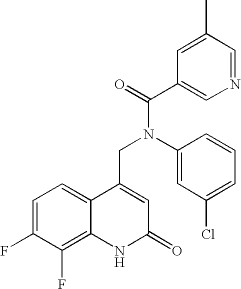

N-(3-Chlorophenyl)-N-((7,8-difluoro-2-oxo-1,2-dihydroquinolin-4-yl)methyl)-5-methylnicotinamide was synthesized as described in EXAMPLE 43 using 4-((3-chlorophenylamino)methyl)-7,8-difluoroquinolin-2(1H)-one and 5-methylnicotinic acid as starting materials. ¹H NMR (400 MHz, DMSO-d₆; TFA salt) δ 12.00 (s, 1H), 8.42 (s, 1H), 8.33 (s, 1H), 7.82 (s, 1H), 7.72-7.65 (m, 1H), 7.50 (s, 1H), 7.35-7.09 (m, 4H), 6.52 (s, 1H), 5.36 (s, 2H), 2.22 (s, 3H). LCMS: 442.2 (M+H)⁺.

### EXAMPLE 156

### N-(3-Chloro-4-fluorophenyl)-N-((7,8-difluoro-2-oxo-1,2-dihydroquinolin-4-yl)methyl)-1-methyl-1H-imidazole-5-carboxamide

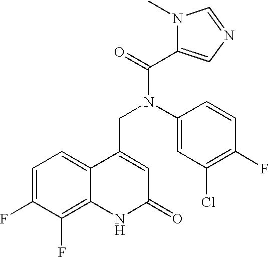

N-(3-Chloro-4-fluorophenyl)-N-((7,8-difluoro-2-oxo-1,2-dihydroquinolin-4-yl)methyl)-1-methyl-1H-imidazole-5-carboxamide was synthesized as described in EXAMPLE 46 using 3-chloro-4-fluoroaniline in the synthesis of Intermediate B and 2-(tert-butyldimethylsilyloxy)-7,8-difluoro-4-(iodomethyl)quinoline as intermediate A. ¹H NMR (400 MHz, DMSO-d₆; HCl salt) δ 9.08 (s, 1H), 7.85 (m, 1H), 7.60 (m, 1H), 7.45-7.10 (m, 3H), 7.05 (s, 1H), 6.54 (s, 1H), 5.30 (s, 2H), 3.99 (s, 3H). LCMS: 447.3 (M+H)⁺.

### EXAMPLE 157

### N-(5-Chloro-2-fluorophenyl)-N-((7,8-difluoro-2-oxo-1,2-dihydroquinolin-4-yl)methyl)-1-methyl-1H-imidazole-5-carboxamide

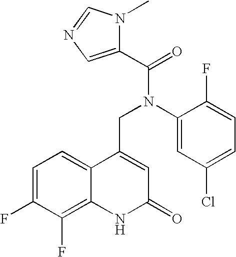

N-(5-Chloro-2-fluorophenyl)-N-((7,8-difluoro-2-oxo-1,2-dihydroquinolin-4-yl)methyl)-1-methyl-1H-imidazole-5-carboxamide was synthesized as described in EXAMPLE 46 using 3-chloro-6-fluoroaniline in the synthesis of Intermediate B and 2-(tert-butyldimethylsilyloxy)-7,8-difluoro-4-(iodomethyl)quinoline as intermediate A. LCMS: 447.3 (M+H)⁺.

### EXAMPLE 158

### N-(3-Chloro-5-fluorophenyl)-N-((7,8-difluoro-2-oxo-1,2-dihydroquinolin-4-yl)methyl)-1-methyl-1H-imidazole-5-carboxamide

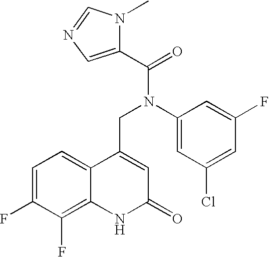

N-(3-Chloro-5-fluorophenyl)-N-((7,8-difluoro-2-oxo-1,2-dihydroquinolin-4-yl)methyl)-1-methyl-1H-imidazole-5-carboxamide was synthesized as described in EXAMPLE 46 using 3-chloro-5-fluoroaniline in the synthesis of Intermediate B and 2-(tert-butyldimethylsilyloxy)-7,8-difluoro-4-(iodomethyl)quinoline as intermediate A. LCMS: 447.3 (M+H)⁺.

### EXAMPLE 159

### N-(3-Chloro-2-fluorophenyl)-N-((7,8-difluoro-2-oxo-1,2-dihydroquinolin-4-yl)methyl)-1-methyl-1H-imidazole-5-carboxamide

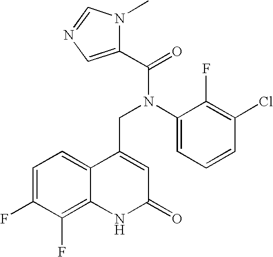

N-(3-Chloro-2-fluorophenyl)-N-((7,8-difluoro-2-oxo-1,2-dihydroquinolin-4-yl)methyl)-1-methyl-1H-imidazole-5-carboxamide was synthesized as described in EXAMPLE 46 using 3-chloro-2-fluoroaniline in the synthesis of Intermediate B and 2-(tert-butyldimethylsilyloxy)-7,8-difluoro-4-(iodomethyl)quinoline as intermediate A. LCMS: 447.3 (M+H)⁺.

### EXAMPLE 160

N-(5-(N-(3-Chlorophenyl)-N-((8-fluoro-2-oxo-1,2-dihydroquinolin-4-yl)methyl)sulfamoyl)-4-methylthiazol-2-yl)acetamide

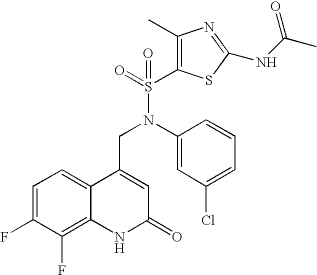

### Step 1: N-(4-Methylthiazol-2-yl)acetamide

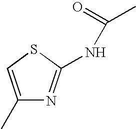

A mixture of 4-methylthiazol-2-amine (30 g, 263.16 mmol), acetic anhydride (54 g, 529.41 mmol), and NaOAc (28 g, 341.46 mmol) in HOAc (300 mL) was refluxed for 18 h. The solvent was removed and the residue was dissolved in EtOAc (500 mL). The resulting mixture was washed with water (4×200 mL) and the organic layer was dried over Na₂SO₄. This resulted in 40 g (crude) of N-(4-methylthiazol-2-yl)acetamide as a yellow solid.

### Step 2: N-(5-(N-(3-Chlorophenyl)-N-((8-fluoro-2-oxo-1,2-dihydroquinolin-4-yl)methyl)sulfamoyl)-4-methylthiazol-2-yl)acetamide

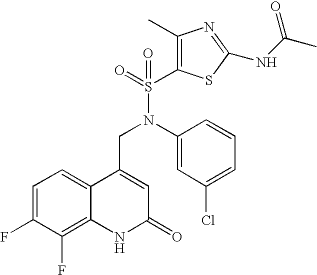

N-(5-(N-(3-Chlorophenyl)-N-((8-fluoro-2-oxo-1,2-dihydroquinolin-4-yl)methyl)sulfamoyl)-4-methylthiazol-2-yl)acetamide was synthesized as described in EXAMPLE 42 using N-(4-methylthiazol-2-yl)acetamide in step 1. LCMS: 521 (M+H)⁺.

### EXAMPLE 161

### 4-((2-(3-Chlorophenyl)-5-oxo-2H-pyrazol-1(5H)-yl)methyl)-8-fluoroquinolin-2(1H)-one

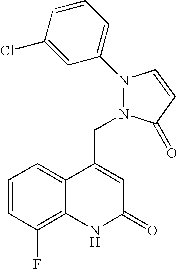

### Step 1: 1-(3-Chlorophenyl)hydrazine hydrochloride

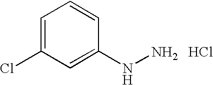

3-Chlorobenzenamine (20 g, 157.48 mmol) was suspended in HCl (12N, 90 mL) and cooled to −20° C. A solution of NaNO₂ (13.04 g) in water (40 mL) was then added dropwise while the temperature was maintained below −20° C. The resulting solution was stirred for 1 h followed by addition of SnCl₂.2H₂O (67.4 g, 314.95 mmol) in HCl (12N, 50 mL). The resulting solution was allowed to react, with stirring, for additional 1 hour while the temperature was maintained at −20° C. The white solid was filtered and dried to afford in 21 g (crude) of 1-(3-chlorophenyl)hydrazine hydrochloride as a white solid.

### Step 2: 1-(3-Chlorophenyl)pyrazolidin-3-one

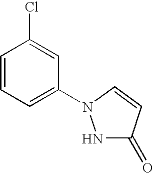

Sodium (162 mg, 7.04 mmol) was added to 2-methylpropan-1-ol (40 mL) followed by addition of 1-(3-chlorophenyl)hydrazine (1 g, 7.04 mmol). To the mixture was added methyl acrylate (850 mg, 9.88 mmol). The resulting solution was refluxed for 7 h. The mixture was concentrated and water was added to dissolve the residue. Adjustment of the pH to 7 was accomplished by the addition of HOAc. The resulting solution was extracted three times with EtOAc (50 mL) and the organics were combined. The residue was purified by column chromatography on silica gel (eluting with a 1:20 EtOAc/PE solvent system). This resulted in 200 mg (15%) of 1-(3-chlorophenyl)pyrazolidin-3-one as a white solid.

### Step 3: 4-((2-(3-Chlorophenyl)-5-oxo-2H-pyrazol-1(5H)-yl)methyl)-8-fluoroquinolin-2(1H)-one

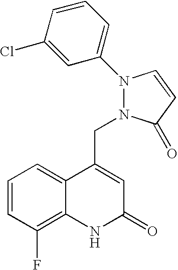

Sodium hydride (9 mg, 0.38 mmol) was added to 1-(3-chlorophenyl)pyrazolidin-3-one (40 mg, 0.21 mmol) in DMF (20 mL). 4-(Bromomethyl)-8-fluoroquinolin-2(1H)-one (52 mg, 0.20 mmol) was then added and the resulting solution was stirred at RT for 3 h. Water was added and the precipitate was filtered and purified by column chromatography on silica gel (eluting with a 1:2 EtOAc/PE solvent system) to afford 40 mg (53%) of 4-((2-(3-chlorophenyl)-5-oxo-2H-pyrazol-1(5H)-yl)methyl)-8-fluoroquinolin-2(1H)-one as a light yellow solid. ¹HNMR (300 MHz, DMSO-d₆) δ 11.79 (s, 1H), 8.48 (d, 1H), 7.84 (s, 1H), 7.69 (d, 1H), 7.59 (d, 1H), 7.47 (m, 2H), 7.25 (m, 2H), 6.71 (s, 1H), 6.23 (d, 1H), 5.57 (s, 2H). LCMS: 370 (M+H)⁺.

### EXAMPLE 162

### 4-((3-(3-Chlorophenyl)-1H-pyrazol-1-yl)methyl)-8-fluoroquinolin-2(1H)-one

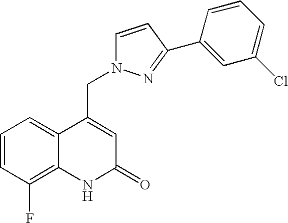

### Step 1: 3-(3-chlorophenyl)-3-oxopropanal

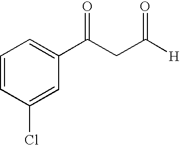

To a solution of NaOMe/MeOH (1.2 eq. 25% in MeOH) in THF with Ethyl formate (402 mg, 1.2 eq.) at room temperature was added dropwise 1-(3-chlorophenyl)ethanone (151.2 mg, 0.98 mmol). The reaction mixture was continuously stirred at RT for 2 h. After removing the solvent, the residue was poured into water and extracted with EtOAc (3×). The aqueous layer was acidified with 1 N HCl (pH=5) followed by extraction with ether. The resulting organic layer was washed with water (2×100 mL) and brine (2×50 mL), then dried over MgSO₄, filtered, and concentrated under reduced pressure. The resulting crude mixture was then purified by column chromatography on silica gel (eluting with 40% EtOAc in hexanes) to afford 3-(3-chlorophenyl)-3-oxopropanal (28%) as a yellow oil. LCMS: 183.0 (M+H)—.

### Step 2: 3-(3-Chlorophenyl)-1H-pyrazole

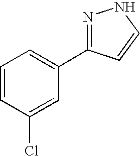

To a solution of 3-(3-chlorophenyl)-3-oxopropanal (220 mg, 1.2 mmol) in ethanol was added (dropwise) a solution of NH₂NH₂ (80 μl). After the reaction was complete (check with LC/MS), the mixture was then concentrated to give yellow solid. The crude product was taken to the next step without further purification.

### Step 3: 4-((3-(3-Chlorophenyl)-1H-pyrazol-1-yl)methyl)-8-fluoroquinolin-2(1H)-one

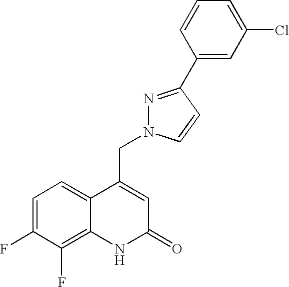

To the solution of 3-(3-chlorophenyl)-1H-pyrazole (250 mg, 1.2 eq) in DMSO was added dropwise a solution of NaOtBu (22 mg, 1.2 eq) and stirred at RT for 5 min. To this solution was added a solution of 4-(bromomethyl)-8-fluoroquinolin-2(1H)-one in DMSO and the reaction mixture was continuously stirred at RT for 20 min. After the reaction was complete (check with LC/MS), the mixture was poured into water and extracted with EtOAc (3×). The combined organic layers were concentrated and dried over Na₂SO₄ to yield a yellow oil. The compound was purified with preparative HPLC to give 11 mg of 4-((3-(3-chlorophenyl)-1H-pyrazol-1-yl)methyl)-8-fluoroquinolin-2(1H)-one as white powder. ¹H NMR (400 MHz, DMSO-d₆) δ 11.82 (s, 1H), 7.99 (s, 1H), 7.83 (s, 1H), 7.80 (d, 1H), 7.65 (d, 1H), 7.43-7.30 (m, 3H), 7.15-7.10 (m, 1H), 6.92 (s, 1H), 5.86 (s, 1H), 5.71 (s, 2H). LCMS: 354.1 (M+H)⁺.

### EXAMPLE 163

### 4-((3-(3-Chlorophenyl)-1H-pyrazol-1-yl)methyl)-7,8-difluoroquinolin-2(1H)-one

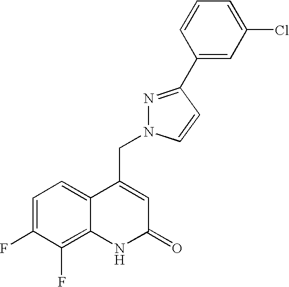

4-((3-(3-Chlorophenyl)-1H-pyrazol-1-yl)methyl)-7,8-difluoroquinolin-2(1H)-one was synthesized as described in EXAMPLE 162 using 4-(bromomethyl)-7,8-fluoroquinolin-2(1H)-one and 3-(3-chlorophenyl)-1H-pyrazole as starting materials. ¹H NMR (400 MHz, DMSO-d₆) δ 12.10 (s, 1H), 7.83 (s, 1H), 7.80-7.42 (m, 3H), 7.41-7.40 (m, 3H), 6.91 (s, 1H), 5.85 (s, 1H), 5.70 (s, 2H). LCMS: 372.1 (M+H)⁺.

### EXAMPLE 164

### 4-((3-(3-Chlorophenyl)-4-methyl-1H-pyrazol-1-yl)methyl)-7,8-difluoroquinolin-2(1H)-one

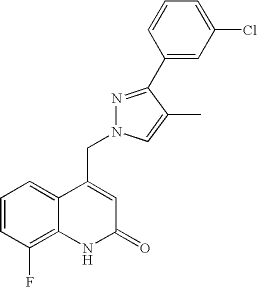

4-((3-(3-Chlorophenyl)-4-methyl-1H-pyrazol-1-yl)methyl)-7,8-difluoroquinolin-2(1H)-one was synthesized as described in EXAMPLE 162 using 4-(bromomethyl)-7,8-fluoroquinolin-2(1H)-one and 3-(3-chlorophenyl)-5-methyl-1H-pyrazole as starting materials. LCMS: 386.1 (M+H)⁺.

### EXAMPLE 165

### 4-((3-(3-Chlorophenyl)-4-methyl-1H-pyrazol-1-yl)methyl)-8-fluoroquinolin-2(1H)-one

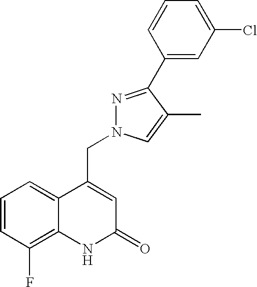

4-((3-(3-Chlorophenyl)-4-methyl-1H-pyrazol-1-yl)methyl)-8-fluoroquinolin-2(1H)-on was synthesized as described in EXAMPLE 162 using 4-(bromomethyl)-8-fluoroquinolin-2(1H)-one and 3-(3-chlorophenyl)-5-methyl-1H-pyrazole as starting materials. LCMS: 368.1 (M+H)⁺.

### EXAMPLE 166

### 4-((3-(3-Chlorophenyl)-5-(4-methylthiazol-5-yl)-1H-pyrazol-4-yl)methyl)-8-fluoroquinolin-2(1H)-one

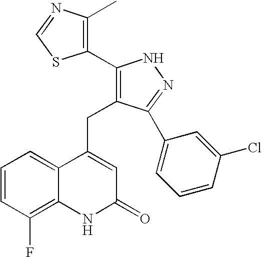

### Step 1: 1-(3-Chlorophenyl)-3-(4-methylthiazol-5-yl)propane-1,3-dione

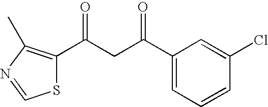

To an LDA (17 mmol, 1.2 eq) solution in THF (20 mL) at −78C was added 3-chloroacetophenone (2.3 g, 15 mmol, 1.0 eq). The solution was warmed to 0° C. over 20 min. To this, was added 4-methylthiazole-5-carbonyl chloride as a 100 mL THF slurry. The reaction mixture was warmed to RT over 10 min. After 15 min at RT, the reaction mixture was poured into a separatory funnel containing 0.3N HCl (100 mL) and DCM (200 mL). The organic layer was concentrated to residue, taken up in EtOAc, filtered to remove undesired salts, and concentrated. Purification by silica gel chromatography (Gradient: 0% to 25% EtOAc:Hexanes) gave 1-(3-chlorophenyl)-3-(4-methylthiazol-5-yl)propane-1,3-dione (550 mg) as a white solid. ¹H NMR (400 MHz, CDCl₃) δ 8.82 (s, 1H), 7.89 (t, 1H), 7.78 (d, 1H), 7.52 (d, 1H), 7.42 (t, 1H), 6.49 (s, 1H), 2.83 (s, 3H). LCMS: 280.3 (M+H)⁺.

### Step 2: 1-(3-Chlorophenyl)-2-((8-fluoro-2-oxo-1,2-dihydroquinolin-4-yl)methyl)-3-(4-methylthiazol-5-yl)propane-1,3-dione

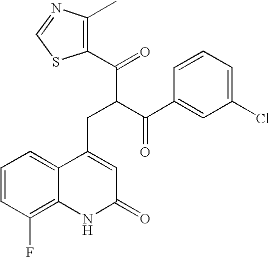

To a solution of 1-(3-chlorophenyl)-3-(4-methylthiazol-5-yl)propane-1,3-dione (140 mg, 0.50 mmol) in THF (2 mL) at −78° C. was added LDA (0.27 mL of 2M solution in THF, 1.1 eq). The reaction mixture was warmed to 5° C., and 2-(tert-butyldimethylsilyloxy)-8-fluoro-4-(iodomethyl)quinoline (see EXAMPLE 46) (240 mg, 0.55 mmol, 1.1 eq) was added in one lot. The solution was warmed to 60° C. After 16 h at 60° C., the reaction mixture was poured into a separatory funnel containing phosphate buffer (50 mL, 1N, pH7) and DCM (50 mL). To the isolated organic layer, was added TBAF (0.50 mmol, 1 eq). The solution was then concentrated. Purification by silica gel chromatography (Gradient: 0% to 100% EtOAc:Hexanes) gave 1-(3-chlorophenyl)-2-((8-fluoro-2-oxo-1,2-dihydroquinolin-4-yl)methyl)-3-(4-methylthiazol-5-yl)propane-1,3-dione (15 mg) as a white solid. ¹H NMR (400 MHz, CDCl₃) δ 9.83 (s, 1H), 8.76 (s, 1H), 7.85-7.15 (m, 7H), 6.50 (s, 1H), 5.23 (t, 1H), 3.60 (m, 2H), 2.70 (s, 3H). LCMS: 454.7 (M+H)⁺.

### Step 3: 4-((3-(3-Chlorophenyl)-5-(4-methylthiazol-5-yl)-1H-pyrazol-4-yl)methyl)-8-fluoroquinolin-2(1H)-one

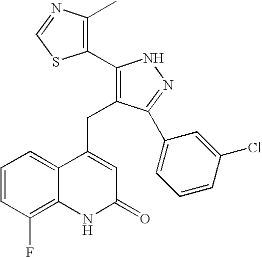

To a solution of 1-(3-chlorophenyl)-2-((8-fluoro-2-oxo-1,2-dihydroquinolin-4-yl)methyl)-3-(4-methylthiazol-5-yl)propane-1,3-dione (15 mg, 33 umol) in EtOH (200 proof, 0.3 mL) was added hydrazine (30 μL, 1 mmol). The reaction mixture was heated at 60° C. for 2 h, then cooled to RT, and diluted with MeOH (1 mL). Purification by preparative HPLC (Gradient: 5% to 100% acetonitrile:water, 0.1% TFA) gave 4-((3-(3-chlorophenyl)-5-(4-methylthiazol-5-yl)-1H-pyrazol-4-yl)methyl)-8-fluoroquinolin-2(1H)-one (2 mg) as a white solid. ¹H NMR (400 MHz, CDCl₃ and CD₃OD) δ 8.60 (s, 1H), 7.45-7.03 (m, 8H), 6.12 (s, 2H), 2.35 (s, 3H). LCMS: 450.5 (M+H)⁺.

### EXAMPLE 167

### 4-{[(3-Chlorophenyl)(ethyl)amino]methyl}-8-fluoroquinolin-2(1H)-one

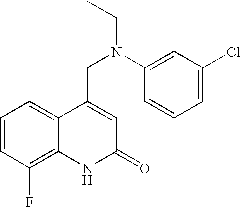

To a suspension of 4-{[(3-chlorophenyl)amino]methyl}-8-fluoroquinolin-2(1H)-one (302 mg, 1 mmol) in EtOH (3 mL) and AcOH (3 mL) was added Na(OAc)₃BH (636 mg, 3 mmol). The reaction mixture was stirred at RT for 18 h and then water (20 mL) added. The mixture was filtered through celite, washed with water (2×20 mL), and dried over Na₂SO₄ and concentrated. The crude reaction mixture was purified by column chromatography on silica gel (Hexane/Ethyl acetate=70/30 to 30/70) to give 4-{[(3-chlorophenyl)(ethyl)amino]methyl}-8-fluoroquinolin-2(1H)-one (20 mg). ¹H NMR (400 MHz, CDCl₃) δ 10.40 (s, 1H), 7.45 (m, 1H), 7.32 (m, 1H), 7.21 (m, 1H), 7.09 (m, 1H), 6.67 (m, 1H), 6.58 (m, 2H), 6.48 (m, 1H), 4.62 (s, 2H), 3.48 (q, 2H), 1.26 (t, 3H). LCMS: 332 (M+H)⁺.

### EXAMPLE 168

### N-(3-Chlorophenyl)-N-[(7,8-difluoro-2-oxo-1,2-dihydroquinolin-4-yl)methyl]-4H-1,2,4-triazole-3-carboxamide

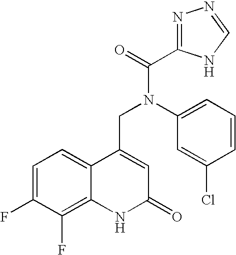

N-(3-Chlorophenyl)-N-[(7,8-difluoro-2-oxo-1,2-dihydroquinolin-4-yl)methyl]-4H-1,2,4-triazole-3-carboxamide was synthesized as described in EXAMPLE 43 using 4-{[(3-chlorophenyl)amino]methyl}-7,8-difluoroquinolin-2(1H)-one and 4H-1,2,4-triazole-3-carboxylic acid as starting materials. ¹H NMR (400 MHz, DMSO-d₆) δ 12.00 (s, 1H), 8.43 (s, 1H), 7.67 (m, 1H), 7.30 (m, 4H), 6.99 (m, 1H), 6.41 (s, 1H), 5.38 (m, 2H). LCMS: 415 (M+H)⁺.

### EXAMPLE 169

### N-(3-Chlorophenyl)-N-[(7,8-difluoro-2-oxo-1,2-dihydroquinolin-4-yl)methyl]-1H-tetrazole-5-carboxamide

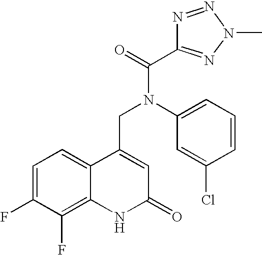

### Step 1: Ethyl 2-methyl-2H-tetrazole-5-carboxylate

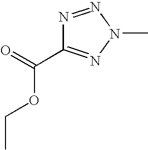

To ethyl 2H-tetrazole-5-carboxylate (1.97 g, 12 mmol) in acetone/DMSO (5 mL/5 mL) was added iodomethane (1.5 mL, 24 mmol). The reaction mixture was heated at 55° C. for 24 h giving a thick precipitate. After cooling to RT, the reaction mixture was poured into 100 mL of EtOAc and was washed with water (100 mL). The organic layer was separated and the aqueous layer washed with EtOAc (3×100 mL). The combined organic layers were dried over Na₂SO₄ and the solvent was removed. The residue was purified by preparative liquid chromatography (YMC column; ACN/H₂O) to give the two methylated regioisomers: (A) 1^{st} eluting fraction (450 mg) and (B) 2^{nd} eluting fraction (316 mg). HMBC NMR experiments indicated that (B) was the desired ethyl 2-methyl-2H-tetrazole-5-carboxylate. ¹H NMR (400 MHz, CDCl₃) δ 4.52 (q, 2H), 4.44 (s, 3H), 1.44 (t, 3H).

### Step 2: 2-Methyl-2H-tetrazole-5-carboxylic acid

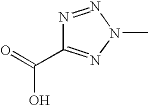

The ethyl 2-methyl-2H-tetrazole-5-carboxylate was then dissolved in ethanol and KOH (1M, 2 equivalents) was added to give an instant precipitate. After stirring for 10 minutes, the ethanol was removed in-vacuo and HCl (1M, 15 mL)/ethyl acetate (20 mL) was added. The organic layer was separated and the aqueous layer washed with EtOAc (5×50 mL). The combined organic layers were dried over Na₂SO₄ and the solvent was removed to give 2-methyl-2H-tetrazole-5-carboxylic acid as a crystalline solid (260 mg).

### Step 3: N-(3-Chlorophenyl)-N-[(7,8-difluoro-2-oxo-1,2-dihydroquinolin-4-yl)methyl]-1H-tetrazole-5-carboxamide

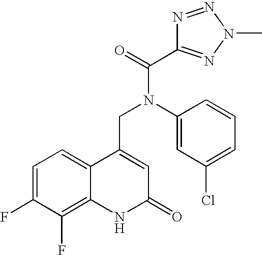

N-(3-Chlorophenyl)-N-[(7,8-difluoro-2-oxo-1,2-dihydroquinolin-4-yl)methyl]-1H-tetrazole-5-carboxamide was synthesized as described in EXAMPLE 43 using 4-{[(3-chlorophenyl)amino]methyl}-7,8-difluoroquinolin-2(1H)-one and 2-methyl-2H-tetrazole-5-carboxylic acid as starting materials. ¹H NMR (400 MHz, DMSO-d₆) δ 12.00 (s, 1H), 7.62 (m, 1H), 7.48 (m, 1H), 7.31 (m, 3H), 7.01 (m, 1H), 6.41 (s, 1H), 5.40 (s, 2H), 4.30 (s, 3H). LCMS: 431 (M+H)⁺.

### EXAMPLE 170

### N-((7,8-Difluoro-2-oxo-1,2-dihydroquinolin-4-yl)methyl)-4-methyl-N-(4-(piperidin-1-yl)phenyl)thiazole-5-carboxamide

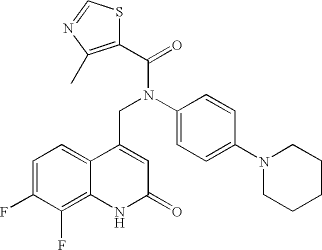

### Step 1: 7,8-Difluoro-4-((4-(piperidin-1-yl)phenylamino)methyl)quinolin-2(1H)-one

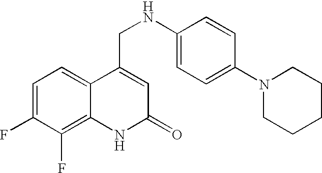

7,8-Difluoro-4-((4-(piperidin-1-yl)phenylamino)methyl)quinolin-2(1H)-one was synthesized as described in EXAMPLE 43, Step 4 using 4-(bromomethyl)-7,8-difluoroquinolin-2(1H)-one and 4-(piperidin-1-yl)aniline as starting materials. ¹H NMR (400 MHz, DMSO-d₆) δ 11.95 (br s, 1H), 7.72-7.68 (m, 1H), 7.39-7.26 (m, 3H), 6.81 (br s, 1H), 6.69 (d, 2H), 6.35 (s, 1H), 4.56 (s, 2H), 3.50-3.35 (m, 4H), 1.90-1.70 (m, 5H), 1.55-1.48 (m, 1H). LCMS: 369.80 (M+H)⁺.

### Step 2: N-((7,8-Difluoro-2-oxo-1,2-dihydroquinolin-4-yl)methyl)-4-methyl-N-(4-(piperidin-1-yl)phenyl)thiazole-5-carboxamide

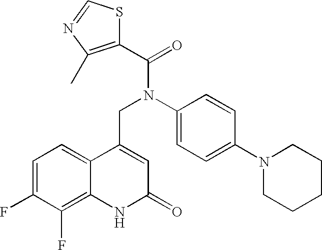

N-((7,8-Difluoro-2-oxo-1,2-dihydroquinolin-4-yl)methyl)-4-methyl-N-(4-(piperidin-1-yl)phenyl)thiazole-5-carboxamide was synthesized as described in EXAMPLE 26 using 7,8-difluoro-4-((4-(piperidin-1-yl)phenylamino)methyl)quinolin-2(1H)-one and 4-methylthiazole-5-carboxylic acid as starting materials. ¹H NMR (400 MHz, DMSO-d₆) δ 12.00 (br s, 1H), 8.89 (s, 1H), 7.72-7.68 (m, 1H), 7.34-7.28 (m, 1H), 6.98-6.96 (m, 2H), 6.90-6.80 (m, 2H), 6.32 (s, 1H), 5.23 (s, 2H), 3.16-3.08 (m, 4H), 2.46 (s, 3H), 1.60-1.46 (m, 6H). LCMS: 496.4 (M+H)⁺.

### EXAMPLE 171

### N-(3-Chlorophenyl)-N-((8-fluoro-2-oxy-1,2-dihydroquinolin-3-yl)methyl)-4-methylthiazole-5-carboxamide

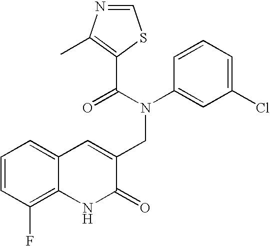

### Step 1: N-(2-Fluorophenyl)acetamide

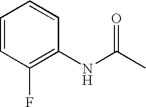

To a solution of 2-fluoroaniline (5.0 g, 45.05 mmol) in DCM (225 mL) was added acetic anhydride (4.09 mL, 45.05 mmol) and triethylamine (7.55 mL, 54.05 mmol). The solution was stirred at RT for 6 h. The reaction mixture was concentrated, diluted with ethyl acetate, and washed with 1N HCl (2×50 mL). The organic solution was dried (Na₂SO₄) and concentrated to provide N-(2-fluorophenyl)acetamide (6.20 g, 90%) as a white solid. LCMS: 153.99 (M+H)⁺.

### Step 2: 3-(Dimethylamino)-N-(2-fluorophenyl)-2-formylacrylamide

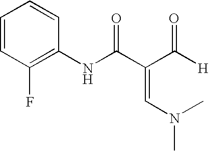

Dimethylformamide (3.79 mL, 49.02 mmol) was cooled to 0° C. in a flask and phosphoryl chloride (19.44 mL, 137.3 mmol) was added dropwise over 10 min. To this solution was added the N-(2-fluorophenyl)acetamide (3.0 g, 19.6 mmol), the reaction was stirred for 10 min at 0° C. then heated to 55° C. for 1.5 h, then cooled to RT and poured into ice water (200 mL) and stirred for 30 min. To the reaction was added a 0° C. solution of 1N sodium hydroxide until the reaction reached pH 9. The reaction was extracted with chloroform (3×50 mL). The combined organic layer was dried (MgSO₄) and evaporated to give a brown oil. Trituration with hexanes provided 3-(dimethylamino)-N-(2-fluorophenyl)-2-formylacrylamide as a brown solid (1.38 g, 32%). LCMS: 236.97 (M+H)⁺.

### Step 3: N-(2-Fluorophenyl)-2-formyl-3-hydroxyacrylamide

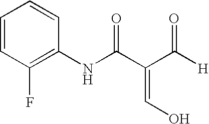

To a solution of 3-(dimethylamino)-N-(2-fluorophenyl)-2-formylacrylamide (1.0 g, 4.24 mmol) in ethanol (10 mL) was added 1N sodium hydroxide (10 mL). The solution was stirred at 90° C. for 5 min. On cooling the mixture was poured onto ice and acidified with conc.HCl. After 30 min the precipitated was filtered, washed with water and dried to provide N-(2-fluorophenyl)-2-formyl-3-hydroxyacrylamide as an off white solid (53.1 mg, 60%). LCMS: 209.96 (M+H)⁺.

### Step 4: 8-Fluoro-2-oxo-1,2-dihydroquinoline-3-carbaldehyde

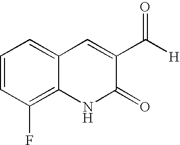

To a sealed vial containing N-(2-fluorophenyl)-2-formyl-3-hydroxyacrylamide (1.0 g, 4.78 mmol) was added polyphosphoric acid (5 g, 60.97 mmol). The reaction was heated to 140° C. for 10 min, then cooled to 70° C., upon which ice was added. The resulting solution was diluted with water (30 mL) and stirred for 30 min. The precipitate was filtered, washed with DCM (10 mL) and then with methanol (10 mL). The precipitate was collected to afford 8-fluoro-2-oxo-1,2-dihydroquinoline-3-carbaldehyde (365.5 mg, 40%). LCMS: 191.93 (M+H)⁺.

### Step 5: 3-((3-Chlorophenylamino)methyl)-8-fluoroquinolin-2(1H)-one

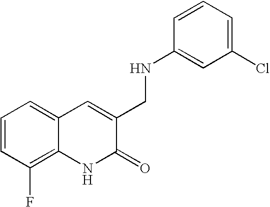

To a solution of 8-fluoro-2-oxo-1,2-dihydroquinoline-3-carbaldehyde (100 mg, 0.5 mmol) in 10% methanol/90% dichloromethane (3 mL) was added 3-chloroaniline (86.4 mg, 0.68 mmol). The reaction mixture was stirred for 1 h and sodium triacetoxy borohydride (276 mg, 1.31 mmol) was added. The solution was stirred at RT for 4 h. The reaction mixture was concentrated, diluted with ethyl acetate, and washed with saturated sodium bicarbonate (2×50 mL) then with 1N HCl (2×50 mL). The organic solution was dried (Na₂SO₄) and concentrated to provide 3-((3-chlorophenylamino)methyl)-8-fluoroquinolin-2-(1H)-one as a tan solid (94.9 mg, 60%). ¹H NMR (400 MHz, CDCl₃) δ 9.64 (s, 1H), 7.73 (s, 1H), 7.31 d, 1H), 7.26-7.22 (m, 2H), 7.15-7.13 (m, 1H), 7.06 (t, 1H), 6.69 (d, 1H), 6.67-6.64 (m, 1H), 6.52 (dd, 1H), 4.37 (s, 2H); LCMS: 302.96 (M+H)⁺.

### Step 6: N-(3-Chlorophenyl)-N-((8-fluoro-2-oxo-1,2-dihydroquinolin-3-yl)methyl)-4-methylthiazole-5-carboxamide

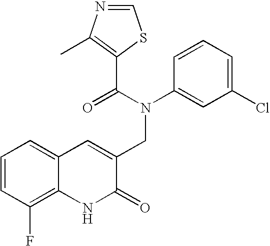

N-(3-Chlorophenyl)-N-((8-fluoro-2-oxo-1,2-dihydroquinolin-3-yl)methyl)-4-methylthiazole-5-carboxamide was synthesized as described in EXAMPLE 26 using 3-((3-chlorophenylamino)methyl)-8-fluoroquinolin-2-(1H)-one and 4-methylthiazole-5-carboxylic acid as starting materials. ¹H NMR (400 MHz, DMSO-d₆) δ 11.93 (s, 1H), 8.92 (s, 1H), 7.94 (s, 1H), 7.63 (s, 1H), 7.57 (d, 1H), 7.39-7.27 (m, 4H), 7.17-7.10 (m, 1H), 4.92 (s, 2H), 2.42 (s, 3H). LCMS: 428 (M+H)⁺.

### EXAMPLE 172

### N-(3-Chlorophenyl)-N-((8-fluoro-2-oxo-1,2-dihydroquinolin-3-yl)methyl)-4-methylnicotinamide

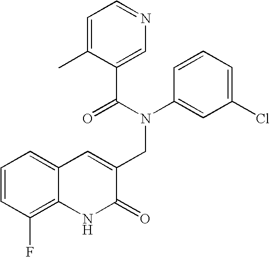

N-(3-Chlorophenyl)-N-((8-fluoro-2-oxo-1,2-dihydroquinolin-3-yl)methyl)-4-methylnicotinamide was synthesized as described in EXAMPLE 26 using 3-((3-chlorophenylamino)methyl)-8-fluoroquinolin-2-(1H)-one and 4-methylpyridine-3-carboxylic acid as starting materials. ¹H NMR (400 MHz, CDCl₃, TFA salt) δ 9.99 (s, 1H), 8.55 (d, 1H), 7.99 (s, 1H), 7.51 (s, 1H), 7.42-7.41 (m, 1H), 7.30-7.24 (m, 2H), 7.20-7.06 (m, 5H), 5.14 (s, 2H), 2.64 (s, 3H). LCMS: 422.42 (M+H)⁺.

### EXAMPLE 173

### N-(3-Chlorophenyl)-N-((8-fluoro-2-oxo-1,2-dihydroquinolin-3-yl)methyl)-5-methylisoxazole-4-carboxamide

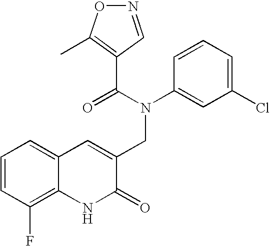

N-(3-Chlorophenyl)-N-((8-fluoro-2-oxo-1,2-dihydroquinolin-3-yl)methyl)-5-methylisoxazole-4-carboxamide was synthesized as described in EXAMPLE 26 using 3-((3-chlorophenylamino) methyl)-8-fluoroquinolin-2-(1H)-one and 5-methylisoxazole-4-carboxylic acid as starting materials. ¹H NMR (400 MHz, CDCl₃) δ 7.97 (s, 1H), 7.40 (d, 1H), 7.35-7.24 (m, 4H), 7.21-7.16 (m, 2H), 7.11 (s, 1H), 5.02 (s, 2H), 2.67 (s, 3H). LCMS: 412.05 (M+H)⁺.

### EXAMPLE 174

### N-((8-Fluoro-2-oxo-1,2-dihydroquinolin-3-yl)methyl)-4-methyl-N-(piperidin-1-yl)phenylthiazole-5-carboxamide

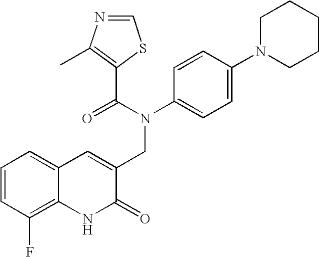

### Step 1: 8-Fluoro-3-((4-(piperidin-1-yl)phenylamino)methyl)quinolin-2(1H)-one

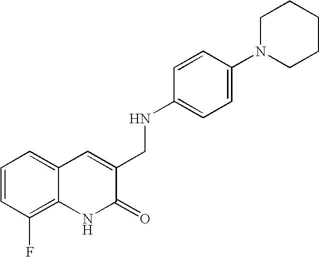

8-Fluoro-3-((4-(piperidin-1-yl)phenylamino)methyl)quinolin-2(1H)-one was synthesized as described in EXAMPLE 171, Step 5 using N-(4-aminophenyl)piperidine and 8-fluoro-2-oxo-1,2-dihydroquinoline-3-carbaldehyde as starting materials. ¹H NMR (400 MHz, CDCl₃) δ 9.15 (s, 1H), 7.75 (s, 1H), 7.30-7.25 (m, 1H), 7.23-7.18 (m, 1H), 7.13-7.08 (m, 1H), 6.85 (d, 2H), 6.61 (d, 2H), 4.33 (s, 2H), 2.98-2.95 (m, 4H), 1.71-1.62 (m, 4H), 1.55-1.50 (m, 3H). LCMS: 351.80 (M+H)⁺.

### Step 2: N-((8-Fluoro-2-oxo-1,2-dihydroquinolin-3-yl)methyl)-4-methyl-N-(piperidin-1-yl)phenylthiazole-5-carboxamide

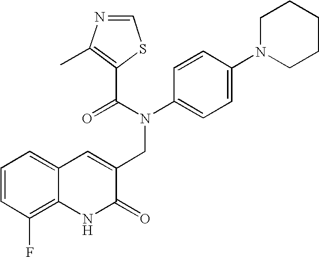

N-((8-Fluoro-2-oxo-1,2-dihydroquinolin-3-yl)methyl)-4-methyl-N-(piperidin-1-yl)phenylthiazole-5-carboxamide was synthesized as described in EXAMPLE 26 using 8-fluoro-3-((4-(piperidin-1-yl)phenylamino)methyl)quinolin-2(1H)-one and 4-methylthiazole-5-carboxylic acid as starting materials. ¹H NMR (400 MHz, DMSO-d₆) δ 11.92 (s, 1H), 8.89 (s, 1H), 7.88 (s, 1H), 7.58 (d, 1H), 7.39-7.30 (m, 1H), 7.26-7.22 (m, 2H), 7.17-7.12 (m, 1H), 7.05-9.60 (m, 1H), 5.74 (s, 2H), 3.20-3.10 (m, 4H), 2.46 (s, 3H), 1.60-1.52 (m, 4H), 1.50-1.45 (m, 3H). LCMS: 477 (M+H)⁺.

### EXAMPLE 175

### N-Phenyl-N-((7,8-fluoro-2-oxo-1,2-dihydroquinolin-4-yl)methyl)-4-methylthiazole-5-carboxamide

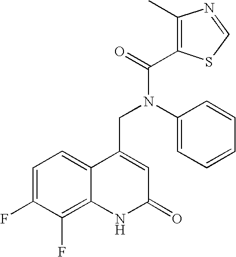

N-Phenyl-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, aniline, and 4-methylthiazole-5-carboxylic acid as starting materials. ¹H NMR (400 MHz, DMSO-d₆) δ 11.95 (s, 1H), 9.88 (s, 1H), 8.94 (s, 1H), 7.75-7.65 (m, 1H), 7.45-7.15 (m, 5H), 6.35 (s, 1H), 5.18 (s, 2H), 2.48 (s, 3H). LCMS: 412.1 (M+H)⁺.

### EXAMPLE 176

### N-(3-Chloro4-methoxylphenyl)-N-((7,8-fluoro-2-oxo-1,2-dihydroquinolin-4-yl)methyl)-4-methylthiazole-5-carboxamide

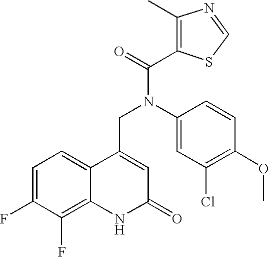

N-(3-Chloro-4-methoxylphenyl)-N-((8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-chloro 4-methoxyaniline, and 4-methylthiazole-5-carboxylic acid as starting materials. ¹H NMR (400 MHz, DMSO-d₆) δ 12.00 (s, 1H), 10.11 (s, 1H), 8.94 (s, 1H), 7.77-7.65 (m, 1H), 7.45 (s, 1H), 7.40-7.31 (m, 1H), 7.00 (s, 1H), 6.35 (s, 1H), 5.17 (s, 2H), 3.72 (s, 3H), 2.48 (s, 3H). LCMS: 476.1 (M+H)⁺.

### EXAMPLE 177

### N-(3-Chloro4-methylphenyl)-N-((7,8-fluoro-2-oxo-1,2-dihydroquinolin-4-yl)methyl)-4-methylthiazole-5-carboxamide

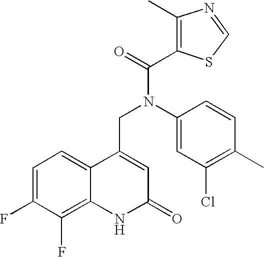

N-(3-Chloro-4-methylphenyl)-N-((8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-chloro 4-methylaniline, and 4-methylthiazole-5-carboxylic acid as starting materials ¹H NMR (400 MHz, DMSO-d₆) δ 12.01 (s, 1H), 8.94 (s, 1H), 7.70-7.65 (m, 1H), 7.77-7.61 (s, 1H), 7.40 (m, 1H), 7.40-7.25 (m, 1H), 6.95 (m, 1H), 6.41 (s, 1H), 5.25 (s, 2H), 2.48 (s, 3H), 2.22 (s, 3H). LCMS: 460.1 (M+H)⁺.

### EXAMPLE 178

### N-(3,5-Difluorophenyl)-N-((7,8-fluoro-2-oxo-1,2-dihydroquinolin-4-yl)methyl)-4-methylthiazole-5-carboxamide

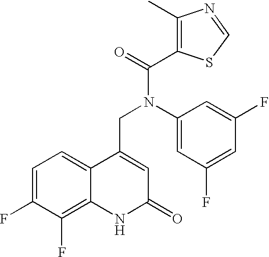

N-(3,5-Difluorophenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3,5-difluoroaniline, and thiazole-5-carboxylic acid as starting materials. ¹H NMR (400 MHz, DMSO-d₆) δ 12.08 (s, 1H), 8.96 (s, 1H), 7.75-7.72 (m, 1H), 7.46-7.38 (m, 1H), 7.18-7.14 (m, 3H), 6.42 (s, 1H), 5.34 (s, 2H), 2.48 (s, 3H). LCMS: 448.1 (M+H)⁺.

### EXAMPLE 179

### N-(1-Naphthalene)-N-((7,8-fluoro-2-oxo-1,2-dihydroquinolin-4-yl)methyl)-4-methylthiazole-5-carboxamide

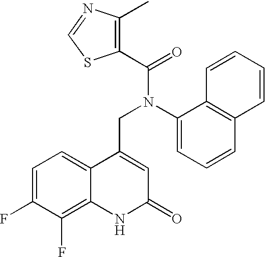

N-(1-Naphthalene)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, naphthalen-1-amine, and 4-methylthiazole-5-carboxylic acid as starting materials. ¹H NMR (400 MHz, DMSO-d₆) δ 12.05 (s, 1H), 8.67 (s, 1H), 7.99-7.95 (d, 1H), 7.85-7.25 (m, 7H), 6.45 (s, 1H), 6.25 (s, 1H), 5.58 (s, 2H), 2.48 (s, 3H). LCMS: 462.1 (M+H)⁺.

### EXAMPLE 180

### N-(3-Methoxylphenyl)-N-((7,8-fluoro-2-oxo-1,2-dihydroquinolin-4-yl)methyl)-4-methylthiazole-5-carboxamide

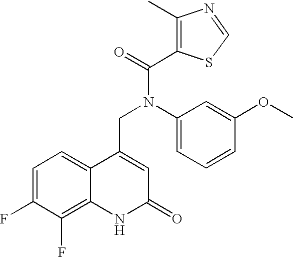

N-(3-Methoxylphenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-methoxylaniline, and thiazole-5-carboxylic acid as starting materials. LCMS: 442.1 (M+H)⁺.

### EXAMPLE 181

### N-(3-Chloro-4-fluorophenyl)-N-((7,8-fluoro-2-oxo-1,2-dihydroquinolin-4-yl)methyl)-4-methylthiazole-5-carboxamide

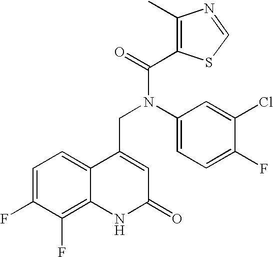

N-(3-Chloro-4-fluorophenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-chloro-4-fluoroaniline, and 4-methylthiazole-5-carboxylic acid as starting materials. ¹H NMR (400 MHz, DMSO-d₆) δ 12.00 (s, 1H), 8.92 (s, 1H), 7.72-7.69 (m, 2H), 7.30-7.29 (m, 2H), 7.14-7.12 (m, 1H), 6.40 (s, 1H), 5.30 (s, 2H), 2.49 (s, 3H). LCMS: 464.0 (M+H)⁺.

### EXAMPLE 182

### N-(3-Chloro-4-cyanophenyl)-N-((7,8-fluoro-2-oxo-1,2-dihydroquinolin-4-yl)methyl)-4-methylthiazole-5-carboxamide

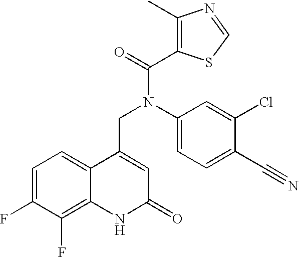

N-(3-Chloro-4-cyanophenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-chloro-4-cyanoaniline, and 4-methylthiazole-5-carboxylic acid as starting materials. ¹H NMR (400 MHz, DMSO-d₆) δ 12.05 (s, 1H), 8.67 (s, 1H), 7.99-7.95 (d, 1H), 7.85-7.25 (m, 4H), 6.25 (s, 1H), 5.28 (s, 2H), 2.47 (s, 3H). LCMS: 471.1 (M+H)⁺.

### EXAMPLE 183

### N-(4-Fluorolphenyl)-N-((7,8-fluoro-2-oxo-1,2-dihydroquinolin-4-yl)methyl)-4-methylthiazole-5-carboxamide

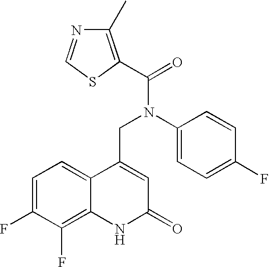

N-(4-Fluorophenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 4-fluoroaniline, and thiazole-5-carboxylic acid as starting materials. LCMS: 430.1 (M+H)⁺.

### EXAMPLE 184

### N-(3-Chloro-2-methylphenyl)-N-((7,8-difluoro-2-oxo-1,2-dihydroquinolin-4-yl)methyl)-4-methylthiazole-5-carboxamide

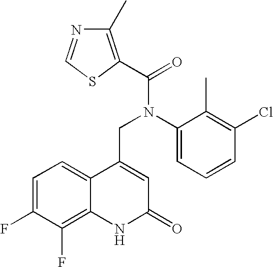

N-(3-Chloro-2-methylphenyl)-N-((7,8-difluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-chloro-2-methylaniline, and thiazole-5-carboxylic acid as starting materials. ¹H NMR (400 MHz, DMSO-d₆) δ 12.06 (s, 1H), 8.86 (s, 1H), 7.80-7.75 (m, 1H), 7.45-7.43 (d, 1H), 7.35-7.30 (m, 1H), 7.20-7.18 (d, 1H), 7.02-7.00 (m, 1H), 6.30 (s, 1H), 5.51 (d, 1H), 4.85 (d, 1H), 2.48 (s, 3H), 2.02 (s, 3H). LCMS: 460.1 (M+H)⁺.

### EXAMPLE 185

### N-(Isopropylamino)-N-((7,8-fluoro-2-oxo-1,2-dihydroquinolin-4-yl)methyl)-4-methylthiazole-5-carboxamide

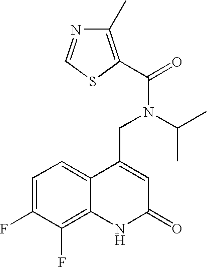

N-(Isopropylamino)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, isopropylamine, and thiazole-5-carboxylic acid as starting materials. ¹H NMR (400 MHz, DMSO-d₆) δ 12.02 (s, 1H), 9.04 (s, 1H), 7.75 (brs, 1H), 7.30-7.28 (dd, 1H), 6.19 (s, 1H), 4.76 (s, 2H), 4.10 (brs, 1H), 2.48 (s, 3H), 1.16 (d, 6H). LCMS: 378 (M+H)⁺.

### EXAMPLE 186

### N-(3-Cyanophenyl)-N-((7,8-difluoro-2-oxo-1,2-dihydroquinolin-4-yl)methyl)-4-methylthiazole-5-carboxamide

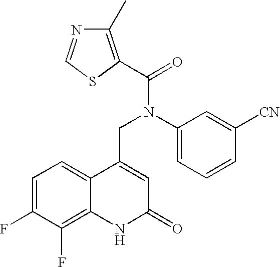

N-(3-Cyanophenyl)-N-((7,8-fluoro-2-oxo-1,2-dihydroquinolin-4-yl)methyl)-4-methylthiazole-5-carboxamide was synthesized as described in EXAMPLE 43 using 4-(bromomethyl)-7,8-difluoroquinolin-2(1H)-one, 3-cyanoaniline, and thiazole-5-carboxylic acid as starting materials. ¹H NMR (400 MHz, DMSO-d₆) δ 12.03 (s, 1H), 8.93 (s, 1H), 7.97 (s, 1H), 7.66-7.78 (m, 2H), 7.45-7.44 (m, 2H), 7.37-7.26 (m, 1H), 6.40 (s, 1H), 5.35 (s, 2H), 2.48 (s, 3H). LCMS: 437.1 (M+H)⁺.

### EXAMPLE 187

### N-(3-Chloro-2-fluorophenyl)-N-((8-fluoro-2-oxo-1,2-dihydroquinolin-4-yl)methyl)-4-methylthiazole-5-carboxamide

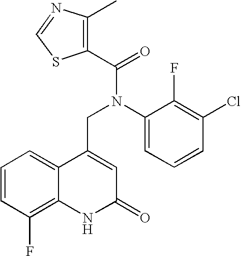

N-(3-Chloro-2-fluorophenyl)-N-((8-fluoro-2-oxo-1,2-dihydroquinolin-4-yl)methyl)-4-methylthiazole-5-carboxamide was synthesized as described in EXAMPLE 43 using 4-(bromomethyl)-8-fluoroquinolin-2(1H)-one, 3-chloro-2-fluoroaniline, and thiazole-5-carboxylic acid as starting materials. LCMS: 446 (M+H)⁺.

### EXAMPLE 188

### N-(3-Chlorophenyl)-N-((7,8-difluoro-2-oxo-1,2-dihydroquinolin-4-yl)methyl)propane-2-sulfonamide

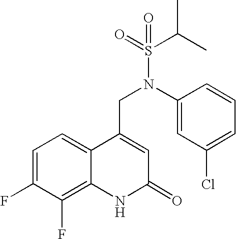

N-(3-Chlorophenyl)-N-((7,8-difluoro-2-oxo-1,2-dihydroquinolin-4-yl)methyl)propane-2-sulfonamide was synthesized as described in EXAMPLE 42 using N-(3-chlorophenyl)propane-2-sulfonamide and 4-(bromomethyl)-7,8-difluoroquinolin-2(1H)-one as starting materials. ¹H NMR (400 MHz, DMSO-d₆) δ 11.95 (s, 1H), 7.79 (m, 1H), 7.60 (m, 1H), 7.44 (m, 1H), 7.35 (m, 3H), 6.43 (s, 1H), 5.29 (s, 2H), 3.50 (m, 1H), 1.30 (d, 6H). LCMS: 426 (M+H)⁺.

### EXAMPLE 189

### N-(3-Chlorophenyl)-N-((7,8-difluoro-2-oxo-1,2-dihydroquinolin-4-yl)methyl)-2,4-dimethylthiazole-5-sulfonamide

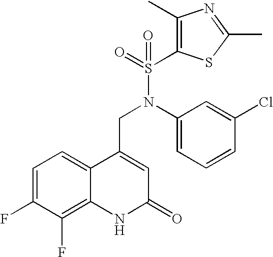

N-(3-Chlorophenyl)-N-((7,8-difluoro-2-oxo-1,2-dihydroquinolin-4-yl)methyl)-2,4-dimethylthiazole-5-sulfonamide was synthesized as described in EXAMPLE 42, Step 3 using 4-(bromomethyl)-7,8-difluoroquinolin-2(1H)-one and N-(3-chlorophenyl)-2,4-dimethylthiazole-5-sulfonamide as starting materials. ¹H NMR (400 MHz, DMSO-d₆) δ 11.95 (s, 1H), 7.83 (s, 1H), 7.46-7.38 (m, 3H), 7.21-7.18 (m, 1H), 6.56-6.42 (d, 2H), 5.12 (s, 2H), 2.68 (s, 3H) 2.15 (s, 3H). LCMS: 496 (M+H)⁺.

### EXAMPLE 190

### 7,8-Difluoro-4-((2-methyl-1H-benzo[d]imidazol-1-yl)methyl)quinolin-2(1H)-one

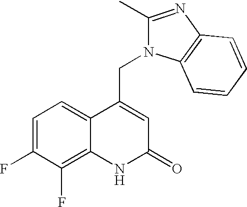

7,8-Difluoro-4-((2-methyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-methyl-1H-benzo[d]imidazole (commercially available) as starting materials. ¹H NMR (400 MHz, DMSO-d₆, HCl salt) δ 12.11 (s, 1H), 7.86 (d, 1H), 7.80 (d, 1H), 7.74-7.72 (m, 1H), 7.60-7.52 (m, 2H), 7.49 (q, 1H), 6.08 (s, 2H), 5.59 (s, 1H), 2.83 (s, 3H). LCMS: 326 (M+H)⁺.

### EXAMPLE 191

### 7,8-Difluoro-4-((2-(thiazol-5-yl)-1H-benzo[d]imidazol-1-yl)methyl)quinolin-2(1H)-one

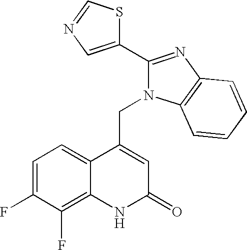

7,8-Difluoro-4-((2-(thiazol-5-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 5-(1H-benzo[d]imidazol-2-yl)thiazole as starting materials. ¹H NMR (400 MHz, DMSO-d₆, HCl salt) δ 9.28 (s, 1H), 8.25 (s, 1H), 7.83-7.80 (m, 2H), 7.69 (d, 1H), 7.44-7.36 (m, 3H), 6.10 (s, 2H), 5.31 (s, 1H). LCMS: 395 (M+H)⁺.

### EXAMPLE 192

### 7,8-Difluoro-4-((2-(methylthio)-1H-benzo[d]imidazol-1-yl)methyl)quinolin-2(1H)-one

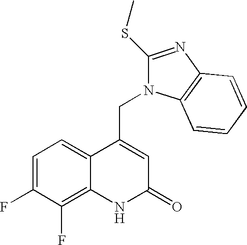

7,8-Difluoro-4-((2-(methylthio)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(methylthio)-1H-benzo[d]imidazole (commercially available) as starting materials. ¹H NMR (400 MHz, DMSO-d₆, HCl salt) δ 12.05 (s, 1H), 7.82 (m, 1H), 7.67 (d, 1H), 7.46 (d, 1H), 7.38 (m, 1H), 7.22-7.16 (m, 2H), 5.71 (s, 2H), 5.18 (s, 1H), 2.72 (s, 3H). LCMS: 357 (M)⁺.

### EXAMPLE 193

### 7,8-Difluoro-4-((2-(thiazol-4-yl)-1H-benzo[d]imidazol-1-yl)methyl)quinolin-2(1H)-one

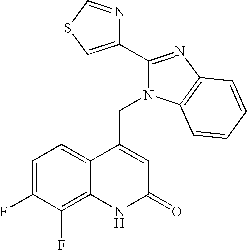

7,8-Difluoro-4-((2-(thiazol-4-yl)-1H-benzo[d]imidazol-1-yl)methyl)quinolin-2(1H)-one one was synthesized as described in EXAMPLE 118 using 4-(bromomethyl)-7,8-difluoroquinolin-2(1H)-one and 4-(1H-benzo[d]imidazol-2-yl)thiazole as starting materials. ¹H NMR (400 MHz, DMSO-d₆, HCl salt) δ 9.24 (s, 1H), 8.82 (s, 1H), 7.85 (d, 2H), 7.73 (d, 1H), 7.47-7.40 (m, 3H), 6.39 (s, 2H), 5.41 (s, 1H). LCMS: 395 (M+H)⁺.

### EXAMPLE 194

### 7,8-Difluoro-4-((2-(morpholinomethyl)-1H-benzo[d]imidazol-1-yl)methyl)quinolin-2(1H)-one

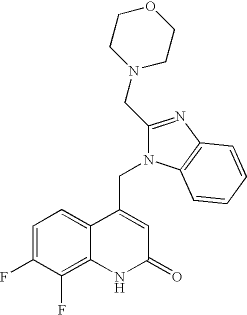

### Step 1: 4-((1H-Benzo[d]imidazol-2-yl)methyl)morpholine

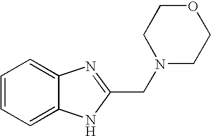

A mixture of 2-(chloromethyl)-1H-benzo[d]imidazole (500 mg, 3 mmol), morpholine (0.26 mL, 3 mmol), and Et₃N (0.6 mL, 4.5 mmol) in DMF (4 mL) was stirred at RT for 3 h. The solvent was removed and the residue was partitioned between EtOAc and brine. The aqueous layer was extracted with EtOAc (3×30 mL). The organics were combined, dried over Na₂SO₄, filtered, and evaporated to dryness. The residue was purified by column chromatography on silica gel (eluting with DCM/MeOH) to afford 317 mg of 4-((1H-benzo[d]imidazol-2-yl)methyl)morpholine as a yellow solid. ¹H NMR (400 MHz, DMSO-d₆) δ 12.21 (s, 1H), 7.52 (d, 1H), 7.41 (d, 1H), 7.13-7.09 (m, 2H), 3.69 (s, 2H), 3.58 (m, 4H), 2.42 (m, 4H).

### Step 2: 7,8-Difluoro-4-((2-(morpholinomethyl)-1H-benzo[d]imidazol-1-yl)methyl)quinolin-2(1H)-one

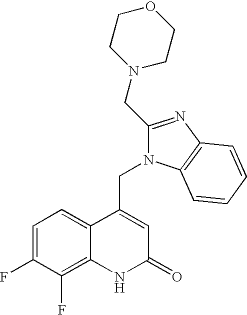

7,8-Difluoro-4-((2-(morpholinomethyl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 4-((1H-benzo[d]imidazol-2-yl)methyl)morpholine as starting materials. ¹H NMR (400 MHz, DMSO-d₆, HCl salt) δ 7.84-7.82 (m, 1H), 7.79-7.76 (m, 1H), 7.64 (m, 1H), 7.50-7.43 (m, 1H), 7.39-7.34 (m, 2H), 6.11 (s, 2H), 5.09 (s, 1H), 7.71 (s, 2H), 3.86 (bs, 4H), 3.39 (bs, 4H). LCMS: 411 (M+H)⁺.

### EXAMPLE 195

### 7,8-Difluoro-4-((2-isobutyl-1H-benzo[d]imidazol-1-yl)methyl)quinolin-2(1H)-one

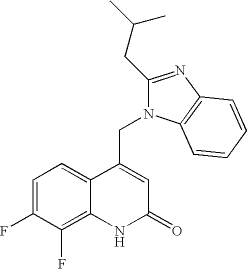

7,8-Difluoro-4-((2-isobutyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-isobutyl-1H-benzo[d]imidazole as starting materials. ¹H NMR (400 MHz, DMSO-d₆, HCl salt) δ 12.21 (s, 1H), 7.89 (d, 1H), 7.81-7.74 (m, 2H), 7.61-7.46 (m, 3H), 6.12 (s, 2H), 5.46 (s, 1H), 3.01 (d, 2H), 2.21 (m, 1H), 0.97 (d, 6H).

### EXAMPLE 196

### 7,8-Difluoro-4-((2-(3-fluorophenyl)-1H-benzo[d]imidazol-1-yl)methyl)quinolin-2(1H)-one

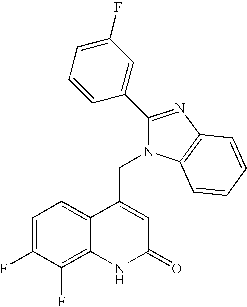

7,8-Difluoro-4-((2-(3-fluorophenyl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(3-fluorophenyl)-1H-benzo[d]imidazole as starting materials. ¹H NMR (400 MHz, DMSO-d₆, HCl salt) δ 7.90 (d, 1H), 7.74-7.47 (m, 8H), 7.45-7.34 (m, 1H), 5.97 (s, 2H), 5.72 (s, 1H). LCMS: 406 (M+H)⁺.

### EXAMPLE 197

### 7,8-Difluoro-4-((2-(4-fluorophenyl)-1H-benzo[d]imidazol-1-yl)methyl)quinolin-2(1H)-one

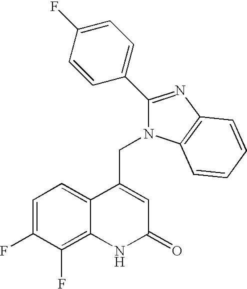

7,8-Difluoro-4-((2-(4-fluorophenyl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(4-fluorophenyl)-1H-benzo[d]imidazole as starting materials. ¹H NMR (400 MHz, DMSO-d₆) δ 7.78-7.72 (m, 3H), 7.42-7.22 (m, 6H), 6.78 (m, 1H), 5.67 (s, 2H), 5.23 (s, 1H).

### EXAMPLE 198

### 4-((2-((Dimethylamino)methyl)-1H-benzo[d]imidazol-1-yl)methyl)-7,8-difluoroquinolin-2(1H)-one

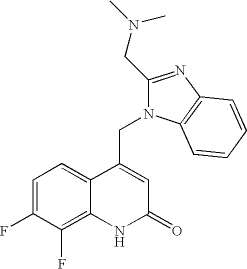

4-((2-((Dimethylamino)methyl)-1H-benzo[d]imidazol-1-yl)methyl)-7,8-difluoroquinolin-2(1H)-one was synthesized as described in EXAMPLE 194 using 4-(bromomethyl)-7,8-difluoroquinolin-2(1H)-one and 1-(1H-benzo[d]imidazol-2-yl)-N,N-dimethylmethanamine as starting materials. ¹H NMR (400 MHz, DMSO-d₆, HCl salt) δ 11.06 (s, 1H), 7.83 (d, 1H), 7.87-75 (m, 1H), 7.62-7.60 (m, 1H), 7.50-7.45 (m, 1H), 7.38-7.32 (m, 2H), 6.05 (s, 2H), 5.07 (s, 1H), 4.73 (s, 2H), 2.93 (s, 6H).

### EXAMPLE 199

### 7,8-Difluoro-4-((2-(3-methylpyridin-4-yl)-1H-benzo[d]imidazol-1-yl)methyl)quinolin-2(1H)-one

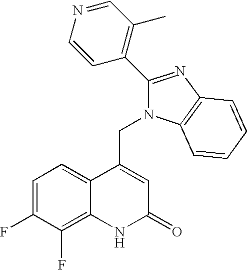

7,8-Difluoro-4-((2-(3-methylpyridin-4-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(3-methylpyridin-4-yl)-1H-benzo[d]imidazole as starting materials. ¹H NMR (400 MHz, DMSO-d₆, HCl salt) δ 8.97 (s, 1H), 8.77 (d, 1H), 7.94-7.90 (m, 2H), 7.70-7.67 (m, 1H), 7.59-7.56 (m, 1H), 7.48-7.44 (m, 2H), 7.36-7.29 (m, 1H), 5.81 (s, 2H), 5.52 (s, 1H), 2.44 (s, 3H). LCMS: 403 (M+H)⁺.

### EXAMPLE 200

### 7,8-Difluoro-4-((2-morpholino-1H-benzo[d]imidazol-1-yl)methyl)quinolin-2(1H)-one

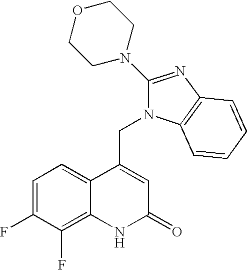

### Step 1: 4-(1H-Benzo[d]imidazol-2-yl)morpholine

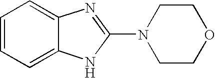

A mixture of 2-chloro-1H-benzo[d]imidazole (500 mg, 3.27 mmol), morpholine (0.57 mL, 6.55 mmol) in DMF (10 mL) was heated in the microwave (15 min, 150° C.). The solvent was removed and the residue was partitioned between EtOAc and brine. The aqueous layer was extracted with EtOAc (3×30 mL). The organics were combined, dried over Na₂SO₄, filtered, and evaporated to dryness. The residue was purified by column chromatography on silica gel (eluting with DCM/MeOH) to afford 235 mg of 4-(1H-benzo[d]imidazol-2-yl)morpholine as a white solid. ¹H NMR (400 MHz, DMSO-d₆) δ 11.41 (s, 1H), 7.18 (d, 2H), 6.91 (d, 2H), 3.70 (m, 4H), 3.43 (m, 4H).

### Step 2: 7,8-Difluoro-4-((2-morpholino-1H-benzo[d]imidazol-1-yl)methyl)quinolin-2(1H)-one

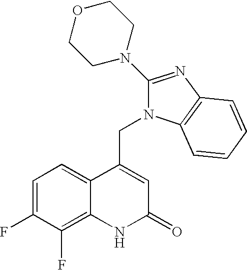

7,8-Difluoro-4-((2-morpholino-1H-benzo[d]imidazol-1-yl)methyl)quinolin-2(1H)-one was synthesized as described in EXAMPLE 118 using 4-(bromomethyl)-7,8-difluoroquinolin-2(1H)-one and 4-(1H-benzo[d]imidazol-2-yl)morpholine as starting materials. ¹H NMR (400 MHz, DMSO-d₆, HCl salt) δ 12.05 (s, 1H), 7.74-7.70 (m, 1H), 7.63 (d, 1H), 7.44-7.31 (m, 4H), 6.33 (s, 1H), 5.71 (s, 2H), 3.75 (m, 4H), 3.53 (m, 4H).

### EXAMPLE 201

### 4-((2-(Dimethylamino)-1H-benzo[d]imidazol-1-yl)methyl)-7,8-difluoroquinolin-2(1H)-one

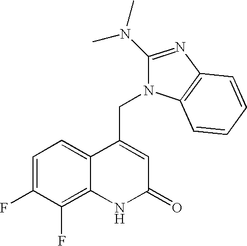

4-((2-(Dimethylamino)-1H-benzo[d]imidazol-1-yl)methyl)-7,8-difluoroquinolin-2(1H)-one was synthesized as described in EXAMPLE 200 using 4-(bromomethyl)-7,8-difluoroquinolin-2(1H)-one and N,N-dimethyl-1H-benzo[d]imidazol-2-amine as starting materials. ¹H NMR (400 MHz, DMSO-d₆, HCl salt) δ 12.18 (s, 1H), 7.74-7.71 (m, 1H), 7.54 (d, 1H), 7.43-7.35 (m, 3H), 7.29-7.25 (m, 1H), 6.26 (s, 1H), 5.76 (s, 2H), 3.19 (s, 6H). LCMS: 355 (M+H)⁺.

### EXAMPLE 202

### 4-((2-tert-Butyl-1H-benzo[d]imidazol-1-yl)methyl)-7,8-difluoroquinolin-2(1H)-one

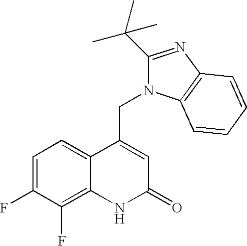

4-((2-tert-Butyl-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-tert-butyl-1H-benzo[d]imidazole (commercially available) as starting materials. ¹H NMR (400 MHz, DMSO-d₆, HCl salt) δ 12.05 (s, 1H), 7.94-7.91 (m, 2H), 7.63-7.55 (m, 2H), 7.51-7.44 (m, 2H), 6.16 (s, 2H), 5.63 (s, 1H), 1.56 (s, 9H). LCMS: 368 (M+H)⁺.

### EXAMPLE 203

### 4-((2-Chloro-1H-benzo[d]imidazol-1-yl)methyl)-7,8-difluoroquinolin-2(1H)-one

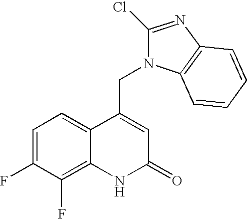

4-((2-Chloro-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-chloro-1H-benzo[d]imidazole (commercially available) as starting materials. ¹H NMR (400 MHz, DMSO-d₆) δ 12.08 (s, 1H), 7.85-7.81 (m, 1H), 7.73-7.71 (m, 1H), 7.62-7.60 (m, 1H), 7.46-7.41 (m, 1H), 7.35-7.31 (m, 2H), 5.89 (s, 2H), 5.17 (s, 1H). LCMS: 346 (M+H)⁺.

### EXAMPLE 204

### 4-((2-Cyclohexyl-1H-benzo[d]imidazol-1-yl)methyl)-7,8-difluoroquinolin-2(1H)-one

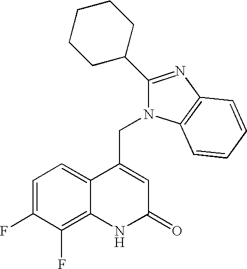

4-((2-Cyclohexyl-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-cyclohexyl-1H-benzo[d]imidazole as starting materials. ¹H NMR (400 MHz, DMSO-d₆, HCl salt) δ 12.15 (s, 1H), 7.90 (d, 1H), 7.81-7.75 (m, 2H), 7.61-7.44 (m, 3H), 6.18 (s, 2H), 5.52 (s, 1H), 3.35 (m, 1H), 1.97-1.68 (m, 7H), 1.38-1.24 (m, 3H). LCMS: 394 (M+H)⁺.

### EXAMPLE 205

### tert-Butyl 4-(1-((7,8-difluoro-2-oxo-1,2-dihydroquinolin-4-yl)methyl)-1H-benzo[d]imidazol-2-yl)piperidine-1-carboxylate

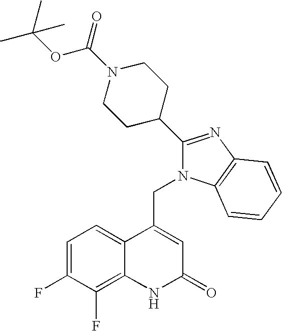

### Step 1: 2-(Piperidin-4-yl)-1H-benzo[d]imidazole

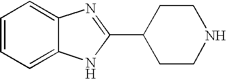

2-(Piperidin-4-yl)-1H-benzo[d]imidazole was synthesized as described in EXAMPLE 118, step 1 using piperidine-4-carboxylic acid as a starting material. LCMS: 202 (M+H)⁺.

### Step 2: tert-Butyl 4-(1H-benzo[d]imidazol-2-yl)piperidine-1-carboxylate

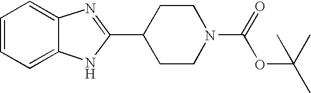

A mixture of 2-(piperidin-4-yl)-1H-benzo[d]imidazole (210 mg, 1.05 mmol), di-tert-butyl dicarbonate (230 mg, 1.05 mmol) in DCM (5 mL) was stirred at RT for 2 h. The solvent was removed and the residue was purified by column chromatography (eluting with EtOAc/Hexanes). LCMS: 302 (M+H)⁺.

### Step 3: tert-Butyl 4-(1-((7,8-difluoro-2-oxo-1,2-dihydroquinolin-4-yl)methyl)-1H-benzo[d]imidazol-2-yl)piperidine-1-carboxylate

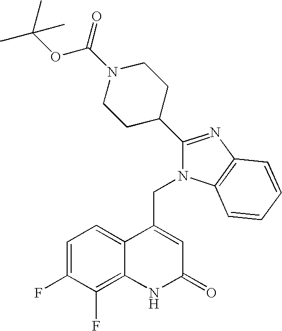

tert-Butyl 4-(1-((7,8-difluoro-2-oxo-1,2-dihydroquinolin-4-yl)methyl)-1H-benzo[d]imidazol-2-yl)piperidine-1-carboxylate was synthesized as described in EXAMPLE 118 using 4-(bromomethyl)-7,8-difluoroquinolin-2(1H)-one and 2-cyclohexyl-1H-benzo[d]imidazole as starting materials. LCMS: 495 (M+H)⁺.

### EXAMPLE 206

### 7,8-Difluoro-4-((2-(2-methylpiperidin-1-yl)-1H-benzo[d]imidazol-1-yl)methyl)quinolin-2(1H)-one

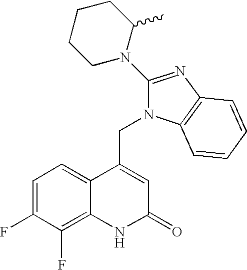

### Step 1: 2-(2-Methylpiperidin-1-yl)-1H-benzo[d]imidazole

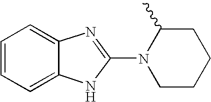

2-(2-Methylpiperidin-1-yl)-1H-benzo[d]imidazole was synthesized as a racemic mixture as described in EXAMPLE 200, step 1 using NMP as a solvent and heating the reaction mixture to 250° C. for 30 min in the microwave. ¹HNMR (400 MHz, DMSO-d₆) δ 11.15 (s, 1H), 7.13 (m, 2H), 6.87 (m, 2H), 4.39 (m, 1H), 3.88 (d, 1H), 3.02 (t, 1H), 1.71-1.44 (m, 6H), 1.16 (d, 3H).

### Step 2: 7,8-Difluoro-4-((2-(2-methylpiperidin-1-yl)-1H-benzo[d]imidazol-1-yl)methyl)quinolin-2(1H)-one

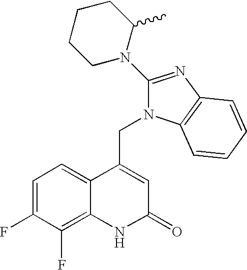

7,8-Difluoro-4-((2-(2-methylpiperidin-1-yl)-1H-benzo[d]imidazol-1-yl)methyl)quinolin-2(1H)-one was synthesized as a racemic mixture as described in EXAMPLE 118 using 4-(bromomethyl)-7,8-difluoroquinolin-2(1H)-one and racemic 2-(2-methylpiperidin-1-yl)-1H-benzo[d]imidazole as starting materials. ¹H NMR (400 MHz, DMSO-d₆, HCl salt) δ 12.21 (s, 1H), 7.75-7.72 (m, 1H), 7.58 (d, 1H), 7.42-7.36 (m, 3H), 7.31-7.27 (m, 1H), 6.27 (s, 1H), 5.66-5.52 (q, 2H), 4.01 (m, 1H), 3.48-3.43 (m, 2H), 1.87 (m, 1H), 1.66-1.46 (m, 5H), 1.27 (d, 3H). LCMS: 409 (M+H)⁺.

### EXAMPLE 207

### 4-((2-Cyclobutyl-1H-benzo[d]imidazol-1-yl)methyl)-7,8-difluoroquinolin-2(1H)-one

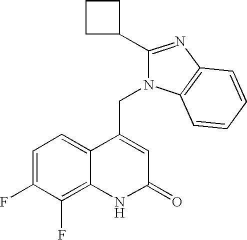

4-((2-Cyclobutyl-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-cyclobutyl-1H-benzo[d]imidazole as starting materials. ¹H NMR (400 MHz, DMSO-d₆, HCl salt) δ 12.11 (s, 1H), 7.93 (d, 1H), 7.81-7.76 (m, 2H), 7.62-7.42 (m, 3H), 6.03 (s, 2H), 5.55 (s, 1H), 4.20 (m, 1H), 2.71-2.61 (m, 2H), 2.36-2.28 (m, 2H), 2.07-1.90 (m, 2H). LCMS: 366 (M+H)⁺.

### EXAMPLE 208

### 7,8-Difluoro-4-((2-(2-methyl-1H-imidazol-1-yl)-1H-benzo[d]imidazol-1-yl)methyl)quinolin-2(1H)-one

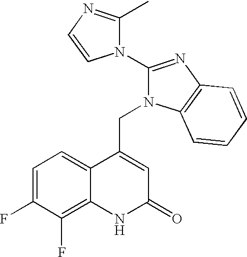

7,8-Difluoro-4-((2-(2-methyl-1H-imidazol-1-yl)-1H-benzo[d]imidazol-1-yl)methyl)quinolin-2(1H)-one was synthesized as described in EXAMPLE 206 using 4-(bromomethyl)-7,8-difluoroquinolin-2(1H)-one and 2-(2-methyl-1H-imidazol-1-yl)-1H-benzo[d]imidazole as starting materials. ¹H NMR (400 MHz, DMSO-d₆, HCl salt) δ. 12.05 (s, 1H), 7.91-7.89 (m, 1H), 7.85 (d, 1H), 7.72-7.69 (m, 2H), 7.57-7.54 (m, 1H), 7.49-7.44 (m, 2H), 7.39-7.32 (m, 1H), 5.81 (s, 2H), 5.63 (s, 1H), 2.51 (s, 3H). LCMS: 393 (M+2H)⁺.

### EXAMPLE 209

### 4-((2-Cyclopentyl-1H-benzo[d]imidazol-1-yl)methyl)-7,8-difluoroquinolin-2(1H)-one

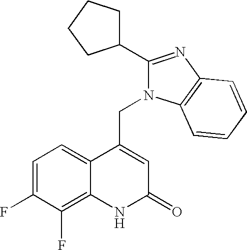

4-((2-Cyclopentyl-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and as starting materials. ¹H NMR (400 MHz, DMSO-d₆, HCl salt) δ 12.11 (s, 1H), 7.89 (d, 1H), 7.81-7.77 (m, 2H), 7.60-7.43 (m, 3H), 6.14 (s, 2H), 5.53 (s, 1H), 3.68 (m, 1H), 2.11-2.07 (m, 2H), 2.04-1.95 (m, 2H), 1.89-1.82 (m, 2H), 1.68-1.63 (m, 2H). LCMS: 380 (M+H)⁺.

### EXAMPLE 210

### (E or Z)-4-((2-(But-2-en-2-yl)-1H-benzo[d]imidazol-1-yl)methyl)-7,8-difluoroquinolin-2(1H)-one

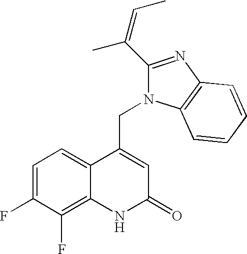

### Step 1: (E or Z)-2-(But-2-en-2-yl)-1H-benzo[d]imidazole

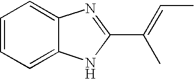

(E or Z)-2-(But-2-en-2-yl)-1H-benzo[d]imidazole was unexpectedly synthesized as described in EXAMPLE 118, Step 1 using 2-(2-methylcyclopropyl)-1H-benzo[d]imidazole as a starting material. ¹H NMR (400 MHz, DMSO-d₆) δ 12.21 (s, 1H), 7.47 (m, 2H), 7.11-7.09 (m, 2H), 6.57 (m, 1H), 2.09 (m, 3H), 1.84 (d, 3H).

### Step 2: (E or Z)-4-((2-(But-2-en-2-yl)-1H-benzo[d]imidazol-1-yl)methyl)-7,8-difluoroquinolin-2(1H)-one

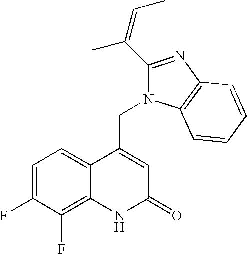

(E or Z)-4-((2-(But-2-en-2-yl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and (E or Z)-2-(but-2-en-2-yl)-1H-benzo[d]imidazole as starting materials. ¹H NMR (400 MHz, DMSO-d₆, HCl salt) δ 12.05 (s, 1H), 7.87 (d, 1H), 7.78-7.70 (m, 2H), 7.61-7.52 (m, 2H), 7.45-7.39 (m, 1H), 6.28 (m, 1H), 5.95 (s, 2H), 5.86 (s, 1H), 2.11 (m, 3H), 1.84 (d, 3H). LCMS: 366 (M+H)⁺.

### EXAMPLE 211

### 7,8-Difluoro-4-((2-isopentyl-1H-benzo[d]imidazol-1-yl)methyl)quinolin-2(1H)-one

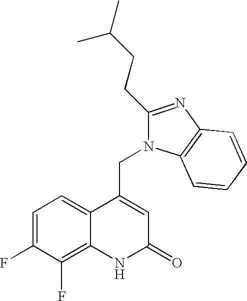

7,8-Difluoro-4-((2-isopentyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-isopentyl-1H-benzo[d]imidazole as starting materials. ¹H NMR (400 MHz, DMSO-d₆, HCl salt) δ 12.10 (s, 1H), 7.89 (d, 1H), 7.81-7.77 (m, 2H), 7.61-7.43 (m, 3H), 6.12 (s, 2H), 5.53 (s, 1H), 3.16 (t, 2H), 1.72 (q, 2H), 1.63 (m, 1H), 0.89 (d, 6H). LCMS: 381 (M)⁺.

### EXAMPLE 212

### 7,8-Difluoro-4-((2-(piperidin-4-yl)-1H-benzo[d]imidazol-1-yl)methyl)quinolin-2(1H)-one

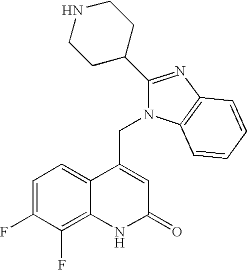

HCl (4M in 1,4-dioxane, 0.5 mL) was added to tert-butyl 4-(1-((7,8-difluoro-2-oxo-1,2-dihydroquinolin-4-yl)methyl)-1H-benzo[d]imidazol-2-yl)piperidine-1-carboxylate (55 mg, 0.11 mmol) dissolved in DCM/MeOH (4:1, 5 mL) and the resulting mixture was stirred at RT for 18 h. The solvent was removed and the residue was purified by preparative HPLC (ACN/H₂O). LCMS: 395 (M+H)⁺.

### EXAMPLE 213

### 4-((2-(2-Cyclohexylethyl)-1H-benzo[d]imidazol-1-yl)methyl)-7,8-difluoroquinolin-2(1H)-one

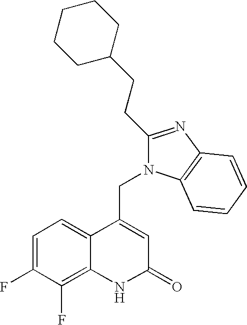

4-((2-(2-Cyclohexylethyl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-(2-cyclohexylethyl)-1H-benzo[d]imidazole as starting materials. ¹H NMR (400 MHz, DMSO-d₆, HCl salt) δ 12.05 (s, 1H), 7.90 (d, 1H), 7.83-7.78 (m, 2H), 7.62-7.45 (m, 3H), 6.17 (s, 2H), 5.53 (s, 1H), 3.24-3.15 (m, 2H), 1.77-1.55 (m, 7H), 1.27-1.05 (m, 4H), 0.90-0.81 (m, 2H). LCMS: 422 (M+H)⁺.

### EXAMPLE 214

### 4-((2-Benzyl-1H-benzo[d]imidazol-1-yl)methyl)-7,8-difluoroquinolin-2(1H)-one

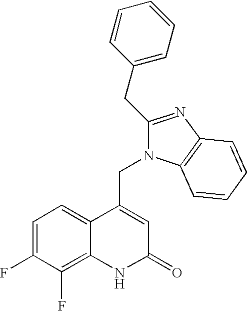

4-((2-Benzyl-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-benzyl-1H-benzo[d]imidazole as starting materials. ¹H NMR (400 MHz, DMSO-d₆, HCl salt) δ 11.96 (s, 1H), 7.89 (d, 1H), 7.75 (d, 1H), 7.71-7.69 (m, 1H), 7.60-7.50 (m, 2H), 7.46-7.41 (m, 1H), 7.33-7.31 (m, 2H), 7.14-7.12 (m, 3H), 6.13 (s, 2H), 5.03 (s, 1H), 4.68 (s, 2H). LCMS: 403 (M+2H)⁺.

### EXAMPLE 215

### 4-((2-(Cyclopentylmethyl)-1H-benzo[d]imidazol-1-yl)methyl)-7,8-difluoroquinolin-2(1H)-one

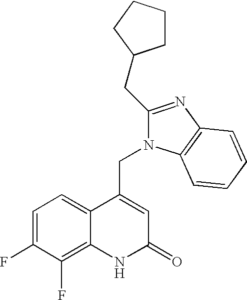

4-((2-(Cyclopentylmethyl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-(cyclopentylmethyl)-1H-benzo[d]imidazole as starting materials. ¹H NMR (400 MHz, DMSO-d₆, HCl salt) δ 12.10 (s, 1H), 7.91 (d, 1H), 7.81-7.77 (m, 2H), 7.63-7.45 (m, 3H), 6.15 (s, 2H), 5.45 (s, 1H), 3.24 (d, 2H), 2.43 (m, 1H), 1.77-1.73 (m, 2H), 1.65-1.61 (m, 2H), 1.51-1.47 (m, 2H), 1.28-1.22 (m, 2H). LCMS: 395 (M+2H)⁺.

### EXAMPLE 216

### 7,8-Difluoro-4-((2-(2-methylbenzyl)-1H-benzo[d]imidazol-1-yl)methyl)quinolin-2(1H)-one

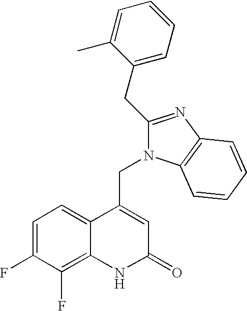

7,8-Difluoro-4-((2-(2-methylbenzyl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(2-methylbenzyl)-1H-benzo[d]imidazole as starting materials. ¹H NMR (400 MHz, DMSO-d₆, HCl salt) δ 7.90-7.86 (m, 2H), 7.69-7.65 (m, 1H), 7.63-7.56 (m, 2H), 7.45-7.39 (m, 1H), 7.16 (d, 1H), 7.09-7.05 (m, 2H), 6.90 (m, 1H), 6.13 (s, 2H), 5.30 (s, 1H), 4.71 (s, 2H), 2.20 (s, 3H). LCMS: 417 (M+2H)⁺.

### EXAMPLE 217

### 7,8-Difluoro-4-((2-(pentan-3-yl)-1H-benzo[d]imidazol-1-yl)methyl)quinolin-2(1H)-one

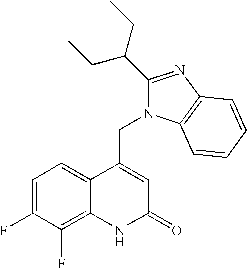

7,8-Difluoro-4-((2-(pentan-3-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(pentan-3-yl)-1H-benzo[d]imidazole as starting materials. ¹H NMR (400 MHz, DMSO-d₆, HCl salt) δ 12.21 (s, 1H), 7.92 (d, 1H), 7.85-7.80 (m, 2H), 7.64-7.44 (m, 3H), 6.19 (s, 2H), 5.40 (s, 1H), 3.40 (m, 1H), 2.00-1.91 (m, 2H), 1.89-1.82 (m, 2H), 0.82 (t, 6H). LCMS: 383 (M+2H)⁺.

### EXAMPLE 218

### 4-((2-(1H-Imidazol-1-yl)-1H-benzo[d]imidazol-1-yl)methyl)-7,8-difluoroquinolin-2(1H)-one

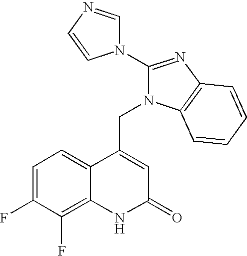

4-((2-(1H-Imidazol-1-yl)-1H-benzo[d]imidazol-1-yl)methyl)-7,8-difluoroquinolin-2(1H)-one was synthesized as described in EXAMPLE 200 using 4-(bromomethyl)-7,8-difluoroquinolin-2(1H)-one and 2-(1H-imidazol-1-yl)-1H-benzo[d]imidazole as starting materials. ¹H NMR (400 MHz, DMSO-d₆, HCl salt) δ 12.05 (s, 1H), 8.93 (s, 1H), 7.86-7.82 (m, 2H), 7.68-7.65 (m, 1H), 7.61-7.58 (m, 1H), 7.51 (s, 1H), 7.43-7.31 (m, 3H), 5.83 (s, 2H), 5.54 (s, 1H). LCMS: 378 (M+H)⁺.

### EXAMPLE 219

### 7,8-Difluoro-4-((2-propyl-1H-benzo[d]imidazol-1-yl)methyl)quinolin-2(1H)-one

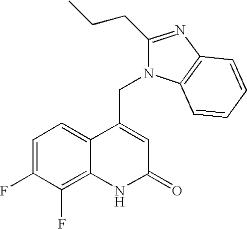

7,8-Difluoro-4-((2-propyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-propyl-1H-benzo[d]imidazole as starting materials. ¹H NMR (400 MHz, DMSO-d₆, HCl salt) δ 12.19 (s, 1H), 7.89 (d, 1H), 7.78 (m, 2H), 7.61-7.43 (m, 3H), 6.12 (s, 2H), 5.53 (s, 1H), 3.17 (t, 2H), 1.85 (m, 2H), 0.98 (t, 3H). LCMS: 354 (M+H)⁺.

### EXAMPLE 220

### 7,8-Difluoro-4-((2-(2-methylbutyl)-1H-benzo[d]imidazol-1-yl)methyl)quinolin-2(1H)-one

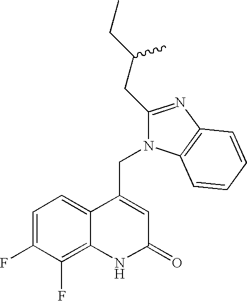

7,8-Difluoro-4-((2-(2-methylbutyl)-1H-benzo[d]imidazol-1-yl)methyl)quinolin-2(1H)-one was synthesized as a racemic mixture as described in EXAMPLE 118 using 4-(bromomethyl)-7,8-difluoroquinolin-2(1H)-one and racemic 2-(2-methylbutyl)-1H-benzo[d]imidazole as starting materials. ¹H NMR (400 MHz, DMSO-d₆, HCl salt) δ 12.05 (s, 1H), 7.86-7.82 (m, 1H), 7.66 (d, 1H), 7.46-7.40 (m, 2H), 7.23-7.15 (m, 2H), 5.81 (s, 2H), 5.06 (s, 1H), 2.81-2.75 (m, 1H), 2.67-2.60 (m, 1H), 1.98 (m, 1H), 1.42-1.37 (m, 1H), 1.23-1.15 (m, 1H), 0.89 (d, 3H), 0.81 (t, 3H). LCMS: 382 (M+H)⁺.

### EXAMPLE 221

### 7,8-Difluoro-4-((2-ethyl-1H-benzo[d]imidazol-1-yl)methyl)quinolin-2(1H)-one

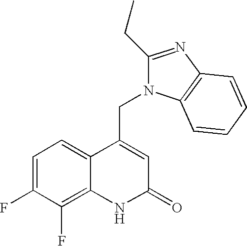

7,8-Difluoro-4-((2-ethyl-1H-benzo[d]imidazol-1-yl)methyl)quinolin-2(1H)-one was synthesized as a racemic mixture as described in EXAMPLE 118 using 4-(bromomethyl)-7,8-difluoroquinolin-2(1H)-one and 2-ethyl-1H-benzo[d]imidazole as starting materials. ¹H NMR (400 MHz, DMSO-d₆, HC salt) δ 12.19 (s, 1H), 7.91 (d, 1H), 7.81 (d, 1H), 7.79-7.76 (m, 1H), 7.63-7.43 (m, 3H), 6.11 (s, 2H), 5.60 (s, 1H), 3.20 (q, 2H), 1.40 (t, 3H).

### EXAMPLE 222

### 7,8-Difluoro-4-((2-(3,3,3-trifluoro-2-methylpropyl)-1H-benzo[d]imidazol-1-yl)methyl)quinolin-2(1H)-one

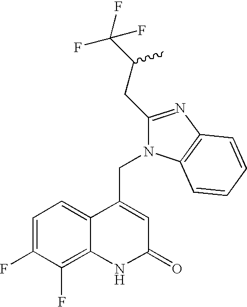

7,8-Difluoro-4-((2-(3,3,3-trifluoro-2-methylpropyl)-1H-benzo[d]imidazol-1-yl)methyl)quinolin-2(1H)-one was synthesized as a racemic mixture as described in EXAMPLE 118 using 4-(bromomethyl)-7,8-difluoroquinolin-2(1H)-one and racemic-(3,3,3-trifluoro-2-methylpropyl)-1H-benzo[d]imidazole as starting materials. ¹H NMR (400 MHz, DMSO-d₆, HCl salt) δ 12.05 (s, 1H), 7.89 (d, 1H), 7.80-7.76 (m, 1H), 7.73 (d, 1H), 7.57-7.43 (m, 3H), 6.17-6.06 (m, 2H), 5.43 (s, 1H), 3.51-3.34 (m, 3H), 1.19 (d, 3H). LCMS: 422 (M+H)⁺.

### EXAMPLE 223

### 4-[(2-cyclopropyl-4H-imidazo[4,5-b]pyridin-4-yl)methyl]-7,8-difluoroquinolin-2(1H)-one and 4-((2-cyclopropyl-3H-imidazo[4,5-b]pyridin-3-yl)methyl)-7,8-difluoroquinolin-2(1H)-one

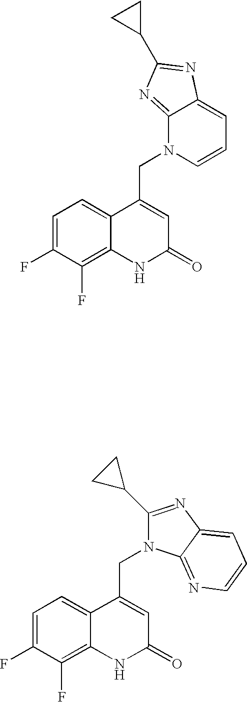

4-[(2-cyclopropyl-4H-imidazo[4,5-b]pyridin-4-yl)methyl]-7,8-difluoroquinolin-2(1H)-one and 4-((2-cyclopropyl-3H-imidazo[4,5-b]pyridin-3-yl)methyl)-7,8-difluoroquinolin-2(1H)-one were synthesized as a mixture as described in EXAMPLE 118 using 2-cyclopropyl-1H-imidazo[4,5-b]pyridine and 4-(bromomethyl)-7,8-difluoroquinolin-2(1H)-one as starting materials. The 2 regioisomers were separated by reverse phase chromatography (ACN/H₂O):

First eluting regioisomer: 4-[(2-cyclopropyl-4H-imidazo[4,5-b]pyridin-4-yl)methyl]-7,8-difluoroquinolin-2(1H)-one. ¹H NMR (400 MHz, DMSO-d₆; TFA salt) δ 12.21 (s, 1H), 8.75 (d, 1H), 8.64 (d, 1H), 7.85-7.73 (m, 2H), 7.43-7.34 (m, 1H), 6.24 (s, 2H), 5.83 (s, 1H), 2.41-2.33 (m, 1H), 1.39-1.21 (m, 4H). LCMS: 353 (M+H)⁺.

Second eluting regioisomer: 4-((2-Cyclopropyl-3H-imidazo[4,5-b]pyridin-3-yl)methyl)-7,8-difluoroquinolin-2(1H)-one. ¹H NMR (400 MHz, DMSO-d₆; TFA salt) δ 12.15 (s, 1H), 8.31 (d, 1H), 8.06 (d, 1H), 7.92 (m, 1H), 7.45-7.32 (m, 2H), 5.97 (s, 2H), 5.45 (s, 1H), 2.31 (m, 1H), 1.30-1.05 (m, 4H). LCMS: 353 (M+H)⁺.

### EXAMPLE 224

### 7,8-Difluoro-4-((2-isopropyl-4-methyl-1H-benzo[d]imidazol-1-yl)methyl)quinolin-2(1H)-one

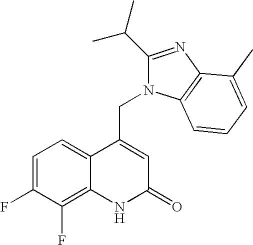

7,8-Difluoro-4-((2-isopropyl-4-methyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-isopropyl-4-methyl-1H-benzo[d]imidazole as starting materials. ¹H NMR (400 MHz, DMSO-d₆ and D₂O, HCl salt) δ 7.73 (m, 1H), 7.50-7.20 (m, 4H), 6.03 (s, 2H), 5.38 (s, 1H), 3.51 (m, 1H), 2.59 (s, 3H), 1.38 (d, 6H). LCMS: 368 (M+H)⁺.

### EXAMPLE 225

### 7,8-Difluoro-4-((2-isobutyl-4-methyl-1H-benzo[d]imidazol-1-yl)methyl)quinolin-2(1H)-one

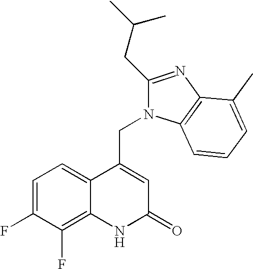

7,8-Difluoro-4-((2-isobutyl-4-methyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-isobutyl-4-methyl-1H-benzo[d]imidazole as starting materials. ¹H NMR (400 MHz, DMSO-d₆, HCl salt) δ 12.12 (s, 1H), 7.78 (m, 1H), 7.55-7.35 (m, 4H), 6.11 (s, 2H), 5.43 (s, 1H), 3.14 (d, 2H), 2.68 (s, 3H), 2.21 (m, 1H), 0.95 (d, 6H). LCMS: 382 (M+H)⁺.

### EXAMPLE 226

### 4-((2-(1,5-Dimethyl-1H-imidazol-4-yl)-1H-benzo[d]imidazol-1-yl)methyl)-7,8-difluoroquinolin-2(1H)-one

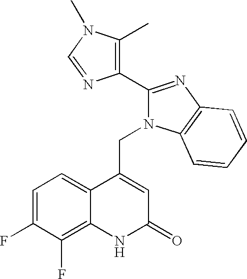

4-((2-(1,5-Dimethyl-1H-imidazol-4-yl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-(1,5-dimethyl-1H-imidazol-4-yl)-1H-benzo[d]imidazole as starting materials. ¹H NMR (400 MHz, DMSO-d₆, HCl salt) δ 12.05 (s, 1H), 7.92 (s, 1H), 7.86 (d, 1H), 7.77 (m, 1H), 7.68 (d, 1H), 7.52-7.33 (m, 3H), 6.27 (s, 2H), 5.50 (s, 1H), 3.64 (s, 3H), 2.59 (s, 3H). LCMS: 406 (M+H)⁺.

### EXAMPLE 227

### 7,8-Difluoro-4-((4-fluoro-2-isopropyl-1H-benzo[d]imidazol-1-yl)methyl)quinolin-2(1H)-one and 7,8-difluoro-4-((7-fluoro-2-isopropyl-1H-benzo[d]imidazol-1-yl)methyl)quinolin-2(1H)-one

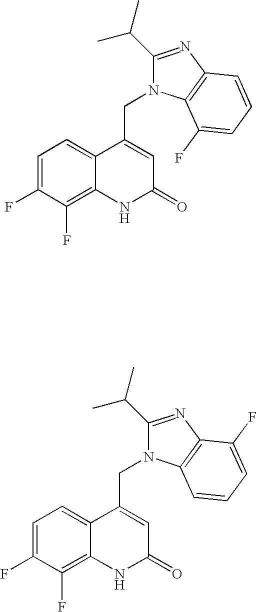

7,8-Difluoro-4-((4-fluoro-2-isopropyl-1H-benzo[d]imidazol-1-yl)methyl)quinolin-2(1H)-one and 7,8-difluoro-4-((7-fluoro-2-isopropyl-1H-benzo[d]imidazol-1-yl)methyl)quinolin-2(1H)-one were synthesized as a mixture as described in EXAMPLE 118 using 4-fluoro-2-isopropyl-1H-benzo[d]imidazole and 4-(bromomethyl)-7,8-difluoroquinolin-2(1H)-one as starting materials. The 2 regioisomers were separated by reverse phase chromatography (ACN/H₂O):

First eluting regioisomer: 7,8-difluoro-4-((4-fluoro-2-isopropyl-1H-benzo[d]imidazol-1-yl)methyl)quinolin-2(1H)-one. ¹H NMR (400 MHz, CD₃OD, TFA salt) δ 7.80 (m, 1H), 7.65 (m, 1H), 7.52 (m, 1H), 7.40-7.20 (m, 2H), 6.10 (s, 2H), 5.70 (s, 1H), 3.60 (m, 1H), 1.50 (d, 6H). LCMS: 372 (M+H)⁺.

Second eluting regioisomer: 7,8-difluoro-4-((7-fluoro-2-isopropyl-1H-benzo[d]imidazol-1-yl)methyl)quinolin-2(1H)-one. ¹H NMR (400 MHz, DMSO-d₆, TFA salt) δ 12.05 (s, 1H), 7.82 (m, 1H), 7.43 (m, 1H), 7.30 (m, 1H), 7.17 (m, 1H), 7.05 (m, 1H), 5.88 (s, 2H), 5.10 (s, 1H), 3.24 (m, 1H), 1.28 (d, 6H). LCMS: 372 (M+H)⁺.

### EXAMPLE 228

### 7,8-Difluoro-4-((5-fluoro-2-isopropyl-1H-benzo[d]imidazol-1-yl)methyl)quinolin-2(1H)-one and 7,8-difluoro-4-((6-fluoro-2-isopropyl-1H-benzo[d]imidazol-1-yl)methyl)quinolin-2(1H)-one

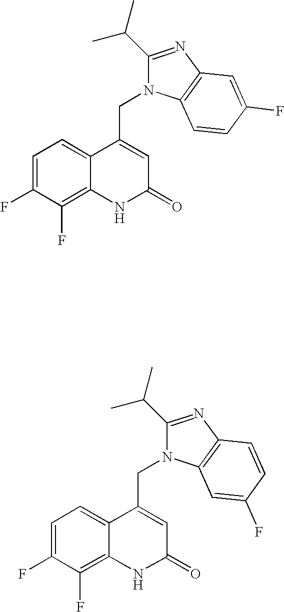

7,8-Difluoro-4-((5-fluoro-2-isopropyl-1H-benzo[d]imidazol-1-yl)methyl)quinolin-2(1H)-one and 7,8-difluoro-4-((6-fluoro-2-isopropyl-1H-benzo[d]imidazol-1-yl)methyl)quinolin-2(1H)-one were synthesized as a mixture as described in EXAMPLE 118 using 5-fluoro-2-isopropyl-1H-benzo[d]imidazole and 4-(bromomethyl)-7,8-difluoroquinolin-2(1H)-one as starting materials. The 2 regioisomers were separated by reverse phase chromatography (ACN/H₂O):

First eluting regioisomer: 7,8-difluoro-4-((5-fluoro-2-isopropyl-1H-benzo[d]imidazol-1-yl)methyl)quinolin-2(1H)-one. ¹H NMR (400 MHz, CD₃OD, TFA salt) δ 7.82 (m, 1H), 7.72 (m, 1H), 7.61 (m, 1H), 7.39-7.30 (m, 2H), 6.10 (s, 2H), 5.73 (s, 1H), 3.63 (m, 1H), 1.53 (d, 6H). LCMS: 372 (M+H)⁺.

First eluting regioisomer: 7,8-difluoro-4-((6-fluoro-2-isopropyl-1H-benzo[d]imidazol-1-yl)methyl)quinolin-2(1H)-one. ¹H NMR (400 MHz, CD₃OD, TFA salt) δ 7.88-7.78 (m, 2H), 7.55 (m, 1H), 7.43-7.28 (m, 2H), 6.06 (s, 2H), 5.64 (s, 1H), 3.54 (m, 1H), 1.48 (d, 6H). LCMS: 372 (M+H)⁺.

### EXAMPLE 229

### 7,8-Difluoro-4-((5,6-difluoro-2-isopropyl-1H-benzo[d]imidazol-1-yl)methyl)quinolin-2(1H)-one

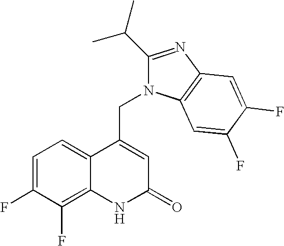

7,8-Difluoro-4-((5,6-difluoro-2-isopropyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 5,6-difluoro-2-isopropyl-1H-benzo[d]imidazole as starting materials. ¹H NMR (400 MHz, DMSO-d₆, TFA salt) δ 12.05 (s, 1H), 7.85-7.78 (m, 3H), 7.45 (m, 1H), 5.88 (s, 2H), 5.14 (s, 1H), 3.25 (m, 1H), 1.29 (d, 6H). LCMS: 390 (M+H)⁺.

### EXAMPLE 230

### 7,8-Difluoro-4-((2-isopropyl-5H-imidazo[4,5-c]pyridin-5-yl)methyl)quinolin-2(1H)-one

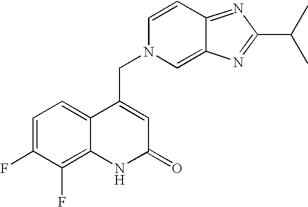

7,8-Difluoro-4-((2-isopropyl-5H-imidazo[4,5-c]pyridin-5-yl)methyl)quinolin-2(1H)-one was unexpectedly synthesized as described in EXAMPLE 118 using 4-(bromomethyl)-7,8-difluoroquinolin-2(1H)-one and 2-isopropyl-1H-imidazo[4,5-c]pyridine as starting materials. ¹H NMR (400 MHz, CD₃OD, TFA salt) δ 9.43 (s, 1H), 8.68 (d, 1H), 8.14 (d, 1H), 7.68 (m, 1H), 7.26 (m, 1H), 6.25 (s, 2H), 5.78 (s, 1H), 3.42 (m, 1H), 1.50 (d, 6H). LCMS: 355 (M+H)⁺.

### EXAMPLE 231

### 7,8-difluoro-4-[(2-isopropyl-4H-imidazo[4,5-b]pyridin-4-yl)methyl]quinolin-2(1H)-one and 7,8-difluoro-4-((2-isopropyl-3H-imidazo[4,5-b]pyridin-3-yl)methyl)quinolin-2(1H)-one

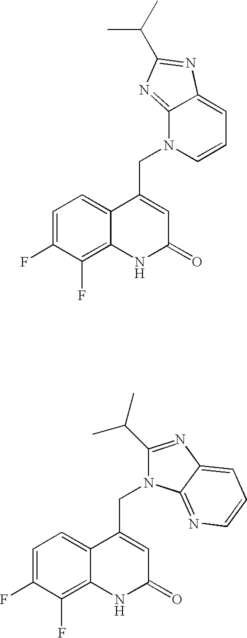

7,8-difluoro-4-[(2-isopropyl-4H-imidazo[4,5-b]pyridin-4-yl)methyl]quinolin-2(1H)-one and 7,8-difluoro-4-((2-isopropyl-3H-imidazo[4,5-b]pyridin-3-yl)methyl)quinolin-2(1H)-one were synthesized as a mixture as described in EXAMPLE 118 using 2-isopropyl-1H-imidazo[4,5-b]pyridine and 4-(bromomethyl)-7,8-difluoroquinolin-2(1H)-one as starting materials. The 2 regioisomers were separated by reverse phase chromatography (ACN/H₂O):

First eluting regioisomer: 7,8-difluoro-4-[(2-isopropyl-4H-imidazo[4,5-b]pyridin-4-yl)methyl]quinolin-2(1H)-one. ¹H NMR (400 MHz, CD₃OD, TFA salt) δ 8.75 (d, 1H), 8.65 (d, 1H), 7.85-7.78 (m, 2H), 7.26 (m, 1H), 6.40 (s, 2H), 5.92 (s, 1H), 3.41 (m, 1H), 1.46 (d, 6H). LCMS: 355 (M+H)⁺. second eluting regioisomer: 7,8-Difluoro-4-((2-isopropyl-3H-imidazo[4,5-b]pyridin-3-yl)methyl)quinolin-2(1H)-one. ¹H NMR (400 MHz, CD₃OD, TFA salt) δ 8.56 (d, 1H), 8.27 (d, 1H), 7.87 (m, 1H), 7.63 (m, 1H), 7.32 (m, 1H), 6.08 (s, 2H), 5.71 (s, 1H), 3.60 (m, 1H), 1.50 (d, 6H). LCMS: 355 (M+H)⁺.

### EXAMPLE 232

### 4-((2-(3,5-Dimethylisoxazol-4-yl)-1H-benzo[d]imidazol-1-yl)methyl)-7,8-difluoroquinolin-2(1H)-one

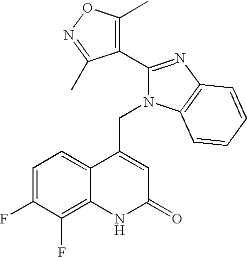

4-((2-(3,5-Dimethylisoxazol-4-yl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 4-(1H-benzo[d]imidazol-2-yl)-3,5-dimethylisoxazole as starting materials. ¹H NMR (400 MHz, CD₃OD. TFA salt) δ 7.94 (d, 1H), 7.74-7.58 (m, 3H), 7.30-7.14 (m, 2H), 5.93 (s, 2H), 5.78 (s, 1H), 2.45 (s, 3H), 2.25 (s, 3H). LCMS: 407 (M+H)⁺.

### EXAMPLE 233

### 7,8-Difluoro-4-((2-(1-methyl-1H-pyrazol-5-yl)-1H-benzo[d]imidazol-1-yl)methyl)quinolin-2(1H)-one

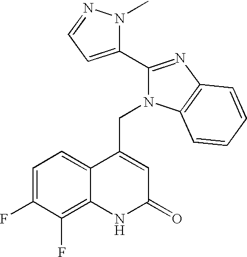

7,8-Difluoro-4-((2-(1-methyl-1H-pyrazol-5-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(1-methyl-1H-pyrazol-5-yl)-1H-benzo[d]imidazole as starting materials. ¹H NMR (400 MHz, CDCl₃) δ 7.90 (d, 1H), 7.60-7.10 (m, 7H), 6.40 (s, 2H), 5.80 (s, 1H), 4.15 (s, 3H). LCMS: 392 (M+H)⁺.

### EXAMPLE 234

### 7,8-Difluoro-4-((2-isopropyl-5-methyl-1H-benzo[d]imidazol-1-yl)methyl)quinolin-2(1H)-one and 7,8-difluoro-4-((2-isopropyl-6-methyl-1H-benzo[d]imidazol-1-yl)methyl)quinolin-2(1H)-one

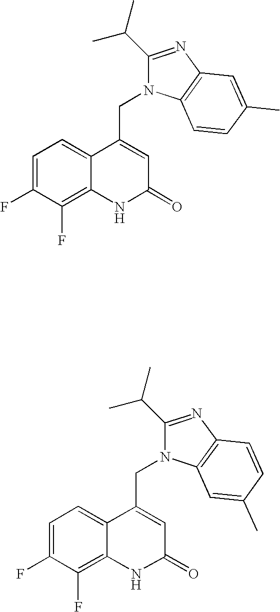

7,8-Difluoro-4-((2-isopropyl-5-methyl-1H-benzo[d]imidazol-1-yl)methyl)quinolin-2(1H)-one and 7,8-difluoro-4-((2-isopropyl-6-methyl-1H-benzo[d]imidazol-1-yl)methyl)quinolin-2(1H)-one were synthesized as a mixture as described in EXAMPLE 118 using 4-(bromomethyl)-7,8-difluoroquinolin-2(1H)-one and 2-isopropyl-5-methyl-1H-benzo[d]imidazole as starting materials. LCMS: 368 (M+H)⁺.

### EXAMPLE 235

### 4-((5-Chloro-2-isopropyl-1H-benzo[d]imidazol-1-yl)methyl)-7,8-difluoroquinolin-2(1H)-one and 4-((6-chloro-2-isopropyl-1H-benzo[d]imidazol-1-yl)methyl)-7,8-difluoroquinolin-2(1H)-one

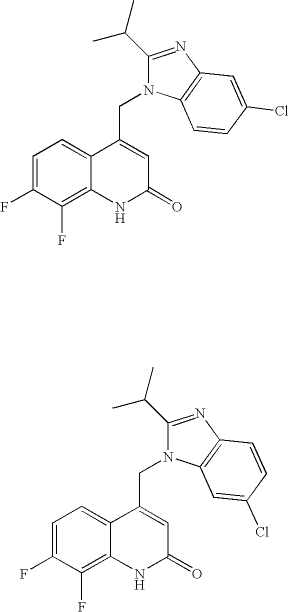

4-((5-Chloro-2-isopropyl-1H-benzo[d]imidazol-1-yl)methyl)-7,8-difluoroquinolin-2(1H)-one and 4-((6-chloro-2-isopropyl-1H-benzo[d]imidazol-1-yl)methyl)-7,8-difluoroquinolin-2(1H)-one were synthesized as a mixture as described in EXAMPLE 118 using 4-(bromomethyl)-7,8-difluoroquinolin-2(1H)-one and 2-isopropyl-5-chloro-1H-benzo[d]imidazole as starting materials. LCMS: 388 (M+H)⁺.

### EXAMPLE 236

### 7,8-difluoro-4-((8-isopropyl-6-methyl-3H-purin-3-yl)methyl)quinolin-2(1H)-one and 7,8-difluoro-4-((8-isopropyl-6-methyl-9H-purin-9-yl)methyl)quinolin-2(1H)-one

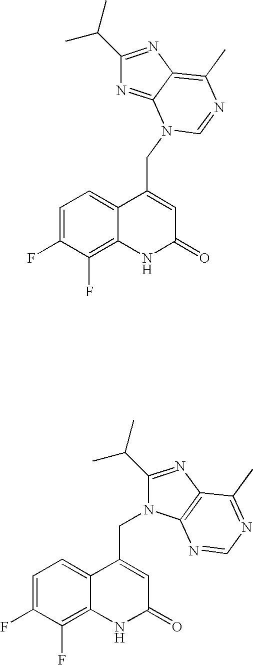

7,8-Difluoro-4-((8-isopropyl-6-methyl-3H-purin-3-yl)methyl)quinolin-2(1H)-one and 7,8-difluoro-4-((8-isopropyl-6-methyl-9H-purin-9-yl)methyl)quinolin-2(1H)-one were synthesized as a mixture as described in EXAMPLE 118 using 8-isopropyl-6-methyl-9H-purine and 4-(bromomethyl)-7,8-difluoroquinolin-2(1H)-one as starting materials. The 2 regioisomers were separated by reverse phase chromatography (ACN/H₂O):

First eluting regioisomer: 7,8-difluoro-4-((8-isopropyl-6-methyl-3H-purin-3-yl)methyl)quinolin-2(1H)-one. ¹H NMR (400 MHz, CD₃OD, TFA salt) δ 9.36 (s, 1H), 7.87 (m, 1H), 7.27 (m, 1H), 6.28 (s, 2H), 6.10 (s, 1H), 3.41 (m, 1H), 3.00 (s, 3H), 1.46 (d, 6H). LCMS: 370 (M+H)⁺.

Second eluting regioisomer: 7,8-difluoro-4-((8-isopropyl-6-methyl-9H-purin-9-yl)methyl)quinolin-2(1H)-one. ¹H NMR (400 MHz, CD₃OD, TFA salt) δ 8.92 (s, 1H), 7.83 (m, 1H), 7.31 (m, 1H), 5.95 (s, 2H), 5.53 (s, 1H), 3.35 (m, 1H), 2.98 (s, 3H), 1.42 (d, 6H). LCMS: 370 (M+H)⁺.

### EXAMPLE 237

### 7,8-Difluoro-4-((2-neopentyl-1H-benzo[d]imidazol-1-yl)methyl)quinolin-2(1H)-one

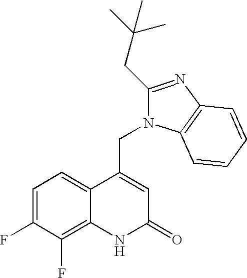

7,8-Difluoro-4-((2-neopentyl-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-neopentyl-1H-benzo[d]imidazole as starting materials. ¹H NMR (400 MHz, DMSO-d₆, HCl salt) δ 7.93 (d, 1H), 7.84-7.80 (m, 1H), 7.68 (d, 1H), 7.64-7.60 (m, 1H), 7.55-7.43 (m, 2H), 6.18 (s, 2H), 5.56 (s, 1H), 3.25 (s, 2H), 1.07 (s, 9H). LCMS: 381 (M)⁺.

### EXAMPLE 238

### 7,8-Difluoro-4-((2-(2-methylcyclopropyl)-1H-benzo[d]imidazol-1-yl)methyl)quinolin-2(1H)-one

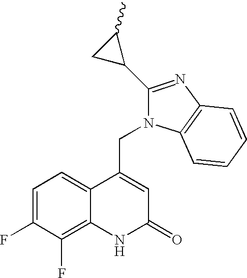

### Step 1: 2-(2-Methylcyclopropyl)-1H-benzo[d]imidazole

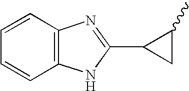

A mixture of benzene-1,2-diamine (500 mg, 4.6 mmol) and racemic 2-methylcyclopropanecarboxylic acid (3.6 mL, 37 mmol) in HCl (3M, 10 mL) was heated to 100° C. for 18 h. The reaction mixture was cooled to RT and poured into 1M NaOH (10 mL). The aqueous layer was extracted with DCM (5×50 mL). The crude was purified by column chromatography on silica gel (50 to 100% EtOAc in Hexanes) to afford 2-(2-methylcyclopropyl)-1H-benzo[d]imidazole as a white solid. ¹H NMR (400 MHz, DMSO-d₆, HCl) δ 12.10 (s, 1H), 7.42-7.31 (m, 2H), 7.05-7.03 (m, 2H), 1.80-1.76 (m, 1H), 1.39-1.34 (m, 1H), 1.21-1.17 (m, 1H), 1.15 (d, 3H), 0.86-0.83 (m, 1H).

### Step 2: 7,8-Difluoro-4-((2-(2-methylcyclopropyl)-1H-benzo[d]imidazol-1-yl)methyl)quinolin-2(1H)-one

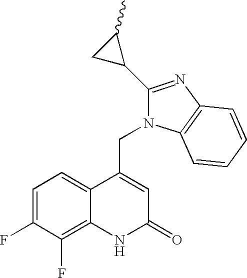

7,8-Difluoro-4-((2-(2-methylcyclopropyl)-1H-benzo[d]imidazol-1-yl)methyl)quinolin-2(1H)-one was synthesized as a racemic mixture as described in EXAMPLE 118, Step 2 using 4-(bromomethyl)-7,8-difluoroquinolin-2(1H)-one and 2-(2-methylcyclopropyl)-1H-benzo[d]imidazole as starting materials. ¹H NMR (400 MHz, DMSO-d₆, HCl salt) δ 12.05 (s, 1H), 7.83-7.78 (m, 3H), 7.57-7.50 (m, 3H), 6.19 (s, 2H), 5.63 (s, 1H), 2.37 (m, 1H), 1.87-1.77 (m, 2H), 1.19 (m, 1H), 1.10 (d, 3H). LCMS: 366 (M)⁺.

### EXAMPLE 239

### 7,8-Difluoro-4-((3-isopropyl-1H-indazol-1-yl)methyl)quinolin-2(1H)-one

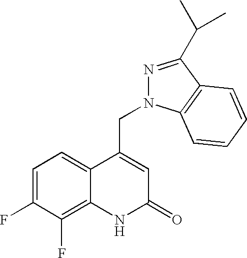

### Step 1: 1-(2-Aminophenyl)-2-methylpropan-1-one

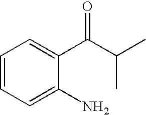

A mixture of 2-aminobenzonitrile (2 g, 17 mmol) and isopropylmagnesium chloride (2M in THF, 25 mL, 50 mmol) in THF (30 mL) was heated to 50° C. for 3 h. The reaction mixture was then cooled to 0° C. followed by addition of HCl (2M, 28 mL). The resulting mixture was then heated to 50° C. for 1 h.

The aqueous layer was neutralized by addition of 1M NaOH and it was extracted with EtOAc. The crude was purified by column chromatography on silica gel (0 to 50% EtOAc in Hexanes) to afford 1-(2-aminophenyl)-2-methylpropan-1-one as a yellow oil. ¹H NMR (400 MHz, DMSO-d₆) δ 7.77 (d, 1H), 7.25 (t, 1H), 6.63 (m, 2H), 6.29 (s, 2H), 3.59 (m, 1H), 1.20 (d, 6H).

### Step 2: 3-Isopropyl-1H-indazole

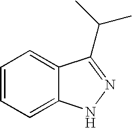

1-(2-Aminophenyl)-2-methylpropan-1-one (1 g, 6.13 mmol) was dissolved in HCl (conc. 10 mL) and cooled to 0° C. Sodium nitrite (466 mg, 6.75 mmol) in H₂O (5 mL) was then added dropwise and the resulting orange solution was stirred at 0° C. for 1 h. A solution of SnCl₂.2H₂O (3.3 g, 14.7 mmol) in HCl (conc., 5 mL) was then added and the reaction mixture was stirred at 0° C. for 2 h. The aqueous solution was extracted with DCM and the crude was purified by column chromatography on silica gel (10 to 50% EtOAc in Hexanes) to afford 630 mg of 3-isopropyl-1H-indazole as a yellow solid. ¹H NMR (400 MHz, DMSO-d₆) δ 7.78 (d, 1H), 7.42 (d, 1H), 7.35 (t, 1H), 7.12 (t, 1H), 3.47 (m, 1H), 1.49 (d, 6H).

### Step 3: 7,8-Difluoro-4-((3-isopropyl-1H-indazol-1-yl)methyl)quinolin-2(1H)-one

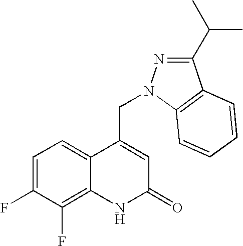

7,8-Difluoro-4-((3-isopropyl-1H-indazol-1-yl)methyl)quinolin-2(1H)-one was synthesized as described in EXAMPLE 118 using 3-isopropyl-1H-indazole and 4-(bromomethyl)-7,8-difluoroquinolin-2(1H)-one as starting materials. ¹H NMR (400 MHz, DMSO-d₆) δ 12.02 (s, 1H), 7.87-7.83 (m, 2H), 7.60 (d, 1H), 7.39-7.33 (m, 2H), 7.14 (t, 1H), 5.91 (s, 2H), 5.50 (s, 1H), 3.41 (m, 1H), 1.38 (d, 6H). LCMS: 353 (M)⁺.

### EXAMPLE 240

### 7,8-Difluoro-4-((2-o-tolyl-1H-benzo[d]imidazol-1-yl)methyl)quinolin-2(1H)-one

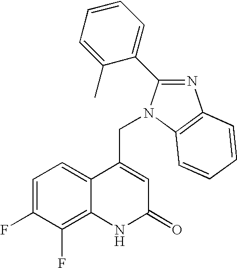

7,8-Difluoro-4-((2-o-tolyl-1H-benzo[d]imidazol-1-yl)methyl)quinolin-2(1H)-one was synthesized as described in EXAMPLE 118 using 2-o-tolyl-1H-benzo[d]imidazole and 4-(bromomethyl)-7,8-difluoroquinolin-2(1H)-one as starting materials. ¹H NMR (400 MHz, DMSO-d₆, TFA salt) δ 12.05 (s, 1H), 7.90-7.85 (d, 1H), 7.71-7.64 (d, 1H), 7.58-7.54 (m, 1H), 7.49-7.43 (m, 3H), 7.38-7.35 (m, 2H), 7.19-7.05 (m, 2H), 5.69 (s, 2H), 5.59 (s, 1H), 2.50 (s, 3H). LCMS: 401.1 (M)⁺.

### EXAMPLE 241

### 4-((2-(2-Chlorophenyl)-1H-benzo[d]imidazol-1-yl)methyl)-7,8-difluoroquinolin-2(1H)-one

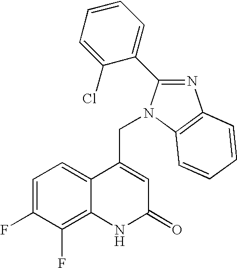

4-((2-(2-Chlorophenyl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-(2-chlorophenyl)-1H-benzo[d]imidazole as starting materials. ¹H NMR (400 MHz, DMSO-d₆, TFA salt) δ 12.05 (s, 1H), 7.85-7.83 (m, 1H), 7.75-7.74 (d, 2H), 7.63-7.21 (m, 7H), 5.62 (s, 2H), 5.40 (s, 1H). LCMS: 422.1 (M)⁺.

### EXAMPLE 242

### 4-((2-(2-(Dimethylamino)phenyl)-1H-benzo[d]imidazol-1-yl)methyl)-7,8-difluoroquinolin-2(1H)-one

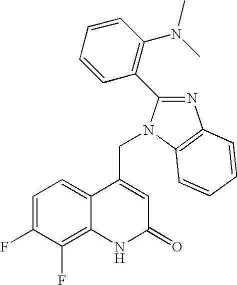

4-((2-(2-(Dimethylamino)phenyl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-(1H-benzo[d]imidazol-2-yl)-N,N-dimethylaniline as starting materials. ¹H NMR (400 MHz, DMSO-d₆, TFA salt) δ 12.03 (s, 1H), 7.88-7.86 (d, 1H), 7.69-7.67 (d, 1H), 7.57-7.51 (m, 1H), 7.48-7.43 (m, 2H), 7.31-7.28 (m, 3H), 7.19-7.12 (m, 2H), 5.70 (s, 2H), 5.61 (s, 1H), 3.76 (s, 3H), 3.57 (s, 3H). LCMS: 430.0 (M)⁺.

### EXAMPLE 243

### 4-((2-(2,5-Dimethylphenyl)-1H-benzo[d]imidazol-1-yl)methyl)-7,8-difluoroquinolin-2(1H)-one

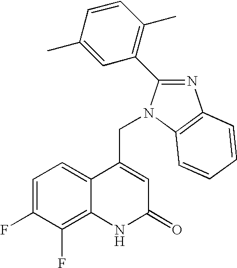

4-((2-(2,5-Dimethylphenyl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-(2,5-dimethylphenyl)-1H-benzo[d]imidazole as starting materials. ¹H NMR (400 MHz, DMSO-d₆, TFA salt) δ 12.03 (s, 1H), 7.88-7.85 (d, 1H), 7.71-7.69 (d, 1H), 7.59-7.54 (m, 2H), 7.43-7.38 (m, 2H), 7.39-7.28 (m, 3H), 5.68 (s, 2H), 5.47 (s, 1H), 2.20 (s, 3H), 2.18 (s, 3H). LCMS: 415.1 (M)⁺.

### EXAMPLE 244

### 7,8-Difluoro-4-((2-(3-fluoropyridin-4-yl)-1H-benzo[d]imidazol-1-yl)methyl)quinolin-2(1H)-one

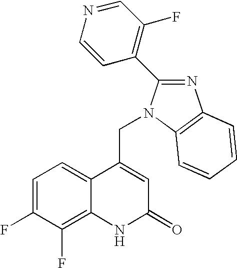

7,8-Difluoro-4-((2-(3-fluoropyridin-4-yl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(3-fluoropyridin-4-yl)-1H-benzo[d]imidazole as starting materials. ¹H NMR (400 MHz, DMSO-d₆, TFA salt) δ 12.07 (s. 1H), 8.78 (s, 1H), 8.56-8.54 (d, 1H), 7.91-7.87 (m, 2H), 7.72-7.53 (m, 3H), 7.39-7.26 (m, 2H), 5.79 (s, 2H), 5.35 (s, 1H). LCMS: 406.1 (M)⁺.

### EXAMPLE 245

### 7,8-Difluoro-4-((2-(2-(trifluoromethyl)phenyl)-1H-benzo[d]imidazol-1-yl)methyl)quinolin-2(1H)-one

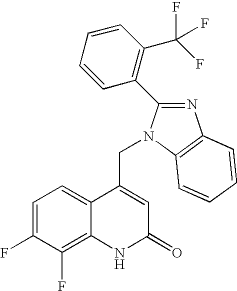

7,8-Difluoro-4-((2-(2-(trifluoromethyl)phenyl)-1H-benzo[d]imidazol-1-yl)methyl)quinolin-2(1H)-one was synthesized as described in EXAMPLE 118 using 4-(bromomethyl)-7,8-difluoroquinolin-2(1H)-one and 2-(2-(trifluoromethyl)phenyl)-1H-benzo[d]imidazole as starting materials. ¹H NMR (400 MHz, DMSO-d₆, TFA salt) δ 12.01 (s, 1H), 7.95-7.92 (m, 2H), 7.79-7.71 (m, 3H), 7.63-7.58 (m, 2H), 7.37-7.35 (d, 1H), 7.29-7.22 (m, 2H), 5.60 (s, 2H), 5.41 (s, 1H). LCMS: 455.1 (M)⁺.

### EXAMPLE 246

### 4-((2-(2,6-Dimethylphenyl)-1H-benzo[d]imidazol-1-yl)methyl)-7,8-difluoroquinolin-2(1H)-one

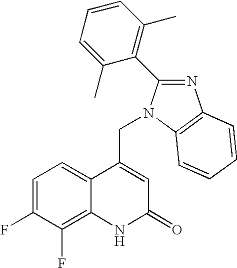

4-((2-(2,6-Dimethylphenyl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-(2,6-dimethylphenyl)-1H-benzo[d]imidazole as starting materials. ¹H NMR (400 MHz, DMSO-d₆, TFA salt) δ 12.03 (s, 1H), 7.82-7.81 (d, 1H), 7.63-7.58 (m, 2H), 7.41-7.37 (m, 3H), 7.35-7.24 (m, 2H), 7.09-7.07 (d, 1H), 5.64 (s, 2H), 5.42 (s, 1H), 2.28 (s, 3H), 2.18 (s, 3H). LCMS: 415.1 (M)⁺.

### EXAMPLE 247

### 4-((2-(2,6-Difluorophenyl)-1H-benzo[d]imidazol-1-yl)methyl)-7,8-difluoroquinolin-2(1H)-one

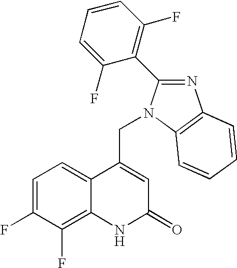

4-((2-(2,6-Difluorophenyl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-(2,6-difluorophenyl)-1H-benzo[d]imidazole as starting materials. ¹H NMR (400 MHz, DMSO-d₆, TFA salt) δ 12.03 (s, 1H), 7.85-7.83 (d, 1H), 7.65-7.50 (m, 3H), 7.32-7.21 (m, 3H), 7.11-7.09 (m, 2H), 5.62-5.50 (m, 2H), 5.40 (s, 1H). LCMS: 423.1 (M)⁺.

### EXAMPLE 248

### 4-((2-(3-Chloropyridin-4-yl)-1H-benzo[d]imidazol-1-yl)methyl)-7,8-difluoroquinolin-2(1H)-one

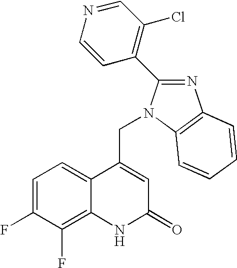

4-((2-(3-Chloropyridin-4-yl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-(3-chloropyridin-4-yl)-1H-benzo[d]imidazole as starting materials. ¹H NMR (400 MHz, DMSO-d₆, TFA salt) δ 12.09 (s, 1H), 9.03-9.02 (d, 1H), 8.65-8.64 (d, 1H), 7.64-7.57 (m, 2H), 7.38-7.36 (m, 3H), 7.10-7.06 (m, 2H), 6.05 (s, 2H), 5.57 (s, 1H). LCMS: 422.1 (M)⁺.

### EXAMPLE 249

### 4-((2-(2-Aminophenyl)-1H-benzo[d]imidazol-1-yl)methyl)-7,8-difluoroquinolin-2(1H)-one and 4-((2-(1H-benzo[d]imidazol-2-yl)phenylamino)methyl)-7,8-difluoroquinolin-2(1H)-one

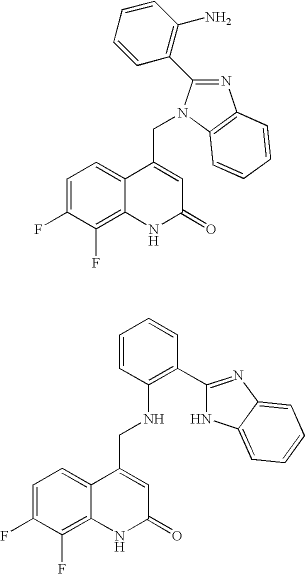

4-((2-(2-Aminophenyl)-1H-benzo[d]imidazol-1-yl)methyl)-7,8-difluoroquinolin-2(1H)-one and 4-((2-(1H-benzo[d]imidazol-2-yl)phenylamino)methyl)-7,8-difluoroquinolin-2(1H)-one were synthesized as a mixture of regioisomers as described in EXAMPLE 118 using 4-(bromomethyl)-7,8-difluoroquinolin-2(1H)-one and 2-(1H-benzo[d]imidazol-2-yl)aniline as starting materials. The two regioisomers were separated by preparative HPLC (ACN/H₂O):

First eluting regioisomer: 4-((2-(2-aminophenyl)-1H-benzo[d]imidazol-1-yl)methyl)-7,8-difluoroquinolin-2(1H)-one. ¹H NMR (400 MHz, DMSO-d₆, TFA salt) δ 12.07 (s, 1H), 7.87-7.86 (d, 1H), 7.85-7.7.71 (m, 2H), 7.58-7.29 (m, 2H), 7.19-7.11 (m, 3H), 6.88-6.86 (d, 1H), 6.62-6.60 (t, 1H), 5.80 (s, 2H), 5.76 (s, 1H), 3.58 (s, 2H). LCMS: 402.1 (M)⁺.

Second eluting regioisomers: 4-((2-(1H-benzo[d]imidazol-2-yl)phenylamino)methyl)-7,8-difluoroquinolin-2(1H)-one. ¹H NMR (400 MHz, DMSO-d₆, TFA salt) δ 12.05 (s, 1H), 11.96 (s, 1H), 7.95-7.90 (m, 1H), 7.80-7.77 (m, 3H), 7.36-7.25 (m, 4H), 6.80-6.66 (m, 2H), 6.42 (s, 1H), 4.86 (s, 2H), 3.51 (m, 1H). LCMS: 402.1 (M)⁺.

### EXAMPLE 250

### 7,8-Difluoro-4-((2-(tetrahydrofuran-2-yl)-1H-benzo[d]imidazol-1-yl)methyl)quinolin-2(1H)-one

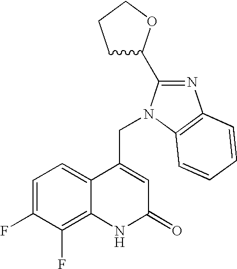

7,8-Difluoro-4-((2-(tetrahydrofuran-2-yl)-1H-benzo[d]imidazol-1-yl)methyl)quinolin-2(1H)-one was synthesized as a racemic mixture as described in EXAMPLE 118 using 4-(bromomethyl)-7,8-difluoroquinolin-2(1H)-one and racemic 2-(tetrahydrofuran-2-yl)-1H-benzo[d]imidazole as starting materials. ¹H NMR (400 MHz, DMSO-d₆, TFA salt) δ 12.09 (s, 1H), 7.77-7.75 (m, 2H), 7.57-7.55 (m, 1H), 7.39-7.33 (m, 3H), 5.94 (s, 2H), 5.30 (s, 1H), 3.82-3.81 (m, 1H), 2.38-2.24 (m, 2H), 2.15-2.05 (m, 2H), 1.95-1.90 (m, 2H). LCMS: 382 (M+H)⁺.

### EXAMPLE 251

### 4-((2-((1H-Imidazol-1-yl)methyl)-1H-benzo[d]imidazol-1-yl)methyl)-7,8-difluoroquinolin-2(1H)-one

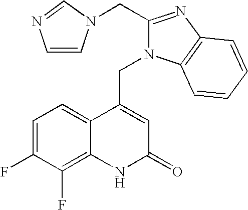

4-((2-((1H-Imidazol-1-yl)methyl)-1H-benzo[d]imidazol-1-yl)methyl)-7,8-difluoroquinolin-2(1H)-one was synthesized as described in EXAMPLE 118 using 4-(bromomethyl)-7,8-difluoroquinolin-2(1H)-one and 2-((1H-imidazol-1-yl)methyl)-1H-benzo[d]imidazole as starting materials. ¹H NMR (400 MHz, DMSO-d₆, TFA salt) δ 12.06 (s, 1H), 9.20 (s, 1H), 7.75-7.74 (m, 3H), 7.63 (s, 1H), 7.56-7.53 (m, 1H), 7.52-7.49 (m, 1H), 7.29-7.27 (m, 2H), 5.97 (s, 1H), 5.84 (s, 2H), 5.01 (s, 2H). LCMS: 391.1 (M)⁺.

### EXAMPLE 252

### 7,8-Difluoro-4-((5-oxo-2-phenylpyrazolidin-1-yl)methyl)quinolin-2(1H)-one

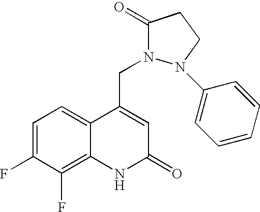

7,8-Difluoro-4-((3-oxo-2-phenylpyrazolidin-1-yl)methyl)quinolin-2(1H)-one was synthesized as described in EXAMPLE 118 using 1-phenylpyrazolidin-3-one and 4-(bromomethyl)-7,8-difluoroquinolin-2(1H)-one as starting materials. ¹H NMR (400 MHz, DMSO-d₆) δ 12.01 (s, 1H), 7.71-7.63 (m, 1H), 7.38-7.35 (m, 2H), 7.16-7.12 (m, 2H), 7.09-7.05 (m, 1H), 7.02-7.00 (d, 2H), 6.30 (s, 2H), 4.75 (s, 2H), 3.89-3.85 (t, 2H). LCMS: 356 (M+H)⁺.

### EXAMPLE 253

### N-(3-Chlorophenyl)-N-(fluoro(8-fluoro-2-oxo-1,2-dihydroquinolin-4-yl)methyl)-4-methylthiazole-5-carboxamide

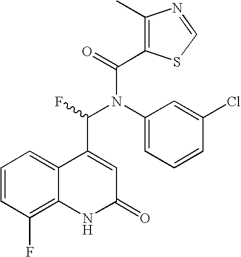

### Step 1: 4-(Bromomethyl)-2-chloro-8-fluoroquinoline

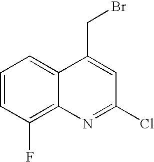

A mixture of POCl₃ (70 mL, 99%) and 4-(bromomethyl)-8-fluoroquinolin-2(1H)-one (5 g, 19.41 mmol) was heated to 90° C. for 4 h. The mixture was concentrated to dryness under reduced pressure and the pH was adjusted to pH=8 by adding Na₂CO₃. The resulting solution was extracted with EtOAc (2×300 mL), the organics were combined, dried over Na₂SO₄, and concentrated to afford 4 g (68%) of 4-(bromomethyl)-2-chloro-8-fluoroquinoline as a yellow solid.

### Step 2: (2-Chloro-8-fluoroquinolin-4-yl)methanol

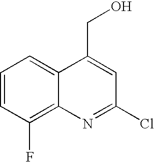

A mixture of 4-(bromomethyl)-2-chloro-8-fluoroquinoline (1 g, 3.48 mmol) and HCOOK (1.5 g, 17.86 mmol) in MeOH/H₂O (2:1, 30 mL) was refluxed for 18 h. The reaction mixture was cooled to RT followed by filtration of the yellow solid to afford 0.7 g (91%) of (2-chloro-8-fluoroquinolin-4-yl)methanol as a yellow solid.

### Step 3: 2-Chloro-8-fluoro-4-(fluoromethyl)quinoline

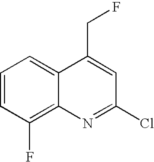

(2-Chloro-8-fluoroquinolin-4-yl)methanol (2.0 g, 9.00 mmol) was added in several batches to a solution of DAST (1.4 g, 8.70 mmol) in DCM (60 mL) maintained at 0° C. The resulting solution was stirred at RT for 2 h. The solvent was removed and the residue was purified by column chromatography on silica gel to afford 1.7 g (87%) of 2-chloro-8-fluoro-4-(fluoromethyl)quinoline as a white solid.

### Step 4: 8-Fluoro-4-(fluoromethyl)-2-methoxyquinoline

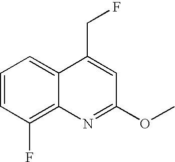

A mixture of 2-chloro-8-fluoro-4-(fluoromethyl)quinoline (1.5 g, 6.69 mmol) and MeONa (730 mg, 13.52 mmol) in MeOH (80 mL) was heated to 80° C. for 4 h. The mixture was concentrated to dryness and the residue obtained was dissolved in EtOAc (150 mL). The organic layer was washed with brine (2×60 mL), dried over Na₂SO₄, and concentrated to dryness. The residue was purified by column chromatography on silica gel to afford 1.3 g (88%) of 8-fluoro-4-(fluoromethyl)-2-methoxyquinoline as a yellow solid. LCMS: 210 (M+H)⁺.

### Step 5: 4-(Bromofluoromethyl)-8-fluoro-2-methoxyquinoline

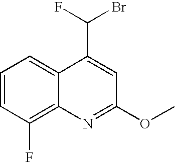

A mixture of 8-fluoro-4-(fluoromethyl)-2-methoxyquinoline (1.3 g, 5.91 mmol), AIBN (cat.), NBS (4.2 g, 23.73 mmol) in CCl₄ (150 mL) was refluxed for 18 h. The solvent was removed and the residue obtained was purified by column chromatography on silica gel (eluting with 1:10 EtOAc:PE) to afford 1.2 g (68%) of 4-(bromofluoromethyl)-8-fluoro-2-methoxyquinoline as a yellow solid. LCMS: 288 (M+H)⁺.

### Step 6: N-(3-Chlorophenyl)-N-(fluoro(8-fluoro-2-methoxyquinolin-4-yl)methyl)-4-methylthiazole-5-carboxamide

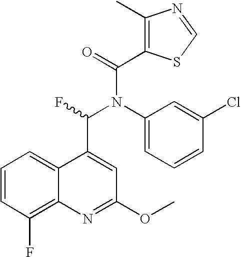

Sodium hydride (60%, 80 mg, 2.00 mmol) was added in several batches to N-(3-chlorophenyl)-4-methylthiazole-5-carboxamide (500 mg, 1.92 mmol) in DMF (20 mL). The resulting solution was stirred at RT for 1 h. To this mixture was added 4-(bromofluoromethyl)-8-fluoro-2-methoxyquinoline (500 mg, 1.74 mmol) and the resulting solution was stirred at RT for 18 h. EtOAc (200 mL) was added and the organic layer was washed with brine (3×60 mL), dried over Na₂SO₄, and concentrated to dryness. The residue was purified by column chromatography on silica gel (eluting with 1:3 EtOAc/PE) to afford 500 mg (62%) of N-(3-chlorophenyl)-N-(fluoro(8-fluoro-2-methoxyquinolin-4-yl)methyl)-4-methylthiazole-5-carboxamide as a yellow solid. ¹H NMR (300 MHz, DMSO-d₆) δ 8.97 (s, 1H), 8.54-8.38 (m, 1H), 7.69-7.55 (m, 3H), 7.33 (d, 1H), 7.19-7.14 (t, 1H), 6.97 (s, 1H), 6.85 (s, 1H), 6.70 (s, 1H), 3.93 (s, 3H), 2.61 (s, 3H). LCMS: 460 (M+H)⁺.

### Step 7: N-(3-Chlorophenyl)-N-(fluoro(8-fluoro-2-oxo-1,2-dihydroquinolin-4-yl)methyl)-4-methylthiazole-5-carboxamide

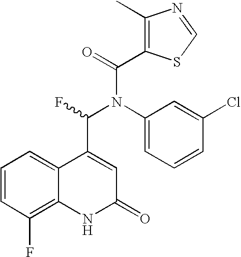

BBr₃ (60 mg, 0.24 mmol) in DCM (10 mL) was added dropwise to a solution of N-(3-chlorophenyl)-N-(fluoro(8-fluoro-2-methoxyquinolin-4-yl)methyl)-4-methylthiazole-5-carboxamide (100 mg, 0.22 mmol) in DCM (20 mL) while maintaining the temperature at −30° C. The resulting solution was stirred at RT for 18 h. It was then diluted with DCM (30 mL), washed with sat. aqueous NaHCO₃ (20 mL), washed with brine (20 mL), dried over Na₂SO₄, and concentrated to dryness. The residue was purified by column chromatography on silica gel (eluting with 1:3 EtOAc/hexane) to afford 80 mg (78%) of N-(3-chlorophenyl)-N-(fluoro(8-fluoro-2-oxo-1,2-dihydroquinolin-4-yl)methyl)-4-methylthiazole-5-carboxamide as a white solid. ¹H NMR (300 MHz, DMSO-d₆) δ 11.9 (s, 1H), 8.97 (s, 1H), 8.34-8.18 (m, 1H), 7.55-7.23 (m, 5H), 7.00 (s, 1H), 6.78 (s, 1H), 6.34 (s, 1H), 2.60 (s, 3H). LCMS: 446 (M+H)⁺.

### EXAMPLE 254

### 7,8-Difluoro-4-((4-isopropyl-2-phenyl-1H-imidazol-1-yl)methyl)quinolin-2(1H)-one

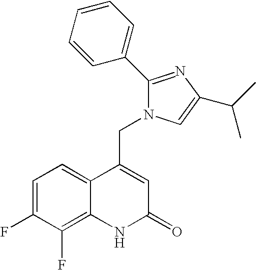

### Step 1: 1-Bromo-3-methylbutan-2-one

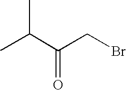

Br₂ (4 g, 25.00 mmol) was added dropwise to a solution of 3-methylbutan-2-one (2.15 g, 25.00 mmol) in MeOH (20 mL) over a period of 2 h while maintaining the temperature at 0-10° C. The resulting solution was stirred at that temperature for 1 h. AcONa (2.05 g, 25.00 mmol) was then added and the solvent was removed. The residue obtained was taken up in EtOAc (120 mL), the organic layer was washed with H₂O (2×40 mL), dried over Na₂SO₄, and evaporated to dryness to afford 3 g (crude) of 1-bromo-3-methylbutan-2-one as a colorless liquid.

### Step 2: 4-Isopropyl-2-phenyl-1H-imidazole

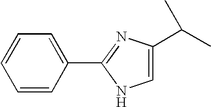

A mixture of 1-bromo-3-methylbutan-2-one (3 g, 10 mmol), K₂CO₃ (1.38 g, 13.66 mmol), and benzamidine hydrochloride (1.56 g, 10 mmol) in MeOH (60 mL) was refluxed for 18 h. The mixture was concentrated and the resulting solution was diluted with EtOAc (200 mL). The organic layer was washed with H₂O (2×50 mL), dried over Na₂SO₄, and concentrated to dryness. The crude was purified by column chromatography on silica gel (1:2 EtOAc/PE) to afford 0.9 g (45%) of 4-isopropyl-2-phenyl-1H-imidazole as a white solid. ¹H NMR (300 MHz, DMSO-d₆) δ 12.15 (s, 1H), 7.92 (d, 2H), 7.42 (t, 2H), 7.30 (t, 1H), 6.82 (s, 1H), 2.88 (m, 1H), 1.22 (d, 6H). LCMS: 187 (M+H)⁺.

### Step 3: 7,8-Difluoro-4-((4-isopropyl-2-phenyl-1H-imidazol-1-yl)methyl)quinolin-2(1H)-one

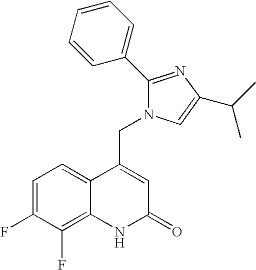

7,8-Difluoro-4-((4-isopropyl-2-phenyl-1H-imidazol-1-yl)methyl)quinolin-2(1H)-one was synthesized as described in EXAMPLE 118, Step 2 using 4-isopropyl-2-phenyl-1H-imidazole and 4-(bromomethyl)-7,8-difluoroquinolin-2(1H)-one as starting materials. ¹H NMR (300 MHz, DMSO-d₆) δ 12.10 (s, 1H), 7.62-7.51 (m, 3H), 7.40-7.29 (m, 4H), 7.09 (s, 1H), 5.62 (s, 1H), 5.55 (s, 2H), 2.87 (m, 1H), 1.23 (d, 6H). LCMS: 380 (M+H)⁺.

### EXAMPLE 255

### 7,8-Difluoro-4-((2-isopropyl-5-phenyl-1H-imidazol-1-yl)methyl)quinolin-2(1H)-one

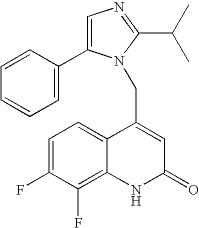

### Step 1: 2-Isopropyl-4,5-dihydro-1H-imidazole

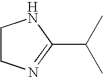

A mixture of isobutyraldehyde (14.4 g, 200 mmol) and ethane-1,2-diamine (12 g, 200 mmol) in DCM (50 mL) was stirred at 0° C. for 4 h. To the mixture was added NBS (35 g, 197.74 mmol) in several batches. The resulting solution was stirred at RT for 3 h. The resulting solution was washed with 10% NaOH, dried over Na₂SO₄, and concentrated to dryness to afford 16 g (64%) of 2-isopropyl-4,5-dihydro-1H-imidazole as colorless liquid.

### Step 2: 2-Isopropyl-1H-imidazole

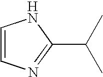

Nickel (1 g, 10%) was added to a solution of 2-isopropyl-4,5-dihydro-1H-imidazole (10 g, 80.36 mmol) in EtOH (20 mL) and it was refluxed for 48 h and then cooled to RT. The insoluble particles were filtered and the filtrate was evaporated to dryness to afford 4 g (41%) of 2-isopropyl-1H-imidazole as a white solid.

### Step 3: 7,8-Difluoro-4-((2-isopropyl-1H-imidazol-1-yl)methyl)quinolin-2(1H)-one

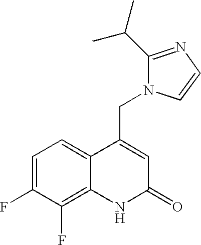

7,8-Difluoro-4-((2-isopropyl-1H-imidazol-1-yl)methyl)quinolin-2(1H)-one was synthesized as described in EXAMPLE 118, Step 2 using 4-(bromomethyl)-7,8-difluoroquinolin-2(1H)-one and 2-isopropyl-1H-imidazole as starting materials.

### Step 4: 7,8-Difluoro-4-((2-isopropyl-5-phenyl-1H-imidazol-1-yl)methyl)quinolin-2(1H)-one

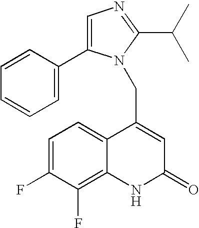

A mixture of 7,8-difluoro-4-((2-isopropyl-1H-imidazol-1-yl)methyl)quinolin-2(1H)-one (1 g, 3.30 mmol), bromobenzene (600 mg, 3.85 mmol), Pd(OAc)₂ (200 mg, 1.04 mmol), triphenylphosphine (200 mg, 0.76 mmol), and Na₂CO₃ (200 mg) in DMF (30 mL) was heated to 140° C. for 18 h. The solvent was removed and the crude was purified by column chromatography on silica gel (100:1 DCM/MeOH) to afford 0.8 g (48%) of 8-difluoro-4-((2-isopropyl-5-phenyl-1H-imidazol-1-yl)methyl)quinolin-2(1H)-one as a yellow solid. LCMS: 380 (M+H)⁺.

### EXAMPLE 256

### 7,8-Difluoro-4((2,4,5-trimethyl-1H-imidazol-1-yl)methyl)quinolin-2(1H)-one

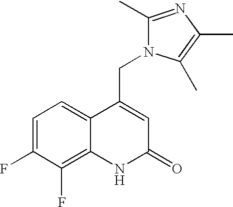

### Step 1: 2,4,5-Trimethyl-1H-imidazole

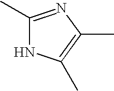

To a solution of acetaldehyde (100 mg, 2.72 mmol) in acetic acid (13 mL) was added 2,3-butanedione (198 μL, 2.72 mmol) and ammonium acetate (700 mg, 9.09 mmol). The solution was heated at 180° C. for 5 min in the microwave. The reaction mixture was added dropwise to a 0° C. solution of ammonium hydroxide, diluted with EtOAc, washed with H₂O (2×50 mL), dried over Na₂SO₄, and concentrated to provide 2,4,5-trimethyl-1H-imidazole (63 mg, 25%) as an off white solid. ¹H NMR (400 MHz, CDCl₃) δ 2.23 (s, 3H), 2.18 (s, 6H); LCMS: 111.12 (M+H)⁺.

### Step 2: 7,8-Difluoro-4((2,4,5-trimethyl-1H-imidazol-1-yl)methyl)quinolin-2(1H)-one

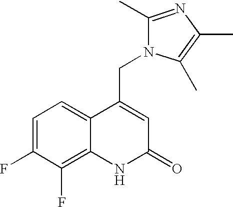

7,8-Difluoro-4((2,4,5-trimethyl-1H-imidazol-1-yl)methyl)quinolin-2(1H)-one was synthesized as describe in EXAMPLE 118, Step 2 using 2,4,5-trimethyl-1H-imidazole and 4-(bromomethyl)-7,8-difluoroquinolin-2(1H)-one as starting materials. ¹H NMR (400 MHz, DMSO-d₆) δ 12.13-12.10 (m, 1H), 7.66-7.63 (m, 1H), 7.45-7.38 (m, 1H), 5.69-5.65 (m, 2H), 5.45 (s, 1H), 2.51 (s, 3H), 2.24 (s, 3H), 2.09 (s, 3H). LCMS: 304 (M+H)⁺.

### EXAMPLE 257

### 4-((2-(3-Chlorophenyl)-4,5-dimethyl-1H-imidazol-1-yl)methyl)-7,8-difluoroquinolin-2(1H)-one

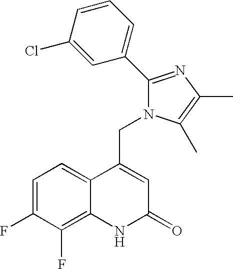

### Step 1: 2-(3-Chlorophenyl)-4,5-dimethyl-1H-imidazole

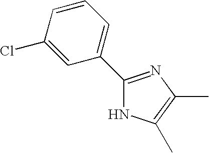

2-(3-Chlorophenyl)-4,5-dimethyl-1H-imidazole was synthesized as described as in EXAMPLE 256, Step 1 using 3-chlorobenzaldehyde as a starting material.

### Step 2: 4-((2-(3-Chlorophenyl)-4,5-dimethyl-1H-imidazol-1-yl)methyl)-7,8-difluoroquinolin-2(1H)-one

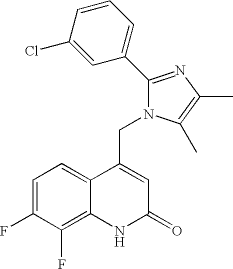

4-((2-(3-Chlorophenyl)-4,5-dimethyl-1H-imidazol-1-yl)methyl)-7,8-difluoroquinolin-2(1H)-one was synthesized as described in EXAMPLE 118, Step 2 using 2-(3-chlorophenyl)-4,5-dimethyl-1H-imidazole and 4-(bromomethyl)-7,8-difluoroquinolin-2(1H)-one as starting materials. ¹H NMR (400 MHz, DMSO-d₆) δ 12.20-11.70 (m, 1H), 7.61-7.59 (m, 1H), 7.58-7.53 (m, 2H), 7.32-7.30 (m, 2H), 7.16-7.09 (m, 1H), 6.02 (s, 1H), 5.73 (s, 2H), 2.09 (s, 6H). LCMS: 399.96 (M+H)⁺.

### EXAMPLE 258

### 7,8-Difluoro-4-((2-isopropyl-5-methyl-4-phenyl-1H-imidazol-1-yul)methyl)quinolin-2(1H)-one

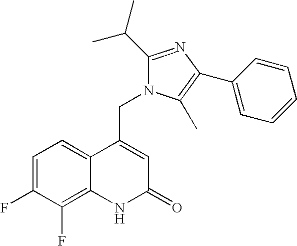

### Step 1: 2-Isopropyl-4-methyl-5-phenyl-1H-imidazole

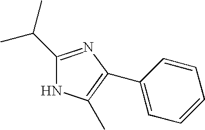

2-Isopropyl-4-methyl-5-phenyl-1H-imidazole was synthesized as described in EXAMPLE 256, Step 1 using isobutyraldehyde and 1-phenylpropane-1,2-dione as starting materials.

### Step 2: 7,8-Difluoro-4-((2-isopropyl-5-methyl-4-phenyl-1H-imidazol-1-yul)methyl)quinolin-2(1H)-one

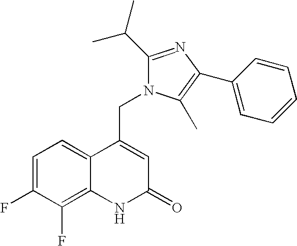

7,8-Difluoro-4-((2-isopropyl-5-methyl-4-phenyl-1H-imidazol-1-yul)methyl)quinolin-2(1H)-one was synthesized as described in EXAMPLE 118, Step 2 using 2-isopropyl-4-methyl-5-phenyl-1H-imidazole and 4-(bromomethyl)-7,8-difluoroquinolin-2(1H)-one as starting materials. ¹H NMR (400 MHz, CD₃OD) δ 7.84-7.79 (m, 1H), 7.58-7.55 (m, 2H), 7.43-7.38 (m, 2H), 7.35-7.26 (m, 2H), 5.85-5.75 (m, 1H), 5.55-5.40 (m, 2H), 3.06-2.90 (m, 1H), 2.24 (s, 3H), 1.32 (d, 6H). LCMS: 395.01 (M+H)⁺.

### EXAMPLE 259

### 7,8-Difluoro-4-((5-methyl-2-(4-methylthiazol-5-yl)-4-phenyl-1H-imidazol-1-yl)methyl)quinolin-2(1H)-one and 7,8-difluoro-4-((4-methyl-2-(4-methylthiazol-5-yl)-5-phenyl-1H-imidazol-1-yl)methyl)quinolin-2(1H)-one

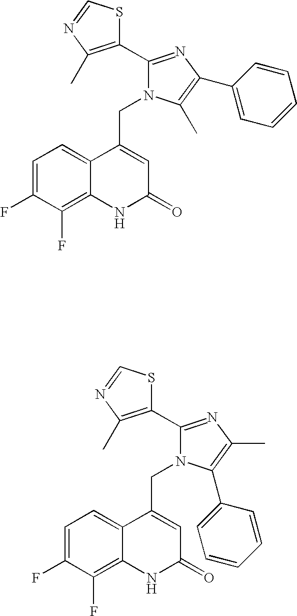

### Step 1: 4-Methyl-5-(5-methyl-4-phenyl-1H-imidazol-2-yl)thiazole

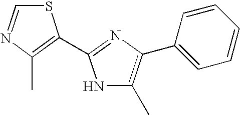

4-Methyl-5-(5-methyl-4-phenyl-1H-imidazol-2-yl)thiazole was synthesized as described in EXAMPLE 56 Step 1 using 4-methylthiazole-5-carbaldehyde and 1-phenylpropane-1,2-dione as starting materials. LCMS: 255.96 (M+H)⁺.

### Step 2: 7,8-difluoro-4-((5-methyl-2-(4-methylthiazol-5-yl)-4-phenyl-1H-imidazol-1-yl)methyl)quinolin-2(1H)-one and 7,8-difluoro-4-((4-methyl-2-(4-methylthiazol-5-yl)-5-phenyl-1H-imidazol-1-yl)methyl)quinolin-2(1H)-one

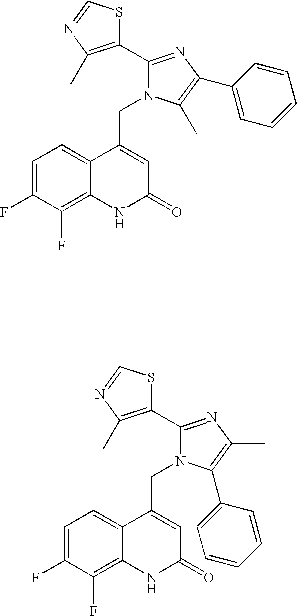

7,8-Difluoro-4-((5-methyl-2-(4-methylthiazol-5-yl)-4-phenyl-1H-imidazol-1-yl)methyl)quinolin-2(1H)-one and 7,8-difluoro-4-((4-methyl-2-(4-methylthiazol-5-yl)-5-phenyl-1H-imidazol-1-yl)methyl)quinolin-2(1H)-one were synthesized as described in EXAMPLE 118, Step 2 using 4-methyl-5-(5-methyl-4-phenyl-1H-imidazol-2-yl)thiazole and 4-(bromomethyl)-7,8-difluoroquinolin-2(1H)-one as starting materials. The two compounds were separated by column chromatography on silica gel (10% MeOH/DCM):

7,8-Difluoro-4-((5-methyl-2-(4-methylthiazol-5-yl)-4-phenyl-1H-imidazol-1-yl)methyl)quinolin-2(1H)-one. ¹H NMR (400 MHz, CD₃OD) δ 9.00 (s, 1H), 7.71-7.66 (m, 2H), 7.64-7.59 (m, 2H), 7.52-7.47 (m, 1H), 7.39-7.35 (m, 1H), 7.25-7.18 (m, 1H), 5.76 (s, 1H), 5.52 (s, 2H), 2.47 (s, 3H), 2.42 (s, 3H). LCMS: 449 (M+H)⁺.

7,8-Difluoro-4-((4-methyl-2-(4-methylthiazol-5-yl)-5-phenyl-1H-imidazol-1-yl)methyl)quinolin-2(1H)-one. ¹H NMR (400 MHz, CD₃OD) δ 9.04 (s, 1H), 7.45-7.42 (m, 5H), 7.38-7.35 (m, 1H), 7.12-7.06 (m, 1H), 5.82 (s, 1H), 5.41 (s, 2H), 2.47 (s, 3H), 2.32 (s, 3H). LCMS: 449 (M+H)⁺.

### EXAMPLE 260

### 7,8-Difluoro-4-((4-phenyl-1H-imidazol-1-yl)methyl)quinolin-2(1H)-one

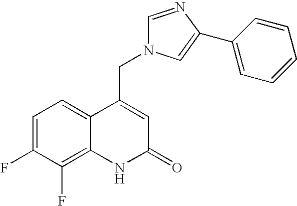

7,8-Difluoro-4-((4-phenyl-1H-imidazol-1-yl)methyl)quinolin-2(1H)-one was synthesized as described in EXAMPLE 118 using 4-(bromomethyl)-7,8-difluoroquinolin-2(1H)-one and 4-phenyl-1H-imidazole as starting materials. ¹H NMR (400 MHz, DMSO-d₆, TFA salt) δ 12.17 (s, 1H), 8.88 (s, 1H), 8.09 (s, 1H), 7.78-7.75 (m, 2H), 7.69-7.65 (m, 1H), 7.46 (t, 2H), 7.38-7.29 (m, 2H), 6.23 (s, 1H), 5.69 (s, 2H). LCMS: 338 (M+H)⁺.

### EXAMPLE 261

### N-(3-chlorophenyl)-N-((7,8-difluoro-2-oxo-1,2-dihydroquinolin-4-yl)methyl)pyridine-3-sulfonamide

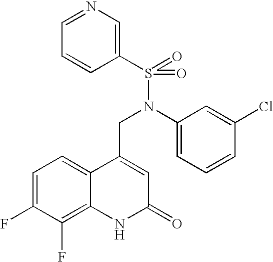

4-(Bromomethyl)-7,8-difluoroquinolin-2(1H)-one (52 mg, 0.190 mmol), N-(3-chlorophenyl)pyridine-3-sulfonamide (43 mg, 0.160 mmol) and potassium carbonate (80 mg, 0.579 mmol) were mixed in ACN (2 mL) and stirred for 2 h at 50° C. The reaction was poured into H₂O and extracted with DCM. The organic layers were dried (Na₂SO₄), filtered, and concentrated on silica gel. The crude mixture was purified using flash column chromatography (SiO₂, gradient eluent-0-100% hexanes in EtOAc) to afford N-(3-chlorophenyl)-N-((7,8-difluoro-2-oxo-1,2-dihydroquinolin-4-yl)methyl)pyridine-3-sulfonamide. ¹H NMR (400 MHz, DMSO-d₆) δ 11.98 (s, 1H), 8.95-8.92 (m, 1H), 8.83 (d, 1H), 8.07-8.03 (m, 1H), 7.87-7.83 (m, 1H), 7.72-7.68 (m, 1H), 7.41-7.31 (m, 3H), 7.22-7.20 (m, 1H), 7.12-7.09 (m, 1H), 6.45 (s, 1H), 5.15 (s, 2H). LCMS: 461.8 (M+H)⁺.

### EXAMPLE 262

### N-((8-Fluoro-2-oxo-1,2-dihydroquinolin-4-yl)methyl)-N-(3-methoxyphenyl)-4-methylthiazole-5-carboxamide

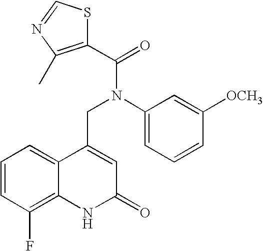

N-((8-Fluoro-2-oxo-1,2-dihydroquinolin-4-yl)methyl)-N-(3-methoxyphenyl)-4-methylthiazole-5-carboxamide was synthesized as described in EXAMPLE 26 using 8-fluoro-4-((3-methoxyphenylamino)methyl)quinolin-2(1H)-one 4-methylthiazole-5-carboxylic acid as starting materials. ¹H NMR (400 MHz, DMSO-d₆) δ 11.78 (s, 1H), 8.89 (s, 1H), 7.71 (d, 1H), 7.47-7.42 (m, 2H), 7.24-7.11 (m, 2H), 6.82 (d, 1H), 6.68-6.60 (d, 1H), 6.39 (s, 1H), 5.32 (s, 2H), 3.63 (s, 3H), 2.42 (s, 3H). LCMS: 424.1 (M+H)⁺.

### EXAMPLE 263

### N-((7,8-Difluoro-2-oxo-1,2-dihydroquinolin-4-yl)methyl)-4-methyl-N-(pyridin-3-yl)thiazole-5-carboxamide

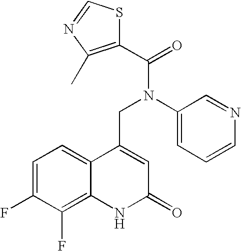

N-((7,8-Difluoro-2-oxo-1,2-dihydroquinolin-4-yl)methyl)-4-methyl-N-(pyridin-3-yl)thiazole-5-carboxamide was synthesized as described in EXAMPLE 26 using 7,8-difluoro-4-((pyridin-3-ylamino)methyl)quinolin-2(1H)-one and 4-methylthiazole-5-carboxylic acid as starting materials. ¹H NMR (400 MHz, DMSO-d₆) δ 12.01 (s, 1H), 8.93 (s, 1H), 8.37-8.36 (d, 2H), 7.70-7.68 (m, 2H), 7.36-7.35 (m, 2H), 6.38 (s, 1H), 5.40 (s, 2H), 2.50 (s, 3H). LCMS: 413.1 (M+H)⁺.

### EXAMPLE 264

### N-((7,8-Difluoro-2-oxo-1,2-dihydroquinolin-4-yl)methyl)-4-methyl-N-(pyridin-2-yl)thiazole-5-carboxamide

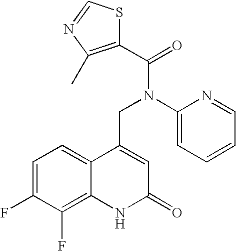

N-((7,8-Difluoro-2-oxo-1,2-dihydroquinolin-4-yl)methyl)-4-methyl-N-(pyridin-2-yl)thiazole-5-carboxamide was synthesized as described in EXAMPLE 26 using 7,8-difluoro-4-((pyridin-2-ylamino)methyl)quinolin-2(1H)-one and 4-methylthiazole-5-carboxylic acid as starting materials. ¹H NMR (400 MHz, DMSO-d₆) δ 12.11 (s, 1H), 8.72 (s, 1H), 8.48-8.42 (m, 1H), 8.27-8.21 (m, 1H), 7.92-7.89 (m, 1H), 7.79-7.78 (m, 1H), 7.43-7.38 (m, 2H), 6.91-6.89 (m, 1H), 5.80 (s, 2H), 2.45 (s, 3H). LCMS: 412.1 (M+H)⁺.

### EXAMPLE 265

### N-(3-Chlorophenyl)-N-((7,8-difluoro-2-oxo-1,2-dihydroquinolin-4-yl)methyl)-1,2-dimethyl-1H-imidazole-4-sulfonamide

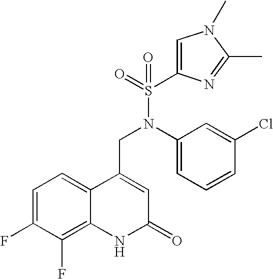

N-(3-chlorophenyl)-N-((7,8-difluoro-2-oxo-1,2-dihydroquinolin-4-yl)methyl)-1,2-dimethyl-1H-imidazole-4-sulfonamide was synthesized as described in EXAMPLE 42, Step 3 using 4-(bromomethyl)-7,8-difluoroquinolin-2(1H)-one and N-(3-chlorophenyl)-1,2-dimethyl-1H-imidazole-4-sulfonamide as starting materials. ¹H NMR (400 MHz, DMSO-d₆) δ 11.95 (s, 1H), 7.82-7.78 (m, 2H), 7.74 (s, 1H), 7.30-7.28 (m, 3H), 7.14 (d, 1H), 6.45 (s, 1H), 5.05 (s, 2H), 3.59 (s, 3H), 2.36 (s, 3H). LCMS: 479.1 (M+H)⁺.

The following compounds can generally be made using the methods described above. It is expected that these compounds when made will have activity similar to those that have been made in the examples above.

The following compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36.
O═C1NC2=C(F)C(F)═CC≡C2C(CN3C(C4=CC═CC═C4)=CC(C(C)C)=N3)=C1
O═C5NC6=C(F)C(F)═CC═C6C(CN7C(C8=CC═CC(C1)=C8)=NC(C(C)C)═C7)=C5
O═C9NC%10=C(F)C(F)═CC═C%10C(CN%11C(C%12=CC═CC═C%12)=NC(CC(C)C)═C%11)=C9
O═C%13NC%14=C(F)C(F)═CC═C%14C(CN%15C(C%16=CC═CC═C%16)=NC(C%17CCC%17)=C%15)=C%13
O═C%18NC%19=C(F)C(F)═CC═C%19C(CN%20C(C%21=CC═CC═C%21)=NC(C%22=C(C)N═CS% 22)=C%20)=C%18
O═C%23NC%24=C(F)C(F)═CC═C%24C(CN%25C═C(C(C)C)N═C%25C(C)C)═C%23
O═C%26NC%27=C(F)C(F)═CC═C%27C(CN%28C═C(C%29=CC═CC═C%29)N═C%28C(C)C)═C%26
O═C%30NC%31=C(F)C(F)═CC═C%31C(CN%32C═C(C(C)C)N═C%32C%33=C(C)N═CS%33)=C%30
O═C%34NC%35=C(F)C(F)═CC═C%35C(CN%36C═C(C%37=CC═CC═C%37)N═C%36C%38CCC%38)=C%34
O═C%39NC%40=C(F)C(F)═CC═C%40C(CN%41C(C%42=CC═CC═C%42C)═NC═C%41C(C)C)═C% 39
O═C%43NC%44=C(F)C(F)═CC═C%44C(CN%45C(C%46=CC═CC═C%46C)═NC═C%45C(C)C)═C% 43
O═C%47NC%48=C(F)C(F)═CC═C%48C(CN%49C(C%50=CC═NC═C%50C)═NC═C%49C(C)C)═C% 47
O═C%51NC%52=C(F)C(F)═CC═C%52C(CN%53C(C%54=CC(F)═CC═C%54)=CC(C(C)C)═N %53)=C%51
O═C%55NC%56=C(F)C(F)═CC═C%56C(CN%57C(C%58=CC═CC═C%58F)═NC═C%57C(C)C)═C% 55
O═C%59NC%60=C(F)C(F)═CC═C%60C(CN%61C(C%62=CC═CC(F)═C%62)=NC(C(C)C)═C%61)=C%59
O═C%63NC%64=C(F)C(F)═CC═C%64C(CN%65C(C%66=CC═CC═N %66)=NC(CC(C)C)═C%65)=C%63
O═C%67NC%68=C(F)C(F)═CC═C%68C(CN%69C(C%70=CC═CN═C%70)=NC(C%71 CCC%71)=C%69)=C%67
O═C%72NC%73=C(F)C(F)═CC═C%73C(CN%74C(C%75=CC═NC═N %75)=NC(C%76=C(C)N═CS% 76)=C%74)=C%72
O═C%77NC%78=C(F)C(F)═CC═C%78C(CN%79C═C(C(C)C)N═C%79CC(C)C)═C%77
O═C%80NC%81=C(F)C(F)═CC═C%81C(CN%82C═C(C%83=CC═CC═C%83)N═C%82CC(C)C)═C% 80
O═C%84NC%85=C(F)C(F)═CC═C%85C(CN%86C═C(CC(C)C)N═C%86C%87=C(C)N═CS%87)=C% 84
O═C%88NC%89=C(F)C(F)═CC═C%89C(CN%90C═C(C%91=CC═CC═C%91C1)N═C%90C%92CCC%92)=C%88
O═C%93NC%94=C(F)C(F)═CC═C%94C(CN%95C(C%96=CC═CC═C%96C)═NC═C%95CC(C)C)═C%93
O═C%97NC%98=C(F)C(F)═CC═C%98C(CN%99C(C%100=CC═CC═C%100C)═NC═C%99CC(C)C)═C%97
O═C%101NC%102=C(F)C(F)═CC═C%102C(CN%103C(C%104=CC═NC═C%104OC)═NC═C%103C C(C)C)═C%101
O═C%105NC%106=C(F)C(F)═CC═C%106C(CN%107C(C%108=CC═CC═C%108)=CC(C%109=C(C)N═CS%109)=N %107)=C%105
O═C%110NC%111=C(F)C(F)═CC═C%111C(CN%112C(C%113=CC═CC═C%113)=NC═C%112C%114=C(C)C═NC═C%114)=C%110
O═C%115NC%116=C(F)C(F)═CC═C%116C(CN%117C(C%118=CC═CC(C1)=C%118)=NC(C%119=C(C)N═CS%119)=C%117)=C%115
O═C%120NC%121=C(F)C(F)═CC═C%121 C(CN%122C(C%123=CC═CC═C%123)=NC(C%124=C(C)N═CS%124)=C%122)=C%120
O═C%125NC%126=C(F)C(F)═CC═C%126C(CN%127C(CC(C)C)═NC(C%128=C(C)N═CS%128)=C%127)=C%125
O═C%129NC%130=C(F)C(F)═CC═C%130C(CN%131C═C(C(C)C)N═C%131C%132=C(C)N═CS%132)=C%129
O═C%133NC%134=C(F)C(F)═CC═C%134C(CN%135C═C(C%136=CC═CC═C%136)N═C%135C%137=C(C)N═CS%137)=C%133
O═C%138NC%139=C(F)C(F)═CC═C%139C(CN%140C═C(C%141CCC%141)N═C%140C%142=C(C)N═CS%142)=C%138
O═C%143NC%144=C(F)C(F)═CC═C%144C(CN%145C═C(C%146=CC═CC═C%146)N═C%145C%147=C(C)N═CS%147)=C%143
O═C%148NC%149=C(F)C(F)═CC═C%149C(CN%150C(C%151=CC═CC═C%151 C)═NC═C%150C% 152=C(C)N═CS%152)=C%148
O═C%153NC%154=C(F)C(F)═CC═C%154C(CN%155C(C%156=CC═CC═C%156C)═NC═C%155C% 157=C(C)N═CS%157)=C%153
O═C%158NC%159=C(F)C(F)═CC═C%159C(CN%160C(C%161=CC═NC═C%161C)═NC═C%160C% 162=C(C)N═CS%162)=C%158
O═C%163NC%164=C(F)C(F)═CC═C%164C(CN%165C(C%166=CC═CC═C%166)=NC(C%167=C(C)
N═CS%167)=C%165)=C%163 o═C1NC2=C(F)C(F)═CC═C2C(CN3C4=CC═CC═C4C(N═C3C(C)C)═O)═C1
O═C5NC6=C(F)C(F)═CC═C6C(CN7C8=CC═CC═C8CCC7C(C)C)═C5
O═C9NC%10=C(F)C(F)═CC═C%10C(CC%11=C(C(C)C)OC%12=CC═CC═C%11%12)=C9
O═C%13NC%14=C(F)C(F)═CC═C%14C(CN%15C(C═CC═C%16)=C%16CC%15=0)═C%13
O═C%17NC%18=C(F)C(F)═CC═C%18C(CN%19C(C═CC═C%20)=C%20C(C(C)C)C%19=O)═C%17
O═C%21NC%22=C(F)C(F)═CC═C%22C(CN%23C(C═CC═C%24)=C%24CCC%23=0)═C%21
O═C%25NC%26=C(F)C(F)═CC═C%26C(CN%27C(C═CC═C%28)=C%28CC(C(C)C)C%27=0)═C%2
O═C%29NC%30=C(F)C(F)═CC═C%30C(CN%31 C%32=CC═CC(C)═C%32C(N═C%31 C(C)C)═O)═C%29
O═C%33NC%34=C(F)C(F)═CC═C%34C(CN%35C%36=CC═CC(C)═C%36CCC%35C(C)C)═C%33
O═C%37NC%38=C(F)C(F)═CC═C%38C(CC%39=C(C(C)C)OC%40=C(C)C═CC═C%39%40)=C%37
O═C%41NC%42=C(F)C(F)═CC═C%42C(CN%43C(C═CC═C%44C)═C%44CC%43=0)═C%41
O═C%45NC%46=C(F)C(F)═CC═C%46C(CN%47C(C═CC═C%48C)═C%48C(C(C)C)C%47=0)═C%45
O═C%49NC%50=C(F)C(F)═CC═C%50C(CN%51 C(C═CC═C%52C)═C%52CCC%51=0)═C%49
O═C%53NC%54=C(F)C(F)═CC═C%54C(CN%55C(C═CC═C%56C)═C%56CC(C(C)C)C%55=0)═C% 53
O═C%57NC%58=C(F)C(F)═CC═C%58C(CN%59C%60=CC(F)═CC═C%60C(N═C%59CC(C)C)═O)═C%57
O═C%61NC%62=C(F)C(F)═CC═C%62C(CN%63C%64=CC(F)═CC═C%64CCC%63CC(C)C)═C%6
O═C%65NC%66=C(F)C(F)═CC═C%66C(CC%67=C(CC(C)C)OC%68=CC═C(F)C═C%67%68)=C%6
O═C%69NC%70=C(F)C(F)═CC═C%70C(CN%71C(C═C(F)C═C%72)=C%72CC%71=0)═C%69
O═C%73NC%74=C(F)C(F)═CC═C%74C(CN%75C(C═C(F)C═C%76)=C%76C(CC(C)C)C%75=0)═C%73
O═C%77NC%78=C(F)C(F)═CC═C%78C(CN%79C(C═C(F)C═C%80)=C%80CCC%79=0)═C%77
O═C%81NC%82=C(F)C(F)═CC═C%82C(CN%83C(C═C(F)C═C%84)=C%84CC(CC(C)C)C%83=O)═C%81
O═C%85NC%86=C(F)C(F)═CC═C%86C(CN%87C%88=CC═C(C1)C═C%88C(N═C%87C%89CCC%89)=0)=C%85
O═C%90NC%91=C(F)C(F)═CC═C%91C(CN%92C%93=CC═C(C1)C═C%93CCC%92C%94CCC%94)=C%90
O═C%95NC%96=C(F)C(F)═CC═C%96C(CC%97=C(C%98CCC%98)OC%99=CC(C1)=CC═C%97%99)=C%95
O═C%1 OONC%101=C(F)C(F)═CC═C%101 C(CN%102C(C═CC(C1)=C%103)=C%103CC%102=0)═C%100
O═C%104NC%105=C(F)C(F)═CC═C%105C(CN%106C(C═CC(C1)=C%107)=C%107C(C%108CCC% 108)C%106=0)═C%104
O═C%109NC%110=C(F)C(F)═CC═C%110C(CN%111C(C═CC(C1)=C%112)=C%112CCC%111=O)═C%109
O═C%113NC%114=C(F)C(F)═CC═C%114C(CN%115C(C═CC(C1)=C%116)=C%116CC(C%117CCC%117)C%115=0)═C%113
O═C1NC2=C(F)C(F)═CC═C2C(CN3C(C4=CC═CC═C4)CCC3=0)═C1
O═C5NC6=C(F)C(F)═CC═C6C(CN7CC(C8=CC═CC═C8)CC7=0)═C5
O═C9NC%10=C(F)C(F)═CC═C%10C(CN%11C(C%12=CC═CC═C%12)CC(C(C)C)C%11=0)═C9
O═C%13NC%14=C(F)C(F)═CC═C%14C(CN(C%15)C(C%16=CC═CC═C%16)CC%15=0)═C%13
O═C%17NC%18=C(F)C(F)═CC═C%18C(CN%19C(C%20=CC═CC═C%20)CNC%19=0)═C%17
O═C%21NC%22=C(F)C(F)═CC═C%22C(CN%23C(C%24=CC═CC═C%24)CN(C(C)C)C%23=)═C%21
O═C%25NC%26=C(F)C(F)═CC═C%26C(CN%27N(C%28=CC═CC═C%28)CC(C(C)C)C%27=0)═C%25
O═C%29NC%30=C(F)C(F)═CC═C%30C(CN%31 C(C%32=CC═CC═C%32F)CCC%31=0)═C%29
O═C%33NC%34=C(F)C(F)═CC═C%34C(CN%35CC(C%36=CC═CN═C%36)CC%35=0)═C%33
O═C%37NC%38=C(F)C(F)═CC═C%38C(CN%39C(C%40=CC═C(OC)C═C%40)CC(C(C)C)C%39=0)═C%37
O═C%41NC%42=C(F)C(F)═CC═C%42C(CN(C%43)C(C%44=CC═NC═C%44)CC%43=0)═C%41
O═C%45NC%46=C(F)C(F)═CC═C%46C(CN%47C(C%48=CC═CC(C1)=C%48)CNC%47=0)═C%45
O═C%49NC%50=C(F)C(F)═CC═C%50C(CN%51 C(C%52=CC═CC═N %52)CN(C(C)C)C%51=0)═C%49
O═C%53NC%54=C(F)C(F)═CC═C%54C(CN%55N(C%56=CC═CC═C%56)CC(CC(C)C)C%55=O)═C%53
O═C%57NC%58=C(F)C(F)═CC═C%58C(CN%59C(C%60=CC═C(F)C═C%60Cl)CCC%59=0)═C%57
O═C%61NC%62=C(F)C(F)═CC═C%62C(CN%63CC(C%64=CC═C(F)C═C%64F)CC%63=0)═C%61
O═C%65NC%66=C(F)C(F)═CC═C%66C(CN%67C(C%68=CC═CC═C%68)CC(C%69=C(C)N═CS%69)C%67=0)═C%65
O═C%70NC%71=C(F)C(F)═CC═C%71C(CN(C%72)C(C%73=CC═CC═C%730C)CC%72=0)═C%70
O═C%74NC%75=C(F)C(F)═CC═C%75C(CN%76C(C%77=CC═NC═N %77)CNC%76=0)═C%74
O═C%78NC%79=C(F)C(F)═CC═C%79C(CN%80C(C%81=CC═CC═C%81)CN(C%82=C(C)N═CS%82)C%80=0)═C%78
O═C%83NC%84=C(F)C(F)═CC═C%84C(CN%85N(C%86=CC═CC═C%86)CC(C%87=C(C)N═CS%87)C%85=O)═C%83
O═C lNC2=C(F)C(F)═CC═C2C(CN3C(C═CC═N4)=C4N═C3C5=C(C)C═NC═C5)=C1
O═C6NC7=C(F)C(F)═CC═C7C(CN8C(C═CN═C9)=C9N═C8C%10=C(C)C═NC═C%10)=C6
O═C%11NC%12=C(F)C(F)═CC═C%12C(CN%13C(C═NC═C%14)=C%14N═C%13C%15=C(C)C═NC═C%15)=C%11
O═C%16NC%17=C(F)C(F)═CC═C%17C(CN%18C(N═CC═C%19)=C%19N═C%18C%20=C(C)C═NC═C%20)=C%16
O═C%21NC%22=C(F)C(F)═CC═C%22C(CN%23C(C═CN═C%24)=C%24N═C%23C(C)C)═C%21
O═C%25NC%26=C(F)C(F)═CC═C%26C(CN%27C(C═NC═C%28)=C%28N═C%27C(C)C)═C%25
O═C1NC2=C(F)C(F)═CC═C2C(CN3C(C═CC═C4C)═C4N═C3C5=C(C)N═CS5)=Cl
O═C6NC7=C(F)C(F)═CC═C7C(CN8C(C═CC(C)═C9)=C9N═C8C%10=C(C)N═CS%10)=C6
O═C%11NC%12=C(F)C(F)═CC═C%12C(CN%13C(C═C(C)C═C%14)=C%14N═C%13C%15=C(C)N═CS%15)=C%11
O═C%16NC%17=C(F)C(F)═CC═C%17C(CN%18C(C(C)═CC═C%19)=C%19N═C%18C%20=C(C)N═CS%20)=C%16
O═C%21NC%22=C(F)C(F)═CC═C%22C(CN%23C(C═CC═C%24C)═C%24N═C%23C%25=CN═CN %25C)═C%21
O═C%26NC%27=C(F)C(F)═CC═C%27C(CN%28C(C═CC(C)═C%29)=C%29N═C%28C%30=CN═CN %30C)═C%26
O═C%31NC%32=C(F)C(F)═CC═C%32C(CN%33C(C═C(C)C═C%34)=C%34N═C%33C%35=CN═CN %35C)═C%31
O═C%36NC%37=C(F)C(F)═CC═C%37C(CN%38C(C(C)═CC═C%39)=C%39N═C%38C%40=CN═CN %40C)═C%36
O═C%41NC%42=C(F)C(F)═CC═C%42C(CN%43C(C═CC═C%44C)═C%44N═C%43C%45=C(C)C═CN═C%45)=C%41
O═C%46NC%47=C(F)C(F)═CC═C%47C(CN%48C(C═CC(C)═C%49)=C%49N═C%48C%50=C(C)C═CN═C%50)=C%46
O═C%51NC%52=C(F)C(F)═CC═C%52C(CN%53C(C═C(C)C═C%54)=C%54N═C%53C%55=C(C)C═CN═C%55)=C%51
O═C%56NC%57=C(F)C(F)═CC═C%57C(CN%58C(C(C)═CC═C%59)=C%59N═C%58C%60=C(C)C═CN═C%60)=C%56
O═C%61NC%62=C(F)C(F)═CC═C%62C(CN%63C(C═CC═C%64C)═C%64N═C%63C%65=C(C)C═N C═C%65)=C%61
O═C%66NC%67=C(F)C(F)═CC═C%67C(CN%68C(C═CC(C)═C%69)=C%69N═C%68C%70=C(C)C═NC═C%70)=C%66
O═C%71NC%72=C(F)C(F)═CC═C%72C(CN%73C(C═C(C)C═C%74)=C%74N═C%73C%75=C(C)C═NC═C%75)=C%71
O═C%76NC%77=C(F)C(F)═CC═C%77C(CN%78C(C(C)═CC═C%79)=C%79N═C%78C%80=C(C)C═NC═C%80)=C%76
O═C%81NC%82=C(F)C(F)═CC═C%82C(CN%83C(C(C)═CC═C%84)=C%84N═C%83C(C)C)═C%81
O═C1NC2=C(F)C(F)═CC═C2C(CN3C(C═CC═C4F)═C4N═C3C5=C(C)N═CS5)=C1
O═C6NC7=C(F)C(F)═CC═C7C(CN8C(C═CC(F)═C9)=C9N═C8C%10=C(C)N═CS%10)=C6
O═C%11NC%12=C(F)C(F)═CC═C%12C(CN%13C(C═C(F)C═C%14)=C%14N═C%13C%15=C(C)N═CS%15)=C%11
O═C%16NC%17=C(F)C(F)═CC═C%17C(CN%18C(C(F)═CC═C%19)=C%19N═C%18C%20=C(C)N═CS%20)=C%16
O═C%21NC%22=C(F)C(F)═CC═C%22C(CN%23C(C═CC═C%24F)═C%24N═C%23C%25=CN═CN %25C)═C%21
O═C%26NC%27=C(F)C(F)═CC═C%27C(CN%28C(C═CC(F)═C%29)=C%29N═C%28C%30=CN═CN %30C)═C%26
O═C%31NC%32=C(F)C(F)═CC═C%32C(CN%33C(C═C(F)C═C%34)=C%34N═C%33C%35=CN═CN %35C)═C%31
O═C%36NC%37=C(F)C(F)═CC═C%37C(CN%38C(C(F)═CC═C%39)=C%39N═C%38C%40=CN═CN %40C)═C%36
O═C%41NC%42=C(F)C(F)═CC═C%42C(CN%43C(C═CC═C%44F)═C%44N═C%43C%45=C(C)C═CN═C%45)=C%41
O═C%46NC%47=C(F)C(F)═CC═C%47C(CN%48C(C═CC(F)═C%49)=C%49N═C%48C%50=C(C)C═CN═C%50)=C%46
O═C%51NC%52=C(F)C(F)═CC═C%52C(CN%53C(C═C(F)C═C%54)=C%54N═C%53C%55=C(C)C═CN═C%55)=C%51
O═C%56NC%57=C(F)C(F)═CC═C%57C(CN%58C(C(F)═CC═C%59)=C%59N═C%58C%60=C(C)C═CN═C%60)=C%56
O═C%61NC%62=C(F)C(F)═CC═C%62C(CN%63C(C═CC═C%64F)═C%64N═C%63C%65=C(C)C═N C═C%65)=C%61
O═C%66NC%67=C(F)C(F)═CC═C%67C(CN%68C(C═CC(F)═C%69)=C%69N═C%68C%70=C(C)C═NC═C%70)=C%66
O═C%71NC%72=C(F)C(F)═CC═C%72C(CN%73C(C═C(F)C═C%74)=C%74N═C%73C%75=C(C)C═NC═C%75)=C%71
O═C%76NC%77=C(F)C(F)═CC═C%77C(CN%78C(C(F)═CC═C%79)=C%79N═C%78C%80=C(C)C═
NC═C%80)=C%76
O═C1NC2=C(F)C(F)═CC═C2C(CN3C(C═CC═C4Cl)═C4N═C3C5=C(C)N═CS5)=C1
O═C6NC7=C(F)C(F)═CC═C7C(CN8C(C(C1)=CC═C9)=C9N═C8C%10=C(C)N═CS%10)=C6
O═C%11NC%12=C(F)C(F)═CC═C%12C(CN%13C(C═CC═C%14Cl)═C%14N═C%13C%15=CN═CN %15C)═C%11
O═C%16NC%17=C(F)C(F)═CC═C%17C(CN%18C(C═CC(C1)=C%19)=C%19N═C%18C%20=CN═CN%20C)═C%16
O═C%21NC%22=C(F)C(F)═CC═C%22C(CN%23C(C═C(C)C═C%24)=C%24N═C%23C%25=CN═CN%25C)═C%21
O═C%26NC%27=C(F)C(F)═CC═C%27C(CN%28C(C(C1)=CC═C%29)=C%29N═C%28C%30=CN═CN%30C)═C%26
O═C%31NC%32=C(F)C(F)═CC═C%32C(CN%33C(C═CC═C%34Cl)═C%34N═C%33C%35=C(C)C═CN═C%35)=C%31
O═C%36NC%37=C(F)C(F)═CC═C%37C(CN%38C(C═CC(C1)=C%39)=C%39N═C%38C%40=C(C)C═CN═C%40)=C%36
O═C%41NC%42=C(F)C(F)═CC═C%42C(CN%43C(C═C(C)C═C%44)=C%44N═C%43C%45=C(C)C═CN═C%45)=C%41
O═C%46NC%47=C(F)C(F)═CC═C%47C(CN%48C(C(C1)=CC═C%49)=C%49N═C%48C%50=C(C)C═CN═C%50)=C%46
O═C%51NC%52=C(F)C(F)═CC═C%52C(CN%53C(C═CC═C%54Cl)═C%54N═C%53C%55=C(C)C═NC═C%55)=C%51
O═C%56NC%57=C(F)C(F)═CC═C%57C(CN%58C(C═CC(C1)=C%59)=C%59N═C%58C%60=C(C)C ═NC═C%60)=C%56
O═C%61NC%62=C(F)C(F)═CC═C%62C(CN%63C(C═C(C)C═C%64)=C%64N═C%63C%65=C(C)C═NC═C%65)=C%61
O═C%66NC%67=C(F)C(F)═CC═C%67C(CN%68C(C(C1)=CC═C%69)=C%69N═C%68C%70=C(C)C ═NC═C%70)=C%66
O═C%71NC%72=C(F)C(F)═CC═C%72C(CN%73C(C═CC═C%74C)═C%74N═C%73C(C)C)═C%71
O═C%75NC%76=C(F)C(F)═CC═C%76C(CN%77C(C(C1)=CC═C%78)=C%78N═C%77C(C)C)═C%75
O═C1NC2=C(F)C(F)═CC═C2C(CN3C(C═CC═C4C#N)═C4N═C3C5=C(C)N═CS5)=C1
O═C6NC7=C(F)C(F)═CC═C7C(CN8C(C═CC(C#N)═C9)=C9N═C8C%10=C(C)N═CS%10)=C6
O═C%11NC%12=C(F)C(F)═CC═C%12C(CN%13C(C═C(C#N)C═C%14)=C%14N═C%13C%15=C(C)N═CS%15)=C%11
O═C%16NC%17=C(F)C(F)═CC═C%17C(CN%18C(C(C#N)═CC═C%19)=C%19N═C%18C%20=C(C)N═CS%20)=C%16
O═C%21NC%22=C(F)C(F)═CC═C%22C(CN%23C(C═CC═C%24C#N)═C%24N═C%23C%25=CN═CN%25C)═C%21
O═C%26NC%27=C(F)C(F)═CC═C%27C(CN%28C(C═CC(C#N)═C%29)=C%29N═C%28C%30=CN═CN%30C)═C%26
O═C%31NC%32=C(F)C(F)═CC═C%32C(CN%33C(C═C(C#N)C═C%34)=C%34N═C%33C%35=CN═CN%35C)═C%31
O═C%36NC%37=C(F)C(F)═CC═C%37C(CN%38C(C(C#N)═CC═C%39)=C%39N═C%38C%40=CN═CN%40C)═C%36
O═C%41NC%42=C(F)C(F)═CC═C%42C(CN%43C(C═CC═C%44C#N)═C%44N═C%43C%45=C(C)C═CN═C%45)=C%41
O═C%46NC%47=C(F)C(F)═CC═C%47C(CN%48C(C═CC(C#N)═C%49)=C%49N═C%48C%50=C(C)C═CN═C%50)=C%46
O═C%51NC%52=C(F)C(F)═CC═C%52C(CN%53C(C═C(C#N)C═C%54)=C%54N═C%53C%55=C(C)C═CN═C%55)=C%51
O═C%56NC%57=C(F)C(F)═CC═C%57C(CN%58C(C(C#N)═CC═C%59)=C%59N═C%58C%60=C(C)C═CN═C%60)=C%56
O═C%61NC%62=C(F)C(F)═CC═C%62C(CN%63C(C═CC═C%64C#N)═C%64N═C%63C%65=C(C)C═NC═C%65)=C%61
O═C%66NC%67=C(F)C(F)═CC═C%67C(CN%68C(C═CC(C#N)═C%69)=C%69N═C%68C%70=C(C)C═NC═C%70)=C%66
O═C%71NC%72=C(F)C(F)═CC═C%72C(CN%73C(C═C(C#N)C═C%74)=C%74N═C%73C%75=C(C)C═NC═C%75)=C%71
O═C%76NC%77=C(F)C(F)═CC═C%77C(CN%78C(C(C#N)═CC═C%79)=C%79N═C%78C%80=C(C)C═NC═C%80)=C%76
O═C%81NC%82=C(F)C(F)═CC═C%82C(CN%83C(C═CC═C%84C#N)═C%84N═C%83C(C)C)═C%81
O═C%85NC%86=C(F)C(F)═CC═C%86C(CN%87C(C═CC(C#N)═C%88)=C%88N═C%87C(C)C)═C% 85
O═C%89NC%90=C(F)C(F)═CC═C%90C(CN%91 C(C═C(C#N)C═C%92)=C%92N═C%91 C(C)C)═C% 89
O═C%93NC%94=C(F)C(F)═CC═C%94C(CN%95C(C(C#N)═CC═C%96)=C%96N═C%95C(C)C)═C% 93
O═C1NC2=C(F)C(F)═CC═C2C(CN3C(C═CC═C40C)═C4N═C3C5=C(C)N═CS5)=C1
O═C6NC7=C(F)C(F)═CC═C7C(CN8C(C═CC(OC)═C9)=C9N═C8C%10=C(C)N═CS%10)=C6
O═C%11NC%12=C(F)C(F)═CC═C%12C(CN%13C(C═C(OC)C═C%14)=C%14N═C%13C%15=C(C)N═CS%15)=C%11
O═C%16NC%17=C(F)C(F)═CC═C%17C(CN%18C(C(OC)═CC═C%19)=C%19N═C%18C%20=C(C)N═CS%20)=C%16
O═C%21NC%22=C(F)C(F)═CC═C%22C(CN%23C(C═CC═C%240C)═C%24N═C%23C%25=CN═CN%25C)═C%21
O═C%26NC%27=C(F)C(F)═CC═C%27C(CN%28C(C═CC(OC)═C%29)=C%29N═C%28C%30=CN═CN%30C)═C%26
O═C%31NC%32=C(F)C(F)═CC═C%32C(CN%33C(C═C(OC)C═C%34)=C%34N═C%33C%35=CN═CN%35C)═C%31
O═C%36NC%37=C(F)C(F)═CC═C%37C(CN%38C(C(OC)═CC═C%39)=C%39N═C%38C%40=CN═CN%40C)═C%36
O═C%41NC%42=C(F)C(F)═CC═C%42C(CN%43C(C═CC═C%44OC)═C%44N═C%43C%45=C(C)C═CN═C%45)=C%41
O═C%46NC%47=C(F)C(F)═CC═C%47C(CN%48C(C═CC(OC)═C%49)=C%49N═C%48C%50=C(C) C═CN═C%50)=C%46
O═C%51NC%52=C(F)C(F)═CC═C%52C(CN%53C(C═C(OC)C═C%54)=C%54N═C%53C%55=C(C) C═CN═C%55)=C%51
O═C%56NC%57=C(F)C(F)═CC═C%57C(CN%58C(C(OC)═CC═C%59)=C%59N═C%58C%60=C(C) C═CN═C%60)=C%56
O═C%61NC%62=C(F)C(F)═CC═C%62C(CN%63C(C═CC═C%64OC)═C%64N═C%63C%65=C(C)C═NC═C%65)=C%61
O═C%66NC%67=C(F)C(F)═CC═C%67C(CN%68C(C═CC(OC)═C%69)=C%69N═C%68C%70=C(C) C═NC═C%70)=C%66
O═C%71NC%72=C(F)C(F)═CC═C%72C(CN%73C(C═C(OC)C═C%74)=C%74N═C%73C%75=C(C)C═NC═C%75)=C%71
O═C%76NC%77=C(F)C(F)═CC═C%77C(CN%78C(C(OC)═CC═C%79)=C%79N═C%78C%80=C(C)C═NC═C%80)=C%76
O═C%81NC%82=C(F)C(F)═CC═C%82C(CN%83C(C═CC═C%84OC)═C%84N═C%83C(C)C)═C%81
O═C%85NC%86=C(F)C(F)═CC═C%86C(CN%87C(C═CC(OC)═C%88)=C%88N═C%87C(C)C)═C%8
O═C%89NC%90=C(F)C(F)═CC═C%90C(CN%91C(C═C(OC)C═C%92)=C%92N═C%91C(C)C)═C%89
O═C%93NC%94=C(F)C(F)═CC═C%94C(CN%95C(C(OC)═CC═C%96)=C%96N═C%95C(C)C)═C%93
O═C1NC2=C(F)C(F)═CC═C2C(CN3C(C═CC═C4C5=CC═CC═C5)=C4N═C3C6=C(C)N═CS6)=Cl
O═C7NC8=C(F)C(F)═CC═C8C(CN9C(C═CC(C%10=CC═CC═C%10)=C%11)=C%11N═C9C%12=C(C)N═CS%12)=C7
O═C%13NC%14=C(F)C(F)═CC═C%14C(CN%15C(C═C(C%16=CC═CC═C%16)C═C%17)=C%17N═C%15C%18=C(C)N═CS%18)=C%13
O═C%19NC%20=C(F)C(F)═CC═C%20C(CN%21C(C(C%22=CC═CC═C%22)=CC═C%23)=C%23N═C%21C%24=C(C)N═CS%24)=C%19
O═C%25NC%26=C(F)C(F)═CC═C%26C(CN%27C(C═CC═C%28C%29=CC═CC═C%29)=C%28N═C%27C%30=CN═CN%30C)═C%25
O═C%31NC%32=C(F)C(F)═CC═C%32C(CN%33C(C═CC(C%34=CC═CC═C%34)=C%35)=C%35N═C%33C%36=CN═CN%36C)═C%31
O═C%37NC%38=C(F)C(F)═CC═C%38C(CN%39C(C═C(C%40=CC═CC═C%40)C═C%41)=C%41N═C%39C%42=CN═CN%42C)═C%37
O═C%43NC%44=C(F)C(F)═CC═C%44C(CN%45C(C(C%46=CC═CC═C%46)=CC═C%47)=C%47N═C%45C%48=CN═CN%48C)═C%43
O═C%49NC%50=C(F)C(F)═CC═C%50C(CN%51C(C═CC═C%52C%53=CC═CC═C%53)=C%52N═C %51C%54=C(C)C═CN═C%54)=C%49
O═C%55NC%56=C(F)C(F)═CC═C%56C(CN%57C(C═CC(C%58=CC═CC═C%58)=C%59)=C%59N═C%57C%60=C(C)C═CN═C%60)=C%55
O═C%61NC%62=C(F)C(F)═CC═C%62C(CN%63C(C═C(C%64=CC═CC═C%64)C═C%65)=C%65N C%63C%66=C(C)C═CN═C%66)=C%61
O═C%67NC%68=C(F)C(F)═CC═C%68C(CN%69C(C(C%70=CC═CC═C%70)=CC═C%71)=C%71N═C%69C%72=C(C)C═CN═C%72)=C%67
O═C%73NC%74=C(F)C(F)═CC═C%74C(CN%75C(C═CC═C%76C%77=CC═CC═C%77)=C%76N═C%75C%78=C(C)C═NC═C%78)=C%73
O═C%79NC%80=C(F)C(F)═CC═C%80C(CN%81C(C═CC(C%82=CC═CC═C%82)=C%83)=C%83N═C%81C%84=C(C)C═NC═C%84)=C%79
O═C%85NC%86=C(F)C(F)═CC═C%86C(CN%87C(C═C(C%88=CC═CC═C%88)C═C%89)=C%89N═C%87C%90=C(C)C═NC═C%90)=C%85
O═C%91NC%92=C(F)C(F)═CC═C%92C(CN%93C(C(C%94=CC═CC═C%94)=CC═C%95)=C%95N═C%93C%96=C(C)C═NC═C%96)=C%91
O═C%97NC%98=C(F)C(F)═CC═C%98C(CN%99C(C═CC═C%100C%101=CC═CC═C%101)=C%100N═C%99C(C)C)═C%97
O═C%102NC%103=C(F)C(F)═CC═C%103C(CN%104C(C═CC(C%105=CC═CC═C%105)=C%106)=C%106N═C%104C(C)C)═C%102
O═C%107NC%108=C(F)C(F)═CC═C%108C(CN%109C(C═C(C%110=CC═CC═C%110)C═C%111)=C%111N═C%109C(C)C)═C%107
O═C%112NC%113=C(F)C(F)═CC═C%113C(CN%114C(C(C%115=CC═CC═C%115)=CC═C%116)=C%116N═C%114C(C)C)═C%112
O═C1NC2=C(F)C(F)═CC═C2C(CN(C(C3=C(C)N═CS3)=O)C4=CC═NC═C4)=C1
O═C5NC6=C(F)C(F)═CC═C6C(CN(C(C7=C(C)N═CS7)=O)C8CCCN(C)C8)=C5
O═C1NC2=C(F)C(F)═CC═C2C(CN3C(C═CC═N4)=C4N═C3C5CCC5)=Cl
O═C6NC7=C(F)C(F)═CC═C7C(CN8C(C═CN═C9)=C9N═C8C%10CCC%10)=C6
O═C%11NC%12=C(F)C(F)═CC═C%12C(CN%13C(C═NC═C%14)=C%14N═C%13C%15CCC%15)=C%11
O═C%16NC%17=C(F)C(F)═CC═C%17C(CN%18C(N═CC═C%19)=C%19N═C%18C%20CCC%20)=C%16
O═C%21NC%22=C(F)C(F)═CC═C%22C(CN%23C(C═CC═N %24)=C%24N═C%23C(CC)CC)═C%21
O═C%25NC%26=C(F)C(F)═CC═C%26C(CN%27C(C═CN═C%28)=C%28N═C%27C(CC)CC)═C%25
O═C%29NC%30=C(F)C(F)═CC═C%30C(CN%31C(C═NC═C%32)=C%32N═C%31C(CC)CC)═C%29
O═C%33NC%34=C(F)C(F)═CC═C%34C(CN%35C(N═CC═C%36)=C%36N═C%35C(CC)CC)═C%33

The activity of the compounds in Examples 1-265 as inhibitors is illustrated in the following assay. The other compounds listed above, which have not yet been tested, are predicted to have activity in this assay as well.

### Biological Activity Assay

### Enzyme Source

The source of nitric oxide synthase (NOS) enzyme can be generated in several ways including induction of endogenous iNOS using cytokines and/or lipopolysaccharide (LPS) in various cell types known in the art. Alternatively, the gene encoding the enzyme can be cloned and the enzyme can be generated in cells via heterologous expression from a transient or stable expression plasmid with suitable features for protein expression as are known in the art. Enzymatic activity (nitric oxide production) is calcium independent for iNOS, while the constitutive NOS isoforms, nNOS and eNOS, become active with the addition of various cofactors added to cellular media or extract as are well known in the art. Enzymes specified in Table 1 were expressed in HEK293 cells transiently transfected with human iNOS.

### DAN Assay

A major metabolic pathway for nitric oxide is to nitrate and nitrite, which are stable metabolites within tissue culture, tissue, plasma, and urine (S Moncada, A Higgs, N Eng J Med 329, 2002 (1993)). Tracer studies in humans have demonstrated that perhaps 50% of the total body nitrate/nitrite originates from the substrate for NO synthesis, L-arginine (P M Rhodes, A M Leone, P L Francis, A D Struthers, S Moncada, Biomed Biophys Res. Commun. 209, 590 (1995); L. Castillo et al., Proc Natl Acad Sci USA 90, 193 (1993). Although nitrate and nitrite are not measures of biologically active NO, plasma and urine samples obtained from subjects after a suitable period of fasting, and optionally after administration of a controlled diet (low nitrate/low arginine), allow the use of nitrate and nitrite as an index of NO activity (C Baylis, P Vallance, Curr Opin Nephrol Hypertens 7, 59 (1998)).

The level of nitrate or nitrite in the specimen can be quantified by any method known in the art which provides adequate sensitivity and reproducibility. A variety of protocols have also been described for detecting and quantifying nitrite and nitrate levels in biological fluids by ion chromatography (e.g., A Everett et al., J. Chromatogr. 706, 437 (1995); J M Monaghan et al., J. Chromatogr. 770, 143 (1997)), high-performance liquid chromatography (e.g., M Kelm et al., Cardiovasc. Res. 41, 765 (1999)), and capillary electrophoresis (MA Friedberg et al., J. Chromatogr. 781, 491 (1997)). For example, 2,3-diaminonaphthalene reacts with the nitrosonium cation that forms spontaneously from NO to form the fluorescent product 1H-naphthotriazole. Using 2,3-diaminonaphthalene (“DAN”), researchers have developed a rapid, quantitative fluorometric assay that can detect from 10 nM to 10 μM nitrite and is compatible with a multi-well microplate format. DAN is a highly selective photometric and fluorometric reagent for Se and nitrite ion. DAN reacts with nitrite ion and gives fluorescent naphthotriazole (MC Carré et al., Analusis 27, 835-838 (1999)). Table 1 provides the test results of various compounds of the subject invention using the DAN assay.

A specimen can be processed prior to determination of nitrate or nitrite as required by the quantification method, or in order to improve the results, or for the convenience of the investigator. For example, processing can involve centrifuging, filtering, or homogenizing the sample. If the sample is whole blood, the blood can be centrifuged to remove cells and the nitrate or nitrite assay performed on the plasma or serum fraction. If the sample is tissue, the tissue can be dispersed or homogenized by any method known in the art prior to determination of nitrate or nitrite. It may be preferable to remove cells and other debris by centrifugation or another method and to determine the nitrate or nitrite level using only the fluid portion of the sample, or the extracellular fluid fraction of the sample. The sample can also be preserved for later determination, for example by freezing of urine or plasma samples. When appropriate, additives may be introduced into the specimen to preserve or improve its characteristics for use in the nitrate or nitrite assay.

The “level” of nitrate, nitrite, or other NO-related product usually refers to the concentration (in moles per liter, micromoles per liter, or other suitable units) of nitrate or nitrite in the specimen, or in the fluid portion of the specimen. However, other units of measure can also be used to express the level of nitrate or nitrite. For example, an absolute amount (in micrograms, milligrams, nanomoles, moles, or other suitable units) can be used, particularly if the amount refers back to a constant amount (e.g., grams, kilograms, milliliters, liters, or other suitable units) of the specimens under consideration. A number of commercially available kits can be used. In certain instances two regioisomers correspond to a single example #, whereby the example # is given in Table 1 with two hiNOS activity values respectively.

TABLE 1
Biological Activity

| | hiNOS EC₅₀ |
|---|---|
| | + indicates ≦5 μM |
| | − indicates >5 μM |
| Example # | NT indicates Not Tested |
| 1 | + |
| 2 | − |
| 3 | − |
| 4 | − |
| 5 | − |
| 6 | − |
| 7 | − |
| 8 | + |
| 9 | + |
| 10 | + |
| 11 | + |
| 12 | NT |
| 13 | + |
| 14 | − |
| 15 | + |
| 16 | + |
| 17 | − |
| 18 | − |
| 19 | + |
| 20 | + |
| 21 | + |
| 22 | + |
| 23 | + |
| 24 | + |
| 25 | + |
| 26 | − |
| 27 | − |
| 28 | − |
| 29 | − |
| 30 | − |
| 31 | − |
| 32 | − |
| 33 | − |
| 34 | − |
| 35 | + |
| 36 | − |
| 37 | + |
| 38 | − |
| 39 | − |
| 40 | − |
| 41 | − |
| 42 | + |
| 43 | − |
| 44 | − |
| 45 | + |
| 46 | − |
| 47 | − |
| 48 | − |
| 49 | − |
| 50 | − |
| 51 | − |
| 52 | − |
| 53 | + |
| 54 | − |
| 55 | − |
| 56 | − |
| 57 | − |
| 58 | − |
| 59 | − |
| 60 | − |
| 61 | − |
| 62 | − |
| 63 | − |
| 64 | − |
| 65 | − |
| 66 | − |
| 67 | − |
| 68 | − |
| 69 | − |
| 70 | − |
| 71 | − |
| 72 | − |
| 73 | − |
| 74 | − |
| 75 | − |
| 76 | − |
| 77 | − |
| 78 | − |
| 79 | − |
| 80 | − |
| 81 | − |
| 82 | − |
| 83 | − |
| 84 | − |
| 85 | + |
| 86 | − |
| 87 | − |
| 88 | + |
| 89 | + |
| 90 | + |
| 91 | + |
| 92 | + |
| 93 | + |
| 94 | + |
| 95 | + |
| 96 | + |
| 97 | + |
| 98 | + |
| 99 | + |
| 100 | + |
| 101 | + |
| 102 | − |
| 103 | − |
| 104 | − |
| 105 | − |
| 106 | − |
| 107 | − |
| 108 | + |
| 109 | + |
| 110 | + |
| 111 | + |
| 112 | − |
| 113 | + |
| 114 | + |
| 115 | + |
| 116 | + |
| 117 | − |
| 118 | − |
| 119 | + |
| 120 | − |
| 121 | − |
| 122 | + |
| 123 | − |
| 124 | + |
| 125 | − |
| 126 | − |
| 168 | + |
| 169 | + |
| 170 | − |
| 171 | − |
| 172 | − |
| 173 | − |
| 174 | − |
| 175 | + |
| 176 | + |
| 177 | + |
| 178 | + |
| 179 | + |
| 180 | + |
| 181 | + |
| 182 | + |
| 183 | + |
| 184 | + |
| 185 | − |
| 186 | + |
| 187 | + |
| 188 | + |
| 189 | + |
| 190 | − |
| 191 | + |
| 192 | + |
| 193 | − |
| 194 | − |
| 195 | + |
| 196 | + |
| 197 | − |
| 198 | − |
| 199 | + |
| 200 | − |
| 201 | − |
| 202 | − |
| 203 | − |
| 204 | − |
| 205 | − |
| 206 | + |
| 207 | + |
| 208 | + |
| 209 | + |
| 210 | + |
| 211 | + |
| 212 | − |
| 213 | − |
| 214 | + |
| 215 | + |
| 216 | − |
| 217 | + |
| 218 | + |
| 219 | + |
| 220 | + |
| 221 | + |
| 222 | + |
| 223 | − |
| | + |
| 224 | + |
| 225 | + |
| 226 | − |
| 227 | − |
| | + |
| 228 | + |
| | + |
| 229 | − |
| 230 | − |
| 231 | − |
| | + |
| 232 | + |
| 233 | + |
| 234 | + |
| 235 | + |
| 236 | − |
| | + |
| 237 | − |
| 238 | − |
| 239 | − |
| 240 | − |
| 241 | + |
| 242 | − |
| 243 | − |
| 244 | + |
| 245 | + |
| 246 | − |
| 247 | − |
| 248 | + |
| 249 | − |
| | − |
| 250 | + |
| 251 | − |
| 252 | + |
| 253 | + |
| 254 | + |
| 255 | − |
| 256 | − |
| 257 | + |
| 258 | − |
| 259 | − |
| | − |
| 260 | − |
| 261 | + |
| 262 | + |
| 263 | + |
| 264 | − |
| 265 | + |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method for achieving an effect in a patient comprising the administration to a patient of a therapeutically effective amount of a compound of Formula I: 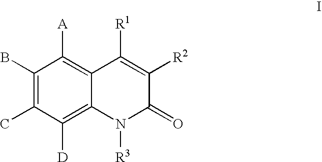
or a salt, ester, or prodrug thereof, wherein:
R¹ is selected from the group consisting of acyl, alkyl, alkylene, aminoalkyl, amidoalkyl, alkynyl, amido, amino, aminoalkyl, aryl, arylalkyl, arylalkoxy, arylamino, arylaminoalkyl, arylthio, carboxy, cycloalkyl, ester, ether, halo, haloalkoxy, haloalkyl, heteroaryl, heteroarylalkyl, heteroarylamino, heteroarylaminoalkyl, heterocycloalkyl, heterocycloalkylalkyl, hydrazinyl, hydrogen, imino, thio, sulfonate, sulfonylamino and sulfonylaminoalkyl, any of which may be optionally substituted;
R² is selected from the group consisting of acyl, alkoxy, alkoxyalkyl, alkyl, alkylene, alkylamino, alkynyl, alkylimino, amido, amino, aryl, carboxy, cyano, cycloalkyl, ester, halo, haloalkyl, heteroaryl, heterocycloalkyl and hydrogen, any of which may be optionally substituted; or, alternatively, R² may combine with R¹ to form heterocycloalkyl, which may be optionally substituted;
R³ is selected from the group consisting of alkyl, amino, arylalkyl, aryl, cycloalkyl, haloalkyl, heteroarylalkyl, heterocycloalkyl and hydrogen, any of which may be optionally substituted; and
A, B, C and D are each independently selected from the group consisting of acyl, alkoxy, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted; or, alternatively, any two or more A, B, C and D may combine to form aryl, cycloalkyl, heteroaryl or heterocycloalkyl, any of which may be optionally substituted;
and wherein the effect is selected from the group consisting of inhibition of iNOS and treatment of an iNOS-mediated disease in a patient in need thereof.

2. The method as recited in claim 1, wherein the effect is inhibition of iNOS.

3. The method as recited in claim 1, wherein the effect is treatment of an iNOS-mediated disease.

4. The method as recited in claim 3, wherein the compound has the Formula II: 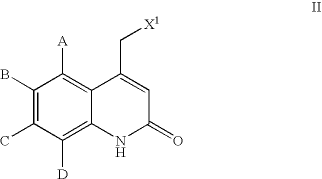
or a salt, ester, or prodrug thereof, wherein:
X¹ is selected from the group consisting of CR⁴R⁵, N(R⁶)(R⁷), S(O)R⁸, S(O)₂R⁹ or OR¹⁰;
R⁴ and R⁵ are each independently selected from the group consisting of alkyl, amino, arylalkyl, aryl, cycloalkyl, haloalkyl, heteroarylalkyl, heterocycloalkyl and hydrogen, any of which may be optionally substituted;
R⁶ and R⁷ are each independently selected from the group consisting of acyl, alkyl, amino, aryl, cycloalkyl, haloalkyl, heteroaryl, heterocycloalkyl, hydrogen and sulfonyl, any of which may be optionally substituted; or, alternatively, R³ and R⁴ may combine to form heterocycloalkyl or heteroaryl, which may be optionally substituted;
R³ and R⁹ are each independently selected from the group consisting of alkyl, amino, arylalkyl, aryl, cycloalkyl, haloalkyl, heteroarylalkyl, heterocycloalkyl and hydrogen, any of which may be optionally substituted;
R¹⁰ is selected from the group consisting of alkyl, amino, arylalkyl, aryl, cycloalkyl, haloalkyl, heteroarylalkyl, heterocycloalkyl and hydrogen, any of which may be optionally substituted; and
A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

5. The method as recited in claim 4, wherein the compound has Formula III: 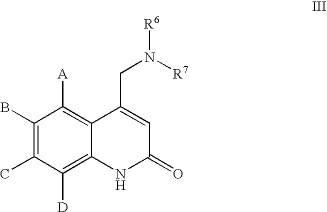
or a salt, ester, or prodrug thereof, wherein:
R⁶ and R⁷ are each independently selected from the group consisting of acyl, alkyl, alkylene, aminoalkyl, alkynyl, amido, amino, aryl, arylthio, carboxy, cycloalkyl, ester, ether, halo, haloalkoxy, haloalkyl, heteroaryl, heterocycloalkyl, hydrogen, thio and sulfonyl, any of which may be optionally substituted; or, alternatively, R¹ and R² may combine to form heterocycloalkyl or 5- to 14-membered heteroaryl, either of which may be optionally substituted; and
A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

6. The method as recited in claim 3, wherein said disease is selected from the group consisting of pruritis, psoriasis, uveitis, type 1 diabetes, diabetic nephropathy, septic shock, inflammatory pain, neuropathic pain, herpes zoster, postherpetic neuralgia, diabetic neuropathy, chronic low back pain, complex regional pain syndrome, fibromyalgia, migraine, rheumatoid arthritis, osteoarthritis, gouty arthritis, inflammatory bowel disease, asthma, COPD, allergic rhinitis, diabetic retinopathy, immune complex diseases, multiple sclerosis, alzheimer's disease, parkinson's disease, ischemic brain edema, toxic shock syndrome, heart failure, ulcerative colitis, atherosclerosis, glomerulonephritis, Paget's disease, osteoporosis, inflammatory sequelae of viral infections, retinitis, oxidant induced lung injury, restless leg syndrome, eczema, periodontal disease, gingivitis, acute allograft rejection and infection caused by invasive microorganisms which produce NO.

7. The method as recited in claim 3, wherein said compound of Formula I is administered in combination with another therapeutic agent.

8. The method as recited in claim 7, wherein:
said disease is selected from the group consisting of pruritis, psoriasis, uveitis, type 1 diabetes, diabetic nephropathy, septic shock, inflammatory pain, neuropathic pain, herpes zoster, postherpetic neuralgia, diabetic neuropathy, chronic low back pain, complex regional pain syndrome, fibromyalgia, migraine, rheumatoid arthritis, osteoarthritis, gouty arthritis, inflammatory bowel disease, asthma, COPD, allergic rhinitis, diabetic retinopathy, immune complex diseases, multiple sclerosis, alzheimer's disease, parkinson's disease, ischemic brain edema, toxic shock syndrome, heart failure, ulcerative colitis, atherosclerosis, glomerulonephritis, Paget's disease, osteoporosis, inflammatory sequelae of viral infections, retinitis, oxidant induced lung injury, restless leg syndrome, eczema, periodontal disease, gingivitis, acute allograft rejection and infection caused by invasive microorganisms which produce NO; and
said other therapeutic agent is selected from the group consisting of corticosteroids, non-steroidal anti-inflammatory drugs, muscle relaxants, anaesthetics, expectorants, antidepressants, anticonvulsants, antihypertensives, opioids, topical counter-irritants and topical cannabinoids.

9. A compound of Formula III: 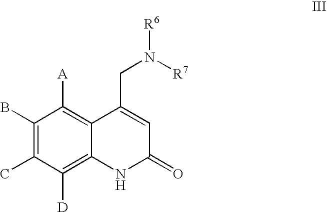
or a salt, ester, or prodrug thereof, wherein:
R⁶ is selected from the group consisting of acyl, alkyl, alkylene, alkynyl, amino, aryl, arylthio, carboxy, cycloalkyl, ester, ether, halo, haloalkoxyalkyl, haloalkyl, heteroaryl, heteroarylamino, heterocycloalkyl, thio, sulfonate and sulfonyl, any of which may be optionally substituted;
R⁷ is selected from the group consisting of acyl, alkylene, aminoalkyl, alkynyl, amino, aminosulfonyl, aryl, arylthio, carboxy, cycloalkyl, ester, ether, halo, haloalkoxy, heteroaryl, heteroarylamino, heterocycloalkyl, thio, sulfonate and sulfonyl, any of which may be optionally substituted; or, alternatively, R¹ and R² may combine to form 5- to 14-membered heteroaryl, which may be optionally substituted with C₂-C₆ alkyl, aryl, arylthio, arylamino, cycloalkyl, heteroaryl, heteroarylamino, heteroarylthio and heterocycloalkyl, any of which may itself be optionally substituted; and
A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

10. The compound as recited in claim 9, wherein the compound has Formula IV: 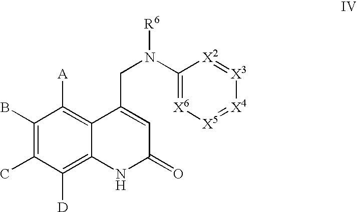
or a salt, ester, or prodrug thereof, wherein:
X² is selected from the group consisting of CR¹² and N;
X³ is selected from the group consisting of CR¹³ and N;
X⁴ is selected from the group consisting of CR¹⁴ and N;
X⁵ is selected from the group consisting of CR¹⁵ and N;
X⁶ is selected from the group consisting of CR¹⁶ and N;
R¹² and R¹⁶ are each independently selected from the group consisting of alkoxy, acyl, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;
R¹³ and R¹⁵ are each independently selected from the group consisting of acyl, C²⁻⁶ alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;
R¹⁴ is selected from the group selected from the group consisting of C³⁻⁶ alkoxy, acyl, C²⁻⁶ alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;
R⁶ is selected from the group consisting of acyl, alkyl, alkylene, alkynyl, aminosulfonyl, arylthio, benzyl, carboxy, cycloalkyl, ester, ether, furanalkyl, furancarbonyl, haloalkyl, heteroaryl, heteroarylalkyl, aminoheteroaryl, heterocycloalkyl, imidazolecarbonyl, isoxazolecarbonyl, oxazolecarbonyl, pyrazinecarbonyl, thiophenecarbonyl, thiazolecarbonyl, thio and sulfonate, any of which may be optionally substituted; and
A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted;
and with the proviso that when R⁶ is 2-furancarbonyl, then A, B, C, D, and R¹²-R¹⁶ cannot all be hydrogen.

11. The compound as recited in claim 10, wherein the compound has Formula V: 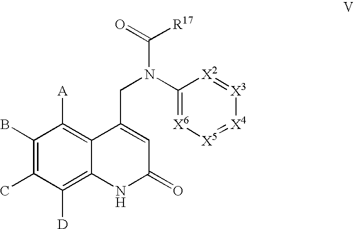
or a salt, ester, or prodrug thereof, wherein:
X² is selected from the group consisting of CR¹² and N;
X³ is selected from the group consisting of CR¹³ and N;
X⁴ is selected from the group consisting of CR¹⁴ and N;
X⁵ is selected from the group consisting of CR¹⁵ and N;
X⁶ is selected from the group consisting of CR¹⁶ and N;
R¹² and R¹⁶ are each independently selected from the group consisting of alkoxy, acyl, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;
R¹³ and R¹⁵ are each independently selected from the group consisting of acyl, C²⁻⁶ alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;
R¹⁴ is selected from the group consisting of C³⁻⁶ alkoxy, acyl, C²⁻⁶ alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;
R¹⁷ is selected from the group consisting of alkyl, aryl, arylthio, cycloalkyl, heterocycloalkyl, benzimidazole, benzthiazole, benzofuran, benzothiophene, benzo[d][1,3]dioxole, 1H-benzo[d][1,2,3]triazole, 2,3-dihydrobenzofuran, 1,4-dioxane, 1,3-dioxalane, 3,4-dihydro-2H-benzo[b][1,4]dioxepine, 2,2-difluorobenzo[d][1,3]dioxole, isoxazole, isothiazole, indolizine, indole, isoindole, 3H-indoline, indoline, 1H-indazole, isoquinoline, imidazole, 2-imidazoline, imidazolidine, isothiazole, naphthalene, oxazole, 1,2,3-oxadiazole, morpholine, 2H-pyran, 4H-pyran, piperidine, pyridazine, pyrazine, piperazine, phenyl, pyridine, pyrimidine, thiophene, pyrrole, 2H-pyrrole, 2-pyrroline, 3-pyrroline, pyrrolidine, purine, thiazole, pyrazole, 2-pyrazoline, pyrazolidine, quinoline, quinazoline, quinaxaline, 1,2,3-triazole, 1,3,4-thiadiazole and 1,3,5-triazine, any of which may be optionally substituted; and
A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

12. The compound as recited in claim 11, or a salt, ester, or prodrug thereof, wherein:
X² is CR¹²; X³ is CR³; X⁴ is CR¹⁴; X⁵ is CR⁵; X⁶ is CR⁶;
R¹²-R¹⁵ are each independently selected from the group consisting of halo, haloalkoxy, haloalkyl and hydrogen, any of which may be optionally substituted;
R⁶ is selected from the group consisting of cycloalkyl, heterocycloalkyl, isothiazole, imidazole, phenyl, pyridine, pyrazole and thiazole, which may be optionally substituted; and
A, B, C and D are each independently selected from the group consisting of halo and hydrogen.

13. The compound as recited in claim 9, wherein the compound has Formula VI: 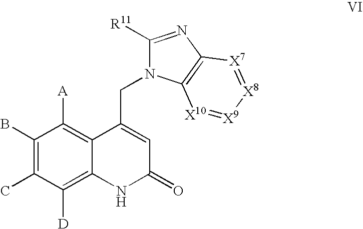
or a salt, ester, or prodrug thereof, wherein:
X⁷ is selected from the group consisting of CR¹⁷ and N;
X⁸ is selected from the group consisting of CR¹⁸ and N;
X⁹ is selected from the group consisting of CR¹⁹ and N;
X¹⁰ is selected from the group consisting of CR²⁰ and N;
R¹¹ is selected from the group consisting of C₂-C₆ alkyl, aryl, arylthio, arylamino, cycloalkyl, heteroarylamino, heteroarylthio, heterocycloalkyl, benzimidazole, benzthiazole, benzofuran, benzothiophene, benzo[d][1,3]dioxole, 1H-benzo[d][1,2,3]triazole, 2,3-dihydrobenzofuran, 1,4-dioxane, 1,3-dioxalane, 3,4-dihydro-2H-benzo[b][1,4]dioxepine, 2,2-difluorobenzo[d][1,3]dioxole, furan, isoxazole, isothiazole, indolizine, indole, isoindole, 3H-indoline, indoline, 1H-indazole, isoquinoline, imidazole, 2-imidazoline, imidazolidine, isothiazole, naphthalene, oxazole, 1,2,3-oxadiazole, morpholine, 2H-pyran, 4H-pyran, piperidine, pyridazine, pyrazine, piperazine, phenyl, pyridine, pyrimidine, thiophene, pyrrole, 2H-pyrrole, 2-pyrroline, 3-pyrroline, pyrrolidine, purine, thiazole, pyrazole, 2-pyrazoline, pyrazolidine, quinoline, quinazoline, quinaxaline, 1,2,3-triazole, 1,3,4-thiadiazole and 1,3,5-triazine, any of which may be optionally substituted;
R¹⁷-R²⁰ are each independently selected from the group consisting of alkoxy, acyl, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted; and
A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

14. The compound as recited in claim 13, or a salt, ester, or prodrug thereof, wherein:
X⁷ is CR¹⁷; X⁸ is CR¹⁸; X⁹ is CR¹⁹; X¹⁰ is CR²⁰;
R¹¹ is selected from the group consisting of alkyl, cycloalkyl, heterocycloalkyl, isothiazole, imidazole, phenyl, pyridine, pyrazole and thiazole, which may be optionally substituted;
R¹⁷-R²⁰ are each independently selected from the group consisting of halo, haloalkoxy, haloalkyl and hydrogen, any of which may be optionally substituted; and
A, B, C and D are each independently selected from the group consisting of halo and hydrogen.

15. The compound as recited in claim 10, wherein the compound has Formula VII: 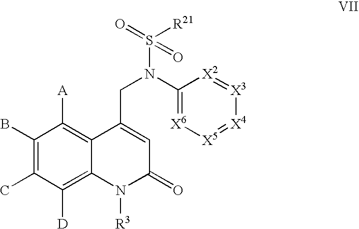
or a salt, ester, or prodrug thereof, wherein:
X² is selected from the group consisting of CR¹² and N;
X³ is selected from the group consisting of CR¹³ and N;
X⁴ is selected from the group consisting of CR¹⁴ and N;
X⁵ is selected from the group consisting of CR¹⁵ and N;
X⁶ is selected from the group consisting of CR¹⁶ and N;
R¹²-R¹⁶ are each independently selected from the group consisting of alkoxy, acyl, alkyl, alkylene, alkylamino, alkynyl, amido, amino, aminosulfonyl, aryl, arylalkoxy, arylamino, arylthio, carboxy, cycloalkyl, ester, ether, halo, haloalkoxy, haloalkyl, heteroaryl, heteroarylamino, heterocycloalkyl, hydrazinyl, hydrogen, imino, thio, sulfonate and sulfonylamino, any of which may be optionally substituted;
R²¹ is selected from the group consisting of alkyl, aryl, arylthio, arylamino, cycloalkyl, heteroarylamino, heteroarylthio, heterocycloalkyl, benzimidazole, benzthiazole, benzofuran, benzothiophene, benzo[d][1,3]dioxole, 1H-benzo[d][1,2,3]triazole, 2,3-dihydrobenzofuran, 1,4-dioxane, 1,3-dioxalane, 3,4-dihydro-2H-benzo[b][1,4]dioxepine, 2,2-difluorobenzo[d][1,3]dioxole, furan, isoxazole, isothiazole, indolizine, indole, isoindole, 3H-indoline, indoline, 1H-indazole, isoquinoline, imidazole, 2-imidazoline, imidazolidine, isothiazole, naphthalene, oxazole, 1,2,3-oxadiazole, morpholine, 2H-pyran, 4H-pyran, piperidine, pyridazine, pyrazine, piperazine, phenyl, pyridine, pyrimidine, thiophene, pyrrole, 2H-pyrrole, 2-pyrroline, 3-pyrroline, pyrrolidine, purine, thiazole, pyrazole, 2-pyrazoline, pyrazolidine, quinoline, quinazoline, quinaxaline, 1,2,3-triazole, 1,3,4-thiadiazole and 1,3,5-triazine, any of which may be optionally substituted; and
A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

16. The compound as recited in claim 15, or a salt, ester, or prodrug thereof, wherein:
X² is CR¹²; X³ is CR¹³; X⁴ is CR¹⁴; X⁵ is CR¹⁵; X⁶ is CR¹⁶;
R¹²-R¹⁶ are each independently selected from the group consisting of halo, haloalkoxy, haloalkyl and hydrogen, any of which may be optionally substituted;
R²¹ is selected from the group consisting of alkyl, cycloalkyl, heterocycloalkyl, isothiazole, imidazole, phenyl, pyridine, pyrazole and thiazole, which may be optionally substituted; and
A, B, C and D are each independently selected from the group consisting of halo and hydrogen.

17. The compound as recited in claim 9, wherein the compound has Formula VIII: 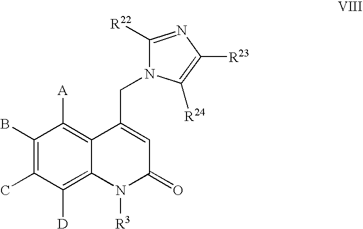
or a salt, ester, or prodrug thereof, wherein:
R²²-R²⁴ are each independently selected from the group consisting of alkyl, aryl, arylthio, arylamino, cycloalkyl, heteroarylamino, heteroarylthio, heterocycloalkyl, benzimidazole, benzthiazole, benzofuran, benzothiophene, benzo[d][1,3]dioxole, 1H-benzo[d][1,2,3]triazole, 2,3-dihydrobenzofuran, 1,4-dioxane, 1,3-dioxalane, 3,4-dihydro-2H-benzo[b][1,4]dioxepine, 2,2-difluorobenzo[d][1,3]dioxole, furan, isoxazole, isothiazole, indolizine, indole, isoindole, 3H-indoline, indoline, 1H-indazole, isoquinoline, imidazole, 2-imidazoline, imidazolidine, isothiazole, naphthalene, oxazole, 1,2,3-oxadiazole, morpholine, 2H-pyran, 4H-pyran, piperidine, pyridazine, pyrazine, piperazine, phenyl, pyridine, pyrimidine, thiophene, pyrrole, 2H-pyrrole, 2-pyrroline, 3-pyrroline, pyrrolidine, purine, thiazole, pyrazole, 2-pyrazoline, pyrazolidine, quinoline, quinazoline, quinaxaline, 1,2,3-triazole, 1,3,4-thiadiazole and 1,3,5-triazine, any of which may be optionally substituted; and
A, B, C and D are each independently selected from the group consisting of acyl, alkoxy, alkyl, alkylene, alkylamino, alkynyl, amido, amino, aminosulfonyl, carboxy, ester, ether, halo, haloalkoxy, haloalkyl, hydrogen, imino, thio, sulfonate and sulfonylamino, any of which may be optionally substituted.

18. The compound as recited in claim 17, or a salt, ester, or prodrug thereof, wherein:
R²²-R²⁴ are each independently selected from the group consisting of alkyl cycloalkyl, heterocycloalkyl, isothiazole, imidazole, phenyl, pyridine, pyrazole and thiazole, which may be optionally substituted; and
A, B, C and D are each independently selected from the group consisting of halo and hydrogen.

19. A compound selected from the group consisting of Examples 1 to 265.

20. A compound as recited in claim 9 for use as a medicament.

21. A compound as recited in claim 9 for use in the manufacture of a medicament for the prevention or treatment of a disease or condition ameliorated by the inhibition of iNOS.

22. A pharmaceutical composition comprising a compound as recited in claim 9 together with a pharmaceutically acceptable carrier.
